(19) United States
(12) Patent Application Publication
(10) Pub. No.: US 2018/0319813 A1
(43) Pub. Date: Nov. 8, 2018

(54) BENZIMIDAZOLE FUSED HETEROARYLS

(71) Applicant: IDEMITSU KOSAN CO., LTD., Chiyoda-ku (JP)

(72) Inventors: Yuichi NISHIMAE, Basel (CH); Thomas SCHAFER, Basel (CH); Yuki NAKANO, Sodegaura-shi (JP); Hideaki NAGASHIMA, Basel (CH); Masahiro KAWAMURA, Basel (CH); Tasuku HAKETA, Sodegaura-shi (JP); Takushi SHIOMI, Sodegaura-shi (JP)

(73) Assignee: IDEMITSU KOSAN CO., LTD, Chiyoda-ku (JP)

(21) Appl. No.: 15/772,908

(22) PCT Filed: Nov. 4, 2016
(86) PCT No.: PCT/JP2016/083449

§ 371 Date: May 2, 2018

(30) Foreign Application Priority Data
Nov. 4, 2015 (EP) 15192932.0

(51) Int. Cl. C07D 491/147; H01L 51/00; C09K 11/06

(52) U.S. Cl. CPC C07D 491/147; H01L 51/0071; H01L 51/0067; H01L 51/0052; H01L 51/0065; H01L 51/5016; C09K 11/06; C09K 2211/1007; C09K 2211/1011; C09K 2211/1018; H01L 51/0054

(57) ABSTRACT

A compound of general formula (I): wherein A¹, A², A³, A⁴, B¹, B², B³, B⁴, C¹, C², C³, C⁴, X¹ and X² have the meanings as defined in the description provides electronic devices, preferably OLEDs, which ensure good efficiencies, good operative lifetimes and a high stability to thermal stress, and a low use and operating voltage of the OLEDs.

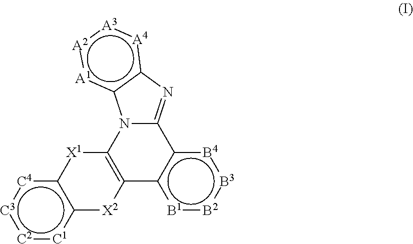

### TECHNICAL FIELD

The present invention relates to an electronic device comprising at least one compound of the general formula (I), to an emitting layer, preferably present in an electronic device, comprising at least one compound of general formula (I), to the use of a compound according to general formula (I) in an electronic device as a host material, a charge transporting material or a dopant without metal species, to specific compounds according to general formula (IV) and to a process for the preparation of these compounds.

### BACKGROUND ART

Benzimidazole fused heteroaryls and their use in electronic devices are known from the prior art.

WO 2014/008982 A1 discloses the use of metal complexes of the general formula M(L)ₙ(L′)ₘ as emitter in electronic devices, in particular in OLEDs (Organic Light Emitting Diode)s. The ligands L and L′ that are present in these complexes correspond—among others—to the following formula:

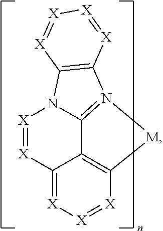

wherein —X═X— may correspond to

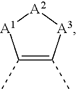

The use of the ligands without any metal cation in electronic devices or the corresponding electronic devices are not disclosed in WO 2014/008982 A1.

CN 1017781312 A discloses a process for the preparation of quinolone or indole derivatives and their use in the bio-chemical, cosmetic, pharmaceutical, materials applications. Among others, compounds of the formula

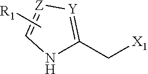

are disclosed, wherein Y, Z may be N or C and R₁ may by alkyl, aryl, etc. Particular compounds that are exemplified in CN 1017781312 A1 are

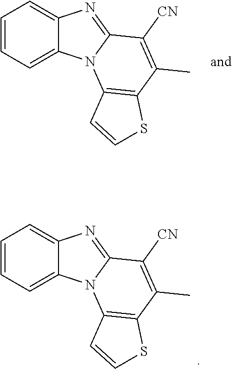 [SMILES: Cc1c(C#N)c2nc3ccccc3n2c2ccsc12 ; Cc1c(C#N)c2nc3ccccc3n2c2ccsc12]

The use of benzimidazole fused heteroaryls in electronic devices is not disclosed in this document.

M. Hranjec et al., J. Med. Chem. 2008, 51, (16), pages 4899-4910 disclose a method for the preparation of compounds of general formula

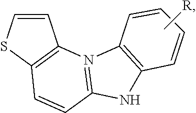

The use of these compounds in electronic devices is not disclosed in this document.

### CITATION LIST

### Patent Literature

Patent Literature 1: WO 2014/008982 A1
Patent Literature 2: CN 1017781312 A

### Non Patent Literature

Non Patent Literature 1: M. Hranjec et al., J. Med. Chem. 2008, 51, (16), pages 4899-4910

### SUMMARY OF INVENTION

### Technical Problem

There remains a need for electronic devices comprising new materials, especially host materials, electron transport materials and/or hole transport materials to provide improved efficiency, stability, manufacturability, driving voltage and/or spectral characteristics of electronic devices.

Accordingly, it is an object of the present invention, with respect to the aforementioned prior art, to provide materials suitable for use in electronic devices, preferably OLEDs, and further applications in organic electronics. More particularly, it should be possible to provide electronic devices comprising new compounds as electron transport materials, as hole transport materials or as host materials. The materials should be suitable especially for OLEDs which comprise at least one emitter, which is preferably a phosphorescence emitter, for example at least one green, red or yellow emitter, especially at least one green emitter or at least one red emitter. The materials should also be suitable especially for OLEDs which comprise at least one emitter, which is preferably a fluorescence emitter, for example at least one blue emitter, especially as an electron transporting material.

Furthermore, the materials should be suitable for providing electronic devices, preferably OLEDs, which ensure good efficiencies, good operative lifetimes and a high stability to thermal stress, and a low use and operating voltage of the OLEDs.

### Solution to Problem

Said object is solved by an electronic device comprising at least one compound of the general formula (I):

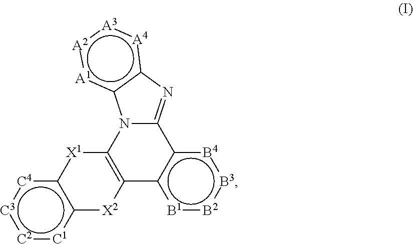

wherein A¹, A², A³, A⁴, B¹, B², B³, B⁴, C¹, C², C³, C⁴, X¹ and X² have the following meanings:

A¹, A², A³, and A⁴ form an aromatic or heteroaromatic six membered ring, wherein A¹ is N or CR¹, A² is N or CR², A³ is N or CR³ and A⁴ is N or CR⁴,

B¹, B², B³, and B⁴ form an aromatic or heteroaromatic five or six membered ring, wherein B¹ is a direct bond, NR⁶, N, O, S, CR⁶ or CR⁷R⁸, B² is a direct bond, NR⁹, N, O, S, CR¹⁰ or CR¹¹R¹², B³ is a direct bond, NR¹³, N, O, S, CR¹⁴ or CR¹⁵R¹⁶ and B⁴ is a direct bond, NR¹⁷, N, O, S, CR¹⁸ or CR¹⁹R²⁰,

C¹, C², C³, and C⁴ form an aromatic or heteroaromatic six membered ring, wherein C¹ is N or CR²¹, C² is N or CR²², C³ is N or CR²³ and C⁴ is N or CR²⁴,

X¹ and X² are each direct bond, O, S, NR²⁵, or CR²⁶R²⁷, wherein one of X¹ and X² is a direct bond and the other one is O, S, NR²⁵, or CR²⁶R²⁷,

wherein R¹, R², R³, and R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,

D is —CO—, —COO—, —S—, —SO—, —SO₂—, —O—, —CR³CR³²—, —NR³³—, —SiR²⁸R²⁹—, —POR³⁴—, or —C≡C—,

E is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, an unsubstituted C₆-C₆₀aryl group, a C₆-C₆₀aryl group which is substituted by J or C₁-C₁₈alkyl, a C₁-C₁₈alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,

J is —CF₃, —CF₂CF₃, —CF₂CF₂CF₃, —CF(CF₃)₂, —(CF₂)₃CF₃ or —C(CF₃)₃,

G is E, a C₁-C₁₈alkyl group, or a C₁-C₁₈alkyl which is interrupted by O,

R²⁸, R²⁹ and R³⁰ are independently of each other a C₁-C₁₈alkyl group, a C₆-C₁₈aryl group, or a C₆-C₁₈aryl group which is substituted by C₁-C₁₈alkyl,

R³¹ and R³² are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,

R³³, R³⁴, R³⁵, and R³⁹ are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,

R³⁶ is H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,

R³⁷, R³⁸, R⁴⁰, R⁴¹, and R⁴² are independently of each other H, a C₆-C₁₈aryl group, a C₆-C₁₈aryl which is substituted by C₁-C₁₈alkyl or C₁-C₁₈alkoxy, a C₁-C₁₈alkyl group, or a C₁-C₁₈alkyl group which is interrupted by —O—,

or R³⁷ and R³⁸ together form a five or six membered ring,

or R⁴¹ and R⁴² together form a five or six membered ring,

or two of R¹, R², R³ and R⁴, if present at adjacent carbon atoms, form a five or six membered, substituted or unsubstituted, saturated or unsaturated, aromatic or heteroaromatic ring,

R⁵, R⁶, R⁷, R⁸, R⁹, R¹⁰, R¹¹, R¹², R¹³, R¹⁴, R¹⁵, R¹⁶, R¹⁷, R¹⁸, R¹⁹, and R²⁰ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,

or two of R⁵, R⁶, R⁷, R⁸, R⁹, R¹⁰, R¹¹, R¹², R¹³, R¹⁴, R¹⁵, R¹⁷, R¹⁸, R¹⁹, and R²⁰, if present at adjacent carbon atoms, form a five or six membered, substituted or unsubstituted, saturated or unsaturated, aromatic or heteroaromatic ring,

R²¹, R²², R²³, R²⁴, R²⁵, R²⁶, R²⁷ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,

or two of R²¹, R²², R²³ and R²⁴, if present at adjacent carbon atoms, form a five or six membered, substituted or unsubstituted, saturated or unsaturated, aromatic or heteroaromatic ring,

or R²⁵ may form a five or six membered, substituted or unsubstituted, saturated or unsaturated, aromatic or heteroaromatic ring with R²¹, R²², R²³ or R²⁴.

In an embodiment of the compound of the general formula (I), A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R¹, R², R³, R⁴, R⁶, R¹⁰, R¹⁴, R¹⁸, R²¹, R²², R²³, and R²⁴ is a substituent other than H (hydrogen), i.e., E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above. The substituent other than H is preferably those of formulae (b) and (b1) to be mentioned below.

The above mentioned objects are further solved by an emitting layer, preferably present in an electronic device, more preferably in an electroluminescence device, particularly preferably in an organic light emitting diode (OLED), comprising at least one compound of general formula (I) according to the present invention.

The above mentioned objects are further solved by the use of a compound according to general formula (I) according to the present invention in an electronic device, preferably in an electroluminescence device, particularly preferably in an organic light emitting diode (OLED), preferably in an emitting layer, as a host material, a charge transporting material, for example an electron transporting material or a hole transporting material, or a dopant without metal species, preferably as a host material.

### DESCRIPTION OF EMBODIMENTS

The electronic device according to the present invention will be explained in detail in the following.

The electronic device according to the present invention comprises at least one compound according to the general formula (I) as mentioned above. According to the present invention, the electronic device may comprise one kind of compound according to the present invention or may comprise a mixture of different compounds according to general formula (I). Further, the electronic device according to the present invention may comprise at least one compound according to general formula (I) in different parts, for example layers, of the device. According to this embodiment, one kind of compound according to general formula (I) may be present in different parts, for example layers. According to another embodiment, different compounds according to general formula (I) may be present in different parts, for example layers.

According to the present invention the terms halogen, alkyl, aryl, aryloxy and heteroaryl generally have the following meaning, if said groups are not further specified in specific embodiments mentioned below:

Halogen is fluorine, chlorine, bromine and iodine.

C₁-C₂₅alkyl, preferably C₁-C₁₈alkyl, is typically linear or branched, where possible. Examples are methyl, ethyl, n-propyl, isopropyl, n-butyl, sec.-butyl, isobutyl, tert.-butyl, n-pentyl, 2-pentyl, 3-pentyl, 2,2-dimethylpropyl, 1,1,3,3-tetramethylpentyl, n-hexyl, 1-methylhexyl, 1,1,3,3,5,5-hexamethylhexyl, n-heptyl, isoheptyl, 1,1,3,3-tetramethylbutyl, 1-methylheptyl, 3-methylheptyl, n-octyl, 1,1,3,3-tetramethylbutyl and 2-ethylhexyl, n-nonyl, decyl, undecyl, dodecyl, tridecyl, tetradecyl, pentadecyl, hexadecyl, heptadecyl, and octadecyl. C₁-C₈alkyl is typically methyl, ethyl, n-propyl, isopropyl, n-butyl, sec.-butyl, isobutyl, tert.-butyl, n-pentyl, 2-pentyl, 3-pentyl, 2,2-dimethyl-propyl, n-hexyl, n-heptyl, n-octyl, 1,1,3,3-tetramethylbutyl or 2-ethylhexyl. C₁-C₄alkyl is typically methyl, ethyl, n-propyl, isopropyl, n-butyl, sec.-butyl, isobutyl, or tert.-butyl.

C₁-C₂₅alkoxy groups, preferably C₁-C₁₈alkoxy groups, are straight-chain or branched alkoxy groups, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy. Examples of C₁-C₈alkoxy are methoxy, ethoxy, n-propoxy, isopropoxy, n-butoxy, sec.-butoxy, isobutoxy, tert.-butoxy, n-pentyloxy, 2-pentyloxy, 3-pentyloxy, 2,2-dimethylpropoxy, n-hexyloxy, n-heptyloxy, n-octyloxy, 1,1,3,3-tetramethylbutoxy and 2-ethylhexyloxy, preferably C₁-C₄alkoxy such as typically methoxy, ethoxy, n-propoxy, isopropoxy, n-butoxy, sec.-butoxy, isobutoxy, and tert.-butoxy.

C₆-C₆₀aryl, preferably C₆-C₂₄aryl, particularly preferably C₆-C₁₈aryl, which optionally can be substituted, is typically phenyl, 4-methylphenyl, 4-methoxyphenyl, naphthyl, especially 1-naphthyl or 2-naphthyl, biphenylyl, terphenylyl, pyrenyl, 2- or 9-fluorenyl, phenanthryl, or anthryl, which may be unsubstituted or substituted. Phenyl, 1-naphthyl and 2-naphthyl are examples of a C₆-C₁₀aryl group.

C₆-C₂₄aryloxy, which optionally can be substituted, is typically C₆-C₁₀aryloxy, which optionally can be substituted by one or more C₁-C₈alkyl and/or C₁-C₈alkoxy groups, such as, for example, phenoxy, 1-naphthoxy, or 2-naphthoxy.

C₂-C₆₀heteroaryl, preferably C₂-C₃₀heteroaryl, particularly preferably C₂-C₁₃ heteroaryl, represents a ring with five to seven ring atoms or a condensed ring system, wherein nitrogen, oxygen or sulfur are the possible hetero atoms, and is typically a heterocyclic group with 5 to 40 atoms having at least six conjugated 7-electrons such as thienyl, benzothiophenyl, dibenzothiophenyl, thianthrenyl, furyl, furfuryl, 2H-pyranyl, benzofuranyl, isobenzofuranyl, dibenzofuranyl, phenoxythienyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, carbolinyl, benzotriazolyl, benzoxazolyl, phenanthridinyl, acridinyl, pyrimidinyl, phenanthrolinyl, phenazinyl, isothiazolyl, phenothiazinyl, isoxazolyl, furazanyl, 4-imidazo[1,2-a]benzimidazoyl, 5-benzimidazo[1,2-a]benzimidazoyl, benzimidazolo[2,1-b][1,3]benzothiazolyl, carbazolyl, or phenoxazinyl, which can be unsubstituted or substituted. Benzimidazo[1,2-a]benzimidazo-5-yl, benzimidazo[1,2-a]benzimidazo-2-yl, carbazolyl and dibenzofuranyl are examples of a C₂-C₁₄heteroaryl group.

C₇-C₂₅aralkyl is for example benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ω-phenyl-butyl, ω,ω-dimethyl-ω-phenyl-butyl, ω-phenyl-dodecyl or -phenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted. Preferred examples are benzyl, 2-phenylethyl, 3-phenylpropyl, naphthylethyl, naphthylmethyl, and cumyl.

C₆-C₁₂cycloalkyl is for example cyclopentyl, cyclohexyl, cycloheptyl, cyclooctyl, cyclononyl, cyclodecyl, cycloundecyl, cyclododecyl, preferably cyclopentyl, cyclohexyl, cycloheptyl, or cyclooctyl, which may be unsubstituted or substituted.

Possible preferred substituents of the above-mentioned groups are C₁-C₈alkyl, a hydroxyl group, a mercapto group, C₁-C₈alkoxy, C₁-C₈alkylthio, halogen, halo-C₁-C₈alkyl, or a cyano group.

In general, the electronic device according to the present invention comprises the at least one compound according to the general formula (I):

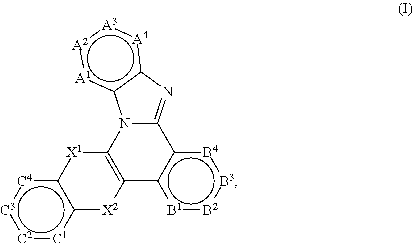

wherein A¹, A², A³, A⁴, B¹, B², B³, B⁴, C¹, C², C³, C⁴, X¹ and X² have the above mentioned meanings.

In general, A¹, A², A³, and A⁴ form an aromatic or heteroaromatic six membered ring, wherein A¹ is N or CR¹, A² is N or CR², A³ is N or CR³ and A⁴ is N or CR⁴.

According to one embodiment A¹ is N, A² is CR², A³ is CR³ and A⁴ is CR⁴, wherein R², R³ and R⁴ have the meanings as mentioned above.

According to another embodiment A¹ is CR¹, A² is N, A³ is CR³ and A⁴ is CR⁴, wherein R¹, R³ and R⁴ have the meanings as mentioned above.

According to another embodiment A¹ is CR¹, A² is CR², A³ is N and A⁴ is CR⁴, wherein R¹, R² and R⁴ have the meanings as mentioned above.

According to another embodiment A¹ is CR¹, A² is CR², A³ is CR³ and A⁴ is N, wherein R¹, R² and R³ have the meanings as mentioned above.

According to a preferred embodiment A¹ is CR¹, A² is CR², A³ is CR³ and A⁴ is CR⁴. According to this preferred embodiment, A¹, A², A³ and A⁴ form a substituted or unsubstituted aromatic six membered ring, wherein R¹, R², R³ and R⁴ have the meanings as mentioned above.

According to a particularly preferred embodiment A¹ is CR¹, A² is CR², A³ is CR³ and A⁴ is CR⁴ and R¹, R², R³, and R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,

D is —CO—, —COO—, —S—, —SO—, —SO₂—, —O—, —CR³¹═CR³²—, —NR³³—, —SiR²⁸R²⁹—, —POR³⁴—, or —C≡C—,

E is J, —OR³⁵, —SR³⁶, —NR³⁷R³⁸, —COR³⁹, —COOR⁴⁰, —CONR⁴¹R⁴², —CN, —SiR²⁸R²⁹R³⁰, halogen, an unsubstituted C₆-C₆₀aryl group, a C₆-C₆₀aryl group which is substituted by J, C₁-C₁₈alkyl, a C₁-C₁₈alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,

J is —CF₃, —CF₂CF₃, —CF₂CF₂CF₃, —CF(CF₃)₂, —(CF₂)₃CF₃ or —C(CF₃)₃,

G is E, a C₁-C₁₈alkyl group, or a C₁-C₁₈alkyl which is interrupted by O,

or two of R¹, R², R³ and R⁴, if present at adjacent carbon atoms, form a five or six membered, substituted or unsubstituted, saturated or unsaturated, aromatic or heteroaromatic ring.

More preferred, A¹ is CR¹, A² is CR², A³ is CR³ and A⁴ is CR⁴ and R¹, R², R³ and R⁴ are independently of each other H, E, a C₆-C₆₀aryl group which is unsubstituted or substituted by at least one group G, or a C₂-C₆₀heteroaryl group which is unsubstituted or substituted by at least one group G, wherein E and G have the meanings as mentioned above.

According to a further preferred embodiment of the present invention A¹ is CR¹, A² is CR², A³ is CR³ and A⁴ is CR⁴ and R¹ and R⁴ are H and R² and R³ are independently of each other H, E, a C₆-C₆₀aryl group which is unsubstituted or substituted by at least one group G, or a C₂-C₆₀heteroaryl group which is unsubstituted or substituted by at least one group G, wherein E and G have the meanings as mentioned above.

Most preferred, A¹ is CR¹, A² is CR², A³ is CR³ and A⁴ is CR⁴ and R¹, R² and R⁴ are H and R³ is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, a C₆-C₆₀aryl group which is unsubstituted or substituted by at least one group G, or a C₂-C₆₀heteroaryl group which is unsubstituted or substituted by at least one group G, wherein R²⁸, R²⁹, R³⁰, R³⁵, R³⁶, R³⁷, R³⁸, R³⁹, R⁴⁰, R⁴¹, R⁴² and G have the same meanings as mentioned above

or

R¹, R³ and R⁴ are H and R² is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, a C₆-C₆₀aryl group which is unsubstituted or substituted by at least one group G, or a C₂-C₆₀heteroaryl group which is unsubstituted or substituted by at least one group G, wherein R²⁸, R²⁹, R³⁰, R³⁵, R³⁶, R³⁷, R³⁸, R³⁹, R⁴⁰, R⁴¹, R⁴² and G have the same meanings as mentioned above.

A particularly preferred meaning of R¹, R², R³ and/or R⁴, preferably R² and/or R³, most preferably R³, is a C₆-C₆₀aryl group which is unsubstituted or substituted by at least one group G, or a C₂-C₆₀heteroaryl group which is unsubstituted or substituted by at least one group G, preferably a C₂-C₆₀heteroaryl group which is unsubstituted or substituted by at least one group G, wherein aromatic and/or heteroaromatic five or six membered rings are fused together or are connected by carbon-carbon-bonds.

Particularly preferred meanings of R¹, R², R³ and/or R⁴ are the substituent of formula (IV) and the further substituents as shown:

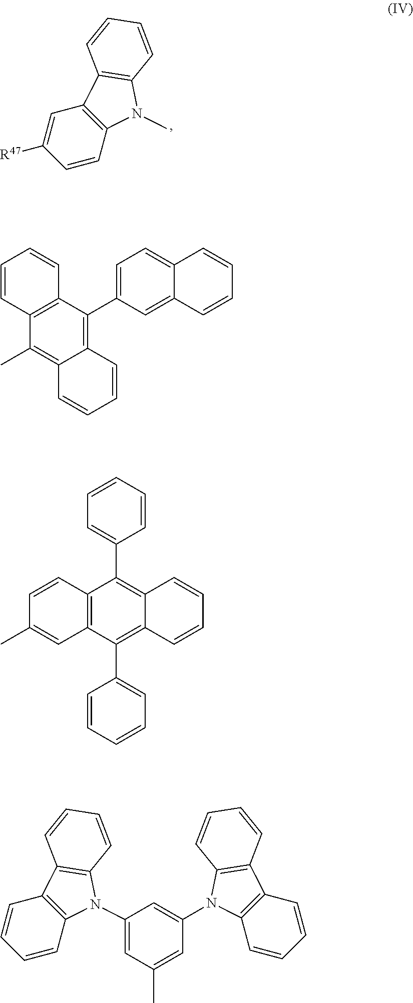

wherein R⁴⁷ may be a substituted or unsubstituted aromatic or heteroaromatic ring or ring system having 2 to 60 carbon atoms and optionally heteroatoms selected from N, O, and S. Optionally present substituents are selected from E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

A particularly preferred R⁴⁷ is shown in the following:

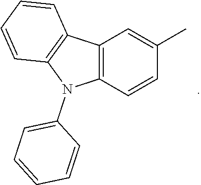 [SMILES: Cc1ccc2c(c1)c1ccccc1n2-c1ccccc1]

According to a particularly preferred embodiment of the present invention, A¹ is CR¹, A² is CR², A³ is CR³ and A⁴ is CR⁴, wherein R¹, R³ and R⁴ are H and R² is —CN or the substituent of formula (IV). According to a particularly preferred embodiment of the present invention R¹, R², and R⁴ are H and R³ is —CN or the substituent of formula (IV).

According to a further preferred embodiment A¹ is CR¹, A² is CR², A³ is CR³ and A⁴ is CR⁴ and R¹, R², R³ and R⁴ are H, meaning that A¹, A², A³ and A⁴ form an unsubstituted phenyl ring.

According to a further preferred embodiment A¹ is CR¹, A² is CR², A³ is CR³ and A⁴ is CR⁴ and two of R¹, R², R³ and R⁴, if present at adjacent carbon atoms, form a five or six membered, substituted or unsubstituted, saturated or unsaturated, aromatic or heteroaromatic, preferably heteroaromatic, ring, whereas the remaining two of R¹, R², R³ and R⁴ are H.

The five or six membered substituted or unsubstituted, saturated or unsaturated, aromatic or heteroaromatic ring that is formed by two of R¹, R², R³ and R⁴ is preferably fused to the six membered substituted or unsubstituted, saturated or unsaturated, aromatic or heteroaromatic ring that is formed by A¹, A², A³ and A⁴.

Preferably, A¹ is CR¹, A² is CR², A³ is CR³ and A⁴ is CR⁴ and R¹ and R² form a five or six membered, substituted or unsubstituted, saturated or unsaturated, aromatic or heteroaromatic ring, and R³ and R⁴ are H, or

R² and R³ form a five or six membered, substituted or unsubstituted, saturated or unsaturated, aromatic or heteroaromatic ring, and R¹ and R⁴ are H, or

R³ and R⁴ form a five or six membered, substituted or unsubstituted, saturated or unsaturated, aromatic or heteroaromatic ring, and R¹ and R² are H.

Preferably two of R¹, R², R³ and R⁴, if present at adjacent carbon atoms, form a five membered substituted heteroaromatic ring.

This five membered substituted heteroaromatic ring is preferably substituted by E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

The substituents that are present at the five membered substituted heteroaromatic ring are particularly preferably fused to the five membered substituted heteroaromatic ring.

Most preferred five membered substituted heteroaromatic rings that are formed by two of R¹, R², R³ and R⁴ are shown in the following:

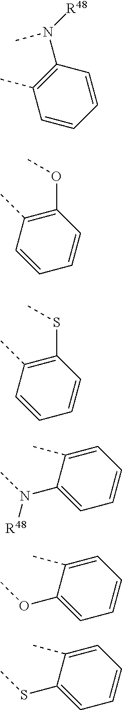

wherein the dashed lines describe the bonds to the ring which is formed by A¹, A², A³ and A⁴ and R⁴⁸ may be a substituted or unsubstituted aromatic or heteroaromatic ring or ring system having 2 to 60 carbon atoms and optionally heteroatoms selected from N, O, and S. Optionally present substituents are selected from E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

Particularly preferred R⁴⁸ are shown in the following:

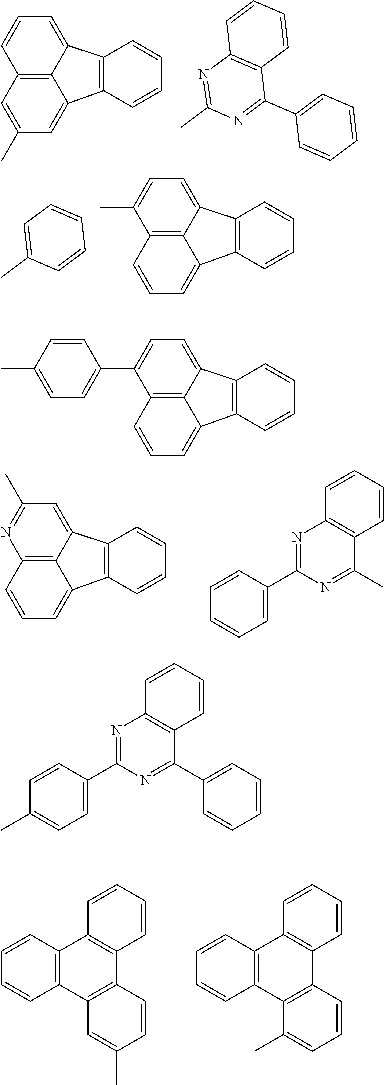

In general, B¹, B², B³, and B⁴ form an aromatic or heteroaromatic five or six membered ring, wherein B¹ is a direct bond, NR⁵, N, O, S, CR⁶ or CR⁷R⁸, B² is a direct bond, NR⁹, N, O, S, CR¹⁰ or CR¹¹R¹², B³ is a direct bond, NR¹³, N, O, S, CR¹⁴ or CR¹⁵R¹⁶ and/or B⁴ is a direct bond, NR¹⁷, N, O, S, CR¹⁸ or CR¹⁹R²⁰, wherein R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ have the meanings as mentioned above, preferably R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ are methyl (—CH₃),

CH₃, phenyl, triphenylene, dibenzofuran,

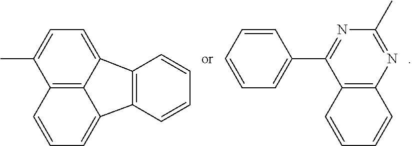

According to one embodiment of the present invention B¹, B², B³, and B⁴ form an aromatic or heteroaromatic five membered ring, wherein B¹ is a direct bond, NR⁵, O, S, CR⁶ or CR⁷R⁸, B² is a direct bond, NR⁹, O, S, CR¹⁰ or CR¹¹R¹², B³ is a direct bond, NR¹³, O, S, CR¹⁴ or CR¹⁵R¹⁶ and/or B⁴ is a direct bond, NR¹⁷, O, S, CR¹⁸ or CR¹⁹R²⁰, wherein R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ have the meanings as mentioned above.

According to another embodiment of the present invention B¹, B², B³, and B⁴ form an aromatic or heteroaromatic six membered ring, wherein B¹ is N, O, S or CR⁶, B² is N, O, S or CR¹⁰, B³ is N, O, S or CR¹⁴ and/or B⁴ is N, O, S or CR¹⁸, wherein R⁶, R¹⁰, R¹⁴ and R¹⁸ have the meanings as mentioned above.

According to a preferred embodiment, B¹, B², B³, and B⁴ form an aromatic or heteroaromatic six membered ring, wherein B¹ is N, B² is CR¹⁰, B³ is CR¹⁴ and B⁴ is CR¹⁸, wherein R¹⁰, R¹⁴ and R¹⁸ have the meanings as mentioned above, or

B¹ is CR⁶, B² is N, B³ is CR¹⁴ and B⁴ is CR¹⁸, wherein R⁶, R¹⁴ and R¹⁸ have the meanings as mentioned above, or

B¹ is CR⁶, B² is CR¹⁰, B³ is N and B⁴ is CR¹⁸, wherein R⁶, R¹⁰ and R¹⁸ have the meanings as mentioned above, or

B¹ is CR⁶, B² is CR¹⁰, B³ is CR¹⁴ and B⁴ is N, wherein R¹⁰, R¹⁴ and R¹⁸ have the meanings as mentioned above.

Particularly preferred, B¹ is CR⁶, B² is CR¹⁰, B³ is CR¹⁴ and B⁴ is CR¹⁸, wherein R⁶, R¹⁰, R¹⁴ and R¹⁸ have the meanings as mentioned above. According to this embodiment, the compound according to general formula (I) that is comprised in the electronic device according to the present invention corresponds to general formula (Ia) as shown in the following:

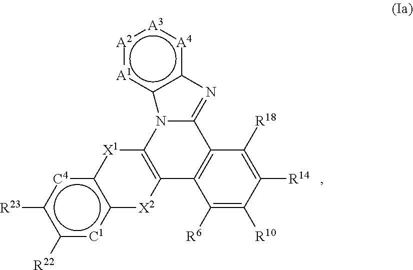

wherein A¹, A², A³, A⁴, C¹, C⁴, R⁶, R¹⁰, R¹⁴, R¹⁸, X¹, X², R²² and R²³ have the same meanings as mentioned above.

In an embodiment of the compound of the general formula (Ia), at least one selected from R⁶, R¹⁰, R¹⁴, R¹⁸, R²² and R²³ is a substituent other than H, preferably at least one of R²² and R²³ is a substituent other than H. The substituent is mentioned above with respect to R⁶, R¹⁰, R¹⁴, R¹⁸, R²² and R²³ of the general formula (I), and preferably represented by formula (b) or (b1) mentioned below.

Preferably, R⁶, R¹⁰, R¹⁴ and R¹⁸ are independently of each other selected from H, —CN,

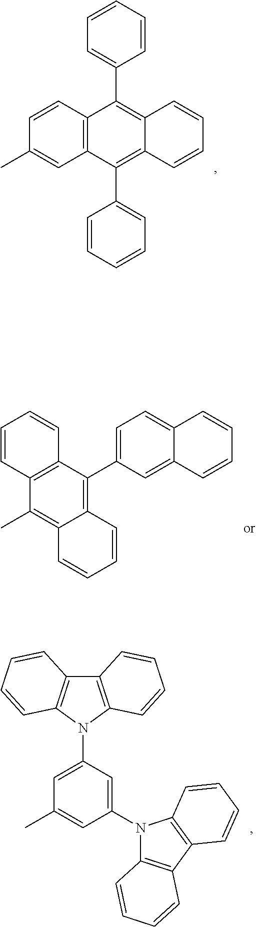

most preferably R⁶, R¹⁰, R¹⁴ and R¹⁸ are H.

According to a further preferred embodiment B¹ is CR⁶, B² is CR¹⁰, B³ is CR¹⁴ and B⁴ is CR¹⁸ and two of R⁶, R¹⁰, R¹⁴ and R¹⁸, if present at adjacent carbon atoms, form a five or six membered, preferably five membered, substituted or unsubstituted, saturated or unsaturated, aromatic or heteroaromatic, preferably heteroaromatic, ring, whereas the remaining two of R⁶, R¹¹, R¹⁴ and R¹⁸ are H.

The five or six membered substituted or unsubstituted, saturated or unsaturated, aromatic or heteroaromatic ring that is formed by two of R⁶, R¹⁰, R¹⁴ and R¹⁸ is preferably fused to the six membered substituted or unsubstituted, saturated or unsaturated, aromatic or heteroaromatic ring that is formed by B¹, B², B³ and B⁴.

Possible preferred substituents of the five or six membered substituted or unsubstituted, saturated or unsaturated, aromatic or heteroaromatic ring that is formed by two of R⁶, R¹⁰, R¹⁴ and R¹⁸ are a C₆-C₆₀aryl group which is unsubstituted or substituted by at least one group G, or a C₂-C₆₀heteroaryl group which is unsubstituted or substituted by at least one group G. These aryl or heteroaryl groups may be fused to the five or six membered substituted or unsubstituted, saturated or unsaturated, aromatic or heteroaromatic ring formed by two of R⁶, R¹⁰, R¹⁴ and R¹⁸ or may be bonded via single carbon-carbon-bonds to this ring.

Preferably, B¹ is CR⁶, B² is CR¹⁰, B³ is CR¹⁴ and B⁴ is CR¹⁸ and R⁶ and R¹⁰ form a five or six membered, substituted or unsubstituted, saturated or unsaturated, aromatic or heteroaromatic ring, and R¹⁴ and R¹⁸ are H, or

R¹⁰ and R¹⁴ form a five or six membered, substituted or unsubstituted, saturated or unsaturated, aromatic or heteroaromatic ring, and R⁶ and R¹⁸ are H, or

R¹⁴ and R¹⁸ form a five or six membered, substituted or unsubstituted, saturated or unsaturated, aromatic or heteroaromatic ring, and R⁶ and R¹⁰ are H.

Particularly preferred, B¹ is CR⁶, B² is CR¹⁰, B³ is CR¹⁴ and B⁴ is CR¹⁸ and R⁶ and R¹⁰ form a five or six membered, substituted or unsubstituted, saturated or unsaturated, aromatic or heteroaromatic ring, and R¹⁴ and R¹⁸ are H, or

R¹⁴ and R¹⁸ form a five or six membered, substituted or unsubstituted, saturated or unsaturated, aromatic or heteroaromatic ring, and R⁶ and R¹⁰ are H.

Preferably, two of R⁶, R¹⁰, R¹⁴ and R¹⁸ form a five membered substituted heteroaromatic ring or ringsystem.

The five membered substituted heteroaromatic ring formed by two of R⁶, R¹⁰, R¹⁴ and R¹⁸ is preferably substituted by H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

The substituents that are present at the five membered substituted heteroaromatic ring that is formed by two of R⁶, R¹⁰, R¹⁴ and R¹⁸ are particularly preferably fused to the five membered substituted heteroaromatic ring or are bond to this five membered substituted heteroaromatic ring via carbon-carbon-bond, most preferably the substituents are fused to the five membered substituted heteroaromatic ring.

Therefore, most preferred five membered substituted heteroaromatic rings that are formed by two of R⁶, R¹⁰, R¹⁴ and R¹⁸ are shown in the following:

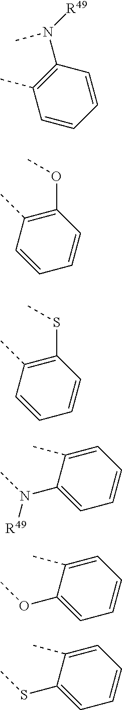

wherein the dashed lines describe the bonds to the ring which is formed by B¹, B², B³ and B⁴ and R⁴⁹ may be an substituted or unsubstituted aromatic or heteroaromatic ring or ringsystem having 2 to 60 carbon atoms and optionally heteroatoms selected from N, O, and S. Optionally present substituents thereof may be selected from E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

Particularly preferred R⁴⁹ are shown in the following:

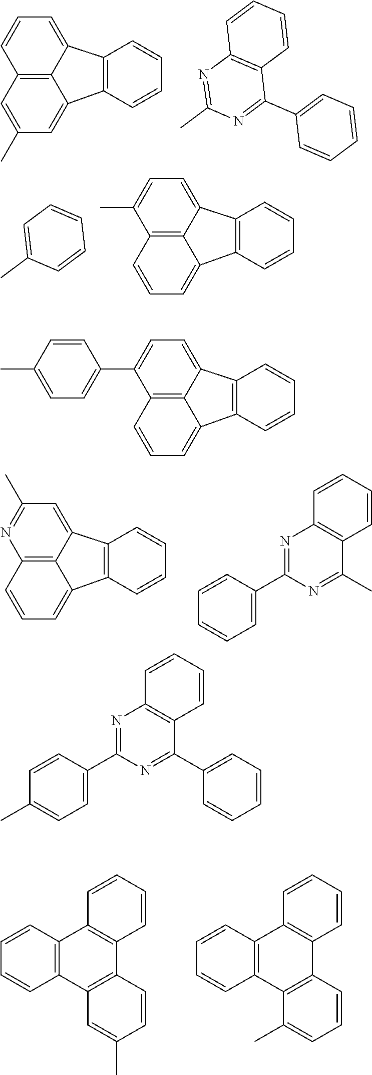

According to a preferred embodiment of the compounds of general formula (Ia), A¹ is preferably CR¹, A² is preferably CR², A³ is preferably CR³, A⁴ is preferably CR⁴, wherein R¹, R², R³ and R⁴ have the same meanings as mentioned above. According to this preferred embodiment, the compound according to general formula (I) or the compound according to general formula (Ia) correspond to the compound according to general formula (II) as shown in the following:

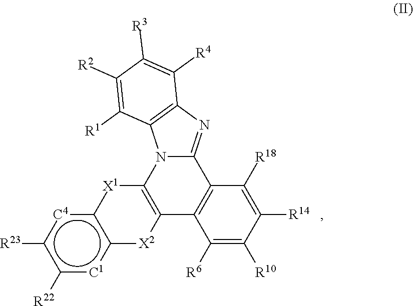

wherein R¹, R², R³, R⁴, R⁶, R¹⁰, R¹⁴, R¹⁸, R²², R²³, X¹, X², C¹ and C⁴ have the meanings as mentioned above.

Therefore, the present invention preferably relates to the electronic device according to the present invention, wherein the compound according to general formula (I) corresponds to general formula (II)

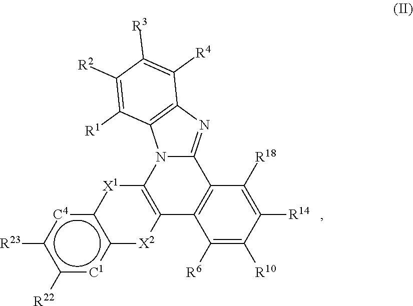

wherein R¹, R², R³, R⁴, R⁶, R¹⁰, R¹⁴, R¹⁸, R²², R²³, X¹, X², C¹ and C⁴ have the meanings as mentioned above.

In an embodiment of the compound of the general formula (II), at least one selected from R¹, R², R³, R⁴, R⁶, R¹⁰, R¹⁴, R¹⁸, R²², and R²³ is a substituent other than H, preferably at least one of R²² and R²³ is a substituent other than H. The substituent is mentioned above with respect to R¹, R², R³, R⁴, R⁶, R¹⁰, R¹⁴, R¹⁸, R²², and R²³ of the general formula (I), and preferably formula (b) or (b1) to be mentioned below.

Particularly preferably, R⁶, R¹⁰, R¹⁴ and R¹⁸ are independently selected from H, —CN,

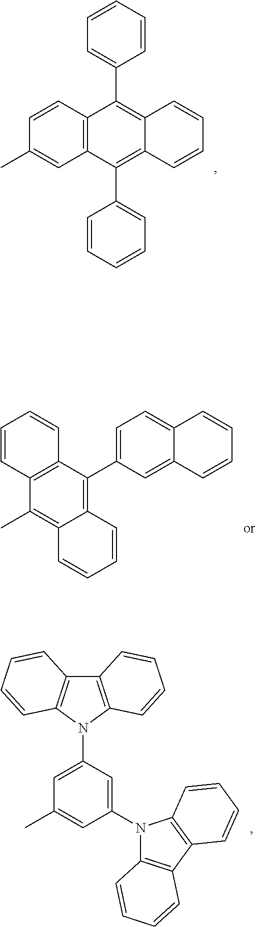

most preferably R⁶, R¹⁰, R¹⁴ and R¹⁸ are H.

As mentioned above, A¹ is preferably CR¹, A² is preferably CR², A³ is preferably CR³, A4 is preferably CR⁴, and R¹, R², R³ and R⁴ are particularly preferably H. According to this preferred embodiment, the compound according to general formula (II) as shown above corresponds to the particularly preferred compound according to general formula (IIa) as shown in the following:

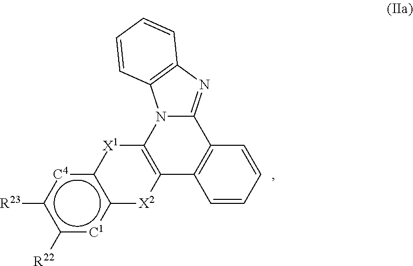

wherein R²², R²³, X¹, X², C¹, and C⁴ have the same meanings as mentioned above.

In an embodiment of the compound of the general formula (IIa), at least one of R²² and R²³ is a substituent other than H. The substituent is mentioned above with respect to R²² and R²³ of the general formula (I), and preferably formula (b) or (b1) to be mentioned below.

According to another preferred embodiment of the present invention, B¹, B², B³, and B⁴ in general formula (I) form an aromatic or heteroaromatic five membered ring, wherein B¹ is a direct bond, NR⁶, O, S, CR⁶ or CR⁷R⁸, B² is a direct bond, NR⁹, O, S, CR¹⁰ or CR¹¹R¹², B³ is a direct bond, NR¹³, O, S, CR¹⁴ or CR¹⁵R¹⁶ and/or B⁴ is a direct bond, NR¹⁷, O, S, CR¹⁸ or CR¹⁹R²⁰, wherein R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ have the meanings as mentioned above.

According to this preferred embodiment, B¹ is a direct bond, B² is NR⁹, O, S, CR¹⁰ or CR¹¹R¹², B³ is NR¹³, O, S, CR¹⁴ or CR¹⁵R¹⁶ and B⁴ is NR¹⁷, O, S, CR¹⁸ or CR¹⁹R²⁰, wherein R⁹, R¹⁰, R¹¹, R¹², R¹³, R¹⁴, R¹⁵, R¹⁶, R¹⁷, R¹⁸, R¹⁹ and R²⁰ have the meanings as mentioned above, or

B¹ is NR⁵, O, S, CR⁶ or CR⁷R⁸, B² is a direct bond, B³ is NR¹³, O, S, CR¹⁴ or CR¹⁵R¹⁶ and B⁴ is NR¹⁷, O, S, CR¹⁸ or CR¹⁹R²⁰, wherein R⁵, R⁶, R⁷, R⁸, R¹³, R¹⁴, R¹⁵, R¹⁶, R¹⁷, R¹⁸, R¹⁹ and R²⁰ have the meanings as mentioned above, or

wherein B¹ is NR⁵, O, S, CR⁶ or CR⁷R⁸, B² is NR⁹, O, S, CR¹⁰ or CR¹¹R¹², B³ is a direct bond and B⁴ is NR¹⁷, O, S, CR¹⁸ or CR¹⁹R²⁰, wherein R⁵, R⁶, R⁷, R⁸, R⁹, R¹⁰, R¹¹, R¹², R¹⁷, R¹⁸, R¹⁹ and R²⁰ have the meanings as mentioned above, or

wherein B¹ is NR⁵, O, S, CR⁶ or CR⁷R⁸, B² is NR⁹, O, S, CR¹⁰ or CR¹¹R¹², B³ is NR¹³, O, S, CR¹⁴ or CR¹⁵R¹⁶ and B⁴ is a direct bond, wherein R⁵, R⁶, R⁷, R⁸, R⁹, R¹⁰, R¹¹, R¹², R¹³, R¹⁴, R¹⁵ and R¹⁶ have the meanings as mentioned above.

According to a preferred embodiment of the present invention, B¹ is a direct bond, B² is NR⁹, 0, S, CR¹⁰ or CR¹¹R¹², B³ is NR¹³, O, S, CR¹⁴ or CR¹⁵R¹⁶ and B⁴ is NR¹⁷, O, S, CR¹⁸ or CR¹⁹R²⁰, wherein R⁹, R¹⁰, R¹, R¹², R¹³, R¹⁴, R¹⁵, R¹⁶, R¹⁷, R¹⁸, R¹⁹ and R²⁰ have the meanings as mentioned above, or

B¹ is NR⁵, O, S, CR⁶ or CR⁷R⁸, B² is NR⁹, O, S, CR¹⁰ or CR¹¹R¹², B³ is NR¹³, O, S, CR¹⁴ or CR¹⁵R¹⁶ and B⁴ is a direct bond, wherein R⁵, R⁶, R⁷, R⁸, R⁹, R¹⁰, R¹¹, R¹², R¹³, R¹⁴, R¹⁵ and R¹⁶ have the meanings as mentioned above.

The particularly preferred embodiment, wherein B¹ is a direct bond, B² is NR⁹, O, S, CR¹⁰ or CR¹¹R¹², B³ is NR¹³, O, S, CR¹⁴ or CR¹⁵R¹⁶ and B⁴ is NR¹⁷, 0, S, CR¹⁸ or CR¹⁹R²⁰, wherein R⁹, R¹⁰, R¹¹, R¹², R¹³, R¹⁴, R¹⁵, R¹⁶, R¹⁷, R¹⁸, R¹⁹ and R²⁰ have the meanings as mentioned above, is shown in the following compound (Ib):

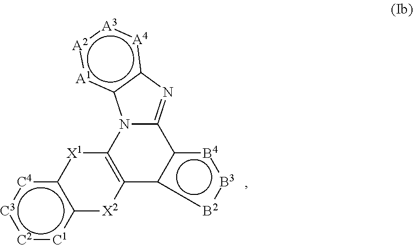

wherein A¹, A², A³, A⁴, C¹, C², C³, C⁴, X¹ and X² have the same meanings as mentioned above.

Preferably, in the compound according to general formula (Ib), B² is NR⁹, O, or S, B³ is CR¹⁴ and B⁴ is CR¹⁸, wherein R⁹, R¹⁴ and R¹⁸ have the meanings as mentioned above, or

B² is CR¹⁰, B³ is NR¹³, O, or S, and B⁴ is CR¹⁸, wherein R¹⁰, R¹³ and R¹⁸ have the meanings as mentioned above, or

B² is CR¹⁰, B³ is CR¹⁴ and B⁴ is NR¹⁷, O, or S, wherein R¹⁰, R¹⁴ and R¹⁷ have the meanings as mentioned above,

wherein A¹, A², A³, A⁴, C¹, C², C³, C⁴, X¹ and X² have the same meanings as mentioned above.

Particularly preferred, in the compound according to general formula (Ib),

B² is NR⁹, O, or S, B³ is CR¹⁴ and B⁴ is CR¹⁸ or

B² is CR¹⁰, B³ is CR¹⁴ and B⁴ is NR¹⁷, O, or S,

wherein R⁹ and R¹⁷ are selected from aromatic or heteroaromatic rings or ring systems having 2 to 60 carbon atoms, and R¹⁴ and R¹⁸ or R¹⁰ and R¹⁴ form a five or six membered, substituted or unsubstituted, saturated or unsaturated, aromatic or heteroaromatic ring.

Most preferably, R⁹ and R¹⁷ are independently of each other selected from the group consisting of

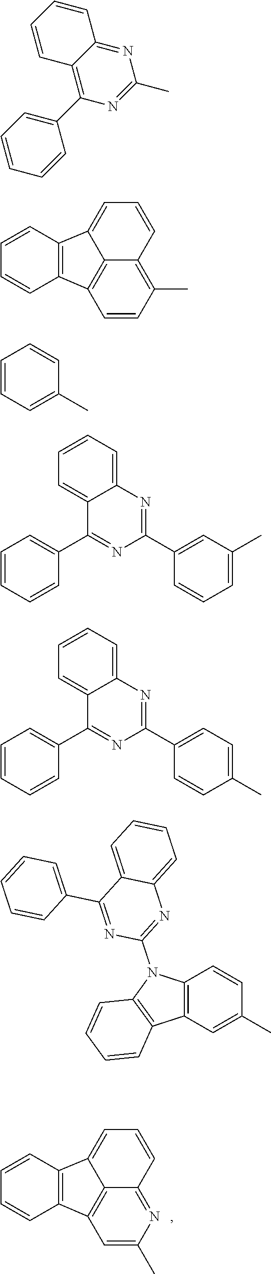

R¹⁴ and R¹⁸ or R¹⁰ and R¹⁴ preferably form a five or six membered, substituted or unsubstituted, saturated or unsaturated, aromatic or heteroaromatic ring, most preferably a six membered, substituted or unsubstituted aromatic ring which corresponds to the following formula (Id)

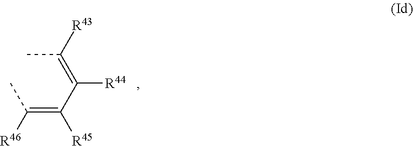

wherein the dashed lines describe the bonding to B¹, B², B³ and/or B⁴, and wherein R⁴³, R⁴⁴, R⁴⁵, and R⁴⁶ are independently of each independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰,

or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,

wherein G, E, D, R²⁸, R²⁹, and R³⁰ have the meanings as defined above.

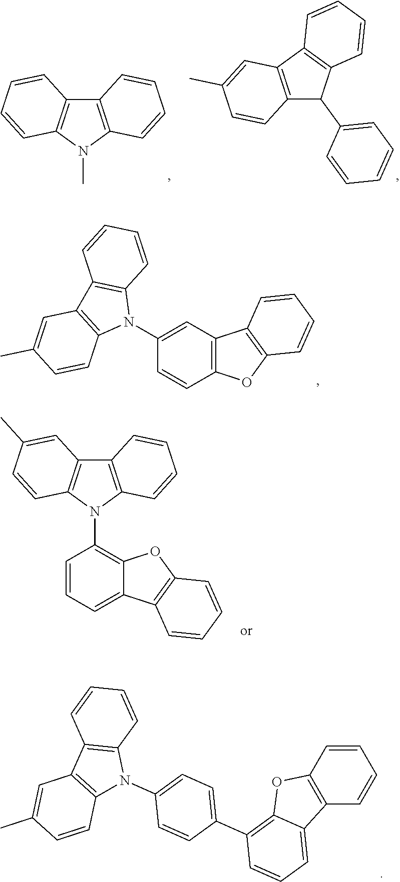

Most preferably R⁴³, R⁴⁴, R⁴⁵, and R⁴⁶ are H,

According to a preferred embodiment of the present invention, X¹ and B¹ are direct bond, X² is O, S, NR²⁵, or CR²⁶R²⁷ and B⁴ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

The present invention therefore preferably relates to the electronic device according to the present invention, wherein X¹ and B¹ are direct bond, X² is O, S, NR²⁵, or CR²⁶R²⁷ and B⁴ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

According to a preferred embodiment of the present invention, X¹ and B⁴ are direct bond, X² is O, S, NR²⁵, or CR²⁶R²⁷ and B¹ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

The present invention therefore preferably relates to the electronic device according to the present invention, wherein X¹ and B⁴ are direct bond, X² is O, S, NR²⁵, or CR²⁶R²⁷ and B¹ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

According to a preferred embodiment of the present invention, X² and B⁴ are direct bond, X¹ is O, S, NR²⁵, or CR²⁶R²⁷ and B¹ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

The present invention therefore preferably relates to the electronic device according to the present invention, wherein X² and B⁴ are direct bond, X¹ is O, S, NR²⁵, or CR²⁶R²⁷ and B¹ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

According to a preferred embodiment of the present invention, X² and B¹ are direct bond, X¹ is O, S, NR²⁵, or CR²⁶R²⁷ and B⁴ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

The present invention therefore preferably relates to the electronic device according to the present invention, wherein X² and B¹ are direct bond, X¹ is O, S, NR²⁵, or CR²⁶R²⁷ and B⁴ is NR¹⁷, N, O, S, CR¹⁸ or CR¹⁹R²⁰, wherein R¹⁷, R¹⁸, R¹⁹, R²⁰, R²⁵, R²⁶ and R²⁷ have the meaning as defined above.

The further particularly preferred embodiment, wherein B¹ is NR⁵, O, S, CR⁶ or CR⁷R⁸, B² is NR⁹, O, S, CR¹⁰ or CR¹¹R¹², B³ is NR¹³, O, S, CR¹⁴ or CR¹⁵R¹⁶ and B⁴ is a direct bond, wherein R⁵, R⁶, R⁷, R⁸, R⁹, R¹⁰, R¹¹, R¹², R¹³, R¹⁴, R¹⁵ and R¹⁶ have the meanings as mentioned above, is shown in the following as compound (Ic):

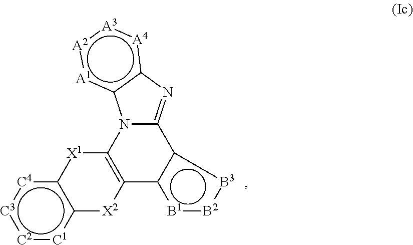

wherein A¹, A², A³, A⁴, C¹, C², C³, C⁴, X¹ and X² have the same meanings as mentioned above.

Preferably, in the compound according to general formula (Ic), B¹ is NR⁵, O, or S, B² is CR¹⁰ and B³ is CR¹⁴, wherein R⁵, R¹⁰ and R¹⁴ have the meanings as mentioned above, or

B¹ is CR⁶, B² is NR⁹ and B³ is CR¹⁴, wherein R⁶, R⁹ and R¹⁴ have the meanings as mentioned above, or

B¹ is CR⁶, B² is CR¹⁰ and B³ is NR¹³, O, or S, wherein R⁶, R¹⁰ and R¹³ have the meanings as mentioned above,

wherein A¹, A², A³, A⁴, C¹, C², C³, C⁴, X¹ and X² have the same meanings as mentioned above.

Particularly preferred, in the compound according to general formula (Ic),

B¹ is NR⁵, O, or S, B² is CR¹⁰ and B³ is CR¹⁴, or

B¹ is CR⁶, B² is CR¹⁰ and B³ is NR¹³, O, or S,

wherein R⁵ and R¹³ are selected from aromatic or heteroaromatic rings or ring systems having 2 to 60 carbon atoms, and R¹⁰ and R¹⁴ or R⁶ and R¹⁰ form a five or six membered, substituted or unsubstituted, saturated or unsaturated, aromatic or heteroaromatic ring.

Most preferably, R⁵ and R¹³ are independently of each other selected from the group consisting of

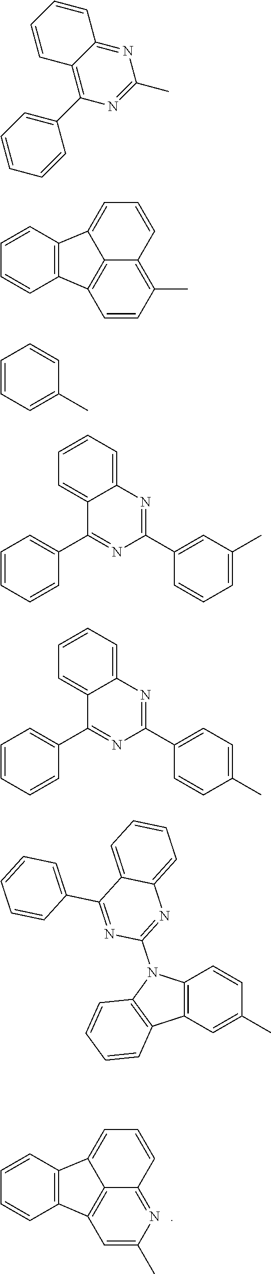

R⁶ and R¹⁰ or R¹⁴ and R¹⁸ preferably form a five or six membered, substituted or unsubstituted, saturated or unsaturated, aromatic or heteroaromatic ring, most preferably a six membered, substituted or unsubstituted aromatic ring which corresponds to the following formula (Id):

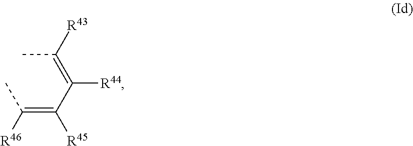

wherein the dashed lines describe the bonding to B¹, B², B³ and/or B⁴, and wherein R⁴³, R⁴⁴, R⁴⁵, R⁴⁶ are independently of each independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰,

or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,

wherein G, E, D, R²⁸, R²⁹, and R³⁰ have the meanings as defined above. Most preferably R⁴³, R⁴⁴, R⁴⁵, R⁴⁶ are H,

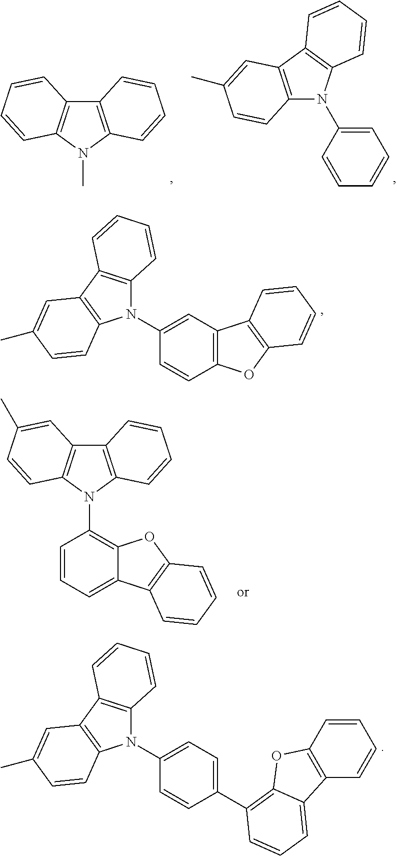

Most preferably, in the compounds according to general formulae (Ib) and (Ic) A¹, A², A³ and A⁴ have the meaning CR¹, CR², CR³ and CR⁴, wherein R¹, R², R³ and R⁴ have the meanings as mentioned above, and B² is CR¹⁰ and B³ is CR¹⁴, wherein R¹⁰ and R¹⁴ form a six membered, substituted or unsubstituted aromatic ring which corresponds to the following formula (Id)

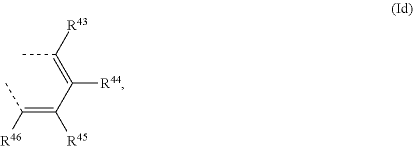

wherein the dashed lines describe the bonding to B² and B³, and wherein R⁴³, R⁴⁴, R⁴⁵, and R⁴⁶ are independently of each independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰,

or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,

wherein G, E, D, R²⁸, R²⁹, and R³⁰ have the meanings as defined above. Most preferably R⁴³, R⁴⁴, R⁴⁵, R⁴⁶ are H,

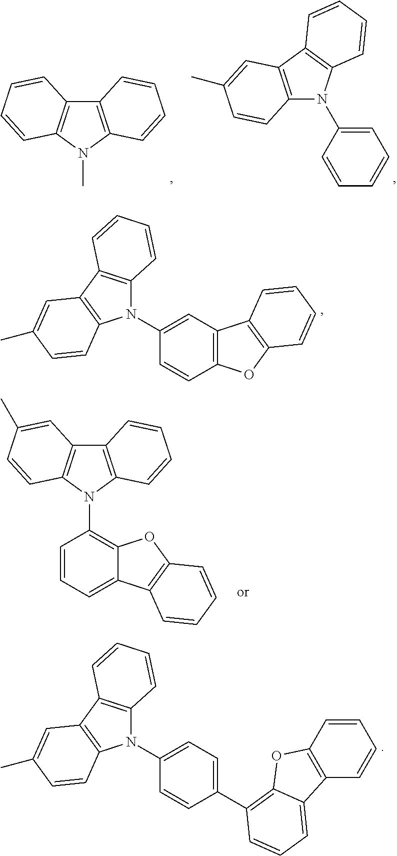

The present invention therefore preferably relates to the electronic device according to the present invention, wherein the compound according to general formula (I) corresponds to general formula (III):

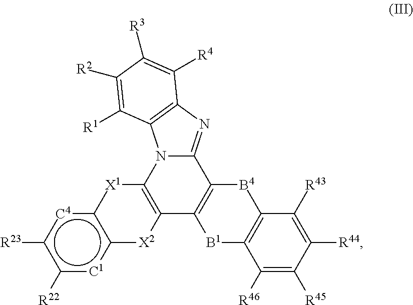

wherein B¹ is a direct bond and B⁴ is NR¹⁷, N, O, S, CR¹⁸ or CR¹⁹R²⁰, or B¹ is NR⁵, N, O, S, CR⁶ or CR⁷R⁸ and B⁴ is a direct bond,

C¹ is N or CR²¹ and C⁴ is N or CR²⁴,

X¹, X² direct bond, O, S, NR²⁵, or CR²⁶R²⁷, wherein one of X¹ and X² is a direct bond and the other one is O, S, NR²⁵, or CR²⁶R²⁷,

R⁴³, R⁴⁴, R⁴⁵, and R⁴⁶ are independently of each independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰,

or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,

wherein G, E, D, R¹, R², R³, R⁴, R⁵, R⁶, R⁷, R⁸, R¹⁷, R¹⁸, R¹⁹, R²⁰, R²¹, R²², R²³, R²⁴, R²⁵, R²⁶, R²⁷, R²⁸, R²⁹, and R³⁰ have the meanings as defined above,

or R²⁵ may form a five or six membered, substituted or unsubstituted, saturated or unsaturated, aromatic or heteroaromatic ring with R²¹, R²², R²³ or R²⁴,

or R⁵ or R¹⁷ may form a five or six membered, substituted or unsubstituted, saturated or unsaturated, aromatic or heteroaromatic ring with R⁴³, R⁴⁴, R⁴⁵ or R⁴⁶.

According to a further preferred embodiment, R⁵ or R¹⁷ form a five or six membered, substituted or unsubstituted, saturated or unsaturated, aromatic or heteroaromatic ring with R⁴³, R⁴⁴, R⁴⁵ or R⁴⁶, if present. Preferably R⁵ or R¹⁷ and R⁴³, R⁴⁴, R⁴⁵ or R⁴⁶ form five membered saturated ring to which aromatic or heteroaromatic rings or ring systems with 4 to 30 carbon atoms that may be fused. Preferably, R⁶ and R⁴⁶ or R¹⁷ and R⁴³ form a five or six membered, substituted or unsubstituted, saturated or unsaturated, aromatic or heteroaromatic ring, particularly preferably form five membered saturated ring to which aromatic or heteroaromatic rings or ring systems with 4 to 30 carbon atoms that may be fused.

In case that B¹ is a direct bond, the mostly preferred the moiety that is formed by R¹⁷ and R⁴³ corresponds to the following formula:

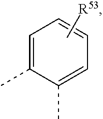

wherein the dashed lines describe the bonding to N in respect to R¹⁷ and to B⁴, and wherein R⁵³ may be one or more, preferably at most 4, substituents selected from H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰, wherein G, E, D, R²⁸, R²⁹ and R³⁰ have independently of each other the meanings as defined above. These substituents may be bonded by carbon-carbon-bonds or may be fused to the aromatic ring.

In case that B⁴ is a direct bond, the mostly preferred the moiety that is formed by R⁵ and R⁴⁶ corresponds to the following formula:

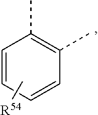

wherein the dashed lines describe the bonding to N in respect to R⁵ and to B⁴, and wherein R⁵⁴ may be one or more, preferably at most 4, substituents selected from H, E, a C₆-C₆₀aryl group which is unsubstituted or substituted by at least one group G, a C₂-C₆₀heteroaryl group which is unsubstituted or substituted by at least one group G, a C₁-C₂₅alkyl group, which is unsubstituted or substituted by at least one group E and/or interrupted by D, a C₆-C₆₀aryloxy group which is unsubstituted or substituted by at least one group G, or —SiR²⁸R²⁹R³⁰, wherein G, E, D, R²⁸, R²⁹ and R³⁰ have independently of each other the meanings as defined above. These substituents may be bonded by carbon-carbon-bonds or may be fused to the aromatic ring.

In the compounds according to general formula (I), in particular according to general formula (Ia), (Ib), (Ic), (II), (IIa), (III) and (IV), C¹, C², C³, and C⁴ form an aromatic or heteroaromatic six membered ring, wherein C¹ is N or CR²¹, C² is N or CR²², C³ is N or CR²³ and C⁴ is N or CR²⁴, wherein R²¹, R²², R²³ and R²⁴ have the meanings as mentioned above.

According to one preferred embodiment, C¹ is N, C² is CR²², C³ is CR²³ and C⁴ is CR²⁴, wherein R²², R²³ and R²⁴ have the meanings as mentioned above, or

C¹ is CR²¹, C² is N, C³ is CR²³ and C⁴ is CR²⁴, wherein R²¹, R²³ and R²⁴ have the meanings as mentioned above, or

C¹ is CR²¹, C² is CR²², C³ is N or CR²³ and C⁴ is CR²⁴, wherein R²¹, R²² and R²⁴ have the meanings as mentioned above, or

C¹ is CR²¹, C² is CR²², C³ is CR²³ and C⁴ is N, wherein R²¹, R²² and R²³ have the meanings as mentioned above.

According to these embodiments, wherein C¹, C², C³ and C⁴ form a N-comprising heteroaromatic cycle, R²¹, R²², R²³ and R²⁴ are preferably selected from H, —CN,

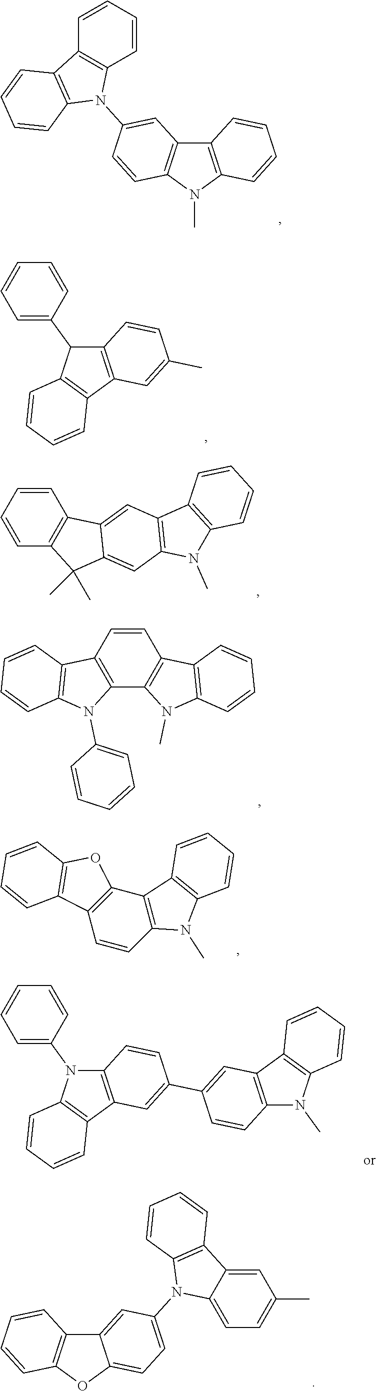

More preferred, C¹ is CR²¹, C² is CR²², C³ is CR²³ and C⁴ is CR²⁴ and R²¹, R²², R²³ and R²⁴ are independently of each other H, E, a C₆-C₆₀aryl group which is unsubstituted or substituted by at least one group G, or a C₂-C₆₀heteroaryl group which is unsubstituted or substituted by at least one group G, wherein E and G have the meanings as mentioned above. Particularly preferred, C¹ is CR²¹, C² is CR²², C³ is CR²³ and C⁴ is CR²⁴ and R²¹, R²², R²³ and R²⁴ are H.

According to a further preferred embodiment of the present invention C¹ is CR²¹, C² is C²², C³ is CR²³ and C⁴ is CR²⁴ and R²¹ is independently of each other E, a C₆-C₆₀aryl group which is unsubstituted or substituted by at least one group G, or a C₂-C₆₀heteroaryl group which is unsubstituted or substituted by at least one group G, wherein E and G have the meanings as mentioned above, and R²², R²³ and R²⁴ are H.

According to a further preferred embodiment of the present invention C¹ is CR²¹, C² is C²², C³ is CR²³ and C⁴ is CR²⁴ and R²² is independently of each other E, a C₆-C₆₀aryl group which is unsubstituted or substituted by at least one group G, or a C₂-C₆₀heteroaryl group which is unsubstituted or substituted by at least one group G, wherein E and G have the meanings as mentioned above, and R²¹, R²³ and R²⁴ are H.

According to a further preferred embodiment of the present invention C¹ is CR²¹, C² is C²², C³ is CR²³ and C⁴ is CR²⁴ and R²³ is independently of each other E, a C₆-C₆₀aryl group which is unsubstituted or substituted by at least one group G, or a C₂-C₆₀heteroaryl group which is unsubstituted or substituted by at least one group G, wherein E and G have the meanings as mentioned above, and R²¹, R²² and R²⁴ are H.

Most preferred, C¹ is CR²¹, C² is CR²², C³ is CR²³ and C⁴ is CR²⁴ and R²¹ is independently of each other —OR³⁵, —SR³⁶, —NR³⁷R³⁸, —COR³⁹, —COOR⁴⁰, —CONR⁴¹R⁴², —CN, —SiR²⁸R²⁹R³⁰, halogen, a C₆-C₆₀aryl group which is unsubstituted or substituted by at least one group G, or a C₂-C₆₀heteroaryl group which is unsubstituted or substituted by at least one group G, and R²², R²³ and R²⁴ have the meanings as mentioned above, wherein R²⁸, R²⁹, R³⁰, R³⁵, R³⁶, R³⁷, R³⁸, R³⁹, R⁴⁰, R⁴¹, R⁴² and G have the same meanings as mentioned above, or

R²² is independently of each other —OR³⁵, —SR³⁶, —NR³⁷R³⁸, —COR³⁹, —COOR⁴⁰, —CONR⁴¹R⁴², —CN, —SiR²⁸R²⁹R³⁰, halogen, a C₆-C₆₀aryl group which is unsubstituted or substituted by at least one group G, or a C₂-C₆₀heteroaryl group which is unsubstituted or substituted by at least one group G, and R²¹, R²³ and R²⁴ have the meanings as mentioned above, wherein R²⁸, R²⁹, R³⁰, R³⁵, R³⁶, R³⁷, R³⁸, R³⁹, R⁴⁰, R⁴¹, R⁴² and G have the same meanings as mentioned above, or

R²³ is independently of each other —OR³⁵, —SR³⁶, —NR³⁷R³⁸, —COR³⁹, —COOR⁴⁰, —CONR⁴¹R⁴², —CN, —SiR²⁸R²⁹R³⁰, halogen, a C₆-C₆₀aryl group which is unsubstituted or substituted by at least one group G, or a C₂-C₆₀heteroaryl group which is unsubstituted or substituted by at least one group G, and R²¹, R²² and R²⁴ have the meanings as mentioned above, wherein R²⁸, R²⁹, R³⁰, R³⁵, R³⁶, R³⁷, R³⁸, R³⁹, R⁴⁰, R⁴¹, R⁴² and G have the same meanings as mentioned above.

A particularly preferred meaning of R²¹, R²², R²³ and/or R²⁴ is the substituent of formula (VI) and the other substituents as shown:

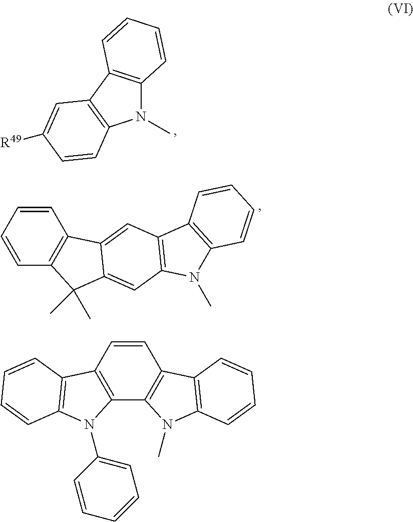

wherein R⁴⁹ may be an substituted or unsubstituted aromatic or heteroaromatic ring or ringsystem having 2 to 60 carbon atoms and optionally heteroatoms selected from N, O, and S. Optionally present substituents are selected from E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above.

A particularly preferred R⁴⁹ is shown in the following:

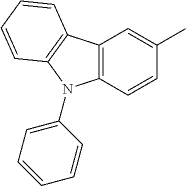 [SMILES: Cc1ccc2c(c1)c1ccccc1n2-c1ccccc1]

Further particularly preferred meanings of R²¹, R²², R²³ and/or R²⁴, in particular of R²³, are the substituents as shown in the following:

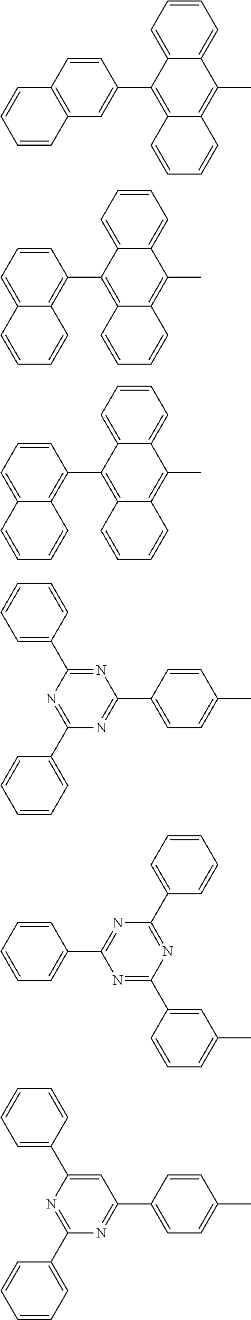

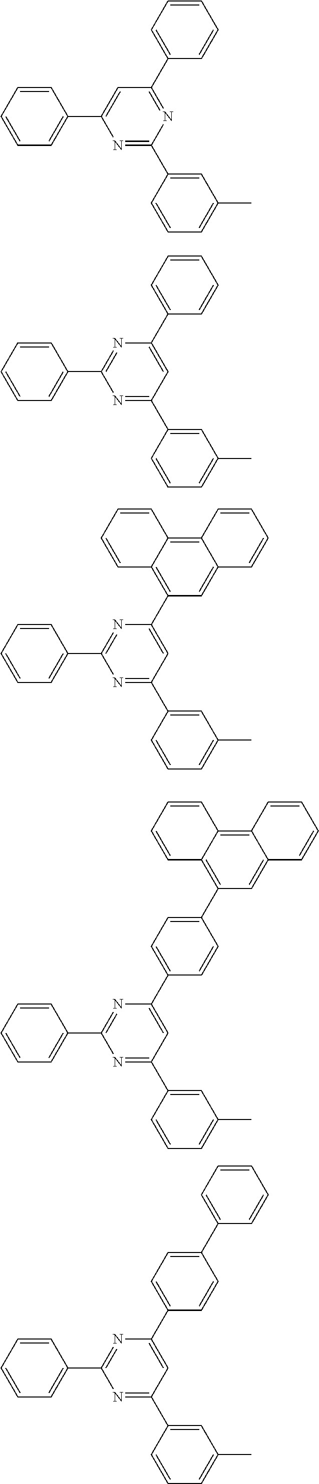

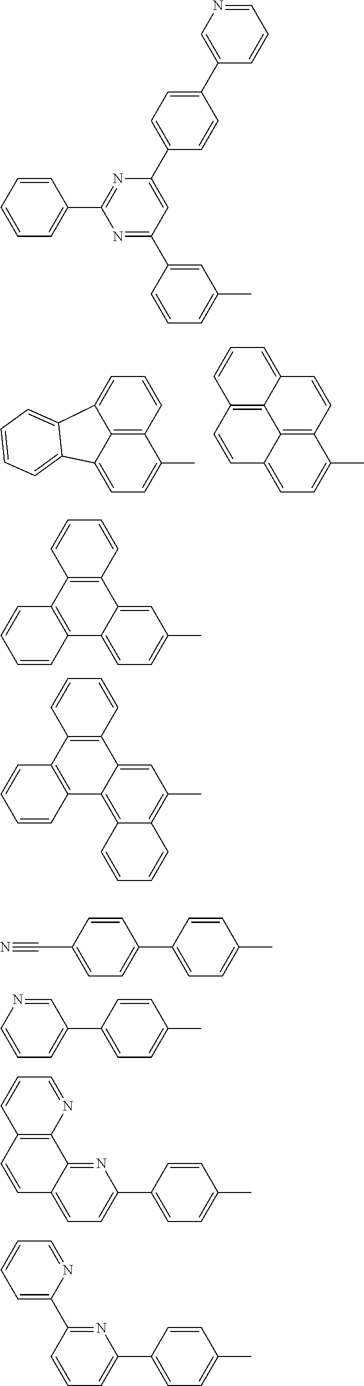

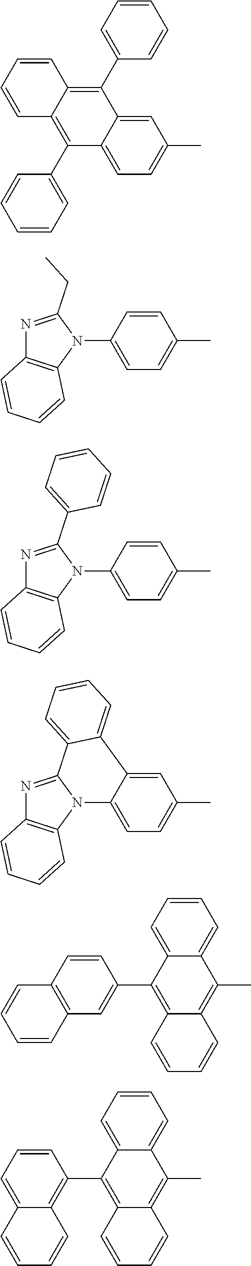

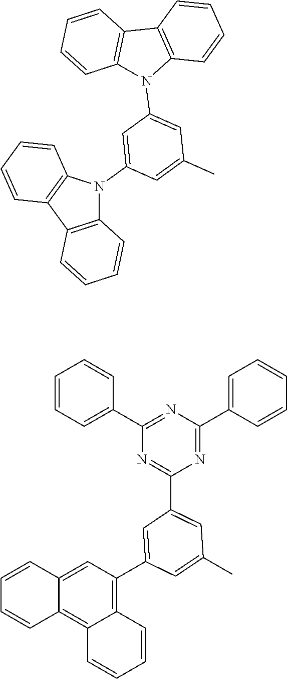 [SMILES: Cc1cc(-n2c3ccccc3c3ccccc32)cc(-n2c3ccccc3c3ccccc32)c1 ; Cc1cc(-c2nc(-c3ccccc3)nc(-c3ccccc3)n2)cc(-c2cc3ccccc3c3ccccc23)c1]

R²¹, R²², R²³ and/or R²⁴, in particular of R²³, are preferably the above mentioned substituents, if A¹ is CR¹, A² is CR², A³ is CR³, A⁴ is CR⁴ and R¹, R², R³ and R⁴ are H, B¹ is CR⁶, B² is CR¹⁰, B³ is CR¹⁴ and B⁴ is CR¹⁸ and R⁶, R¹⁰, R¹⁴ and R¹⁸ are H, and C¹ is CR²¹, C² is CR²², C₃ is CR²³ and C⁴ is CR²⁴. According to this embodiment, if only one of R²¹, R²², R²³ and R²⁴, preferably R²³, is a substituent as mentioned above, the remaining ones are preferably H.

A further particularly preferred meaning of R²¹, R²², R²³ and/or R²⁴ is —ON.

According to a preferred embodiment C¹ is N or CR²¹, C² is N or CR²², C³ is N or CR²³ and C⁴ is N or CR²⁴ and two of R²¹, R²², R²³ and R²⁴, if present at adjacent carbon atoms, form a five or six membered, substituted or unsubstituted, saturated or unsaturated, aromatic or heteroaromatic, preferably heteroaromatic, ring, whereas the remaining two of R²¹, R²², R²³ and R²⁴ are H.

The five or six membered substituted or unsubstituted, saturated or unsaturated, aromatic or heteroaromatic ring that is formed by two of R²¹, R²², R²³ and R²⁴ is preferably fused to the six membered substituted or unsubstituted, saturated or unsaturated, aromatic or heteroaromatic ring that is formed by C¹, C², C³ and C⁴.

Preferably, C¹ is CR²¹, C² is CR²², C³ is CR²³ and C⁴ is CR²⁴ and R²¹ and R²² form a five or six membered, substituted or unsubstituted, saturated or unsaturated, aromatic or heteroaromatic ring, and R²³ and R²⁴ are H, or

R²² and R²³ form a five or six membered, substituted or unsubstituted, saturated or unsaturated, aromatic or heteroaromatic ring, and R²¹ and R²⁴ are H, or

R²³ and R²⁴ form a five or six membered, substituted or unsubstituted, saturated or unsaturated, aromatic or heteroaromatic ring, and R²¹ and R²² are H.

The embodiment, wherein C¹ is CR²¹, C² is CR²², C³ is CR²³ and C⁴ is CR²⁴ and R²¹ and R²² form a five or six membered, substituted or unsubstituted, saturated or unsaturated, aromatic or heteroaromatic ring, and R²³ and R²⁴ are H, is particularly preferred.

Preferably R²¹, R²², R²³ and R²⁴ form a five membered substituted heteroaromatic ring.

Most preferred five membered substituted heteroaromatic rings that are formed by two of R²¹, R²², R²³ and/or R²⁴ are shown in the following:

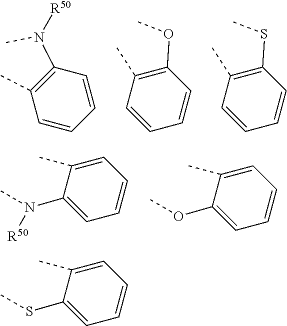

wherein the dashed lines describe the bonds to the ring which is formed by C¹, C², C³ and C⁴ and R⁵⁰ may be a substituted or unsubstituted aromatic or heteroaromatic ring or ringsystem having 2 to 60 carbon atoms and optionally heteroatoms selected from N, O, and S. Optionally present substituents are selected from E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, or —SiR²⁸R²⁹R³⁰, wherein G, E, D, R²⁸, R²⁹ and R³⁰ have independently of each other the meanings as defined above.

Particularly preferred R⁵⁰ are shown in the following:

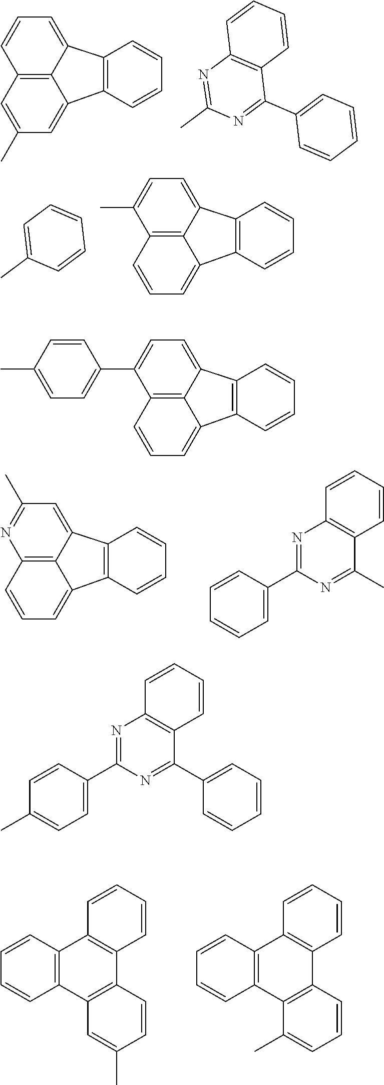

In the compounds according to general formula (I), in particular according to general formula (Ia), (Ib), (Ic), (III) and (IV), X¹ and X² are direct bond, O, S, NR²⁵, or CR²⁶R²⁷, wherein one of X¹ and X² is a direct bond and the other one is O, S, NR²⁵, or CR²⁶R²⁷.

The compounds according to the present invention therefore preferably correspond to the compound according to formula (Ie):

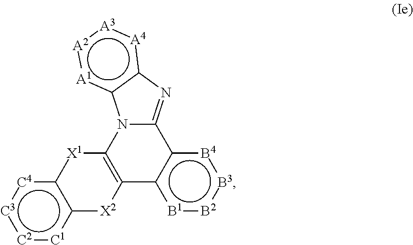

wherein A¹, A², A³, A⁴, B¹, B², B³, B⁴, C¹, C², C³ and C⁴ have the above mentioned meanings and X¹ is direct bond and X² is O, S, NR²⁵, CR²⁶R²⁷ or X¹ is O, S, NR²⁵, CR²⁶R²⁷ and X² is a direct bond.

In an embodiment of the compound of the general formula (Ie), A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R¹, R², R³, R⁴, R⁶, R¹⁰, R¹⁴, R¹⁸, R²¹, R²², R²³, and R²⁴ is a substituent other than H. The substituent is mentioned above with respect to R¹, R², R³, R⁴, R⁶, R¹⁰, R¹⁴, R¹⁸, R²¹, R²², R²³, and R²⁴ of the general formula (I), and preferably represented by the following formula (b) or (b1).

In an embodiment of the compound of the general formula (Ie), at least one selected from R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴, R²⁵, R²⁶, and R²⁷ is a substituent of general formula (b):

(R⁵⁶)ₜ-L- (b)

wherein

L is a direct bond, a C₆-C₆₀arylene group which is unsubstituted or substituted by at least one group G, or a C₂-C₆₀heteroarylene group which is unsubstituted or substituted by at least one group G, preferably a divalent residue of benzene, biphenyl, terphenyl, naphthalene, anthracene, phenanthrene, triphenylene, dibenzofuran, and dibenzothiophene,

R⁵⁶ is a C₆-C₆₀aryl group which is unsubstituted or substituted by at least one selected from a C₆-C₆₀aryl group, a C₂-C₆₀heteroaryl group and a cyano group; a C₂-C₆₀heteroaryl group which is unsubstituted or substituted by at least one selected from a C₆-C₆₀aryl group and a C₂-C₆₀heteroaryl group; and a cyano group,

t is an integer of 1 to 5, preferably 1 to 3, more preferably 1 or 2, further preferably 1, and

two groups R⁵⁶ at adjacent carbon atoms may form a substituted or unsubstituted, saturated or unsaturated, aromatic or heteroaromatic ring.

In an embodiment of the compound of the general formula (Ie), the substituent of general formula (b) corresponds to general formula (b1):

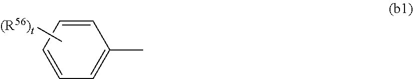

wherein R⁵⁶ and t have the same meanings as defined above.

In an embodiment of the compound of the general formula (Ie), A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R²¹, R²², R²³ and R²⁴, preferably R²³ is the substituent of general formula (b) or (b1).

In the above embodiment of the compound of the general formula (Ie), R¹, R², R³ and R⁴ are hydrogen atoms.

In the above embodiment of the compound of the general formula (Ie), R⁶, R¹⁰, R¹⁴ and R¹⁸ are hydrogen atoms.

In the above embodiment of the compound of the general formula (Ie), any of R²¹, R²², R²³ and R²⁴ not the substituent of general formula (b) or (b1), preferably R²¹, R²², and R²⁴ are hydrogen atoms.

Preferred meanings of R⁵⁶ are a nitrogen-comprising C₂-C₆₀heteroaryl group which is unsubstituted or substituted by at least one selected from a C₆-C₆₀aryl group and a C₂-C₆₀heteroaryl group, preferably a pyridyl group, a pyrimidyl group, a triazinyl group, a quinolinyl group, an iso quinolinyl group, or a phenanthrolinyl group, each of which may be substituted by at least one selected from a C₆-C₆₀aryl group and a C₂-C₆₀heteroaryl group.

Preferred meaning of R⁵⁶ is a cyano group.

Preferred meanings of R⁵⁶ are a fused C₆-C₆₀aryl group which is unsubstituted or substituted by at least one selected from a C₆-C₆₀aryl group, a C₂-C₆₀heteroaryl group and a cyano group, preferably a naphthyl group, an anthryl group, a triphenylenyl group, a pyrenyl group, a phenanthrenyl group, a bonzophenanthrenyl group, a benzochrysenyl group, a benzanthryl group, a fluorenyl group, a benzofluorenyl group, a 9,9-dimethylfluorenyl group, a 9,9-diphenylfluorenyl group, and a 9,9′-spirobifluorenyl group, each of which may be substituted by at least one selected from a C₆-C₆₀aryl group, a C₂-C₆₀heteroaryl group and a cyano group.

Preferred meanings of R⁵⁶ are a carbazolyl group which is unsubstituted or substituted by at least one selected from a C₆-C₆₀aryl group and a C₂-C₆₀heteroaryl group, preferably

a carbazolyl group wherein at least one of the two benzene rings forms a fused, aromatic or heteroaromatic ring, for example,

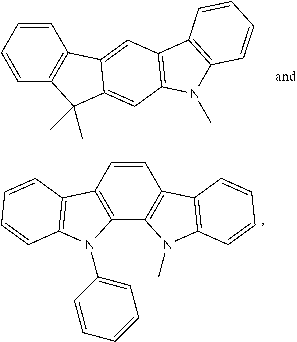

or a substituent of the following formula:

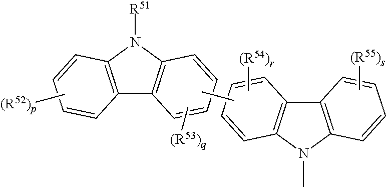

wherein

R⁵¹ is a C₆-C₆₀aryl group, preferably phenyl group

R⁵², R⁵³, R⁵⁴, and R⁵⁵ are independently of each other a C₁-C₂₅alkyl group, a C₆-C₆₀aryl group,

or a C₂-C₆₀heteroaryl group,

p is an integer of 0 to 4, preferably 0,

q is an integer of 0 to 2, preferably 0,

r is an integer of 0 to 2, preferably 0,

s is an integer of 0 to 4, preferably 0,

(R⁵²)₀, (R⁵³)₀, (R⁵⁴)₀, and (R⁵⁵)₀ mean the absence of R⁵², R⁵³, R⁵⁴, and R⁵⁵, respectively.

In the preferred compounds according to general formula (Ie), particularly preferred formulae (b) and (b1) are shown below.

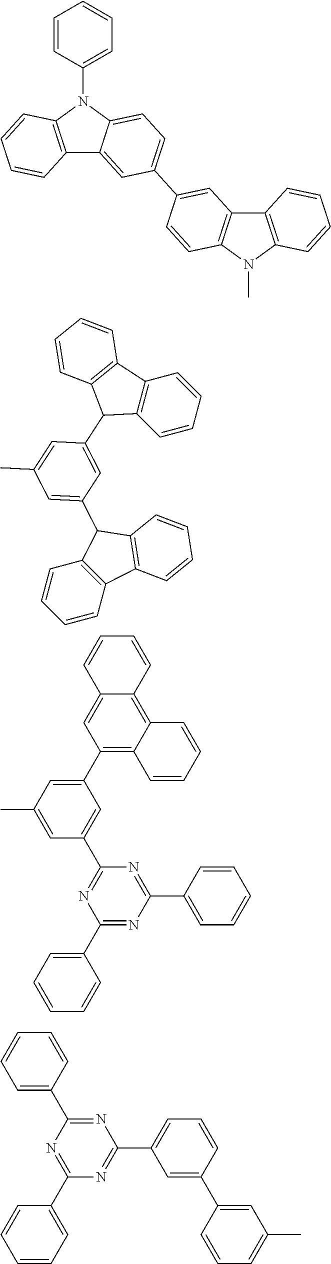

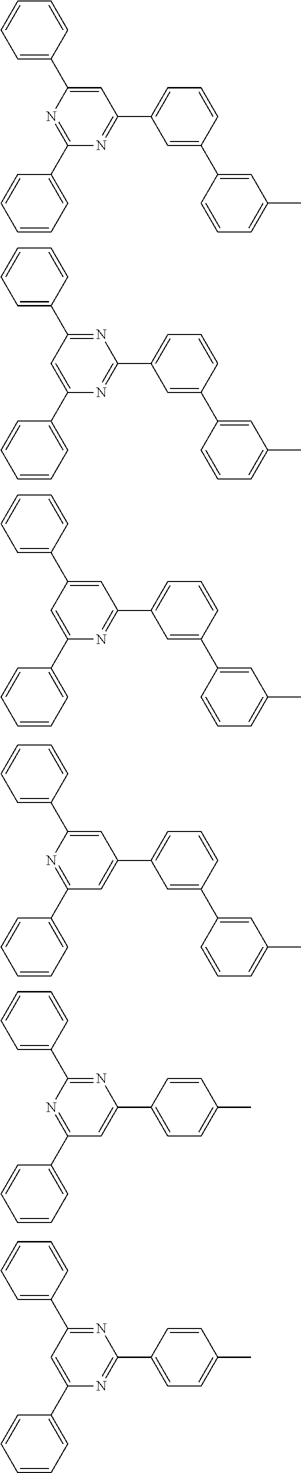

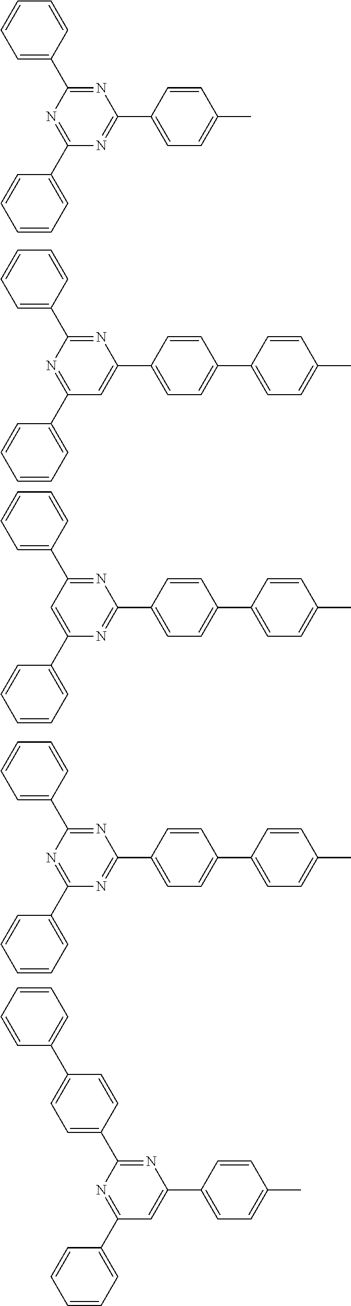

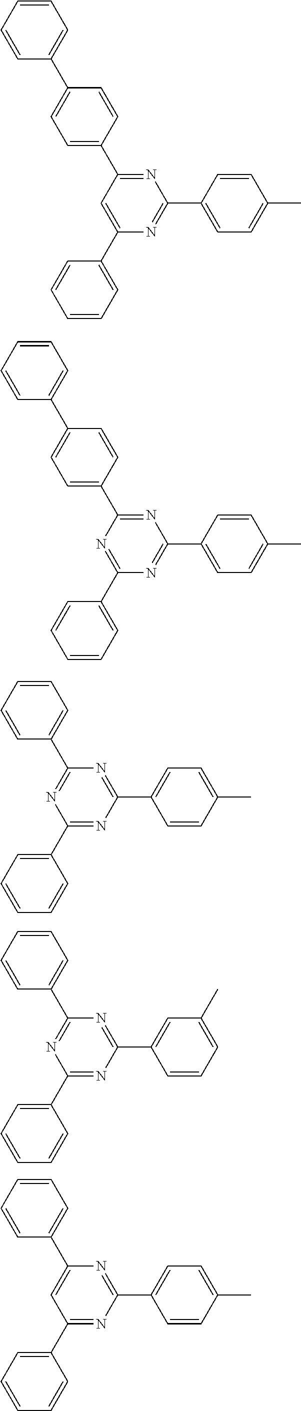

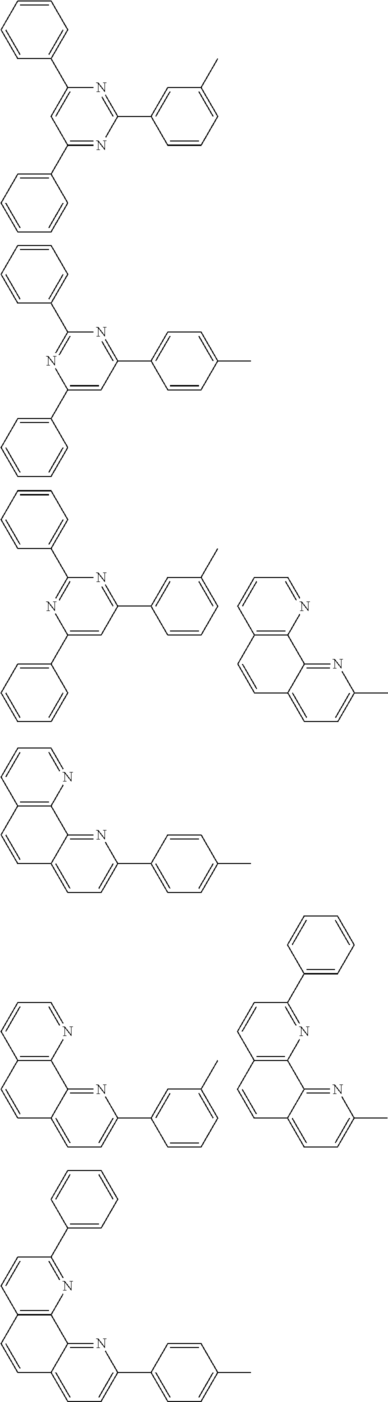

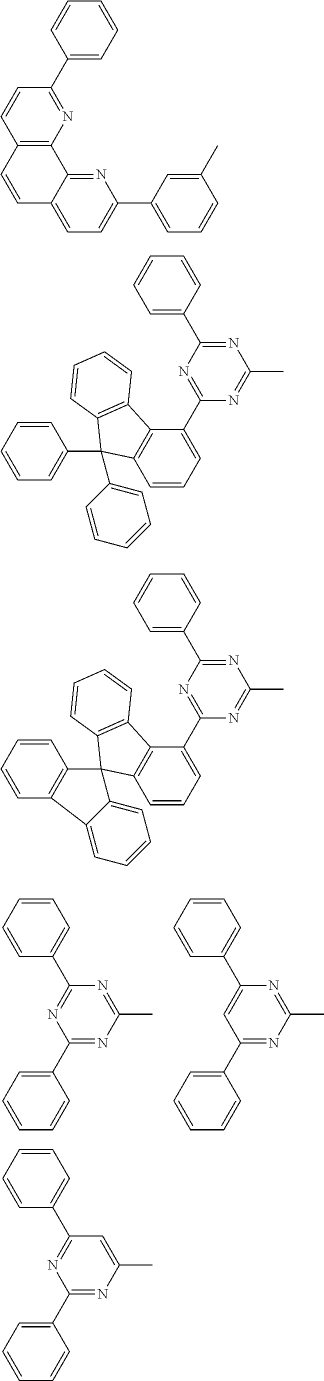

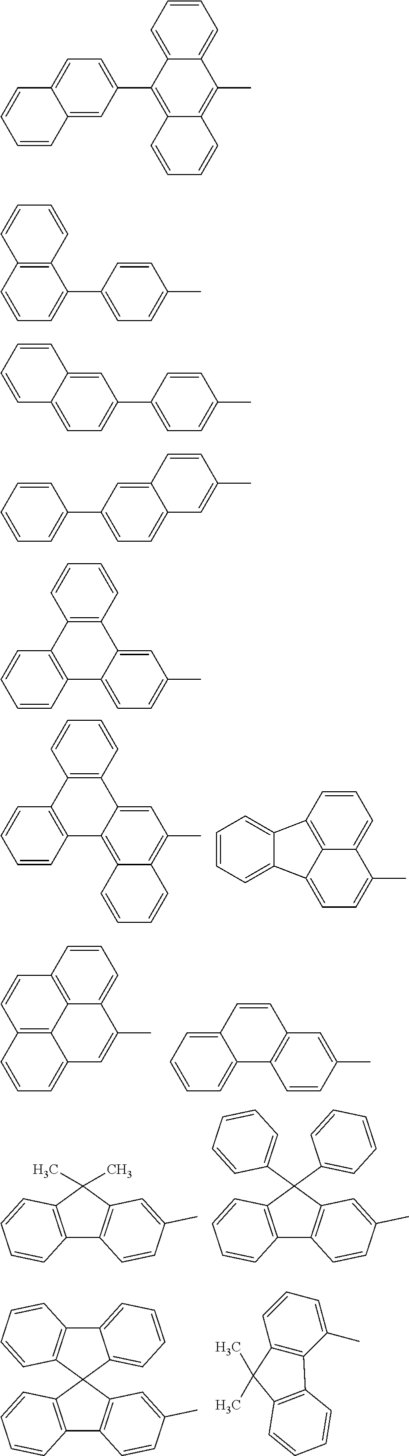

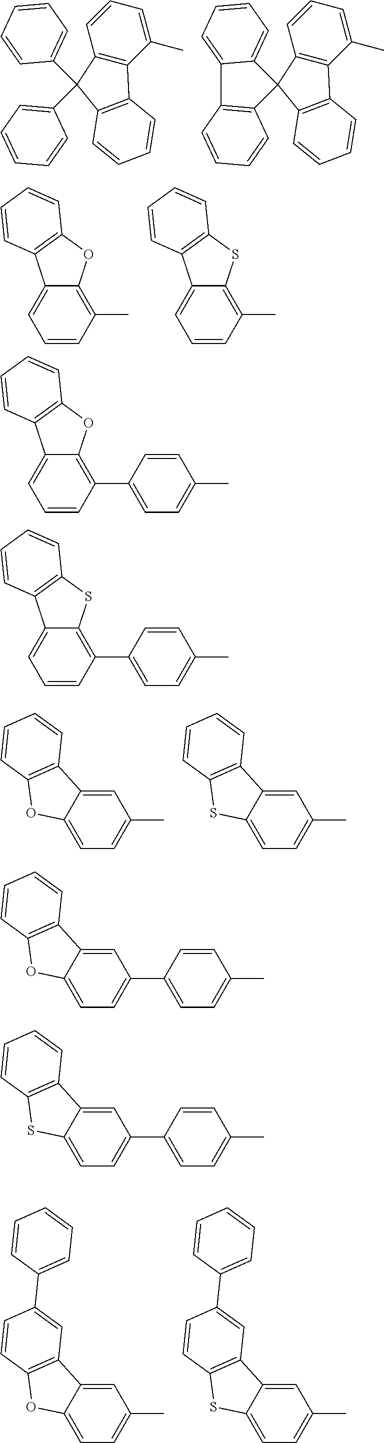

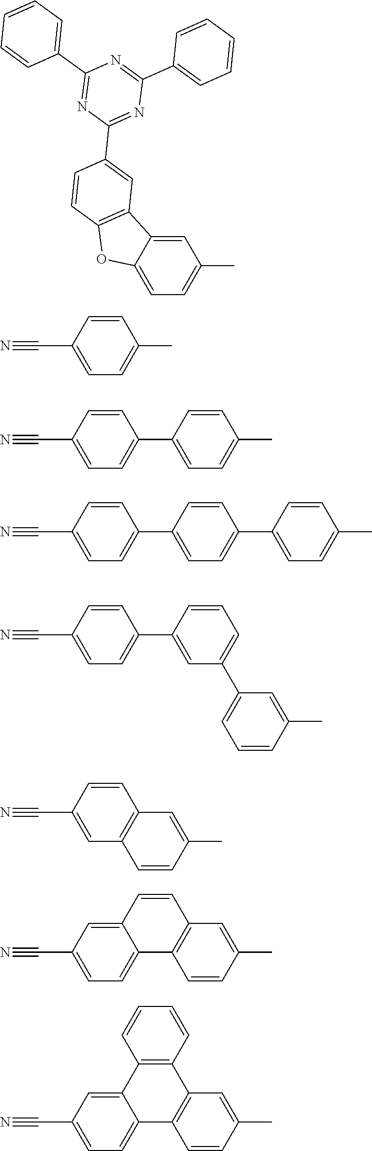

Particularly preferred formula (b) are shown below.

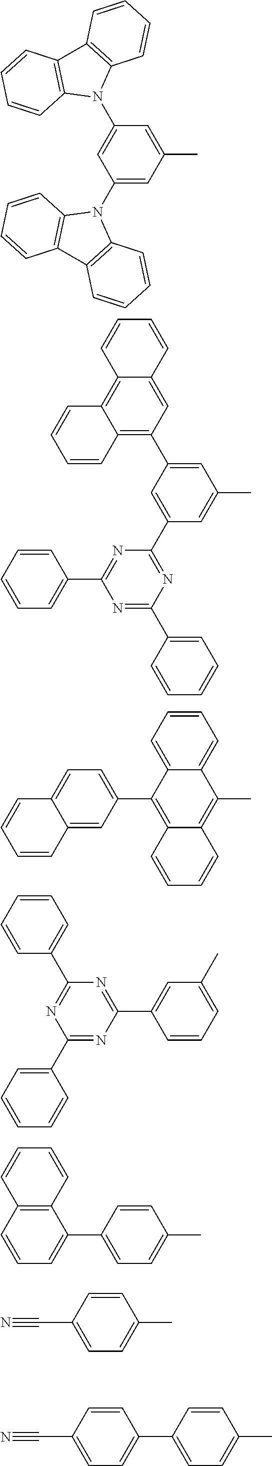

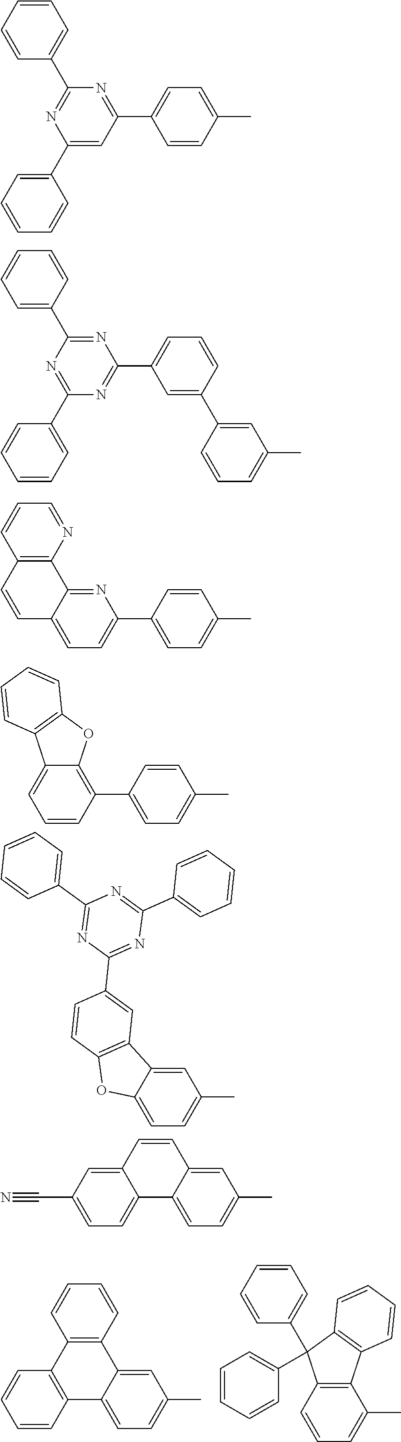

The embodiment, wherein X¹ is direct bond and X² is O, S, NR²⁵, or CR²⁶R²⁷ therefore corresponds to general formula (if):

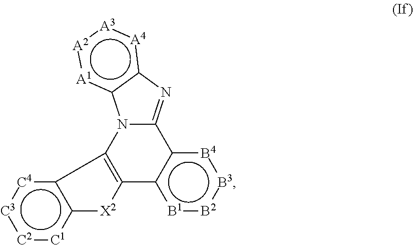

wherein A¹, A², A³, A⁴, B¹, B², B³, B⁴, C¹, C², C³ and C⁴ have the above mentioned meanings.

In an embodiment of the compound of the general formula (If), A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R¹, R², R³, R⁴, R⁶, R¹⁰, R¹⁴, R¹⁸, R²¹, R²², R²³, and R²⁴ is a substituent other than H. The substituent is mentioned above with respect to R¹, R², R³, R⁴, R⁶, R¹⁰, R¹⁴, R¹⁸, R²¹, R²², R²³, and R²⁴ of the general formula (I), and preferably represented by formula (b) or (b1).

The present invention therefore preferably relates to the electronic device according to the present invention, wherein X¹ is a direct bond and X² is O, S or NR²⁵, wherein R²⁵ has the same meaning as defined above.

Preferably, X² is O, S or NR²⁵, more preferably X² is O.

The preferred embodiment, wherein X¹ is O, S, NR²⁵, or CR²⁶R²⁷, preferably O, S or NR²⁵ and X² is a direct bond corresponds to general formula (Ig):

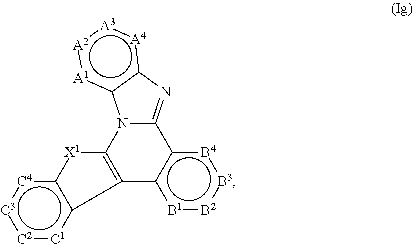

wherein A¹, A², A³, A⁴, B¹, B², B³, B⁴, C¹, C², C³ and C⁴ have the above mentioned meanings.

In an embodiment of the compound of the general formula (Ig), A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R¹, R², R³, R⁴, R⁶, R¹⁰, R¹⁴, R¹⁸, R²¹, R²², R²³ and R²⁴ is a substituent other than H. The substituent is mentioned above with respect to R¹, R², R³, R⁴, R⁶, R¹⁰, R¹⁴, R¹⁸, R²¹, R²², R²³, and R²⁴ of the general formula (I), and preferably represented by formula (b) or (b1).

The present invention therefore preferably relates to the electronic device according to the present invention, wherein X¹ is O, S or NR²⁵ and X² is a direct bond, wherein R²⁵ has the same meaning as defined above.

Preferably, X¹ is O, S or NR²⁵, more preferably X¹ is O.

Preferred meanings of R²⁵ are H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G,

or —SiR²⁸R²⁹R³⁰, wherein G, E, D, R²⁸, R²⁹ and R³⁰ have independently of each other the meanings as defined above.

Particularly preferred meanings of R²⁵ are a C₆-C₆₀aryl group which is unsubstituted or substituted by at least one group G, or a C₂-C₆₀heteroaryl group which is unsubstituted or substituted by at least one group G, wherein G has the meaning as mentioned above and the C₂-C₆₀heteroaryl group preferably comprises N as heteroatom.

Particularly preferred meanings of R²⁵ are a fused C₆-C₆₀aryl group, a nitrogen-comprising C₂-C₆₀heteroaryl group, a C₆-C₆₀aryl group which is substituted by at least one of a fused C₆-C₆₀aryl group and a nitrogen-comprising C₂-C₆₀heteroaryl group, or a C₂-C₆₀heteroaryl group which is substituted by at least one of a fused C₆-C₆₀aryl group and a nitrogen-comprising C₂-C₆₀heteroaryl group.

Particularly preferred meanings of R²⁵ are shown in the following:

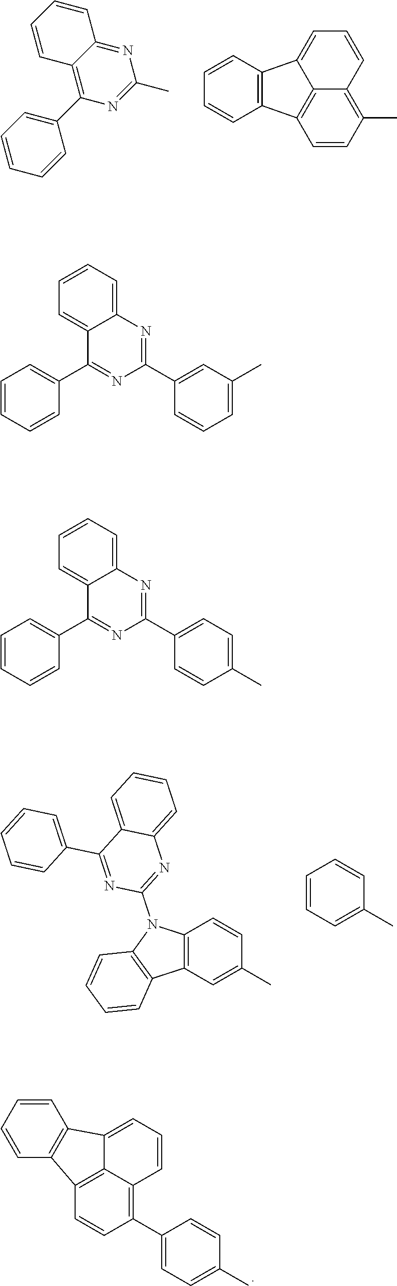

In the preferred compounds according to general formulae (Ie), (If) and (Ig), particularly preferred CR²⁶R²⁷ is C(CH₃)₂ and particularly preferred R²⁵ is shown below.

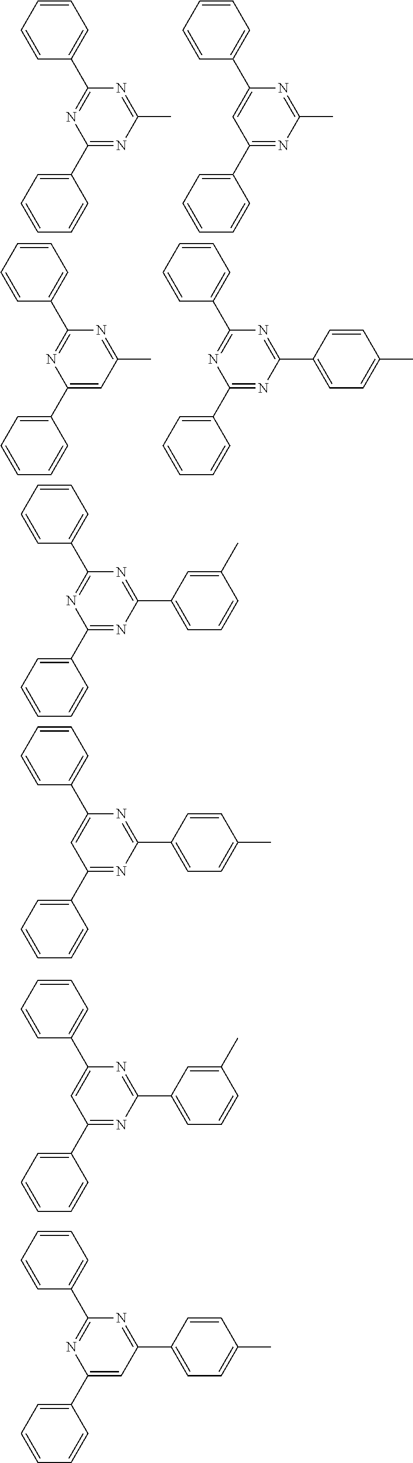

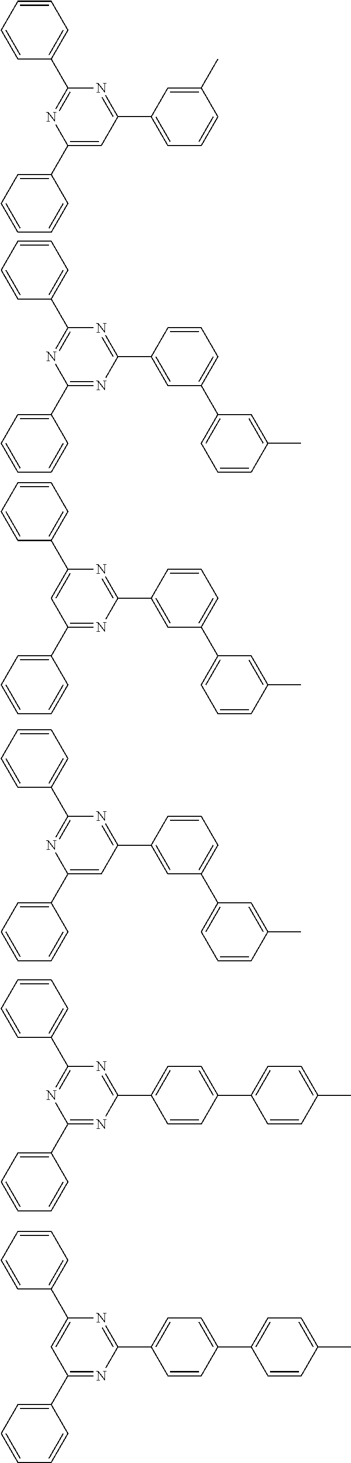

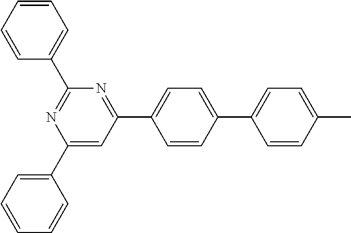

According to a further preferred embodiment, R²⁵ forms a five or six membered, substituted or unsubstituted, saturated or unsaturated, aromatic or heteroaromatic ring with R²¹, R²², R²³ or R²⁴, if present. Preferably R²⁵ and R²¹, R²², R²³ or R²⁴ form five membered saturated ring to which aromatic or heteroaromatic rings or ring systems with 4 to 30 carbon atoms may be fused. Preferably, R²⁵ and R²¹ or R²⁵ and R²⁴ form a five or six membered, substituted or unsubstituted, saturated or unsaturated, aromatic or heteroaromatic ring, particularly preferably form five membered saturated ring to which aromatic or heteroaromatic rings or ring systems with 4 to 30 carbon atoms may be fused.

In case that X¹ is a direct bond, the mostly preferred the moiety that is formed by R²⁵ and R²¹ corresponds to the following formula:

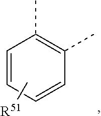

wherein the dashed lines describe the bonding to N in respect to R²⁵ and to C¹, and wherein R⁵¹ may be one or more, preferably at most 4, substituents selected from H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰, wherein G, E, D, R²⁸, R²⁹ and R³⁰ have independently of each other the meanings as defined above. These substituents may be bonded by carbon-carbon-bonds or may be fused to the aromatic ring.

In case that X² is a direct bond, the mostly preferred the moiety that is formed by R²⁵ and R²⁴ corresponds to the following formula:

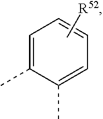

wherein the dashed lines describe the bonding to N in respect to R²⁵ and to C⁴, and wherein R⁶¹ may be one or more, preferably at most 4, substituents selected from H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, or —SiR²⁸R²⁹R³⁰, wherein G, E, D, R²⁸, R²⁹ and R³⁰ have independently of each other the meanings as defined above. These substituents may be bonded by carbon-carbon-bonds or may be fused to the aromatic ring.

Beside the electronic device according to the present invention the above mentioned objects are also solved by compounds according to general formula (IV):

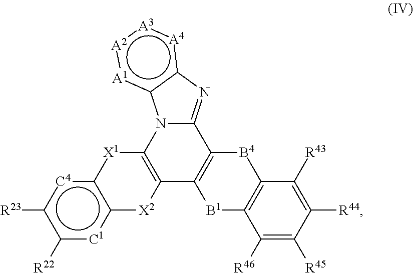

wherein

A¹, A², A³, A⁴ form an aromatic or heteroaromatic six membered ring, wherein A¹ is N or CR¹, A² is N or CR², A³ is N or CR³ and A⁴ is N or CR⁴,

B¹ is a direct bond and B⁴ is NR¹⁷, N, O, S, CR¹⁸ or CR¹⁹R²⁰, or B¹ is NR⁵, N, O, S, CR⁶ or CR⁷R⁸ and B⁴ is a direct bond,

C¹ is N or CR²¹ and C⁴ is N or CR²⁴,

X¹, X² direct bond, O, S, NR²⁵, CR²⁶R²⁷, wherein one of X¹ and X² is a direct bond and the other one is O, S, NR²⁵, or CR²⁶R²⁷,

wherein R¹, R², R³, and R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, D is —CO—, —COO—, —S—, —SO—, —SO₂—, —O—, —CR³¹═CR³²—, —NR³³—, —SiR²⁸R²⁹—, —POR³⁴—, or —C≡C—,

E is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, an unsubstituted C₆-C₆₀aryl group, a C₆-C₆₀aryl group which is substituted by J or C₁-C₁₈alkyl, a C₁-C₁₈alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,

J is —CF₃, —CF₂CF₃, —CF₂CF₂CF₃, —CF(CF₃)₂, —(CF₂)₃CF₃ or —C(CF₃)₃,

G is E, a C₁-C₁₈alkyl group, or C₁-C₁₈alkyl which is interrupted by O,

R²⁸, R²⁹ and R³⁰ are independently of each other a C₁-C₁₈alkyl group, a C₆-C₁₈aryl group, or a C₆-C₁₈aryl group which is substituted by C₁-C₁₈alkyl,

R³¹ and R³² are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,

R³³, R³⁴, R³⁵, and R³⁹ are independently of each other H, a C₆-C₁₈aryl group, a C₆-C₁₈aryl group which is substituted by C₁-Cₐ₈alkyl or C₁-C₁₈alkoxy, a C₁-C₁₈alkyl group, or a C₁-C₁₈alkyl group which is interrupted by —O—,

R³⁶ is H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,

R³⁷, R³⁸, R⁴⁰, R⁴¹, and R⁴² are independently of each other H, a C₆-C₁₈aryl group, a C₆-C₁₈aryl which is substituted by C₁-C₁₈alkyl or C₁-C₁₈alkoxy, a C₁-C₁₈alkyl group, or a C₁-C₁₈alkyl group which is interrupted by —O—,

or R³⁷, R³⁸ together form a five or six membered ring,

or R⁴¹, R⁴² together form a five or six membered ring,

or two of R¹, R², R³ and R⁴, if present at adjacent carbon atoms, form a five or six membered, substituted or unsubstituted, saturated or unsaturated, aromatic or heteroaromatic ring,

R⁵, R⁶, R⁷, R⁸, R¹⁷, R¹⁸, R¹⁹, R²⁰, R²¹, R²², R²³, R²⁴, R²⁵, R²⁶, and R²⁷ are independently of each other H, E, a C₆-C₆₀aryl group which is unsubstituted or substituted by at least one group G, a C₂-C₅₆heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,

or two of R²¹, R²², R²³ and R²⁴, if present at adjacent carbon atoms, form a five or six membered, substituted or unsubstituted, saturated or unsaturated, aromatic or heteroaromatic ring,

R⁴³, R⁴⁴, R⁴⁵, and R⁴⁶ are independently of each independently of each other H, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,

or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,

or R²⁵ may form a five or six membered, substituted or unsubstituted, saturated or unsaturated, aromatic or heteroaromatic ring with R²¹, R²², R²³ or R²⁴,

or R⁵ or R¹⁷ may form a five or six membered, substituted or unsubstituted, saturated or unsaturated, aromatic or heteroaromatic ring with R⁴³, R⁴⁴, R⁴⁵ or R⁴⁶.

The compounds according to general formula (IV) are a specific selection of the compounds according to general formula (I) that are present in the electronic device according to the present invention. The common feature of compounds according to general formula (IV) is that B¹, B⁴, together with the adjacent carbon atoms, form an aromatic or heteroaromatic five membered ring. According to a preferred embodiment, the electronic device according to the present invention comprises at least one compound of general formula (IV).

In respect of A¹, A², A³, A⁴, B¹, B⁴, C¹, C⁴, X¹, X², R²², R²³, R⁴³, R⁴⁴, R⁴⁵ and R⁴⁶ in formula (IV) the general and preferred embodiments as outlined in respect of compounds of formula (I) apply correspondingly.

According to the present invention, the compounds of general formulae (I), (II) or (III) comprise at least six, preferably at least seven, more preferably at least eight, more preferably at least nine, aromatic or heteroaromatic, saturated or unsaturated rings that are connected by direct bonds or fused together.

Therefore, the present invention preferably relates to the electronic device according to the present invention, wherein the compound of general formulae (I), (II) or (III) comprises at least six, preferably at least seven, more preferably at least eight, more preferably at least nine, aromatic or heteroaromatic, saturated or unsaturated rings that are connected by direct bonds or fused together.

Particularly preferred compounds according general formulae (I), (Ia), (Ib), (Ic), (II), (IIa), (III) and (IV) are shown in the following:

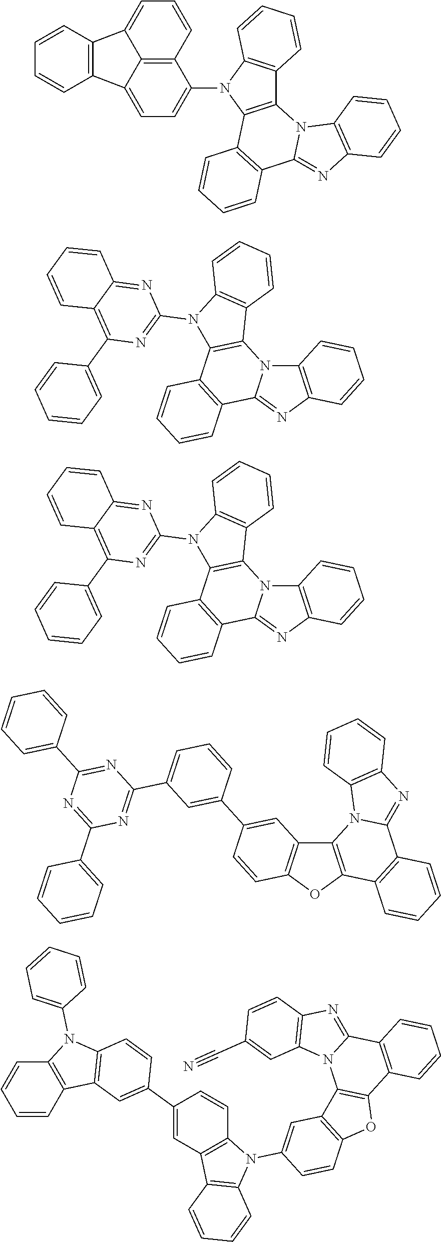

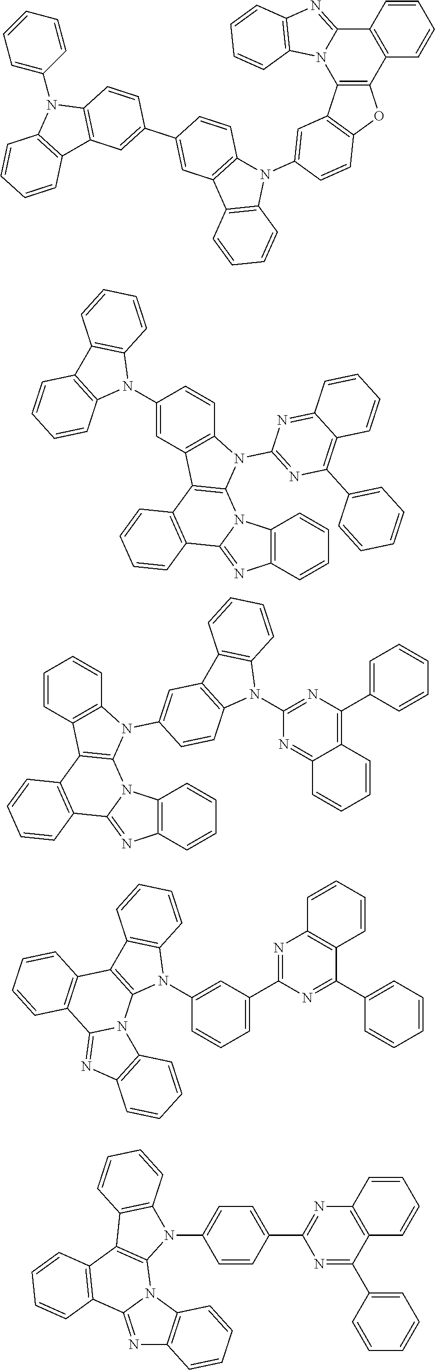

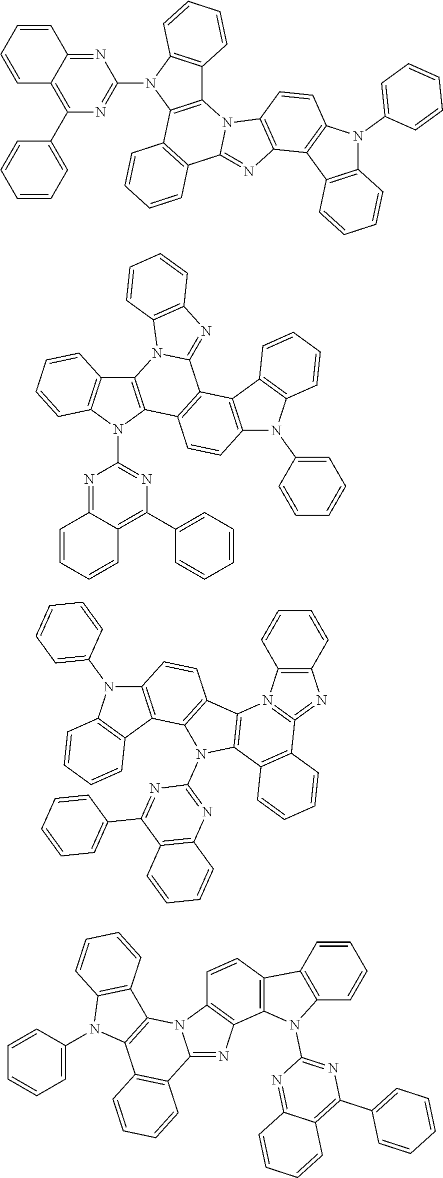

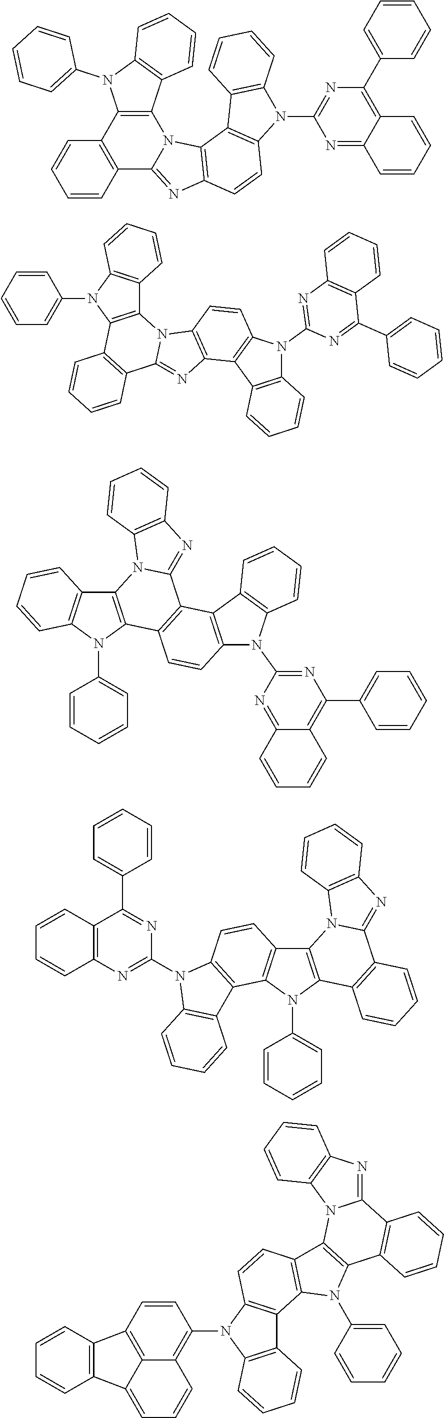

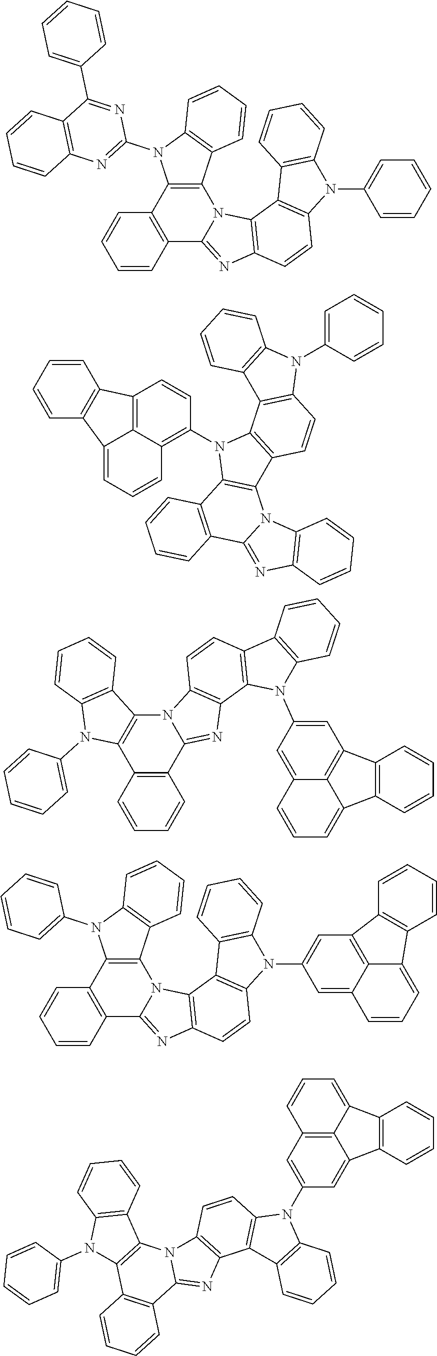

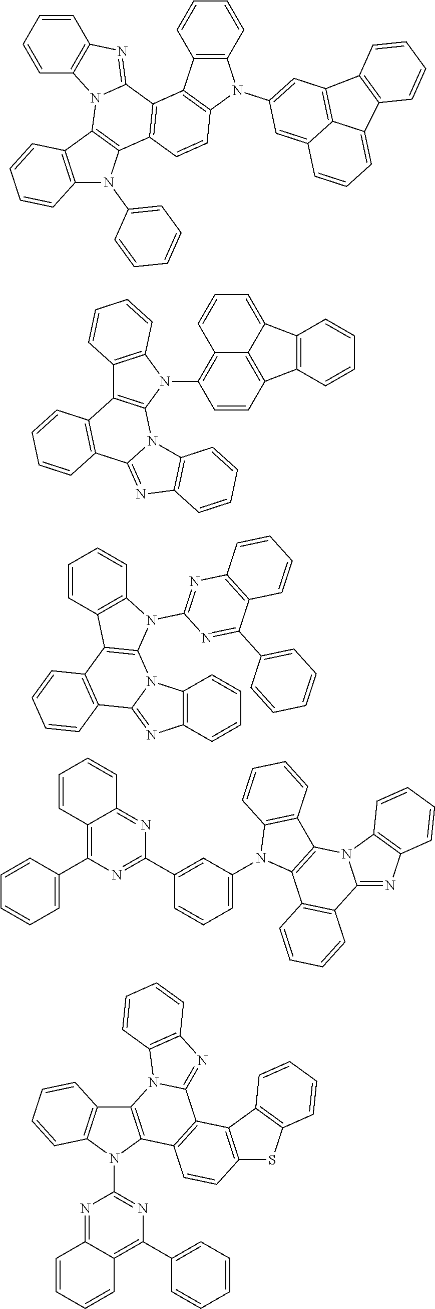

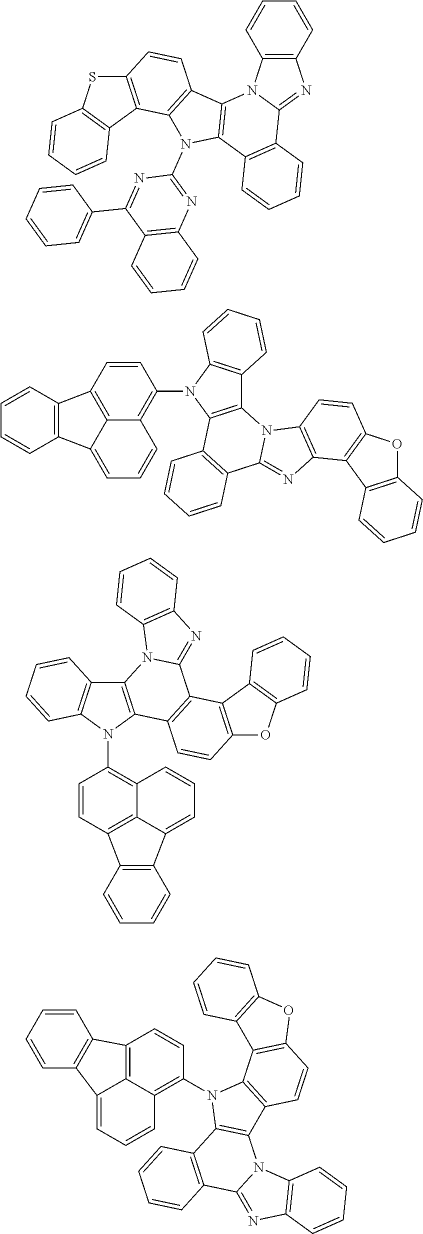

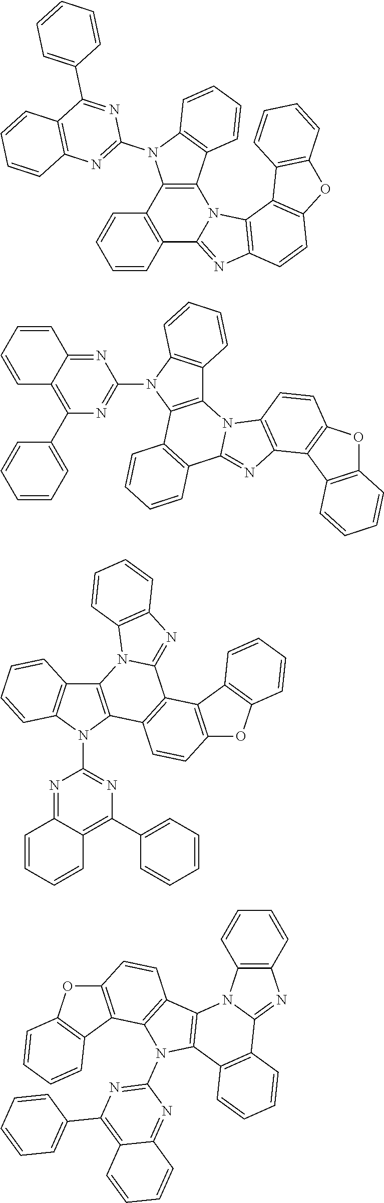

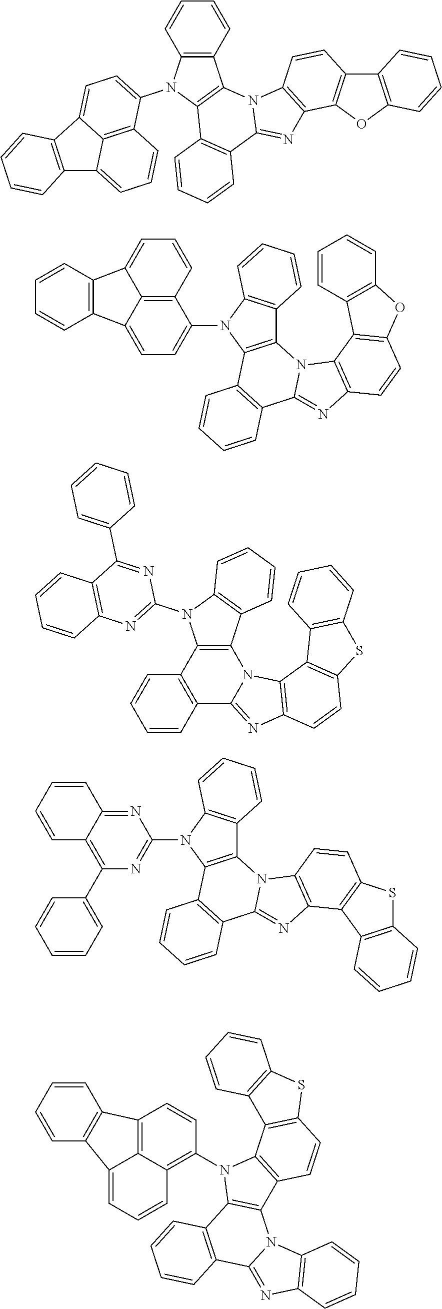

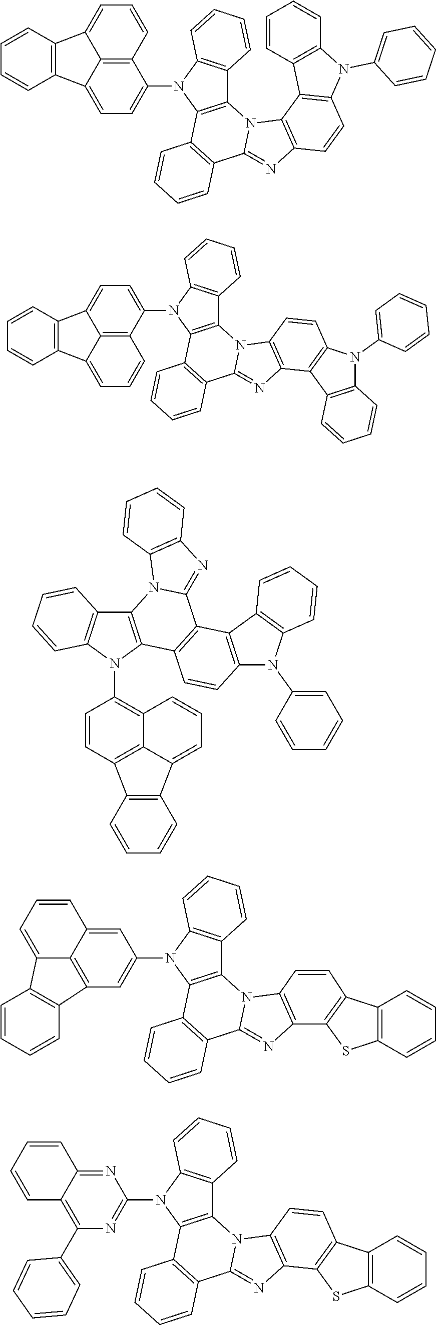

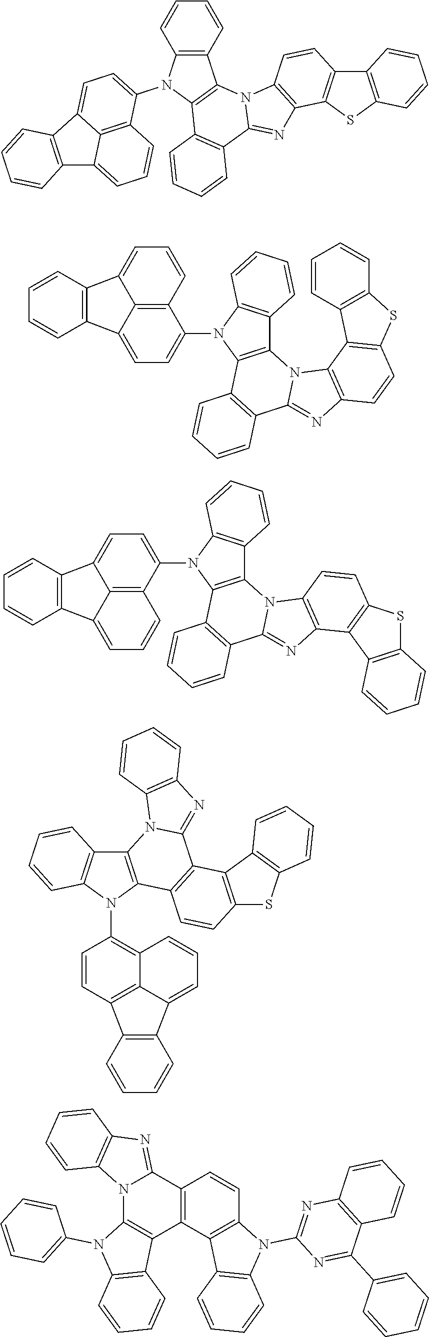

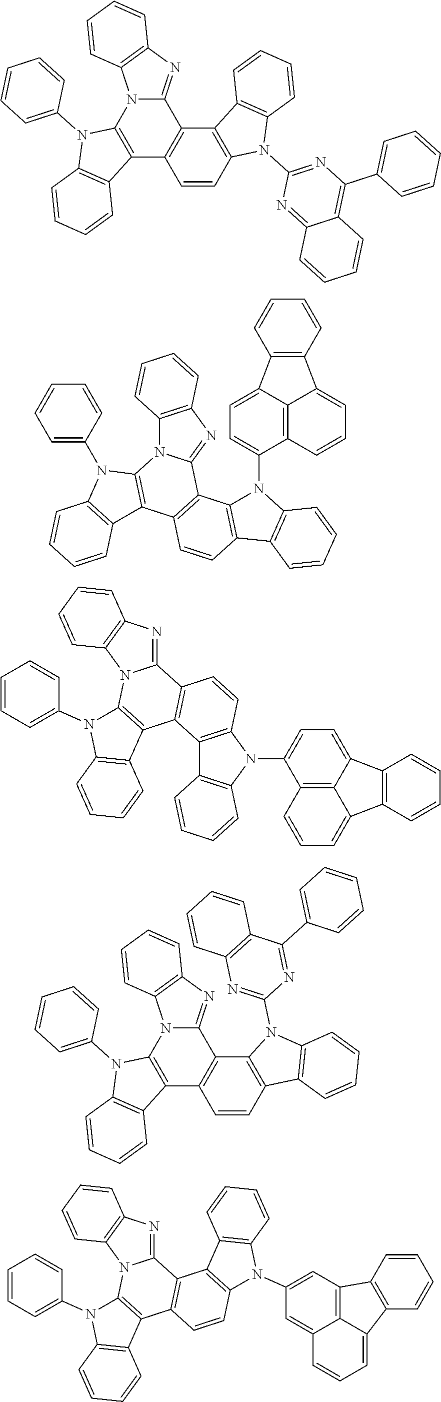

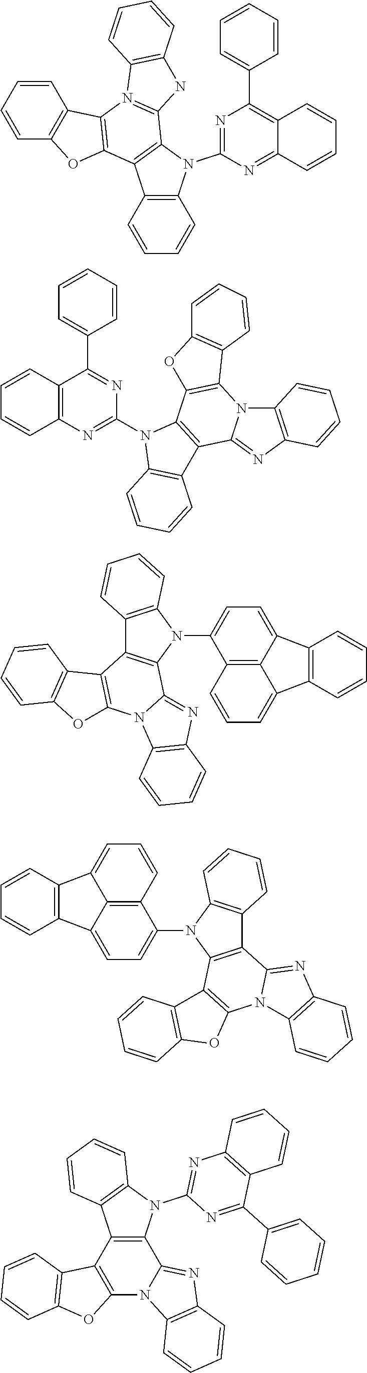

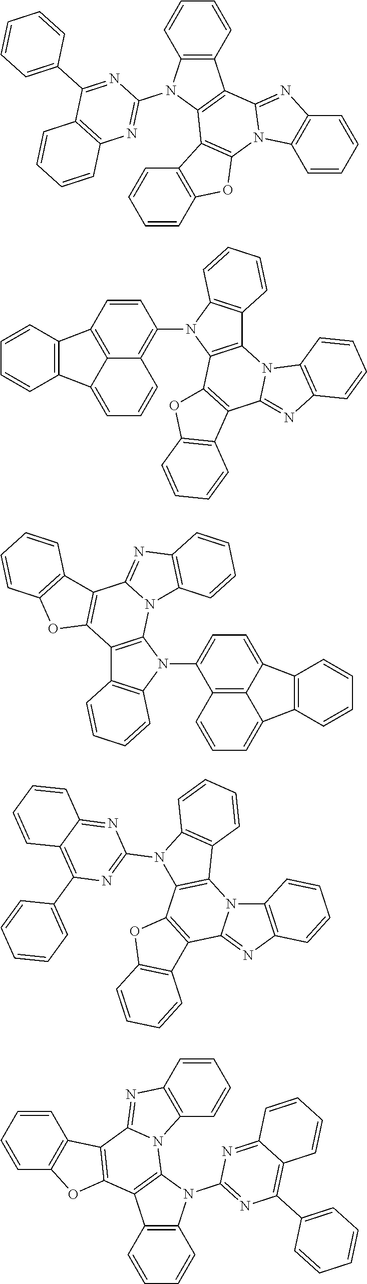

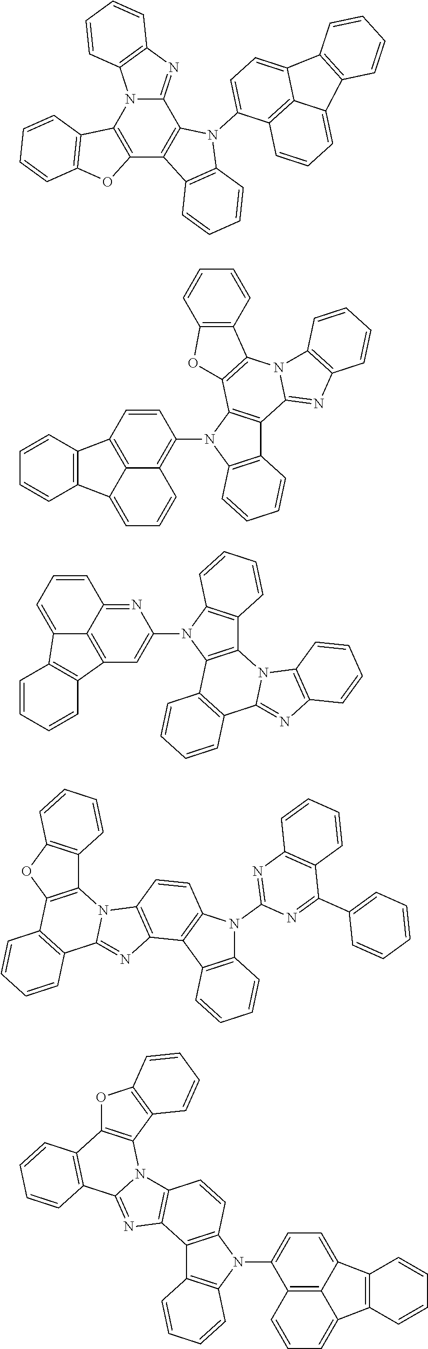

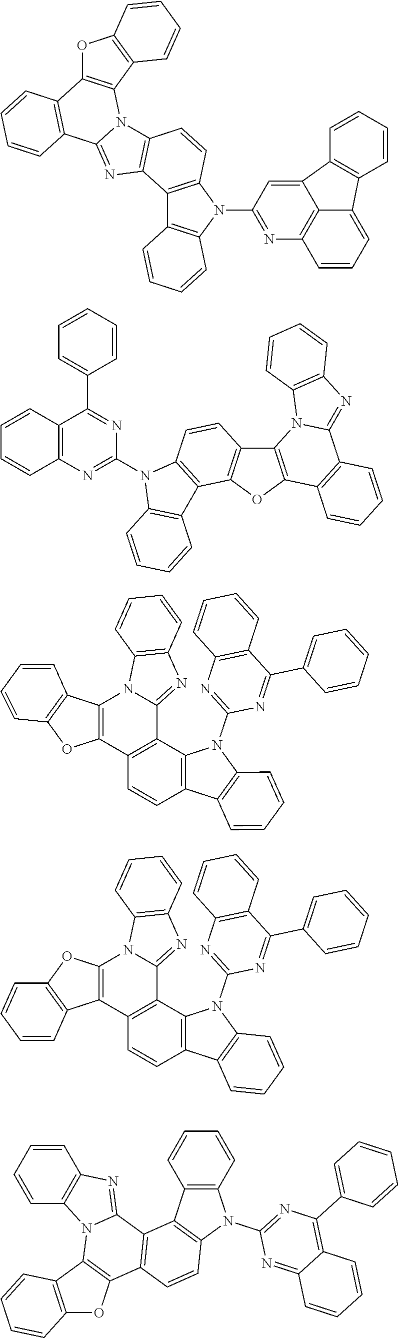

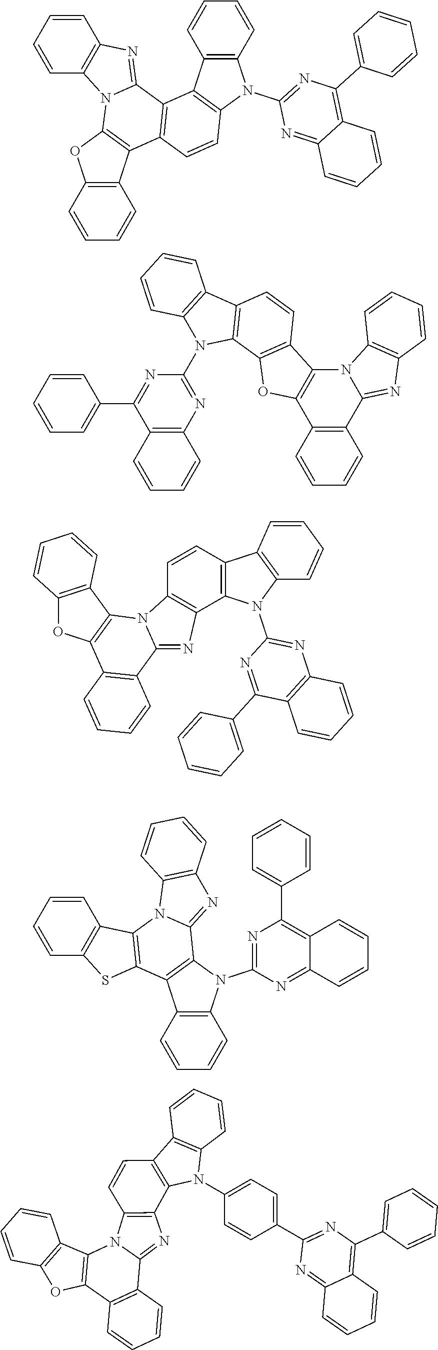

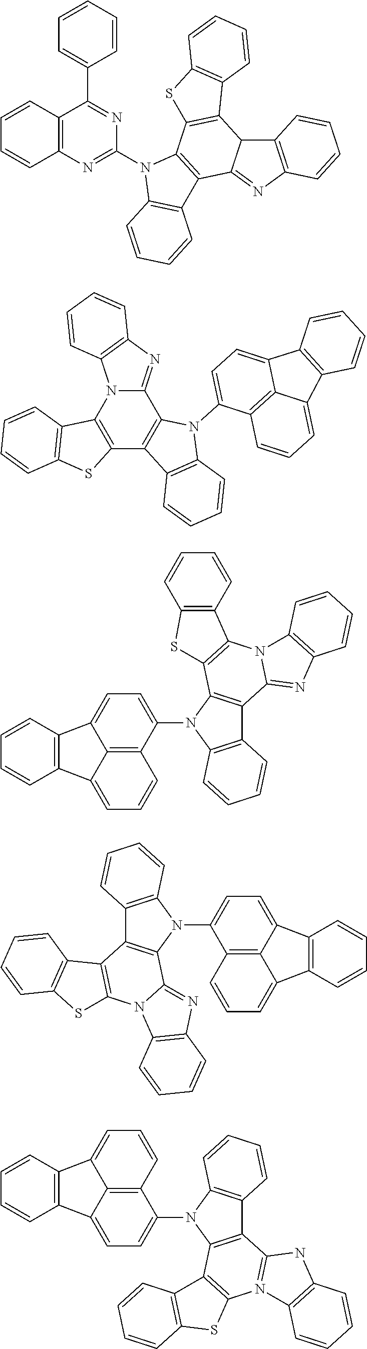

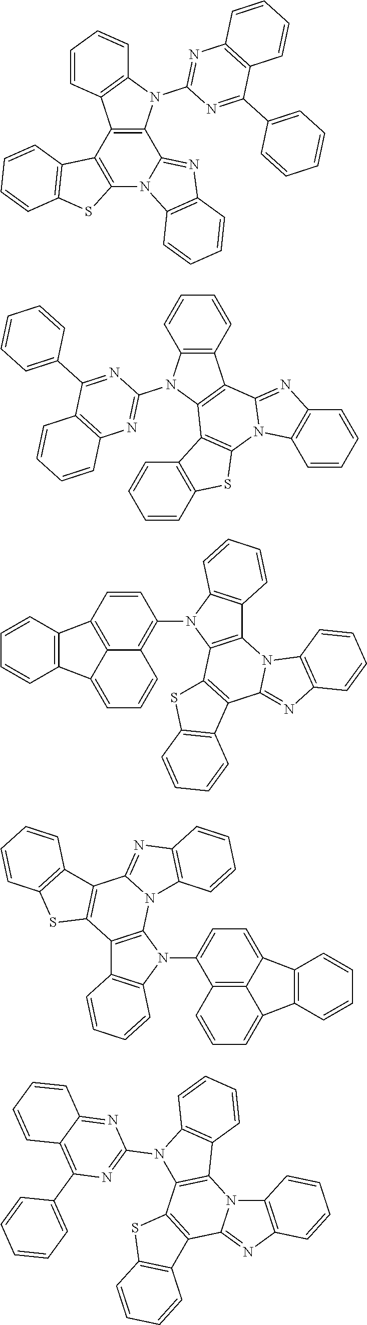

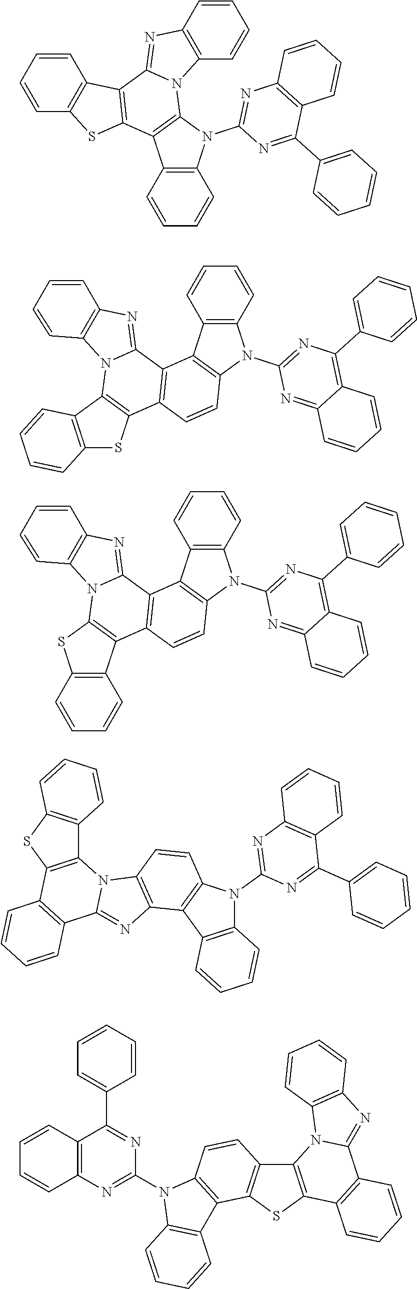

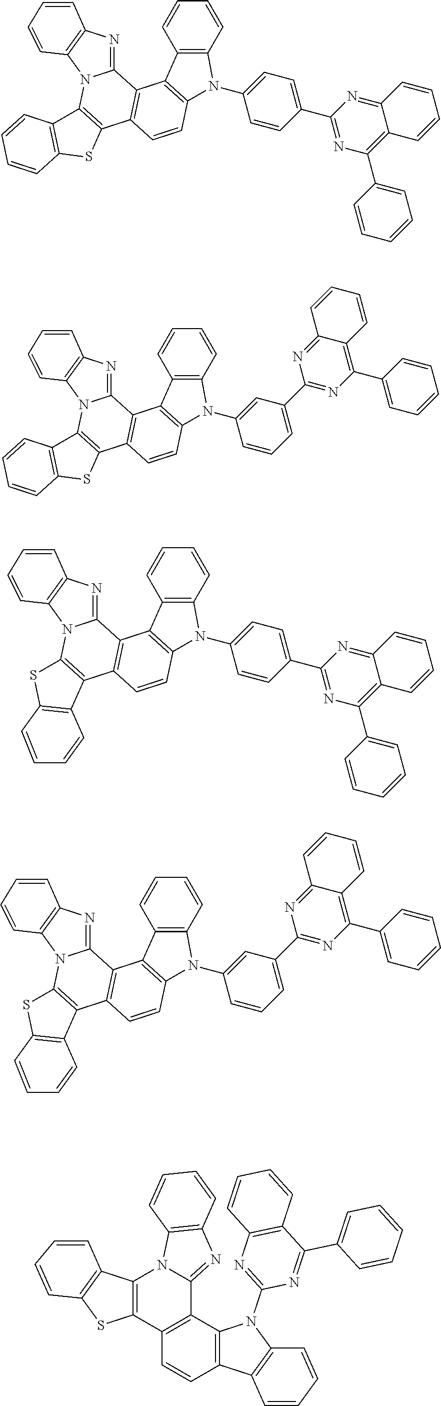

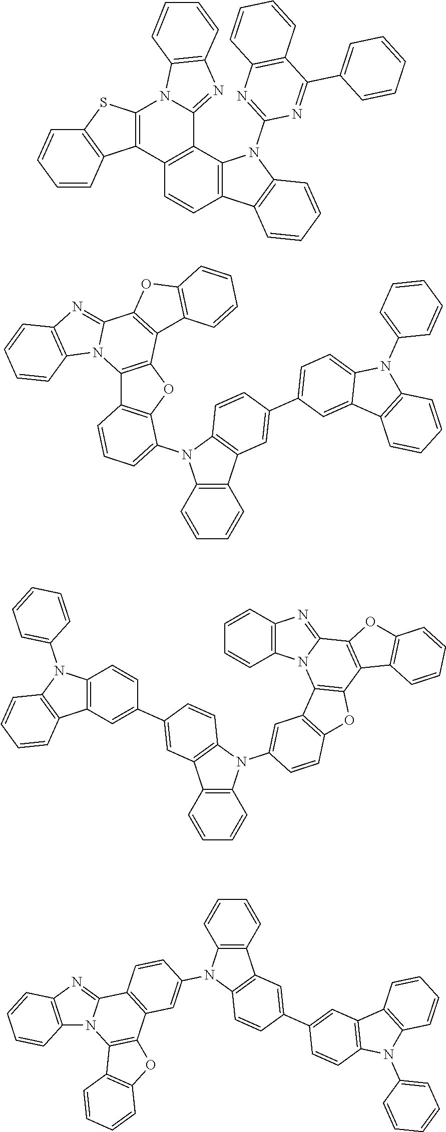

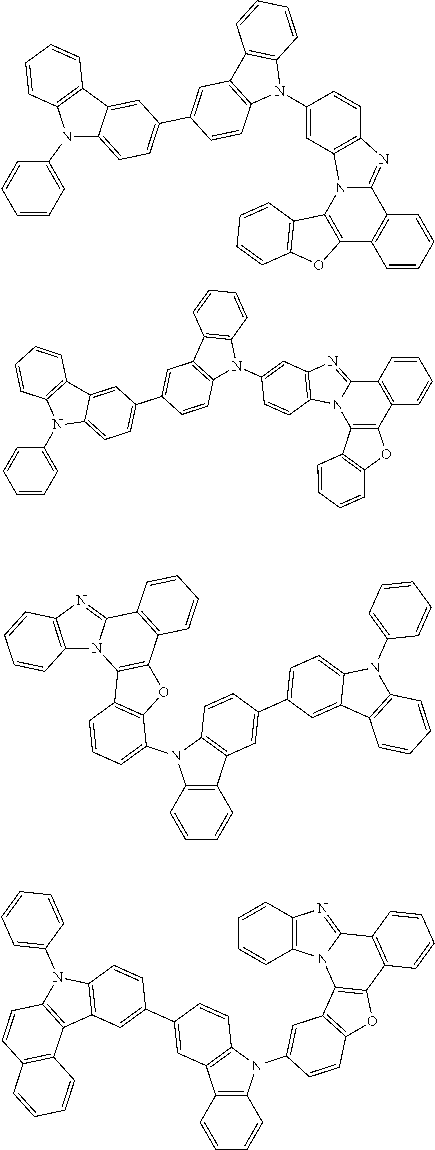

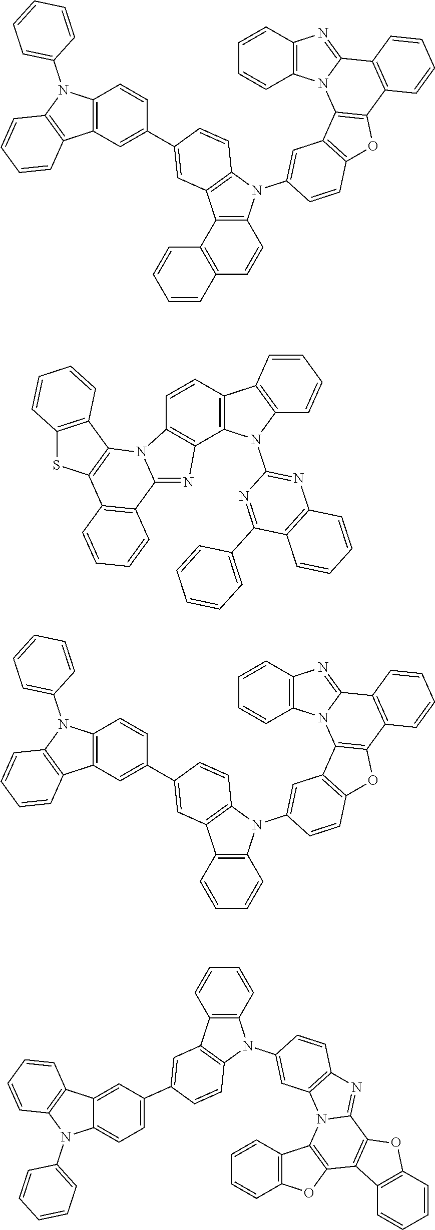

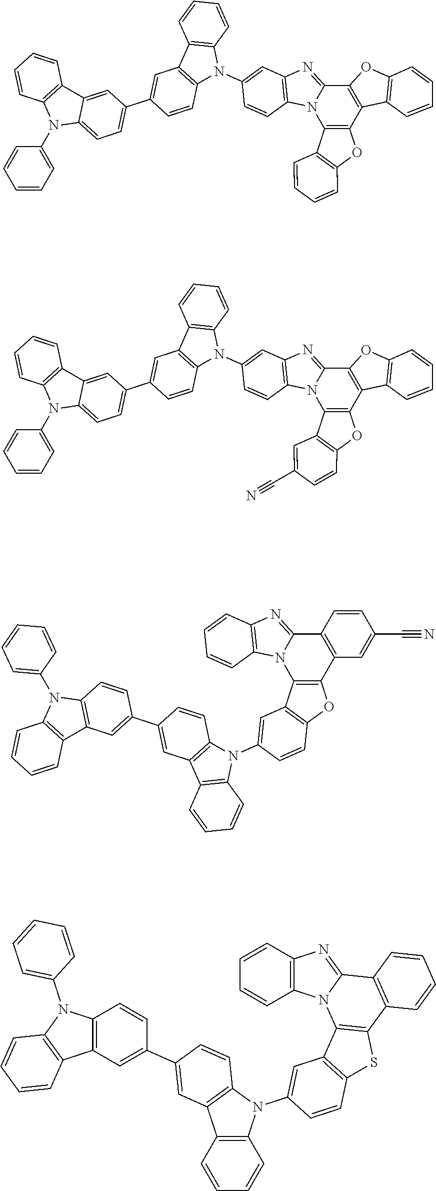

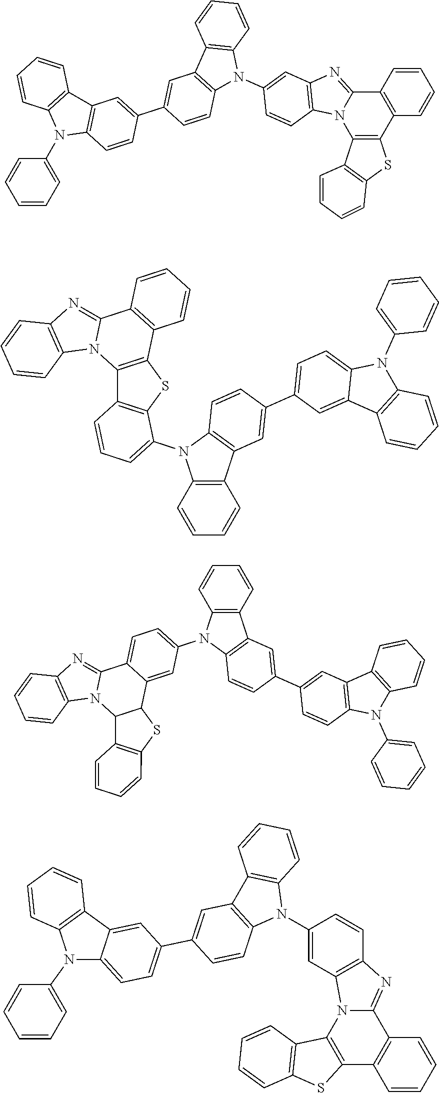

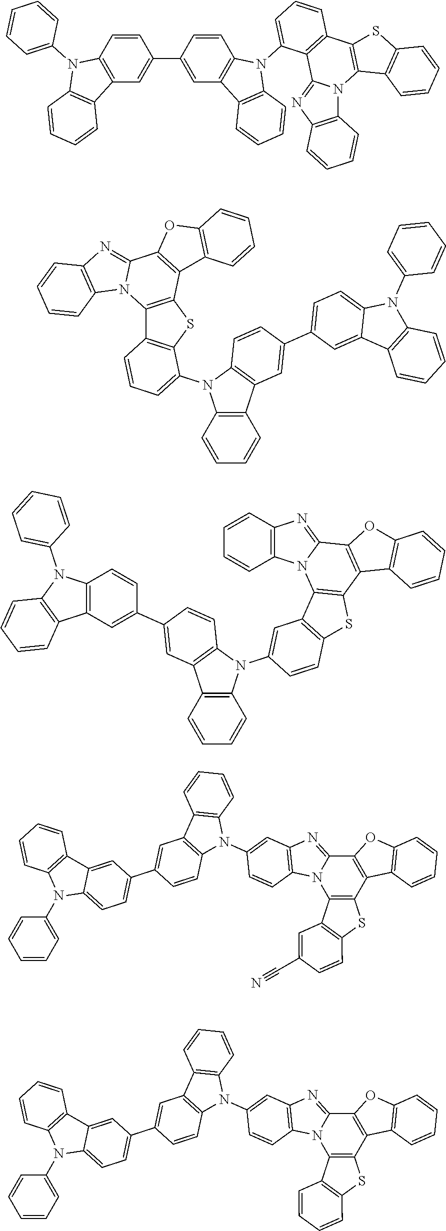

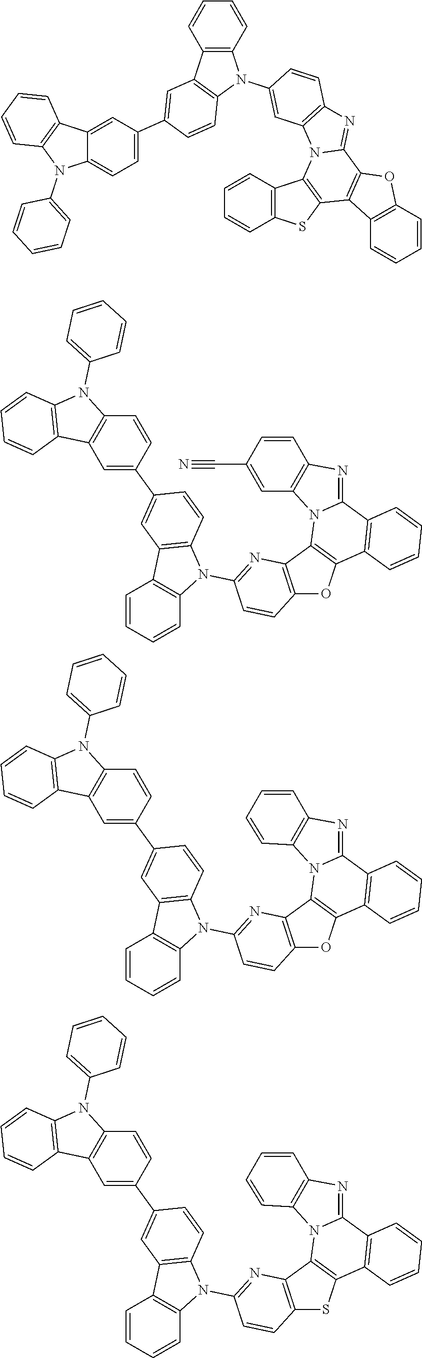

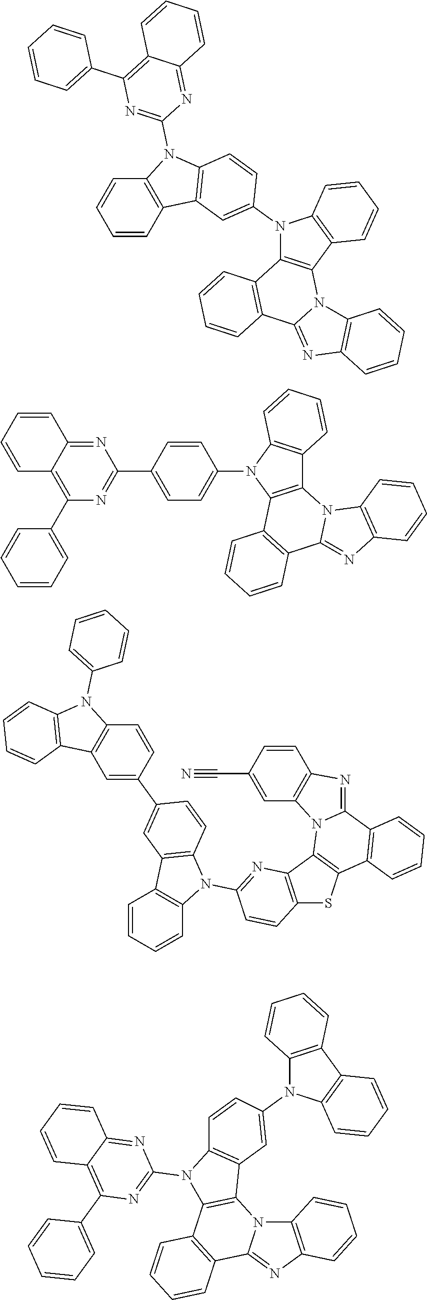

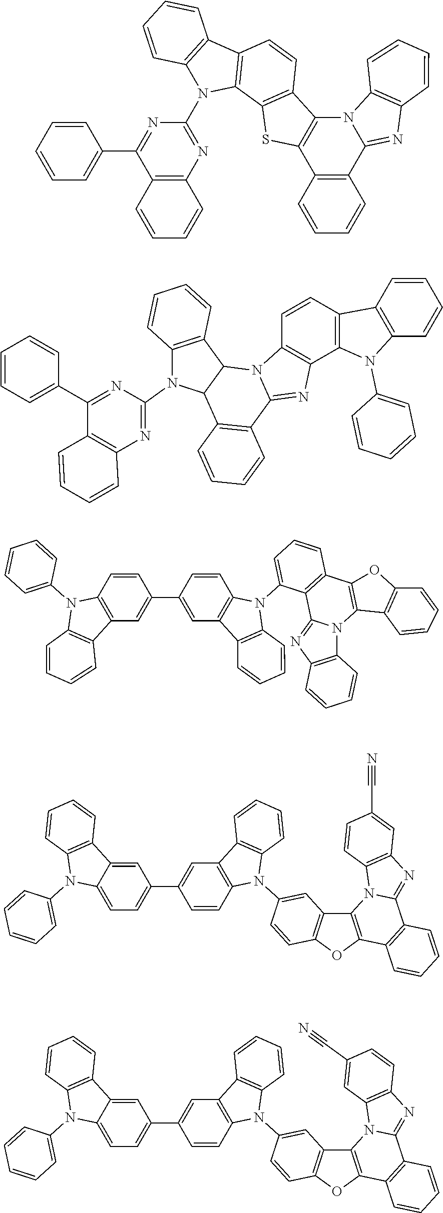

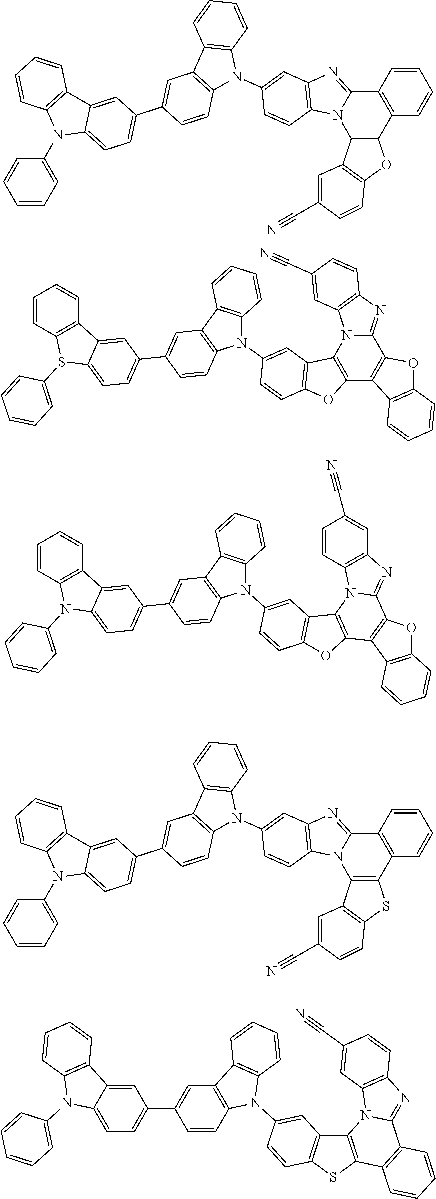

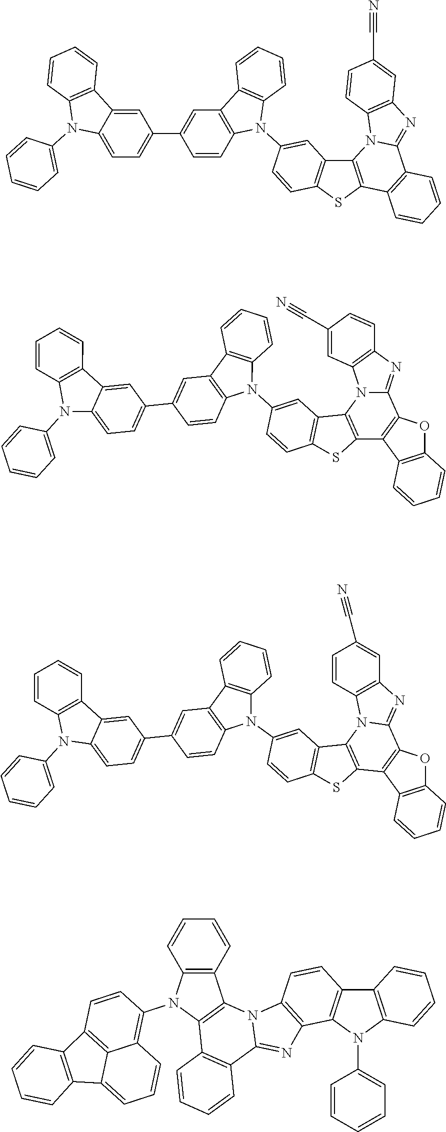

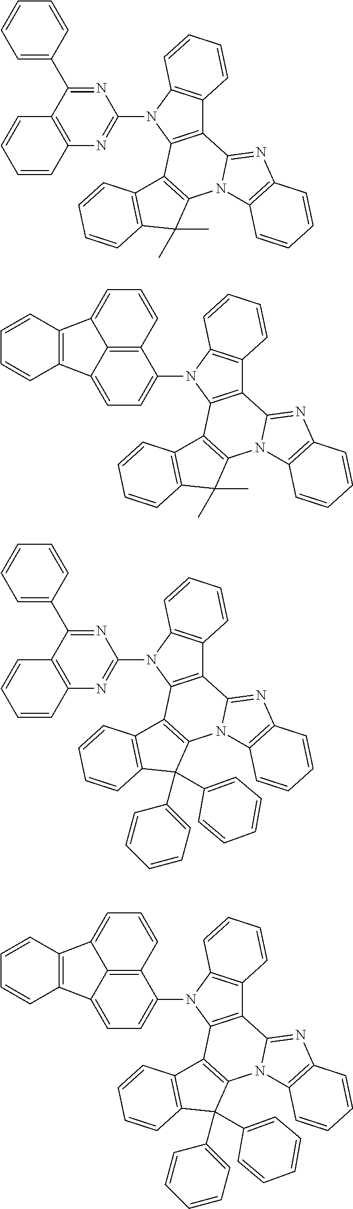

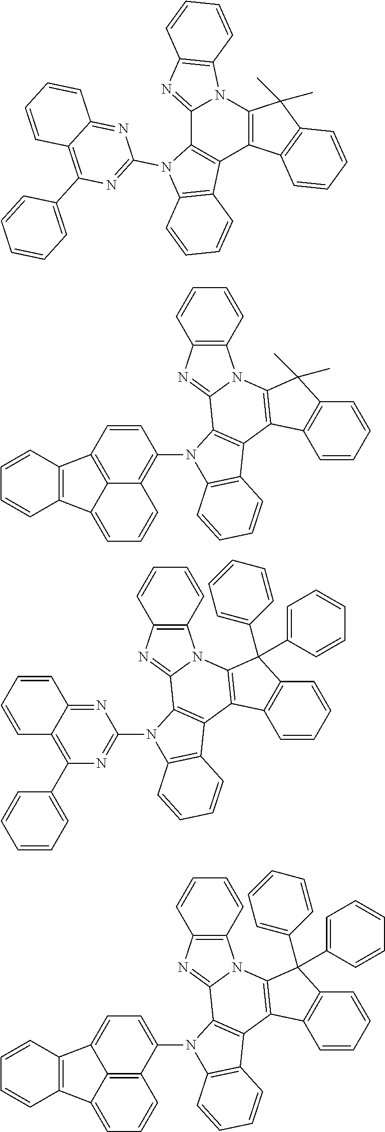

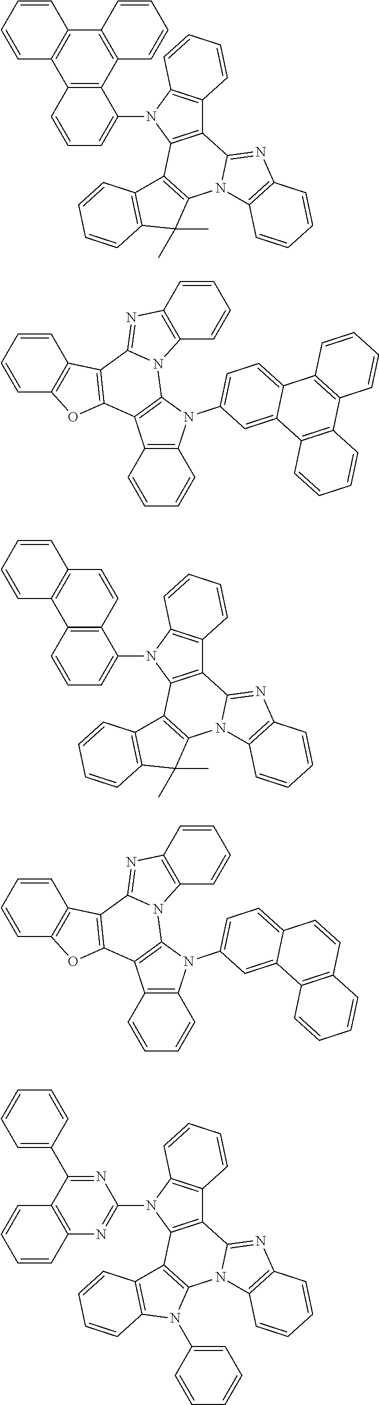

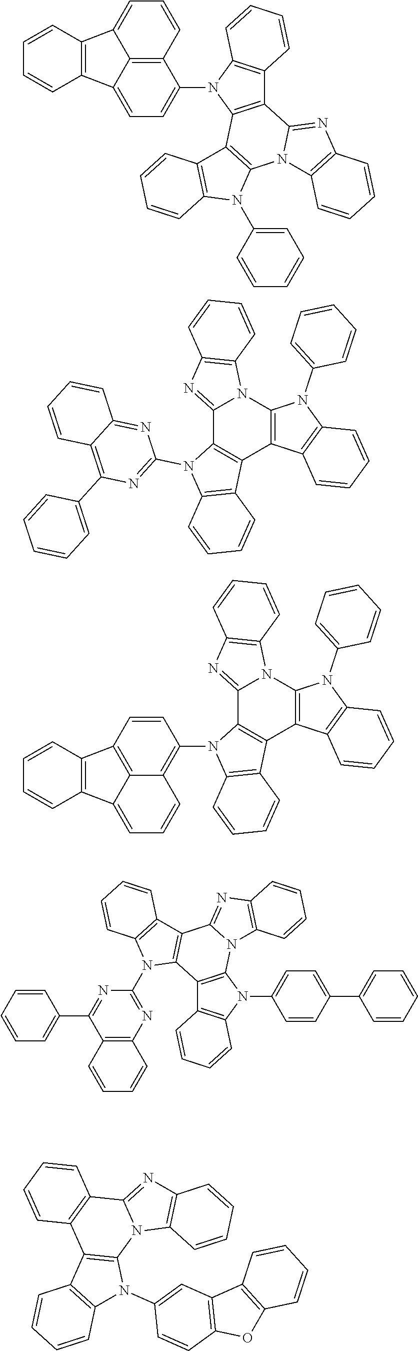

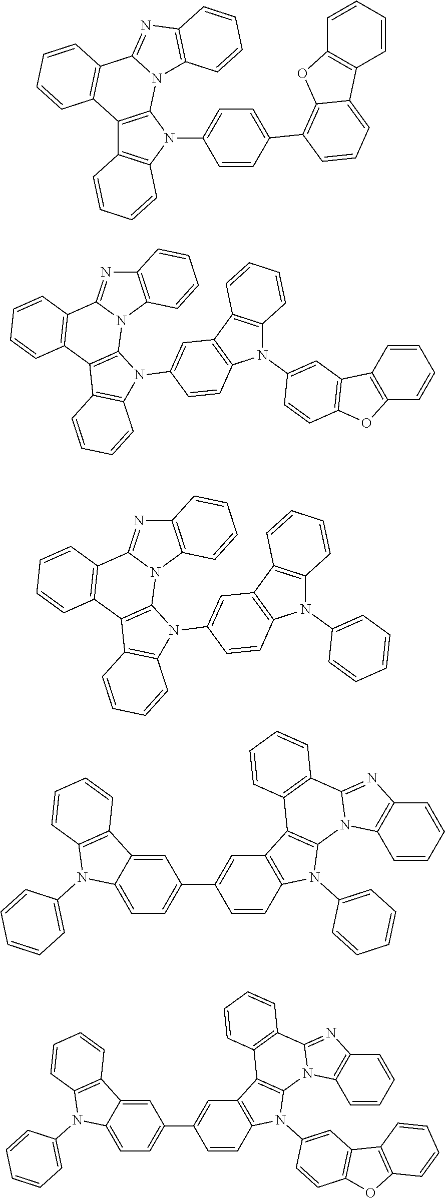

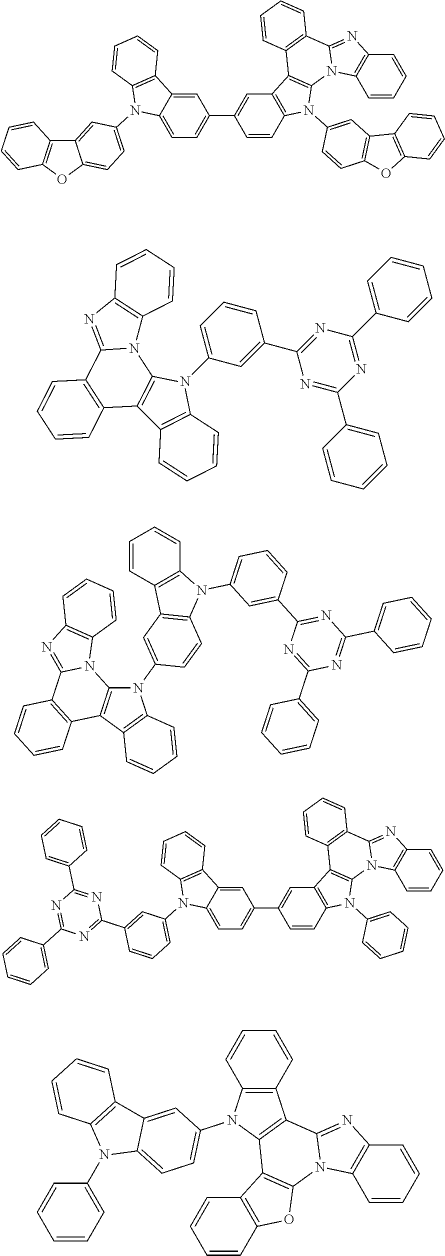

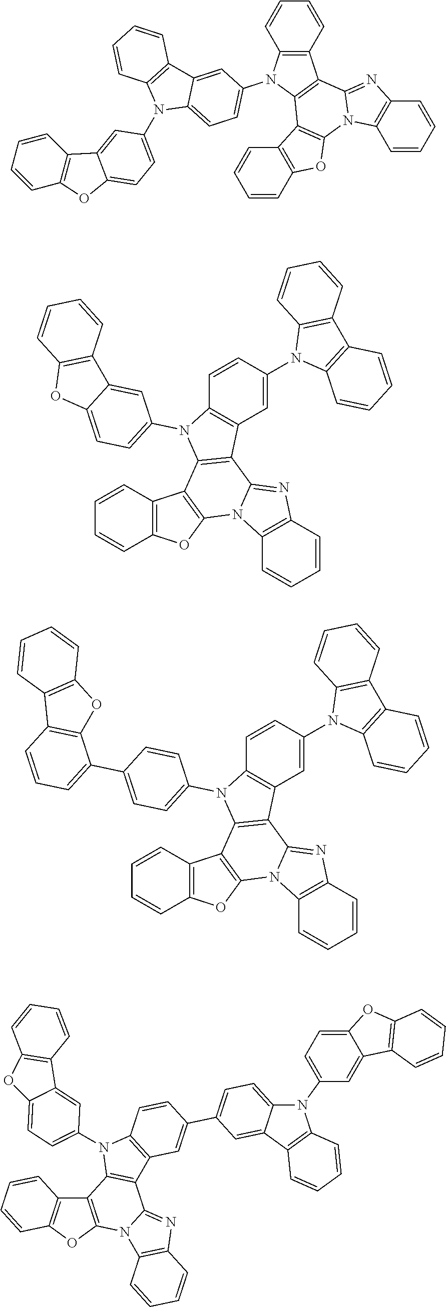

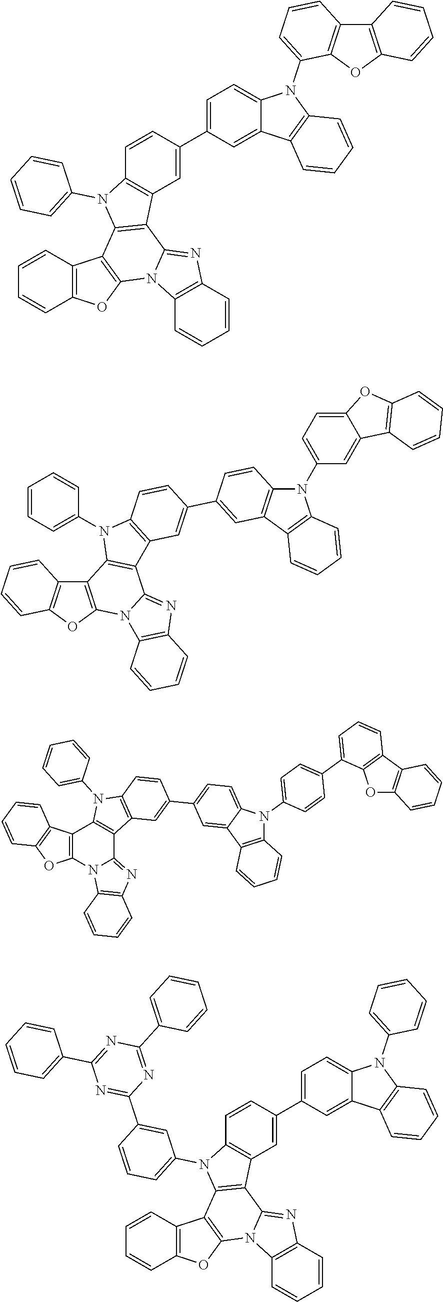

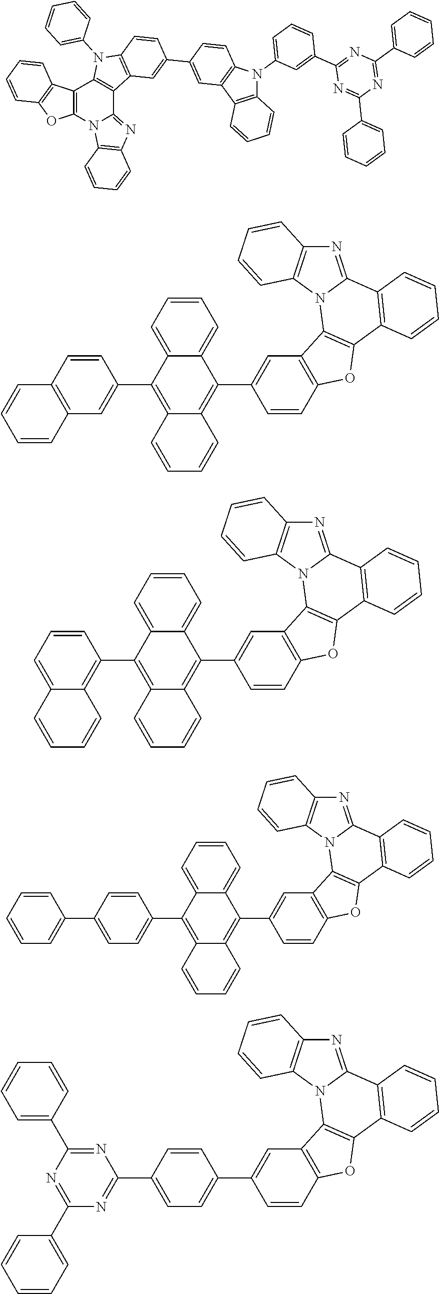

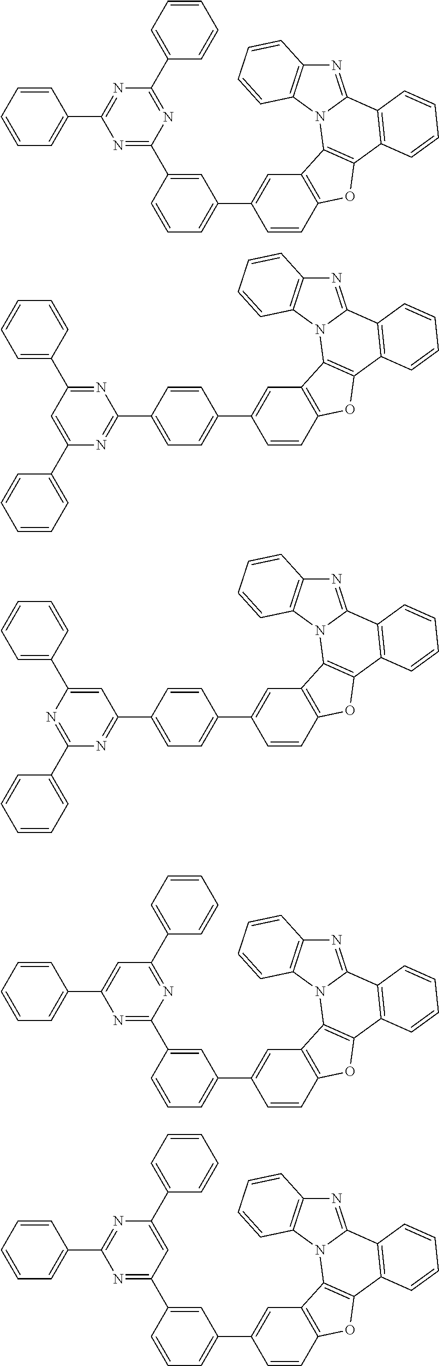

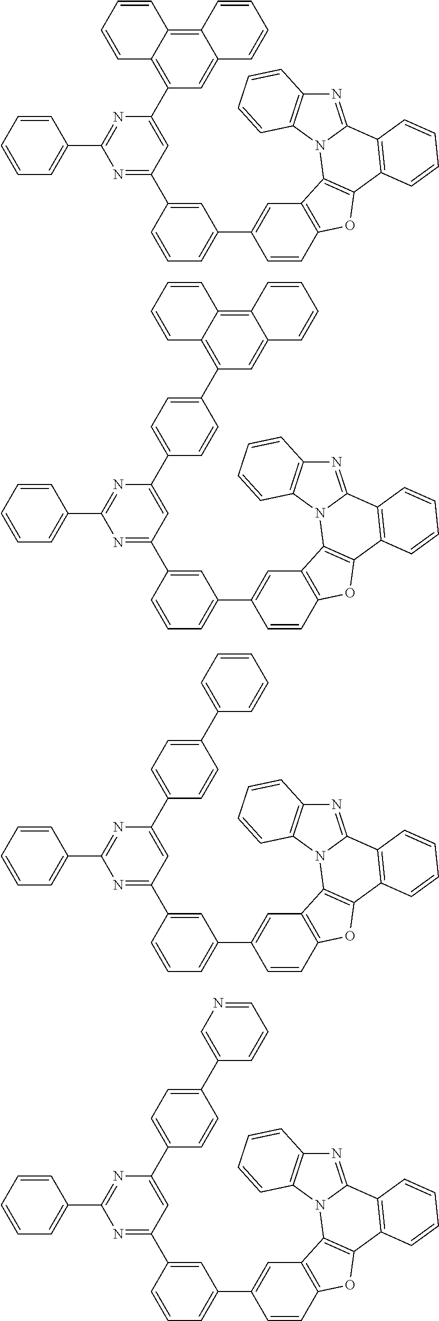

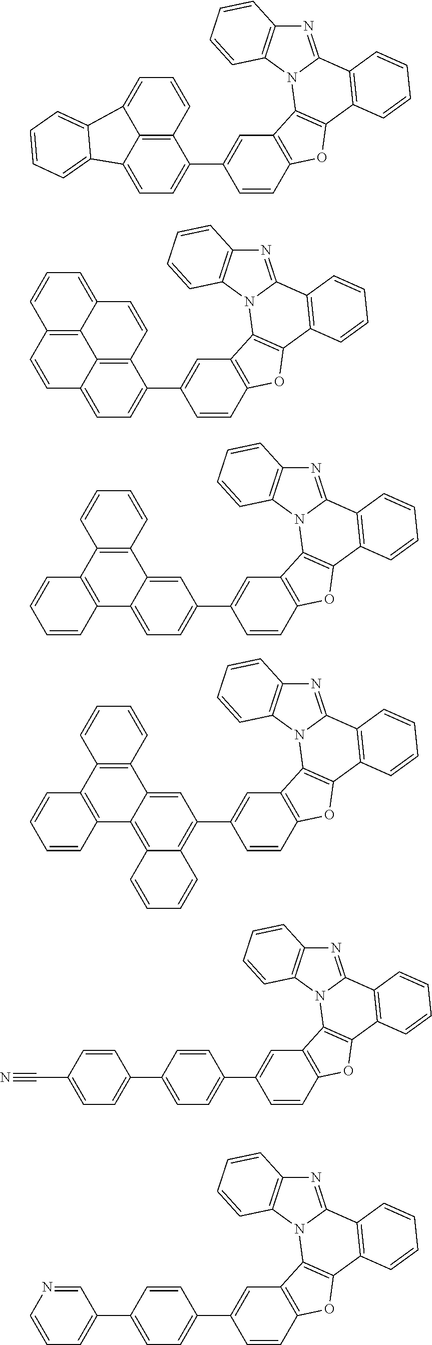

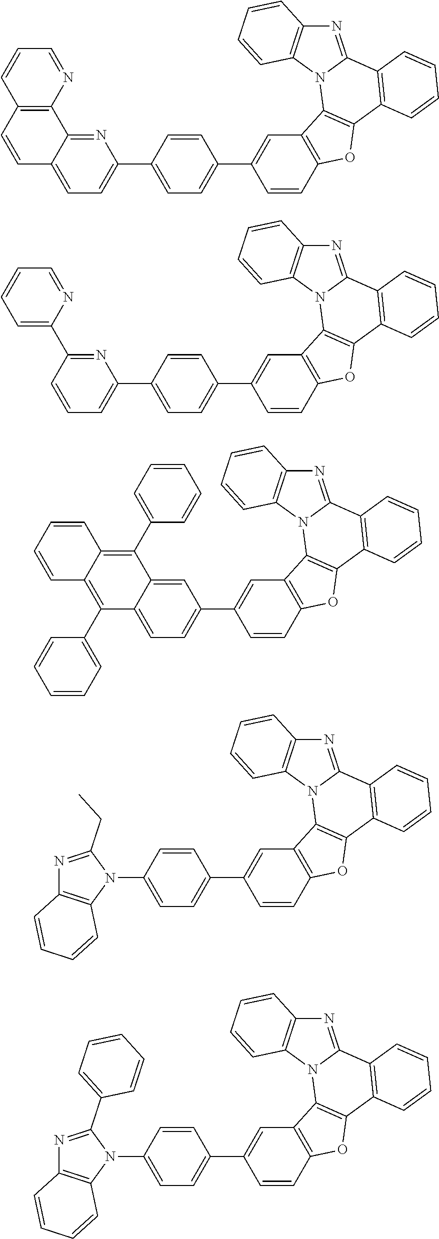

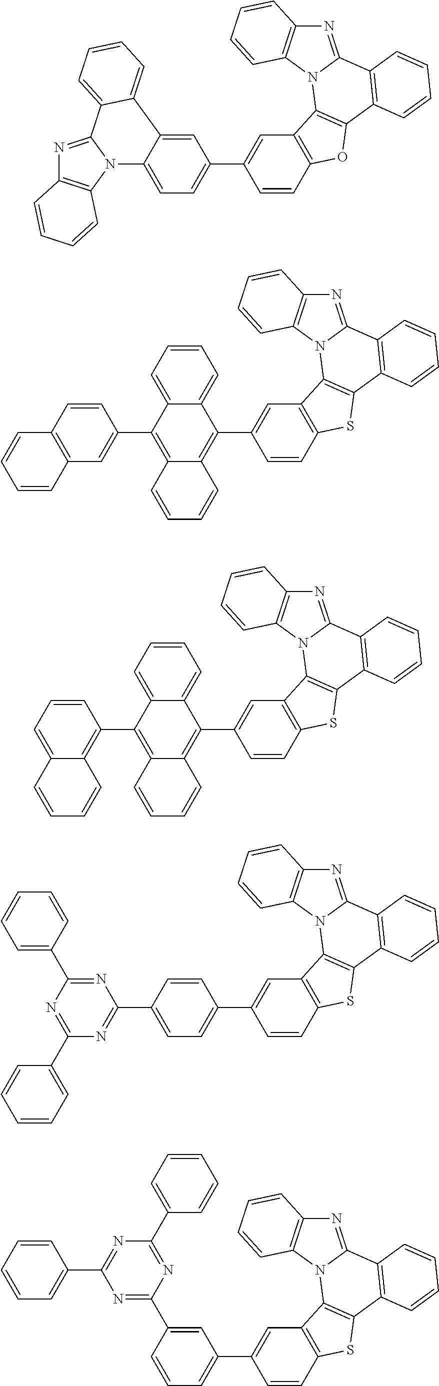

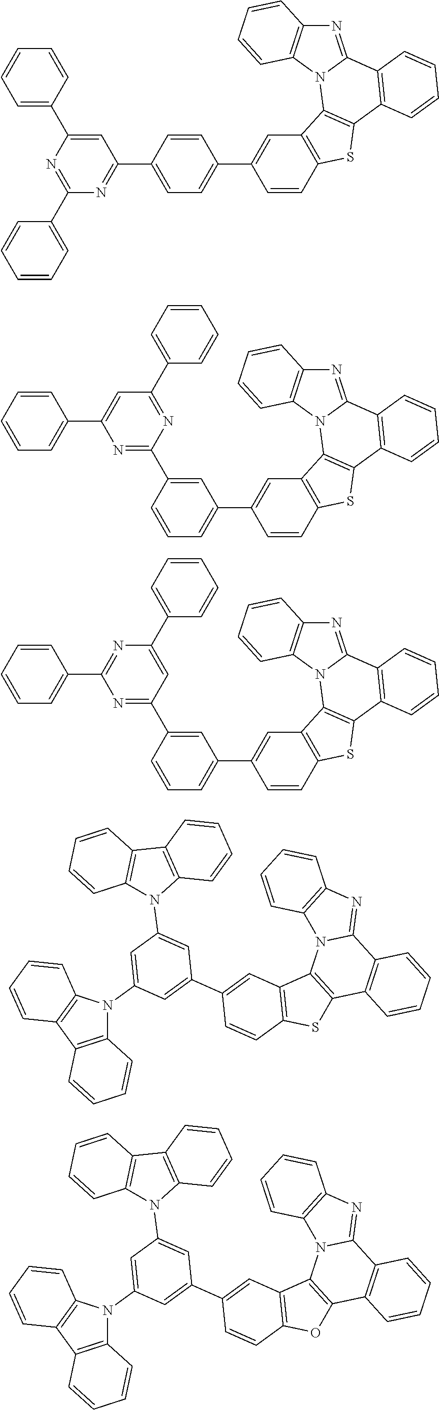

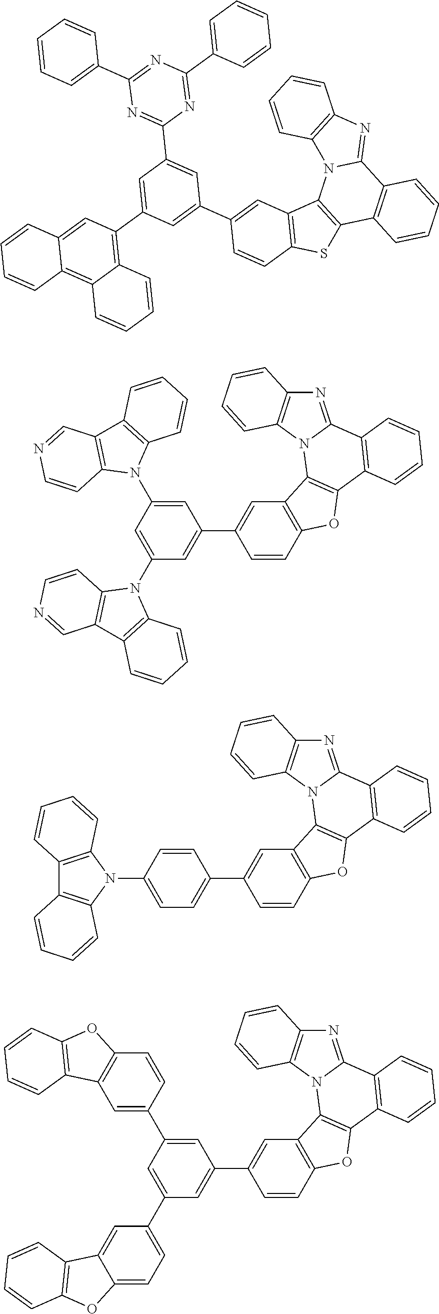

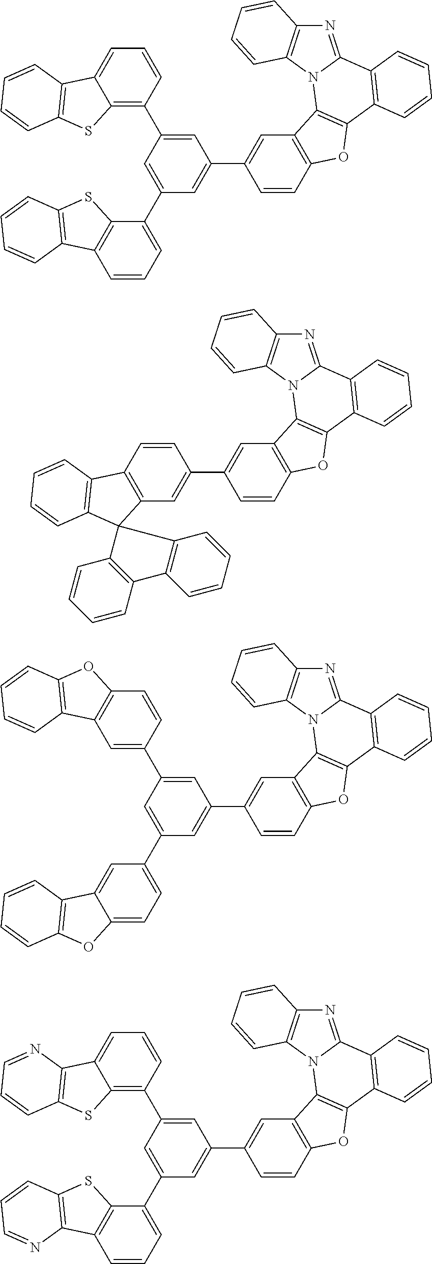

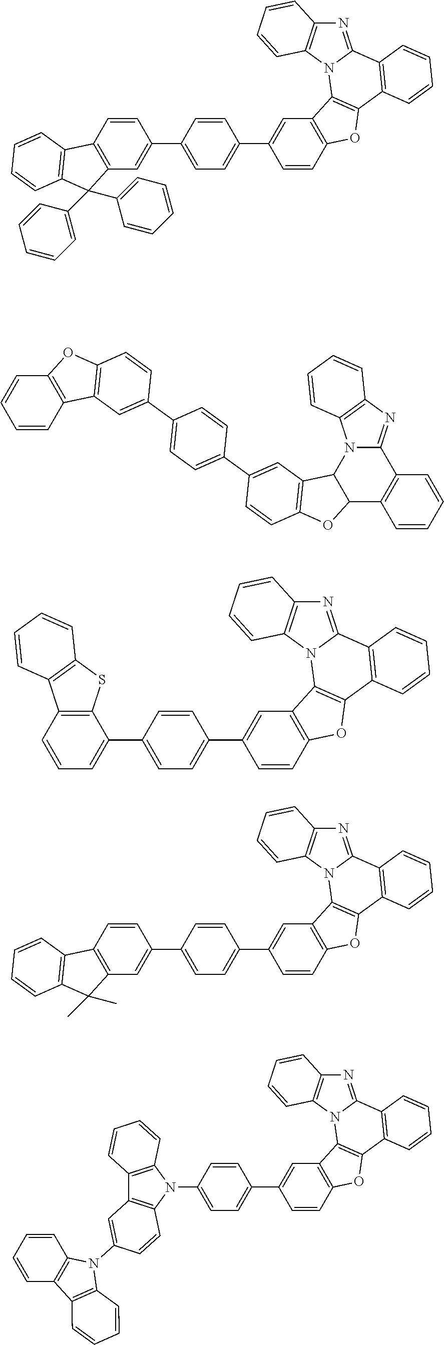

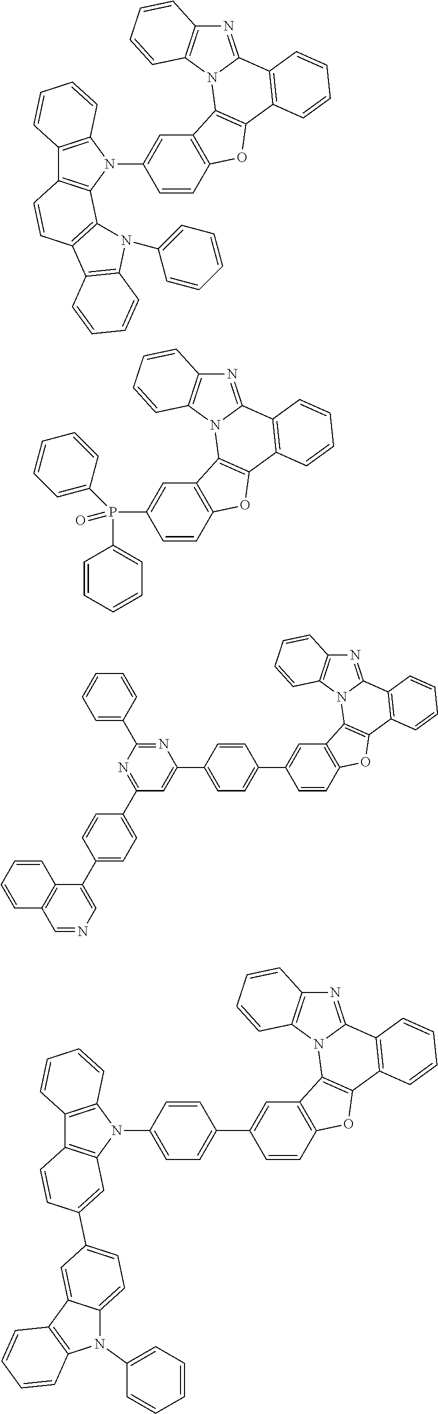

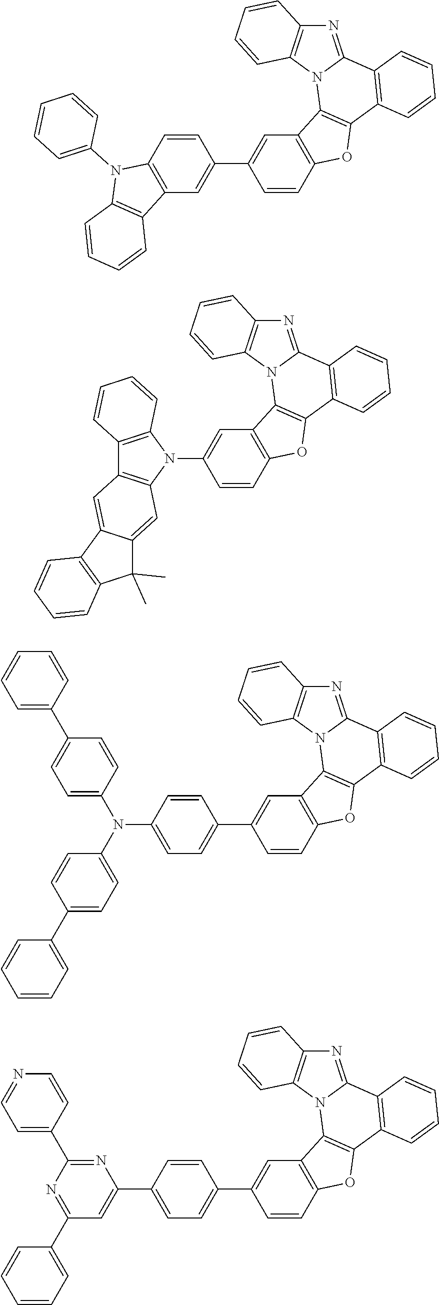

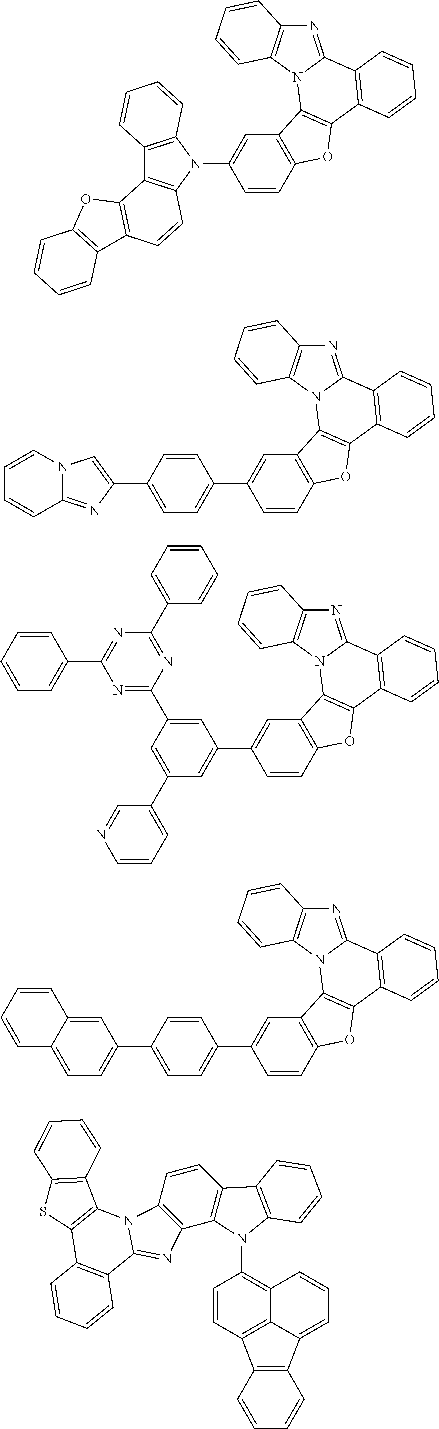

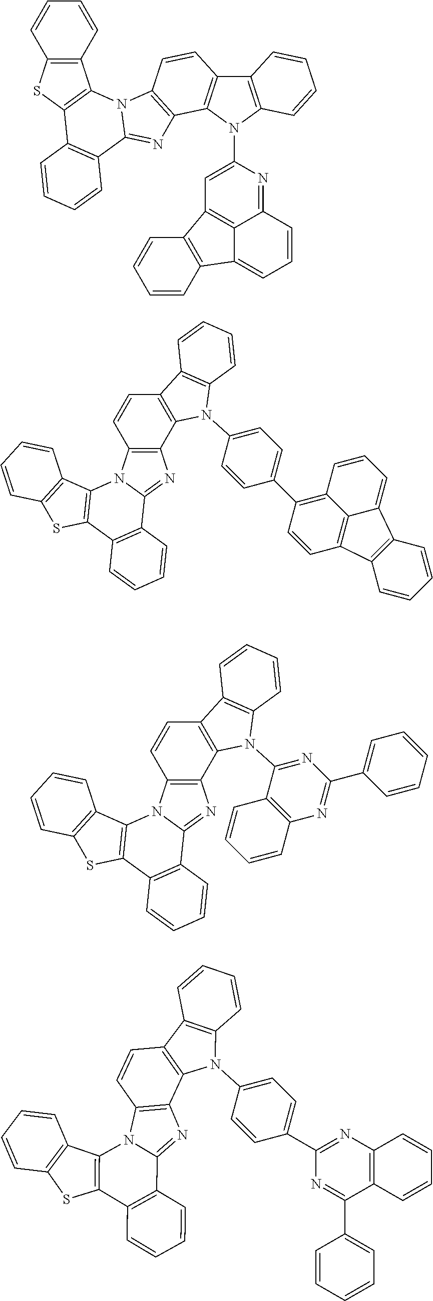

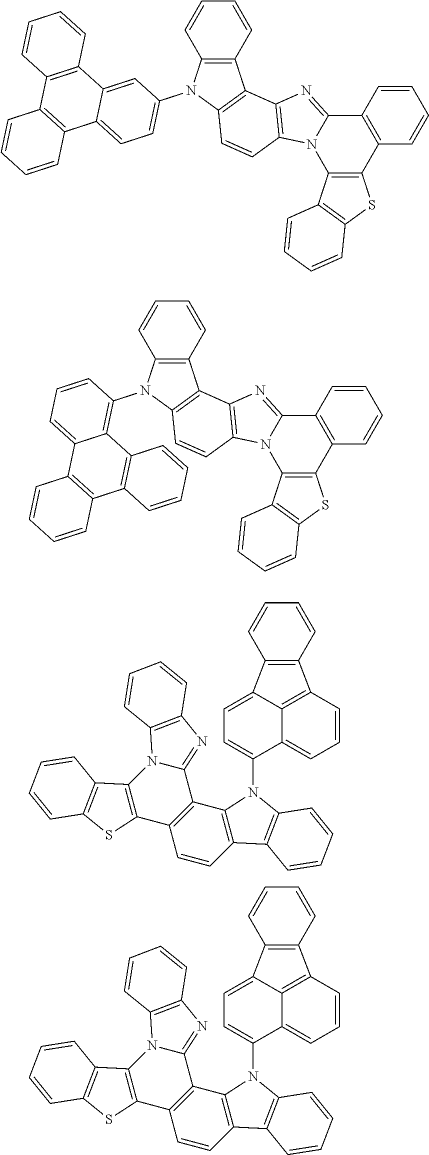

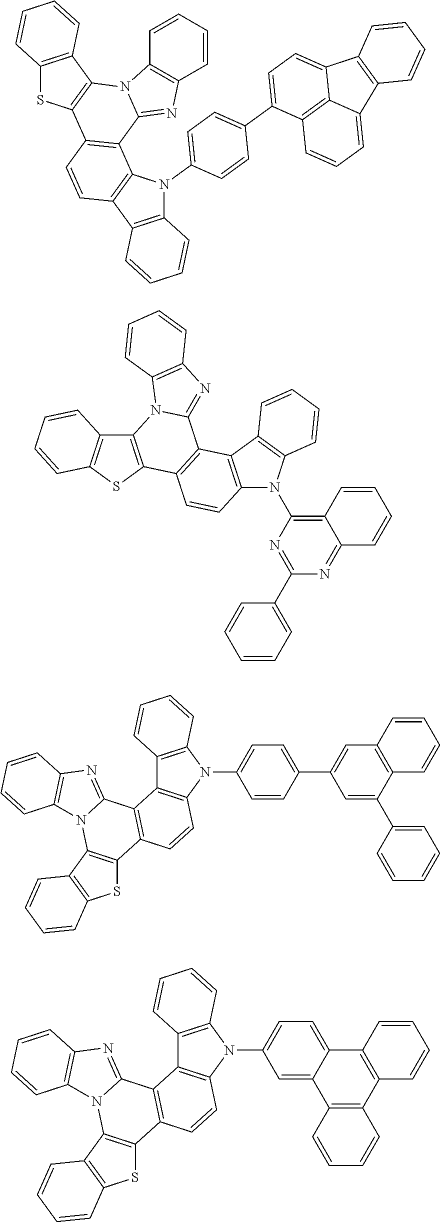

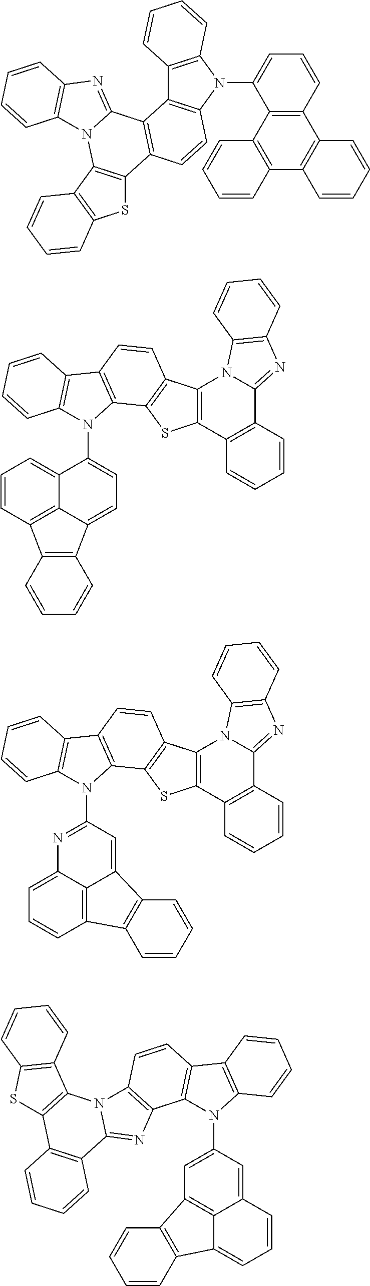

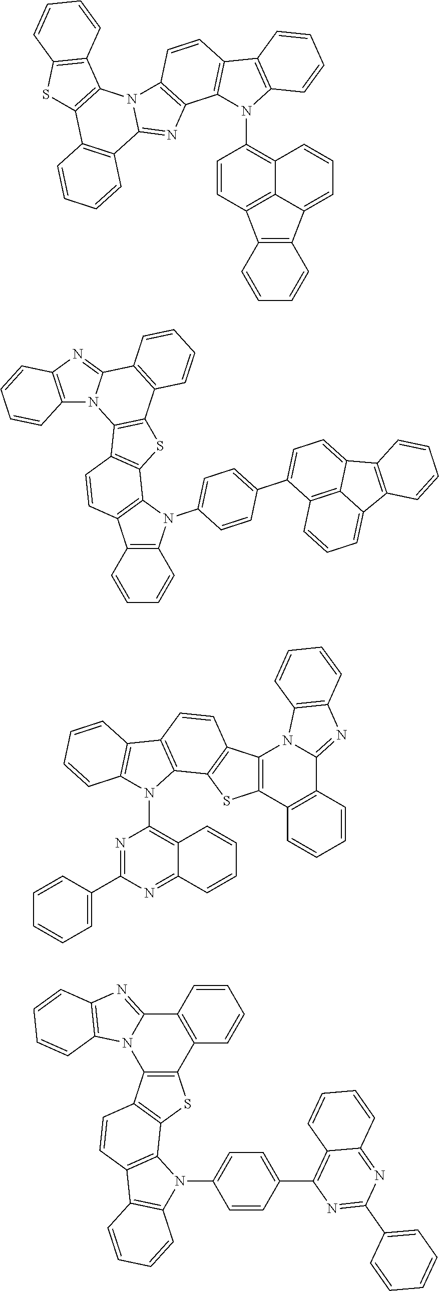

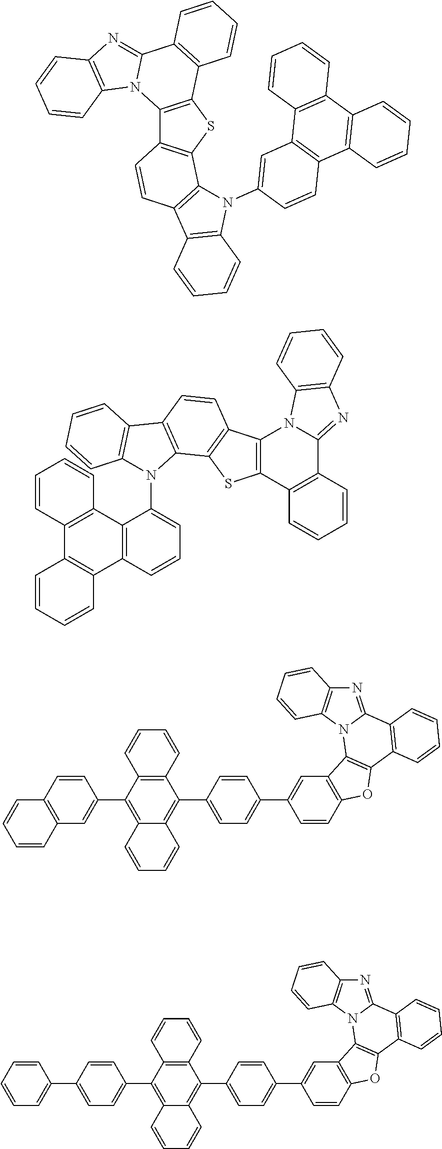

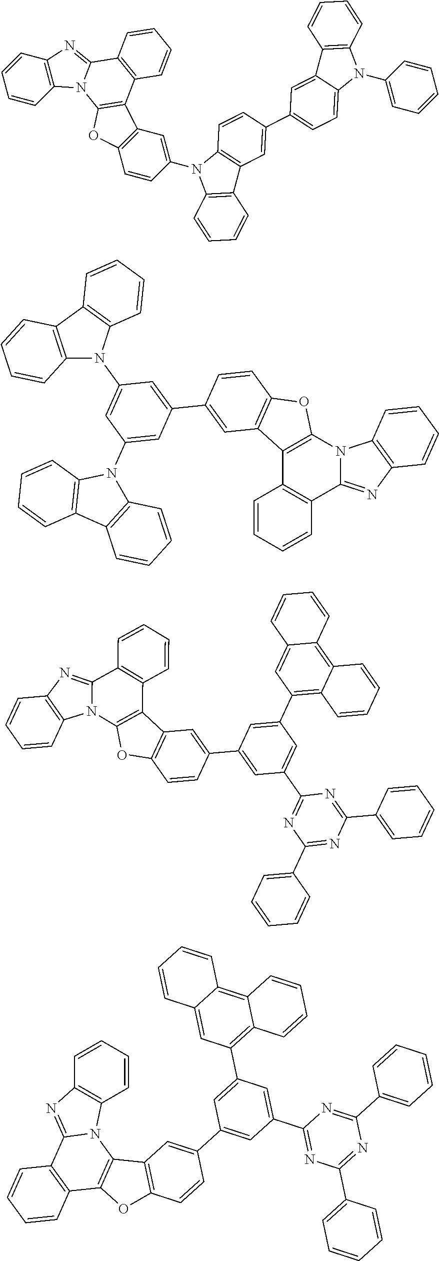

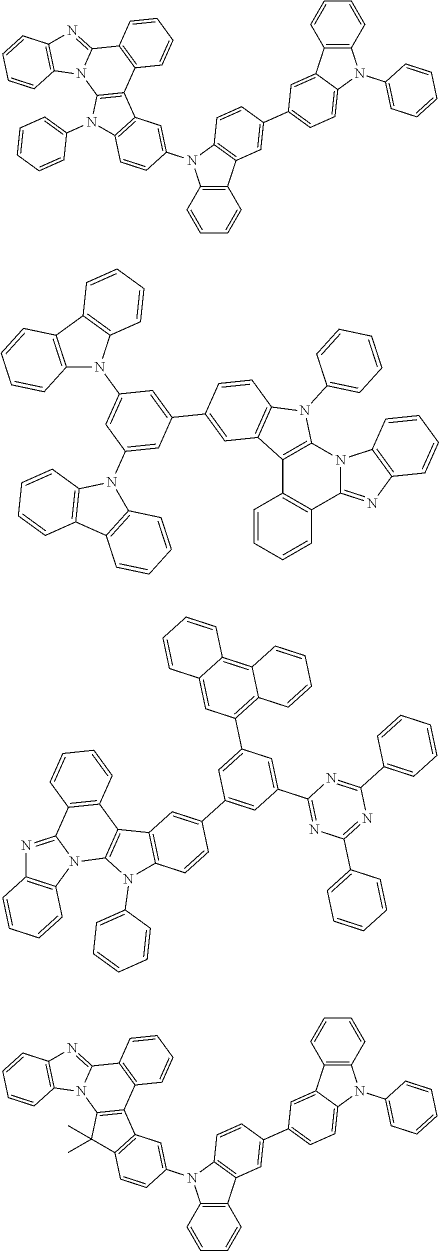

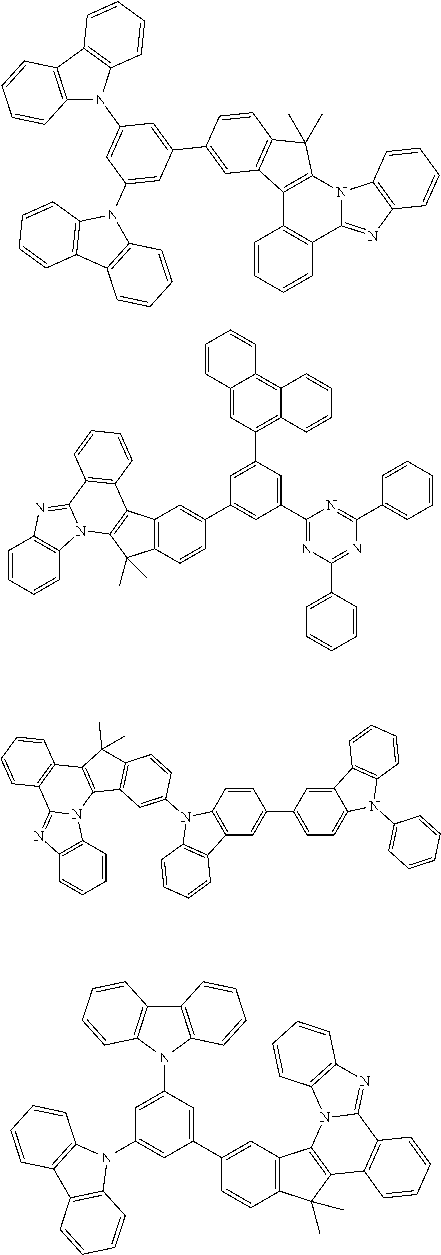

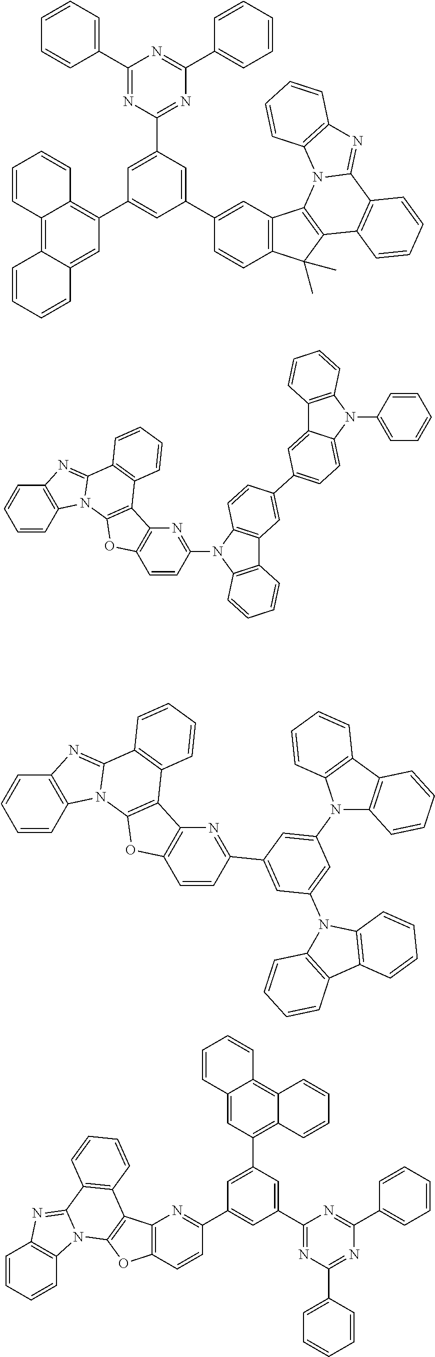

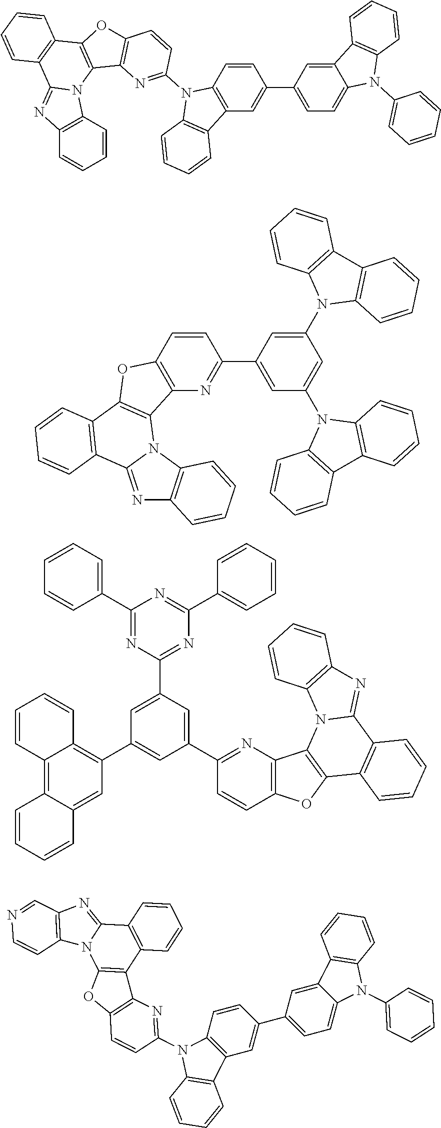

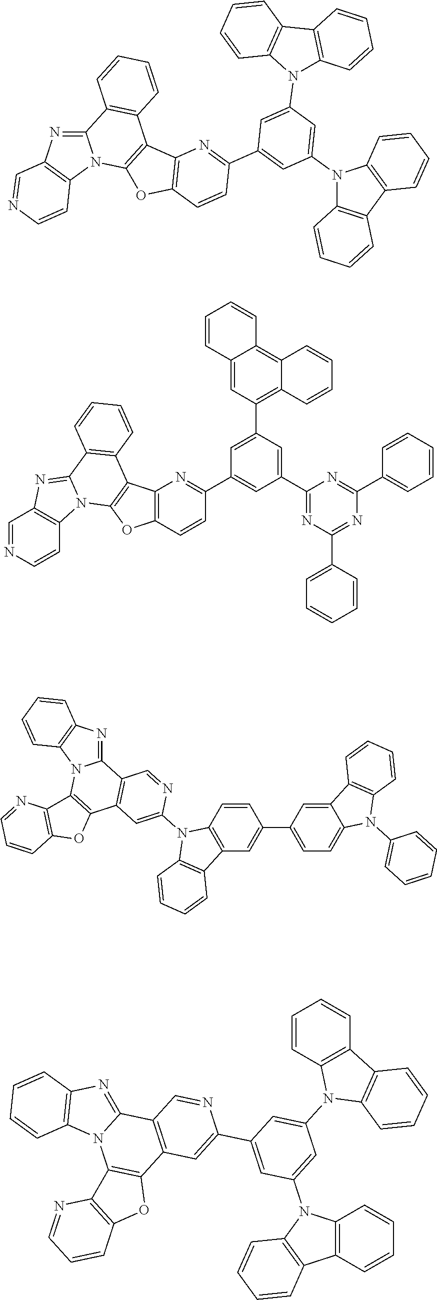

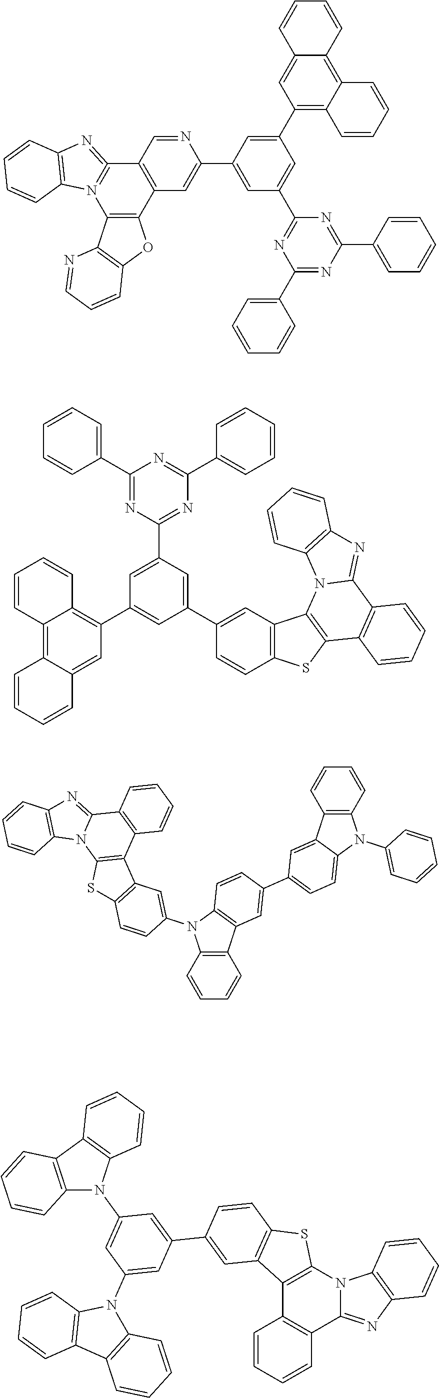

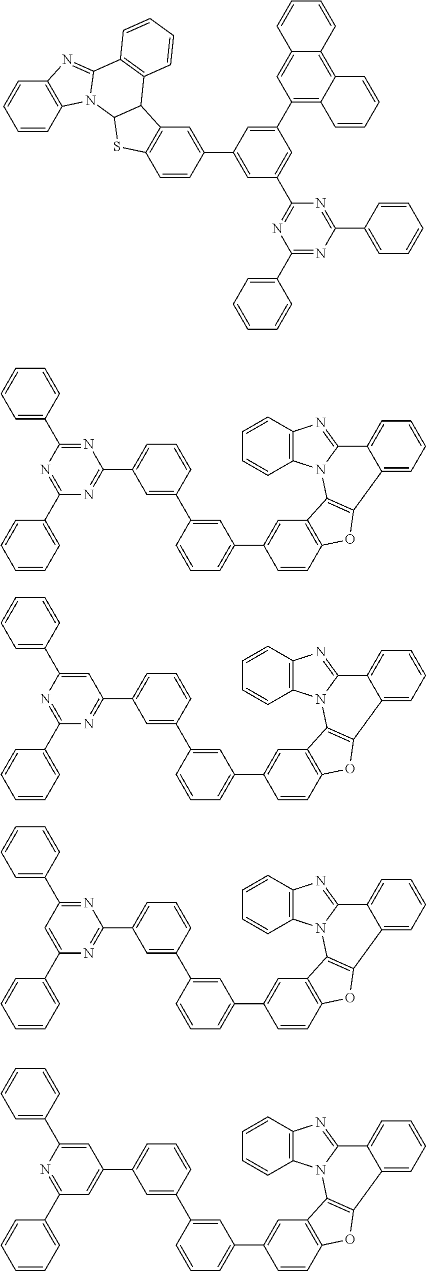

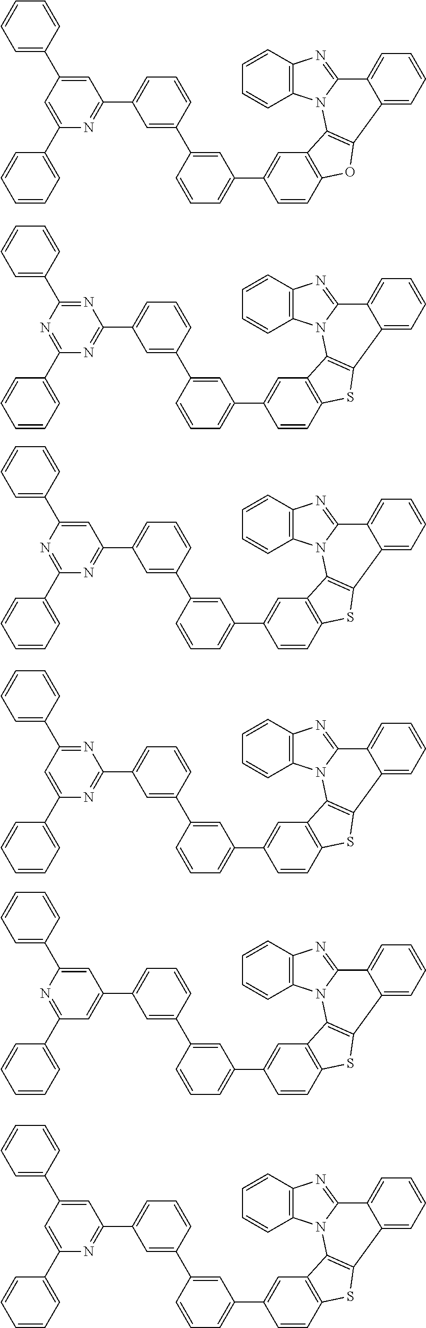

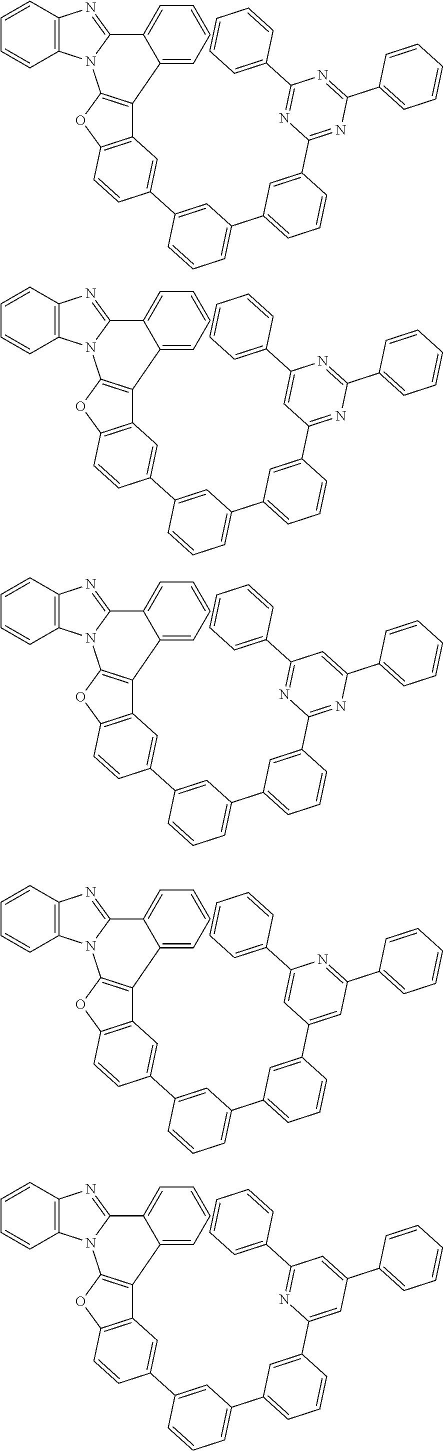

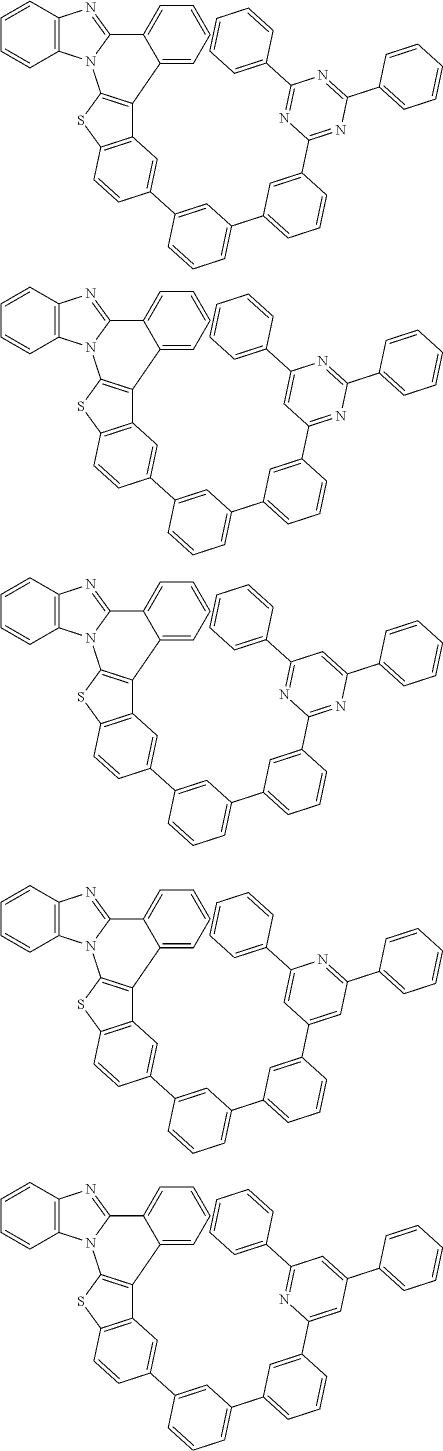

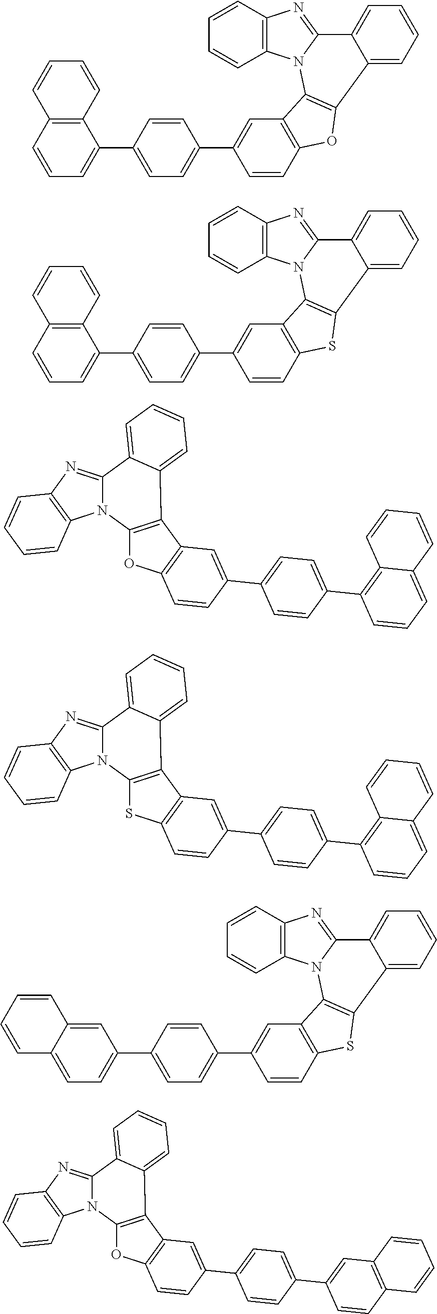

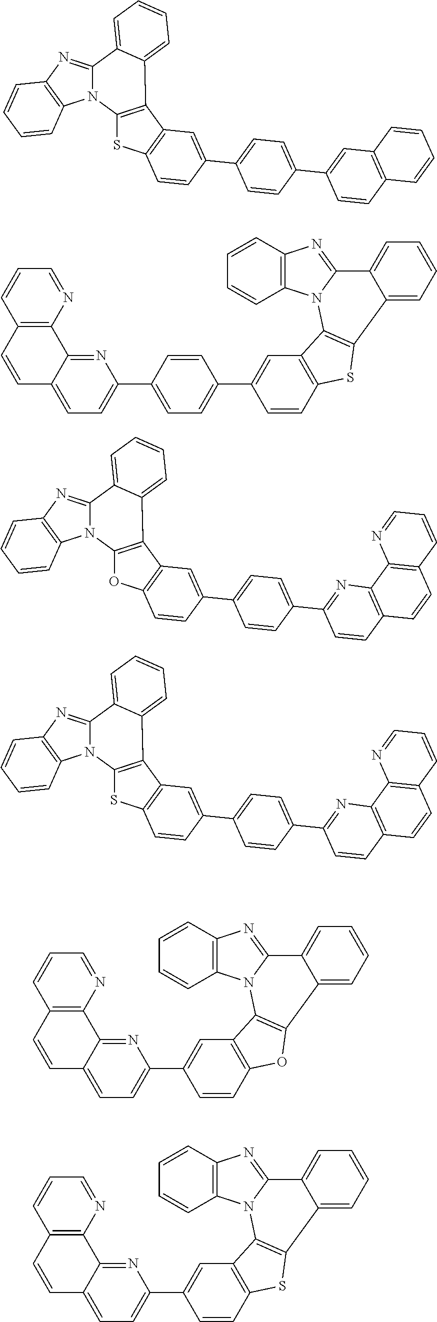

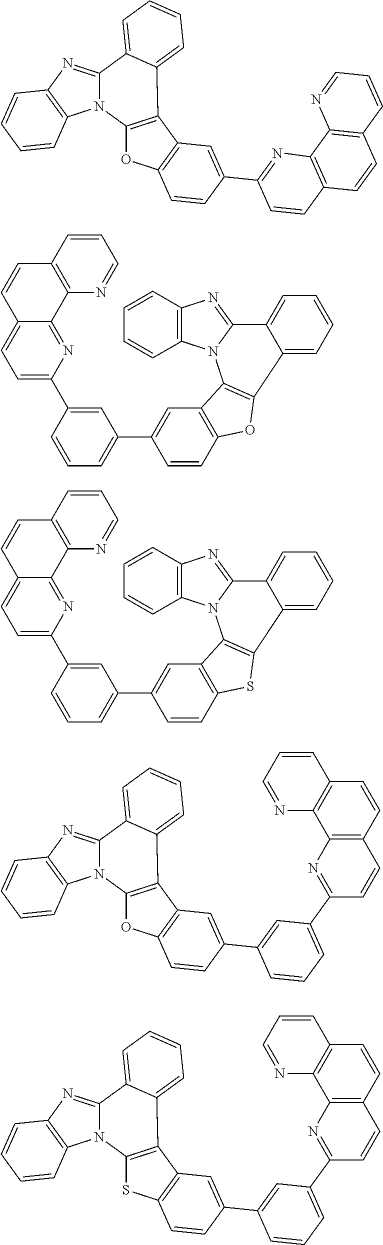

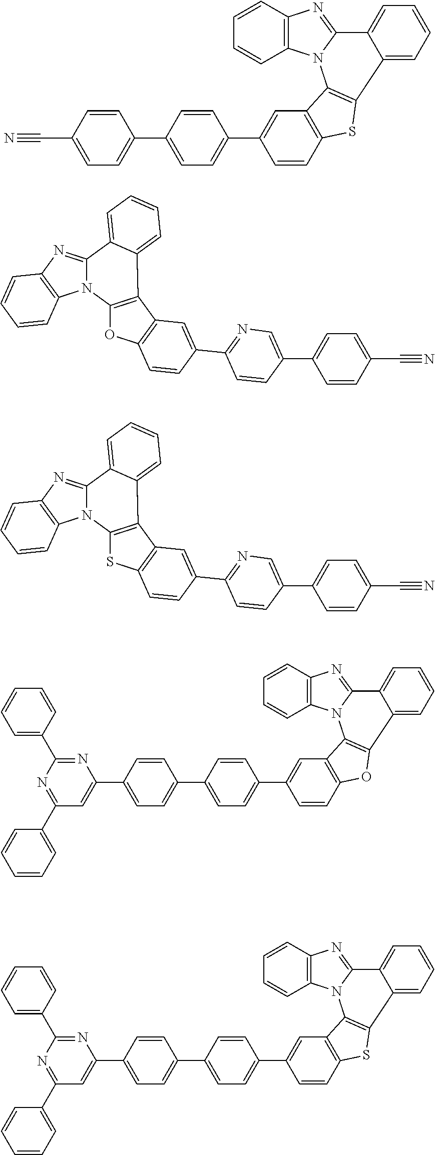

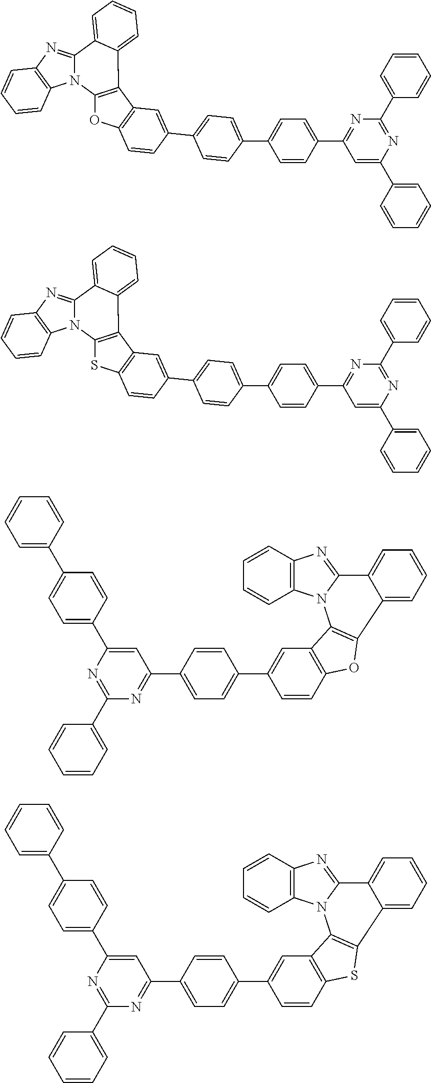

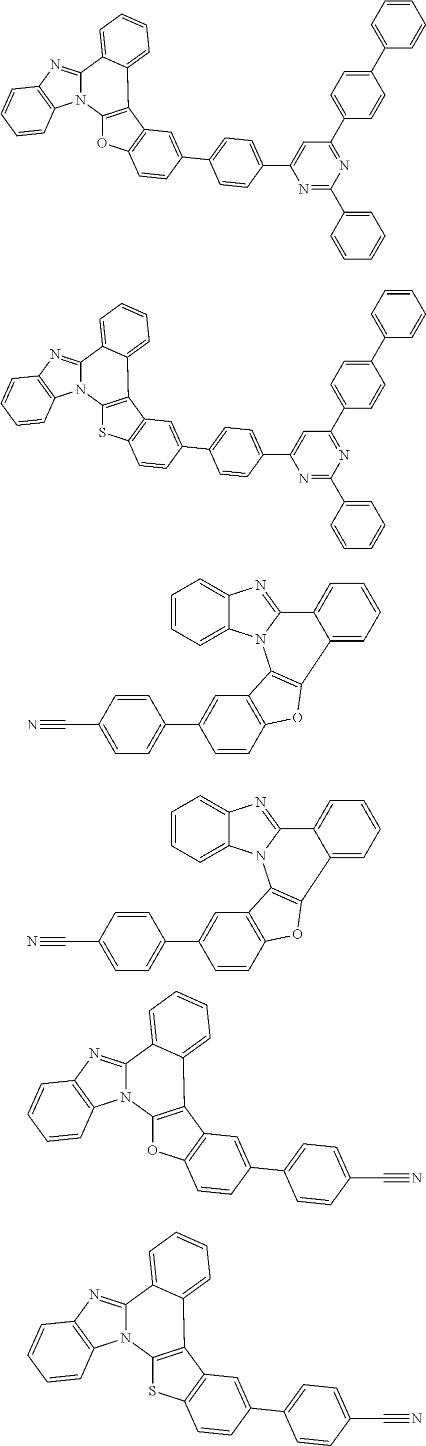

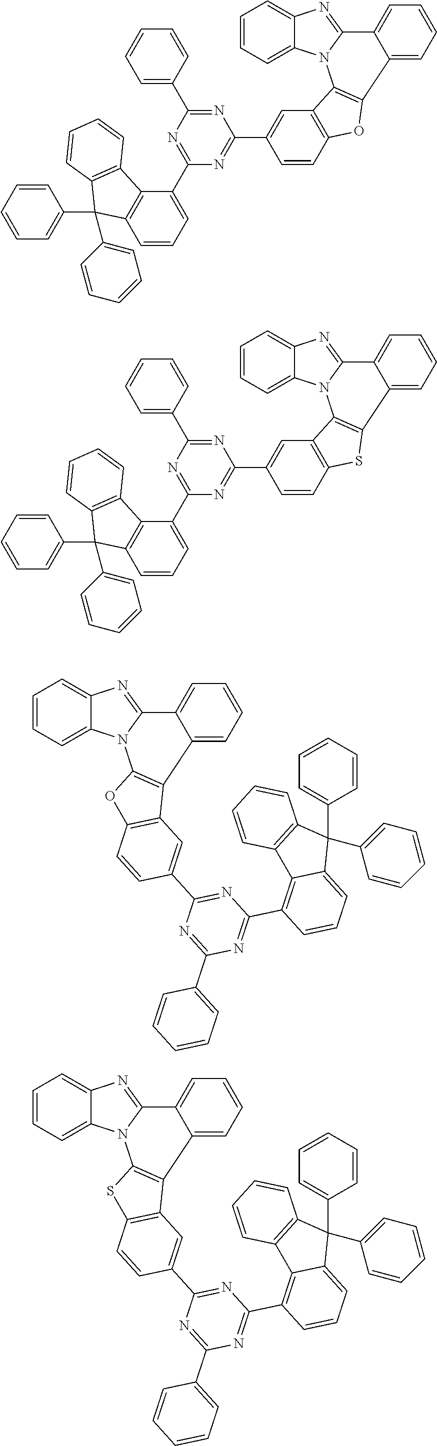

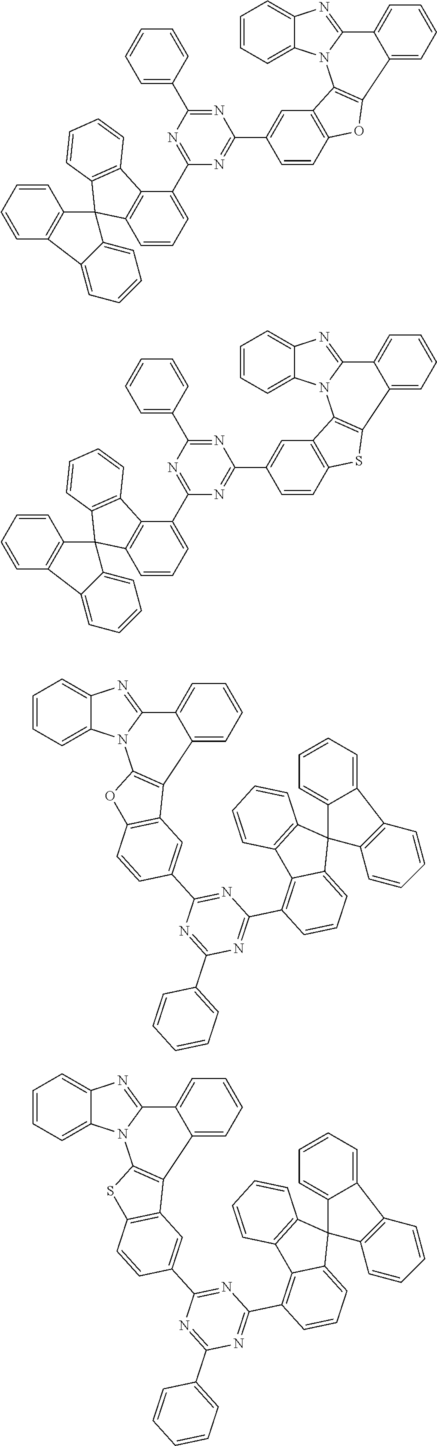

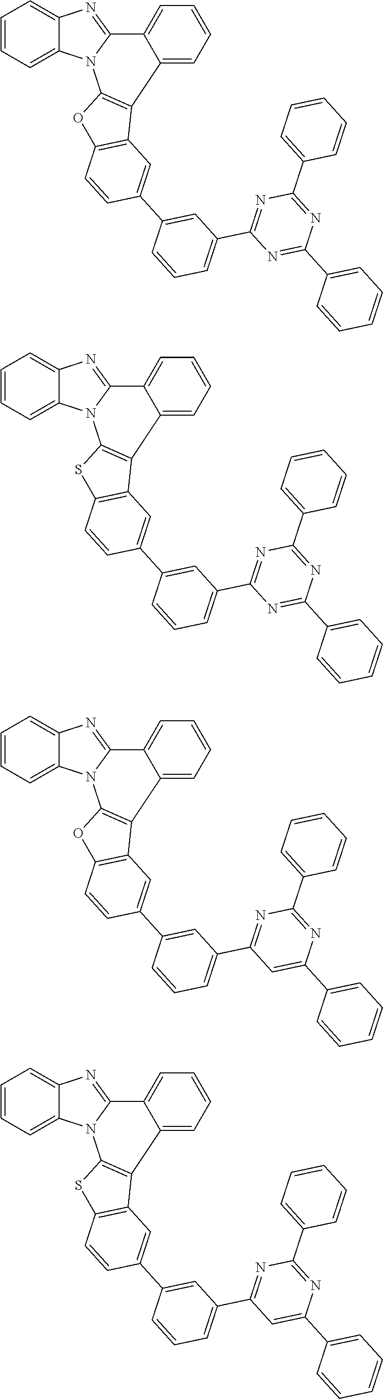

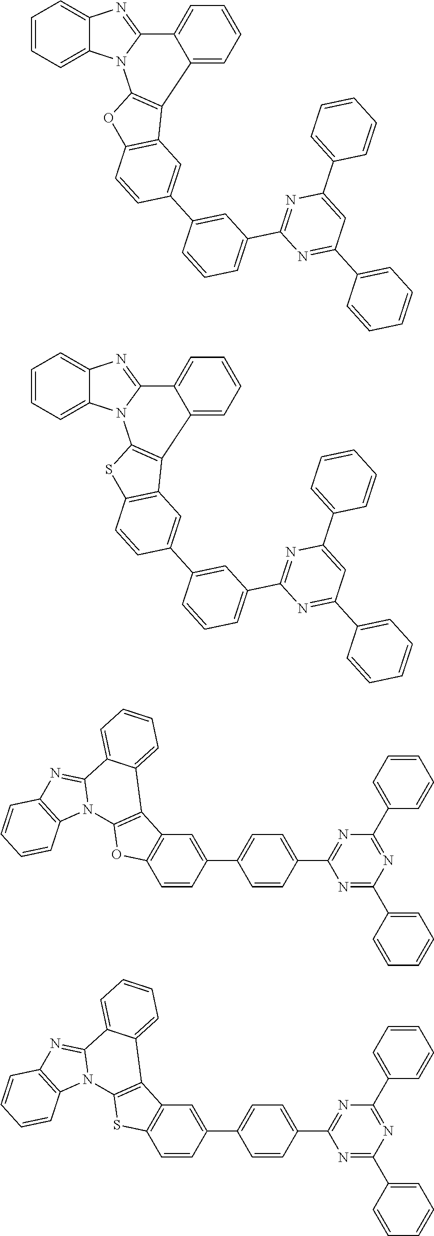

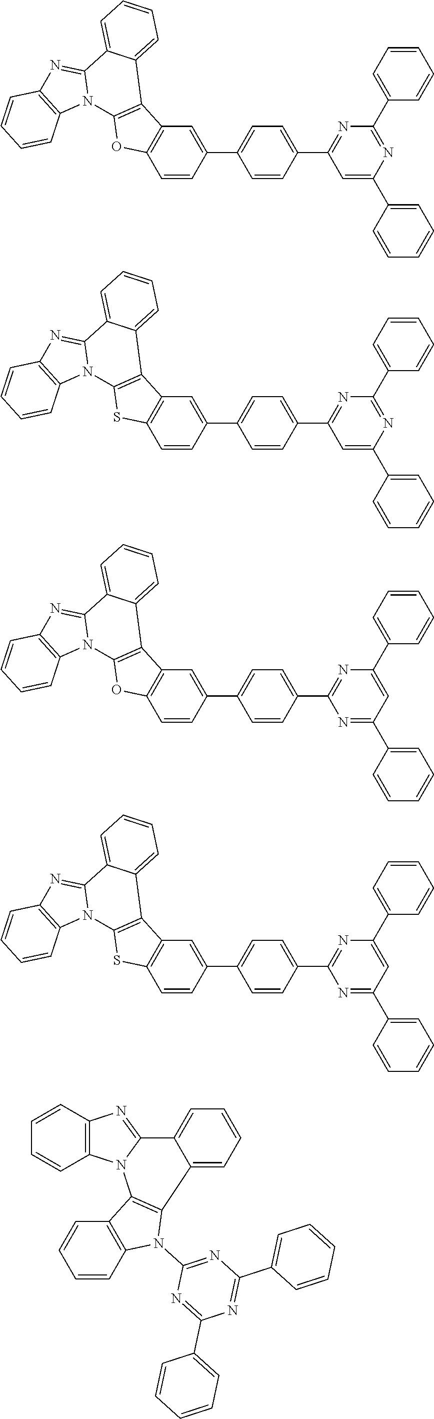

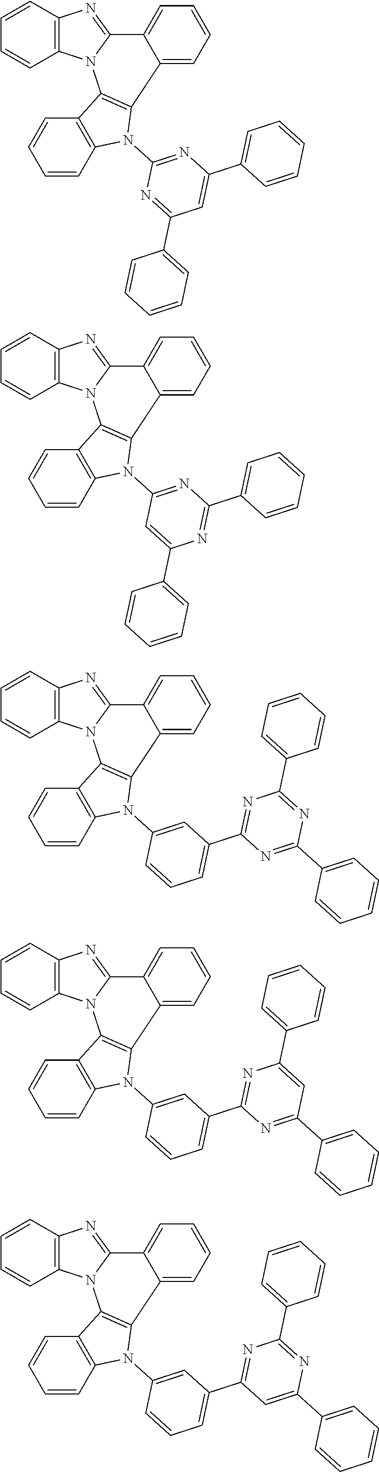

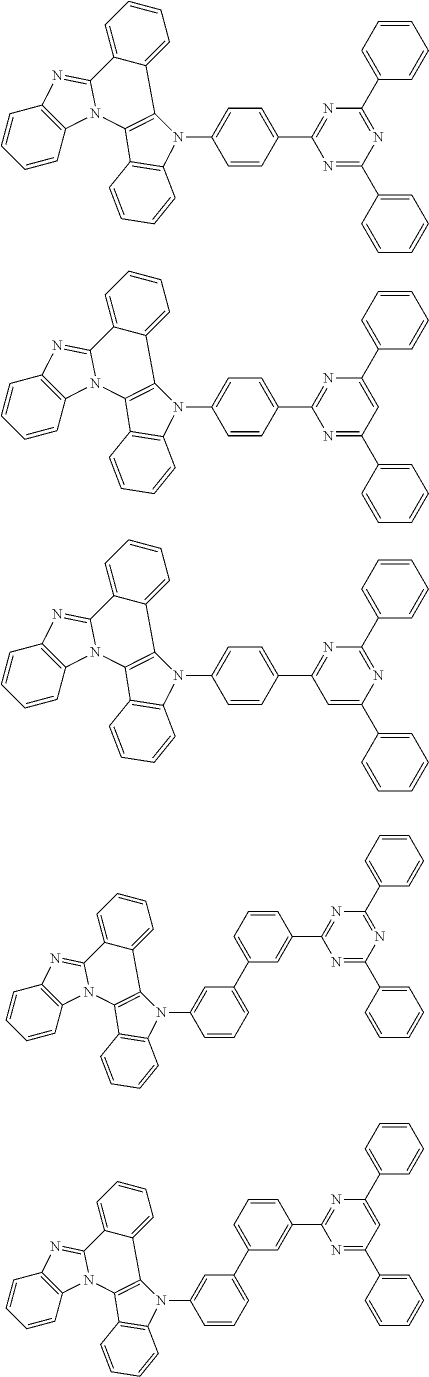

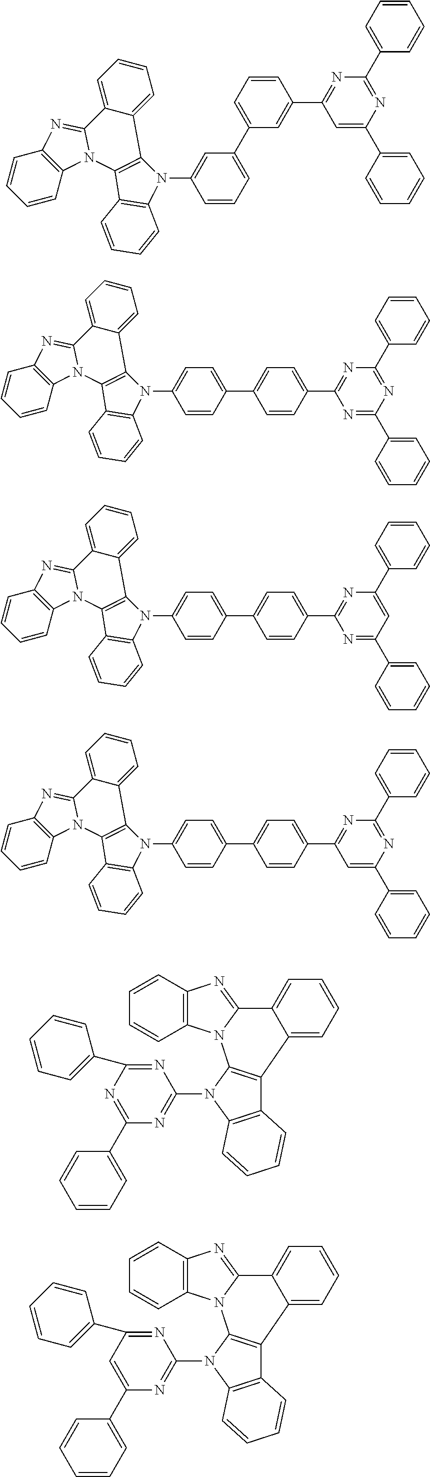

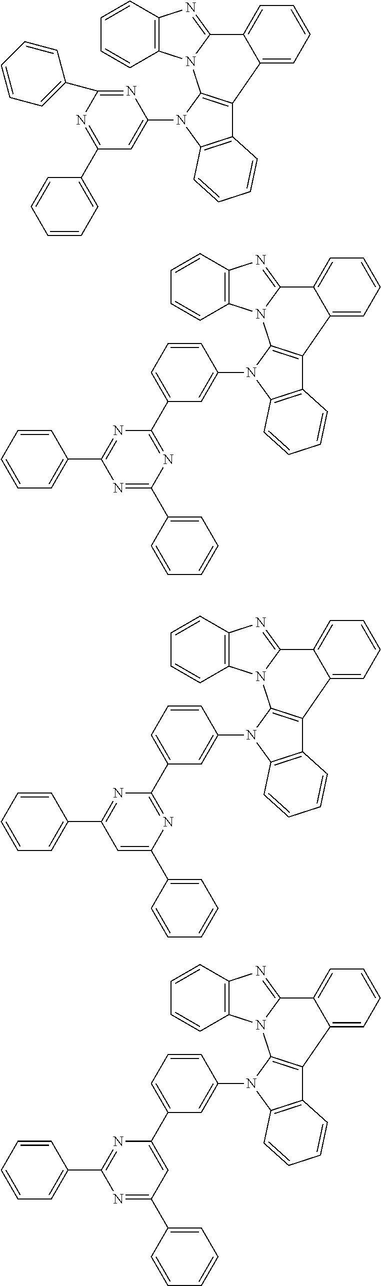

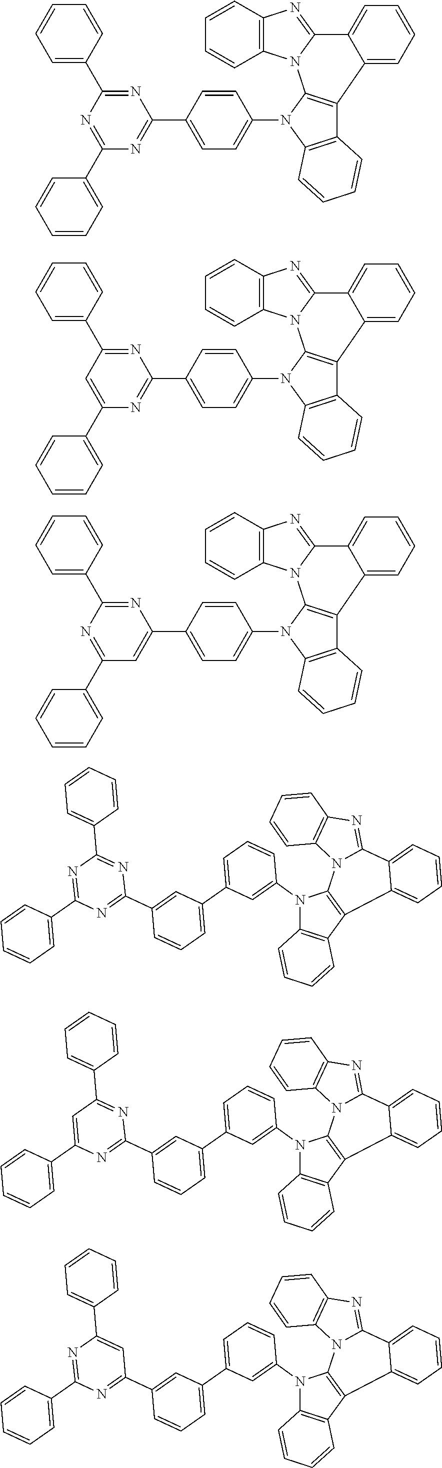

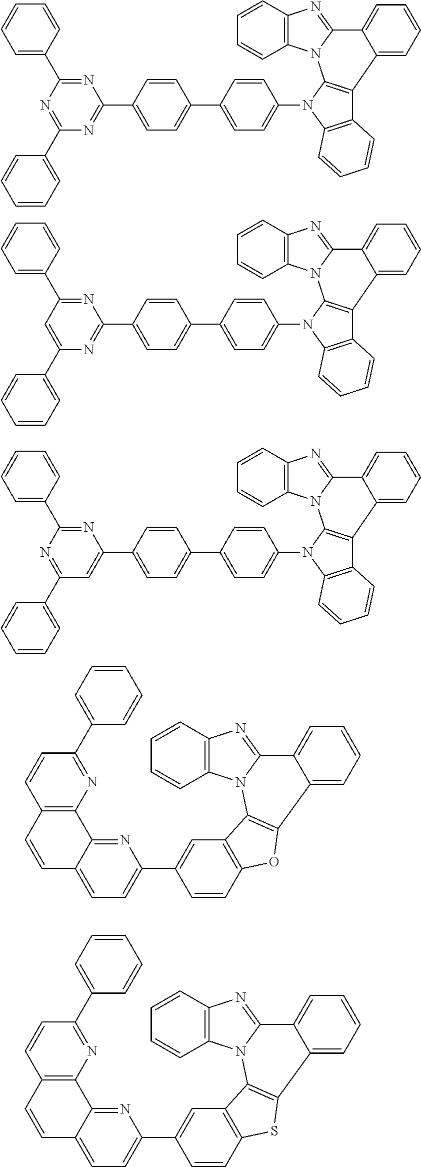

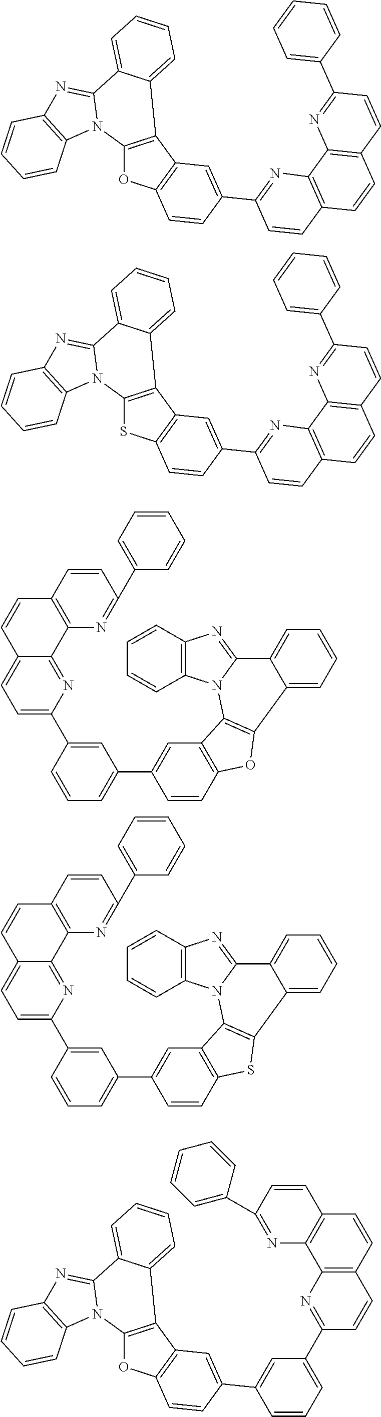

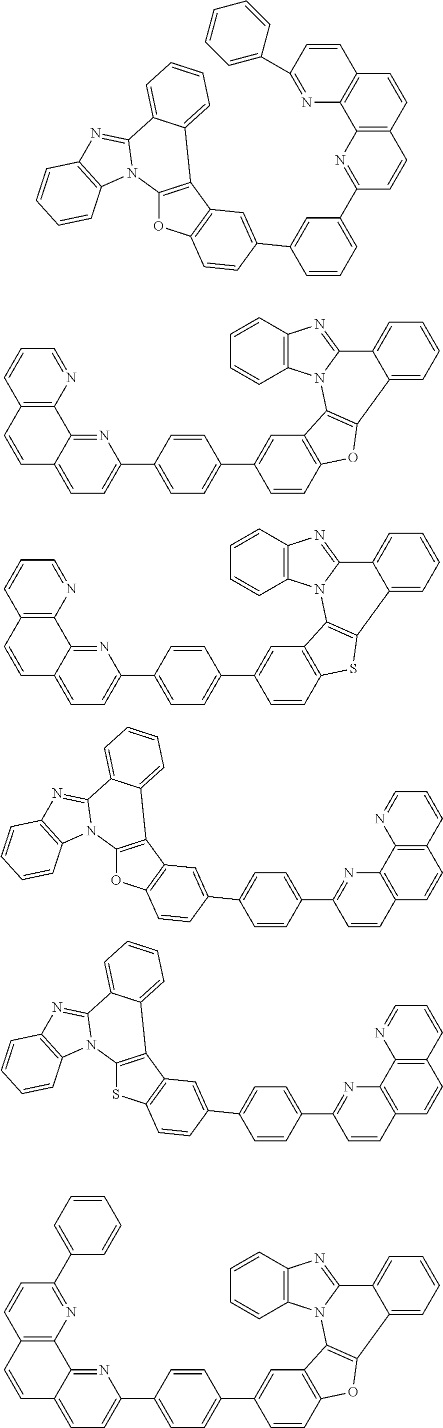

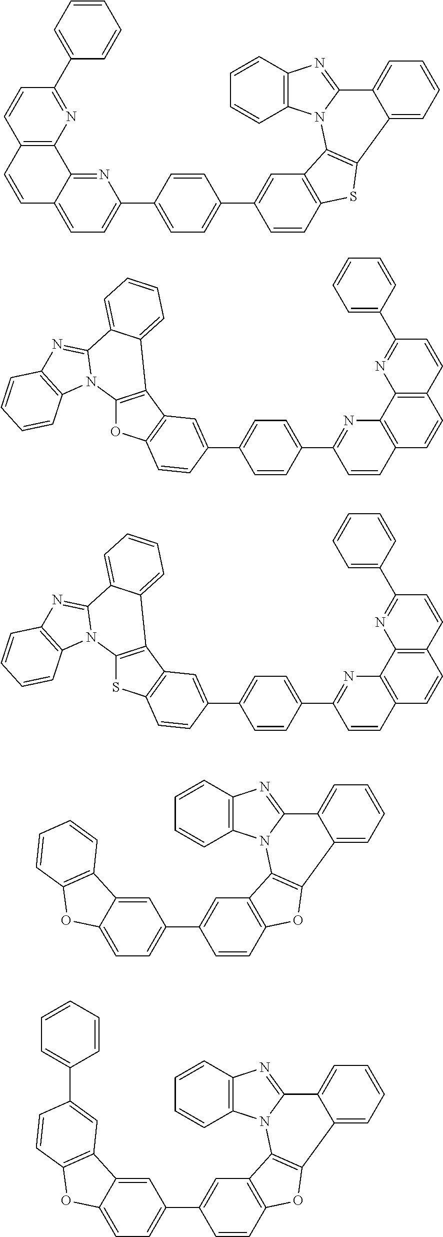

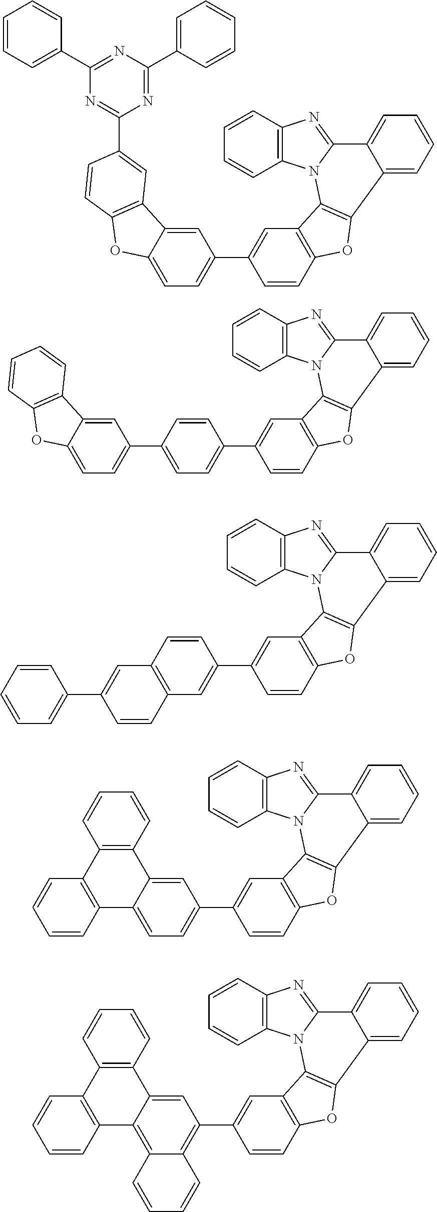

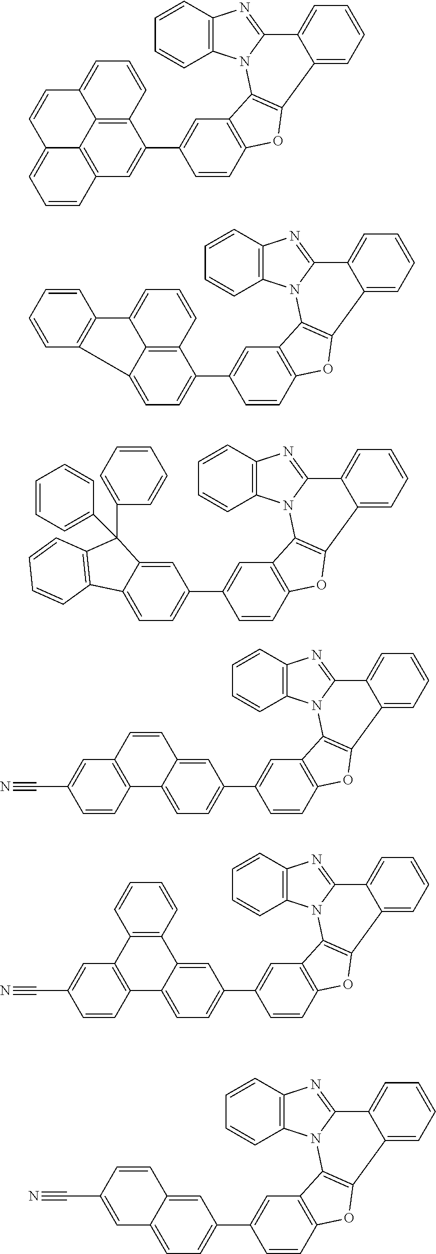

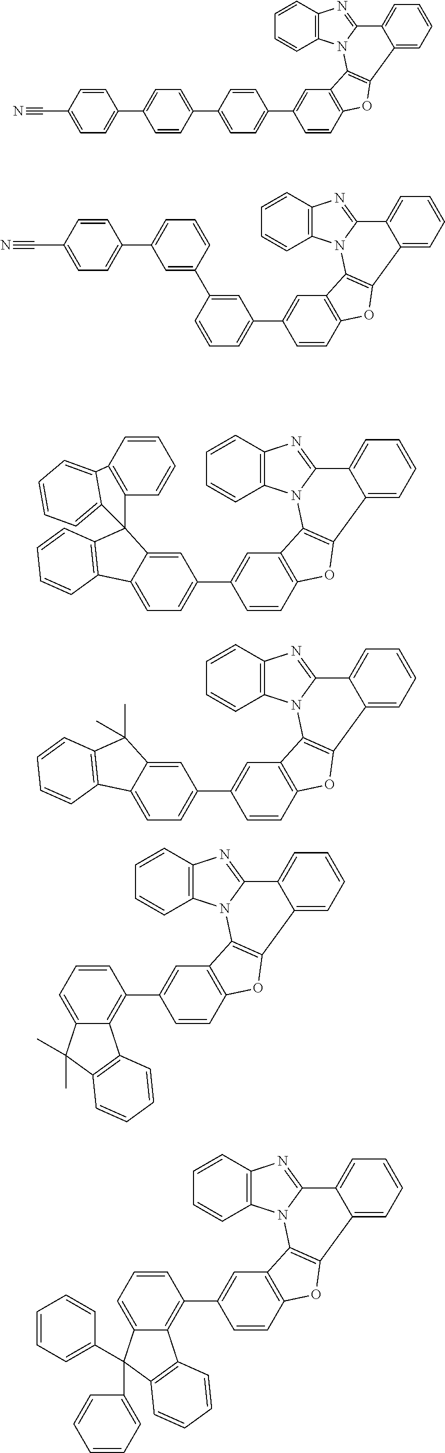

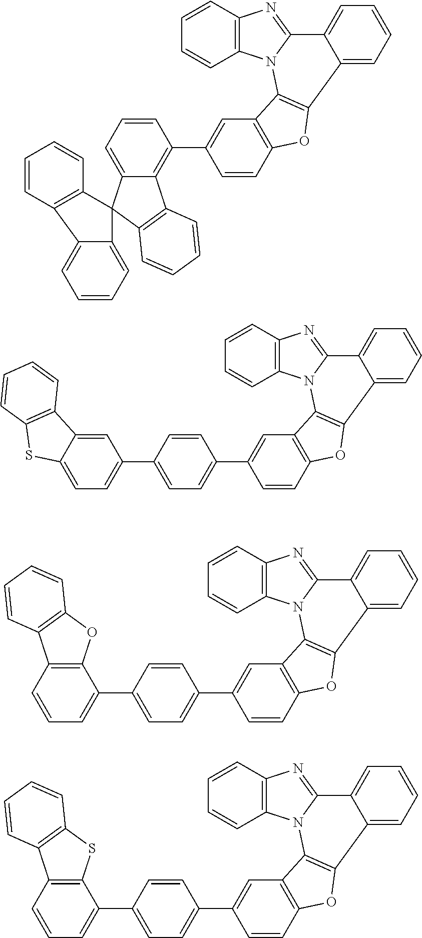

The present invention further relates to a process for the preparation of the compounds according to general formula (I), in particular for the preparation of the compounds of general formulae (Ia), (Ib), (Ic), (II), (IIa), (III) and (IV), most preferred for the preparation of the compounds of general formula (IV).

According to a first embodiment, in the compound according to formula (I), X¹ is direct bond and X² is O, S, NR²⁵, or CR²⁶R²⁷, wherein R²⁵, R²⁶ and R²⁷ have the same meanings as defined above. Compounds according to this first embodiment are prepared by the process comprising the following steps (a), (b), (c) and (d).

According to step (a) of the process, a compound according to general formula (VI) is reacted with a compound according to general formula (VII) in the presence of a base to obtain a compound according to general formula (VIII):

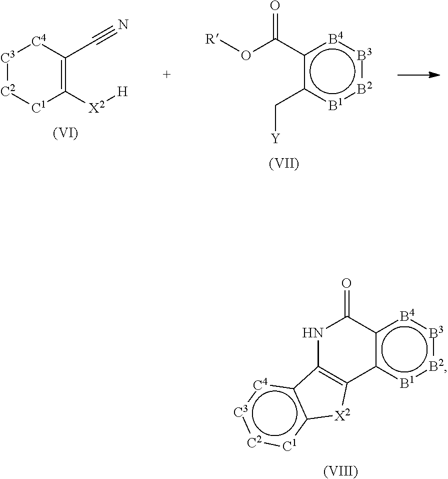

wherein X², C¹, C², C³, C⁴, B¹, B², B³ and B⁴ have the same meanings as mentioned above, R′ is C₁-C₁₈alkyl, C₁-C₁₈ alkyl which is interrupted by —O—, C₃-C₂₅cycloalkyl, C₆-C₁₈aryl group, or a heteroaryl group having 5 to 22 ring atoms, and Y is F, Cl, Br, or I.

The reaction conditions, bases, and solvents suitable are in general known by a person skilled in the art. Suitable bases are preferably selected from the group consisting of alkali metal and alkaline earth metal hydroxides such as NaOH, KOH, or Ca(OH)₂, alkali metal hydrides such as NaH or KH, alkali metal amides such as NaNH₂, alkali metal or alkaline earth metal carbonates such as K₂CO₃ or Cs₂CO₃, alkali metal phosphates such as K₃PO₄, and alkali metal alkoxides such as NaOMe, NaOEt, or KOtBu. In addition, mixtures of the aforementioned bases are suitable.

Suitable solvents are, for example, (polar) aprotic solvents such as THF, dioxane, dimethyl sulfoxide (DMSO), diethylformamide (DMF), N-methyl-2-pyrrolidone (NMP), and tridecane or alcohols such as ethanol.

Particularly preferable conditions, bases, and solvents are disclosed in Tetrahedron letters, 2011, 52, 1557, and Tetrahedron letters, 2011, 52, 1574.

The compound according to general formula (VIII) which is the product of step (a) of the process according to the present invention is introduced into step (b).

Step (b) of the process according to the present invention comprises the reaction of the compound according to general formula (VIII) with a compound according to general formula (IX) in the presence of a base to obtain the compound according to general formula (X):

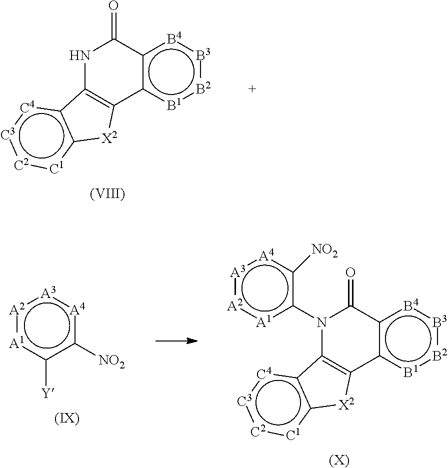

wherein X², A¹, A², A³, A⁴, C¹, C², C³, C⁴, B¹, B², B³ and B⁴ have the same meanings as mentioned above, and Y′ is F, Cl, Br, or I.

The reaction conditions, bases and solvents suitable are in general known by a person skilled in the art.

Suitable bases are preferably selected from the group consisting of alkali metal and alkaline earth metal hydroxides such as NaOH, KOH, or Ca(OH)₂, alkali metal hydrides such as NaH or KH, alkali metal amides such as NaNH₂, alkali metal or alkaline earth metal carbonates such as K₂CO₃ or Cs₂CO₃, alkali metal phosphates such as K₃PO₄, and alkali metal alkoxides such as NaOMe or NaOEt. In addition, mixtures of the aforementioned bases are suitable.

More preferable bases are NaH, KOH, NaOH, K₃PO₄, and Cs₂CO₃. Particular preference is given to K₃PO₄ and K₂CO₃.

Suitable solvents are, for example, (polar) aprotic solvents such as THF, dioxane, dimethyl sulfoxide (DMSO), diethylformamide (DMF), N-methyl-2-pyrrolidone (NMP), or tridecane or alcohols such as ethanol.

More preferred solvents are THF, dioxane, DMF, and DMSO.

The reaction temperature of step (b) is preferably in the range from room temperature (i.e. 20° C.) to 100° C.

The reaction time is preferably from 4 to 12 hours.

The compound according to general formula (X) which is the product of step (b) of the process according to the present invention is introduced into step (c) of the process according to the present invention.

Step (c) of the process according to the present invention comprises the reduction of the compound according to general formula (X) with a reducing reagent or with H₂ in the presence of a catalyst like Pd/C in a solvent to obtain a compound according to general formula (XI):

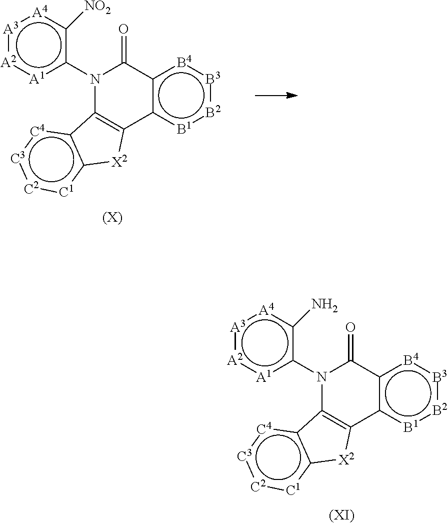

wherein X², A¹, A², A³, A⁴, C¹, C², C³, C⁴, B¹, B², B³ and B⁴ have the same meanings as mentioned above.

The reaction conditions and solvents suitable for step (c) are known by a person skilled in the art. Preferred reducing reagents are for example Sn, Zn, and/or Fe. If one of those reducing agents is used, preferred solvents are alcohols such as ethanol, isopropanol, acetic acid, and/or THF. Additionally, HCl (concentrated or diluted with water) may be added. The reaction temperature preferably ranges from room temperature to 130° C. The reaction time is preferably between 4 to 12 hours.

The reaction conditions and solvents suitable for the reduction with H₂ in the presence of a catalyst like Pd/C are in general known by a person skilled in the art. Suitable solvents for this embodiment are, for example, (polar) aprotic solvents such as THF, dioxane, dimethyl sulfoxide (DMSO), diethylformamide (DMF), N-methyl-2-pyrrolidone (NMP), tridecane or alcohols such as ethanol. More preferred solvents are THF, ethanol, and/or DMF.

The reaction temperature is preferably in the range from room temperature (i.e. 20° C.) to 50° C.

The reaction time is preferably from 1 to 5 hours.

The pressure of hydrogen is preferably from 1 to 5 bars.

The compound according to general formula (XI) which is the product of step (c) of the process according to the present invention is introduced into step (d) of the process according to the present invention.

Step (d) comprises the reaction of the compound according to general formula (XI) in the presence of a catalyst to obtain the compound according to general formula (I):

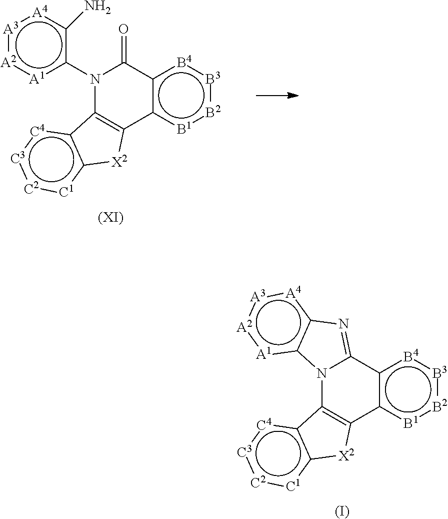

wherein X², A¹, A², A³, A⁴, C¹, C², C³, C⁴, B¹, B², B³ and B⁴ have the same meanings as mentioned above.

The reaction conditions and solvents suitable in step (d) of the process according to the present invention are in general known by a person skilled in the art.

Preferred catalysts are polyphosphoric acid, p-toluenesulfonic acid and/or trifluoroacetic acid.

Step (d) may be carried out in the presence or in the absence of a solvent. Preferably, no solvent is used in step (d) of the process according to the present invention.

In the case that a solvent is used, preferred solvents are aromatic solvents such as toluene and xylene.

The temperature is preferably in the range from room temperature (i.e. 20° C.) to 250° C., preferably 150 to 220° C.

The reaction time is preferably from 5 to 24 hours.

According to a second embodiment, in the compound according to formula (I), X¹ is O, S, NR²⁵,

or CR²⁶R²⁷ and X² is direct bond, wherein R²⁵, R²⁶ and R²⁷ have the same meanings as defined above. Compounds according to this second embodiment are prepared by the process comprising step (e).

According to step (e) of the process, a compound according to general formula (XII) is reacted with a compound according to general formula (XIII) to obtain a compound according to general formula (I):

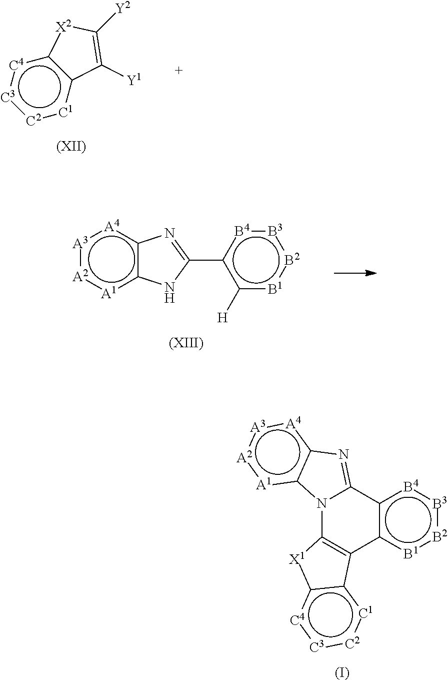

wherein X¹, A¹, A², A³, A⁴, C¹, C², C³, C⁴, B¹, B², B³ and B⁴ have the same meanings as mentioned above, and (i) Y¹ is I and Y² is Br or Cl or (ii) Y is Br and Y² is Cl or (iii) Y¹ and Y² are the same halogen.

Step (e) of the process according to the present invention is preferably conducted in the presence of a base and a catalyst. The reaction conditions, bases and solvents suitable are in general known by a person skilled in the art and are disclosed, for example in Org. Biomol. Chem. 2013, 11, 7966.

Suitable bases are preferably selected from the group consisting of alkali metal and alkaline earth metal hydroxides such as NaOH, KOH, or Ca(OH)₂, alkali metal hydrides such as NaH or KH, alkali metal amides such as NaNH₂, alkali metal or alkaline earth metal carbonates such as K₂CO₃ or Cs₂CO₃, alkali metal phosphates such as K₃PO₄, and alkali metal alkoxides such as NaOMe or NaOEt. In addition, mixtures of the aforementioned bases are suitable.

More preferable bases are NaH, KOH, NaOH, K₃PO₄, and Cs₂CO₃. Particular preference is given to Cs₂CO₃.

Suitable solvents are, for example, (polar) aprotic solvents such as THF, dioxane, dimethyl sulfoxide (DMSO), diethylformamide (DMF), N-methyl-2-pyrrolidone (NMP), or tridecane or alcohols such as ethanol.

More preferred solvents are THF, dioxane, DMF, and DMSO, particularly preferred DMF.

The reaction temperature of step (e) is preferably in the range from room temperature (i.e. 20° C.) to 200° C., preferably 100 to 160° C.

The reaction time is preferably from 12 to 36 hours.

Compounds of formulae (I), (II) or (III) in organic electronics applications

It has been found that the compounds of the formulae (I), (II) or (III) are particularly suitable for use in applications in which charge carrier conductivity is required, especially for use in organic electronics applications, for example selected from switching elements such as organic transistors, e.g. organic FETs and organic TFTs, organic solar cells and organic light-emitting diodes (OLEDs).

The organic transistorgenerally includes a semiconductor layer formed from an organic layer with charge transport capacity; a gate electrode formed from a conductive layer; and an insulating layer introduced between the semiconductor layer and the conductive layer. A source electrode and a drain electrode are mounted on this arrangement in order thus to produce the transistor element. In addition, further layers known to those skilled in the art may be present in the organic transistor. The layers with charge transport capacity may comprise the compounds of formulae (I), (II) or (III).

The organic solar cell (photoelectric conversion element) generally comprises an organic layer present between two plate-type electrodes arranged in parallel. The organic layer may be configured on a comb-type electrode. There is no particular restriction regarding the site of the organic layer and there is no particular restriction regarding the material of the electrodes. When, however, plate-type electrodes arranged in parallel are used, at least one electrode is preferably formed from a transparent electrode, for example an ITO electrode or a fluorine-doped tin oxide electrode. The organic layer is formed from two sublayers, i.e. a layer with p-type semiconductor properties or hole transport capacity, and a layer formed with n-type semiconductor properties or charge transport capacity. In addition, it is possible for further layers known to those skilled in the art to be present in the organic solar cell. The layers with charge transport capacity may comprise the compounds of formulae (I), (II) or (III).

The compounds of the formulae (I), (II) or (III) being particularly suitable in OLEDs for use as a host (=matrix) material, preferably in a light-emitting layer, and/or as a charge and/or excition blocker material, and/or as a charge transport material, for example as a hole transport material and/or as a charge transport material, preferably as an electron transporting material, especially in combination with a phosphorescence emitter and/or as a dopant without metal species. Further preferred, the compounds of the formulae (I), (II) or (III) are particularly suitable in OLEDs for use as an electron transporting material, preferably in a light-emitting layer, especially in combination with a fluorescence emitter.

The present invention therefore preferably relates to the use of a compound according to general formulae (I), (II) or (III) as defined above in an electronic device, preferably in an electroluminescence device, particularly preferably in an organic light emitting diode (OLED), preferably in an emitting layer, as a host material, a charge transporting material, for example as a hole transport material or an electron transport material, preferably as an electron transporting material, and/or a dopant without metal species, preferably as a host material or an electron transporting material.

In the case of use of the inventive compounds of the formulae (I), (II) or (III) in OLEDs, OLEDs which have good efficiencies and a long lifetime and which can be operated especially at a low use and operating voltage are obtained. The inventive compounds of the formulae (I), (II) or (III) are suitable especially for use as matrix and/or charge transport and/or charge blocking materials for green, red and yellow, preferably green and red, more preferably red emitters. The inventive compounds of the formulae (I), (II) or (III) are further suitable especially for use as electron transporting material for blue emitters. Furthermore, the compounds of the formulae (I), (II) or (III) can be used as conductor/complementary materials in organic electronics applications selected from switching elements and organic solar cells. According to the present application, the terms matrix and host are used interchangeable.

In the emission layer or one of the emission layers of an OLED, it is also possible to combine an emitter material with at least one matrix material of the compound of the formulae (I), (II) or (III) and one or more, preferably one, further matrix materials (co-host). This may achieve a high quantum efficiency, low driving voltage and/or long lifetime of these devices.

According to one preferred embodiment of the present invention the compounds according to general formulae (I), (II) or (III) are used as host materials, preferably in emitting layers comprising red light-emitting compounds. According to this embodiment, preferably no further host material is present in the light-emitting layer.

According to another preferred embodiment of the present invention the compounds according to general formulae (I), (II) or (III) are used as host materials, preferably in emitting layers comprising green light-emitting compounds. According to this embodiment, the compounds according to the present invention are preferably used in the presence of at least one further host material, i.e. as a co-host. Further host materials are mentioned in the following.

It is likewise possible that the compounds of the formulae (I), (II) or (III) are present in two or three of the following layers: in the light-emitting layer (preferably as host material) and/or in the transport layer (as electron transport material).

When a compound of the formulae (I), (II) or (III) is used as matrix (host) material in an emission layer and additionally as electron transport material, owing to the chemical identity or similarity of the materials, an improved interface between the emission layer and the adjacent material, which can lead to a decrease in the voltage with equal luminance and to an extension of the lifetime of the OLED. Moreover, the use of the same material as electron transport material and/or as matrix of an emission layer allows the production process of an OLED to be simplified, since the same source can be used for the vapor deposition process of the material of one of the compounds of the formula the compound of the formulae (I), (II) or (III).

Suitable structures of organic electronic devices, especially organic light-emitting diodes (OLED), are known to those skilled in the art and are specified below.

For example, the electronic device, preferably an organic electroluminescence device, more preferably an organic light emitting diode (OLED), according to the present invention comprises a cathode, an anode, and a plurality of organic thin film layers provided between the cathode and the anode, the organic thin film layers comprising an emitting layer comprising the at least one compound of general formulae (I), (II) or (III), preferably as a host material, a charge transporting material, for example as a hole transport material or an electron transport material, preferably as an electron transporting material, and/or a dopant without metal species as, particularly preferably as a host material or as an electron transporting material.

The present invention therefore preferably relates to the electronic device, preferably an organic electroluminescence device, more preferably an organic light emitting diode (OLED), according to the present invention, comprising a cathode, an anode, and a plurality of organic thin film layers provided between the cathode and the anode, the organic thin film layers comprising an emitting layer comprising the at least one compound of general formulae (I), (II) or (III), preferably as a host material, a charge transporting material, for example as a hole transport material or an electron transport material, preferably as an electron transporting material, and/or a dopant without metal species as, particularly preferably as a host material or as an electron transporting material.

More preferably, the present invention provides an organic light-emitting diode (OLED) comprising an anode (a) and a cathode (i) and a light-emitting layer (e) arranged between the anode (a) and the cathode (i), and if appropriate at least one further layer selected from the group consisting of at least one blocking layer for holes/excitons, at least one blocking layer for electrons/excitons, at least one hole injection layer, at least one hole transport layer, at least one electron injection layer and at least one electron transport layer, wherein the at least one compound of the formulae (I), (II) or (III) is present in the light-emitting layer (e) and/or in at least one of the further layers. The at least one compound of the formulae (I), (II) or (III) is preferably present in the light-emitting layer and/or hole/exciton blocking layer and/or the charge blocking layer, i.e. the electron or hole transport layer.

In a preferred embodiment of the present invention, at least one compound of the formulae (I), (II) or (III) is used as electron transport material. Examples of preferred compounds of the formulae (I), (II) or (III) are shown above.

In another preferred embodiment of the present invention, at least one compound of the formulae (I), (II) or (III) is used as charge/exciton blocker material. Examples of preferred compounds of the formulae (I), (II) or (III) are shown above.

The present application further relates to a light-emitting layer, preferably present in an electronic device, more preferably in an electroluminescence device, particularly preferably in an organic light emitting diode (OLED), comprising at least one compound of general formulae (I), (II) or (III) as defined above, preferably as host material or co-host material. Examples of preferred compounds of the formulae (I), (II) or (III) are shown above.

Most preferably, the electronic device according to the present invention is an organic light emitting diode (OLED).

Structure of the Inventive OLED

The inventive organic light-emitting diode (OLED) thus generally has the following structure: an anode (a) and a cathode (i) and a light-emitting layer (e) arranged between the anode (a) and the cathode (i).

The inventive OLED may, for example—in a preferred embodiment—be formed from the following layers:

1. Anode (a)

2. Hole transport layer (c)

3. Light-emitting layer (e)

4. Blocking layer for holes/excitons (f)

5. Electron transport layer (g)

6. Cathode (i)

Layer sequences different than the aforementioned structure are also possible, and are known to those skilled in the art. For example, it is possible that the OLED does not have all of the layers mentioned; for example, an OLED with layers (a) (anode), (e) (light-emitting layer) and (i) (cathode) is likewise suitable, in which case the functions of the layers (c) (hole transport layer) and (f) (blocking layer for holes/excitons) and (g) (electron transport layer) are assumed by the adjacent layers. OLEDs which have layers (a), (c), (e) and (i), or layers (a), (e), (f), (g) and (i), are likewise suitable. In addition, the OLEDs may have a blocking layer for electrons/excitons (d) between the hole transport layer (c) and the light-emitting layer (e).

It is additionally possible that a plurality of the aforementioned functions (electron/exciton blocker, hole/exciton blocker, hole injection, hole conduction, electron injection, electron conduction) are combined in one layer and are assumed, for example, by a single material present in this layer. For example, a material used in the hole transport layer, in one embodiment, may simultaneously block excitons and/or electrons.

Furthermore, the individual layers of the OLED among those specified above may in turn be formed from two or more layers. For example, the hole transport layer may be formed from a layer into which holes are injected from the electrode, and a layer which transports the holes away from the hole-injecting layer into the light-emitting layer. The electron transport layer may likewise consist of a plurality of layers, for example a layer in which electrons are injected by the electrode, and a layer which receives electrons from the electron injection layer and transports them into the light-emitting layer. These layers mentioned are each selected according to factors such as energy level, thermal resistance and charge carrier mobility, and also energy difference of the layers specified with the organic layers or the metal electrodes. The person skilled in the art is capable of selecting the structure of the OLEDs such that it is matched optimally to the organic compounds used in accordance with the invention.

In a preferred embodiment the OLED according to the present invention comprises in this order:

(a) an anode,

(b) optionally a hole injection layer,

(c) optionally a hole transport layer,

(d) optionally an exciton blocking layer

(e) an emitting layer,

(f) optionally a hole/exciton blocking layer

(g) optionally an electron transport layer,

(h) optionally an electron injection layer, and

(i) a cathode.

In a particularly preferred embodiment the OLED according to the present invention comprises in this order:

(a) an anode,

(b) optionally a hole injection layer,

(c) a hole transport layer,

(d) an exciton blocking layer

(e) an emitting layer,

(f) a hole/exciton blocking layer

(g) an electron transport layer, and

(h) optionally an electron injection layer, and

(i) a cathode.

The properties and functions of these various layers, as well as example materials are known from the prior art and are described in more detail below on basis of preferred embodiments.

Anode (a):

The anode is an electrode which provides positive charge carriers. It may be composed, for example, of materials which comprise a metal, a mixture of different metals, a metal alloy, a metal oxide or a mixture of different metal oxides. Alternatively, the anode may be a conductive polymer. Suitable metals comprise the metals of groups 11, 4, 5 and 6 of the Periodic Table of the Elements, and also the transition metals of groups 8 to 10. When the anode is to be transparent, mixed metal oxides of groups 12, 13 and 14 of the Periodic Table of the Elements are generally used, for example indium tin oxide (ITO). It is likewise possible that the anode (a) comprises an organic material, for example polyaniline, as described, for example, in Nature, Vol. 357, pages 477 to 479 (Jun. 11, 1992). Preferred anode materials include conductive metal oxides, such as indium tin oxide (ITO) and indium zinc oxide (IZO), aluminum zinc oxide (AlZnO), and metals. Anode (and substrate) may be sufficiently transparent to create a bottom-emitting device. A preferred transparent substrate and anode combination is commercially available ITO (anode) deposited on glass or plastic (substrate). A reflective anode may be preferred for some top-emitting devices, to increase the amount of light emitted from the top of the device. At least either the anode or the cathode should be at least partly transparent in order to be able to emit the light formed. Other anode materials and structures may be used.

Hole Injection Layer (b):

Generally, injection layers are comprised of a material that may improve the injection of charge carriers from one layer, such as an electrode or a charge generating layer, into an adjacent organic layer. Injection layers may also perform a charge transport function. The hole injection layer may be any layer that improves the injection of holes from anode into an adjacent organic layer. A hole injection layer may comprise a solution deposited material, such as a spin-coated polymer, or it may be a vapor deposited small molecule material, such as, for example, CuPc or MTDATA. Polymeric hole-injection materials can be used such as poly(N-vinylcarbazole) (PVK), polythiophenes, polypyrrole, polyaniline, self-doping polymers, such as, for example, sulfonated poly(thiophene-3-[2[(2-methoxyethoxy)ethoxy]-2,5-diyl) (Plexcore® OC Conducting Inks commercially available from Plextronics), and copolymers such as poly(3,4-ethylenedioxythiophene)/poly(4-styrenesulfonate) also called PEDOT/PSS.

An example for a suitable hole injection material is:

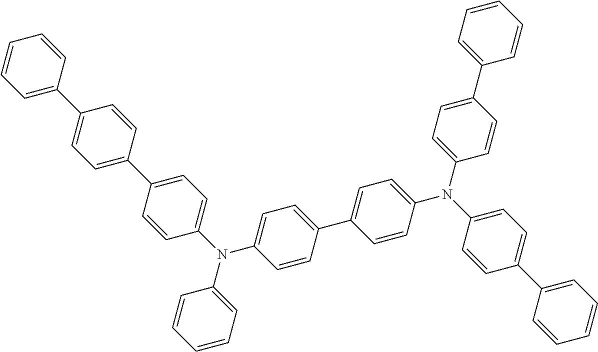 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

(see also hole-transporting molecules)

Hole Transport Layer (c):

According to a preferred embodiment the OLED according to the present invention comprises at least one compound according to general formulae (I), (II) or (III) or their preferred embodiments as a charge transporting material, preferably as a hole transporting layer. In addition to the compounds according to general formulae (I), (II) or (III) or without these compounds either hole-transporting molecules or polymers may be used as the hole transport material. Suitable hole transport materials for layer (c) of the inventive OLED are disclosed, for example, in Kirk-Othmer Encyclopedia of Chemical Technology, 4th Edition, Vol. 18, pages 837 to 860, 1996, US20070278938, US2008/0106190, US2011/0163302 (triarylamines with (di)benzothiophen/(di)benzofuran; Nan-Xing Hu et al. Synth. Met. 111 (2000) 421 (indolocarbazoles), WO2010002850 (substituted phenylamine compounds) and WO2012/16601 (in particular the hole transport materials mentioned on pages 16 and 17 of WO2012/16601). Combination of different hole transport material may be used. Reference is made, for example, to WO2013/022419, wherein

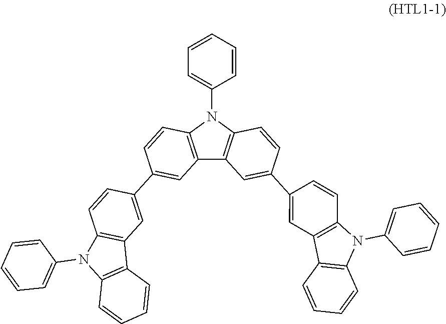 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

and

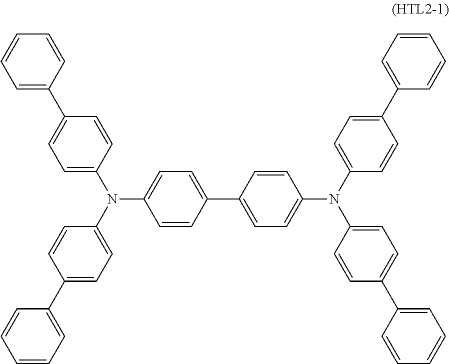 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

constitute the hole transport layer.

Customarily used hole-transporting molecules are selected from the group consisting of

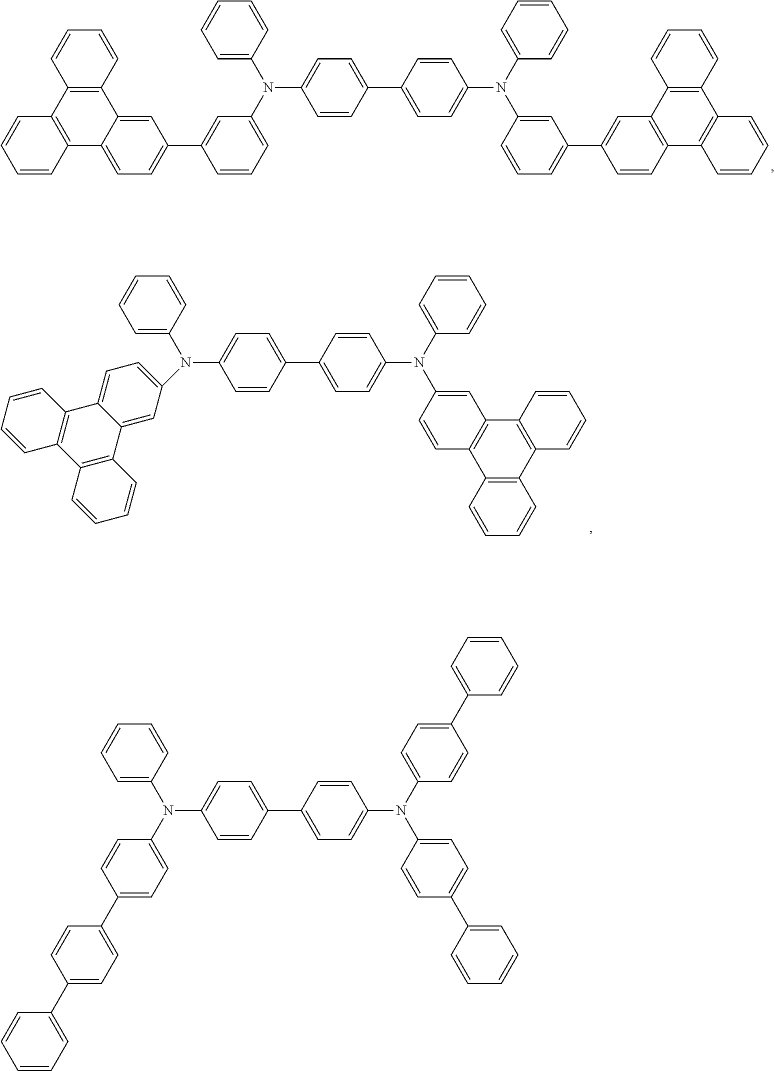 [SMILES: c1ccc(N(c2ccc(-c3ccc(N(c4ccccc4)c4cccc(-c5ccc6c7ccccc7c7ccccc7c6c5)c4)cc3)cc2)c2cccc(-c3ccc4c5ccccc5c5ccccc5c4c3)c2)cc1 ; c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

(4-phenyl-N-(4-phenylphenyl)-N-[4-[4-(N-[4-(4-phenylphenyl)phenyl]anilino)phenyl]phenyl]aniline),

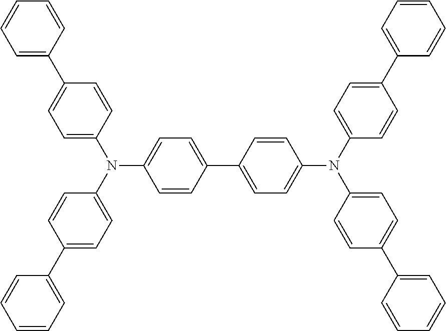 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

(4-phenyl-N-(4-phenylphenyl)-N-[4-[4-(4-phenyl-N-(4-phenylphenyl)anilino)phenyl]phenyl]aniline),

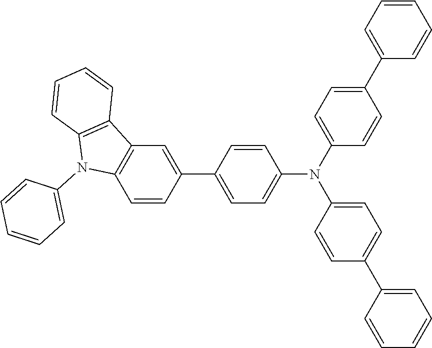 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

(4-phenyl-N-[4-(9-phenylcarbazol-3-yl)phenyl]-N-(4-phenylphenyl)aniline),

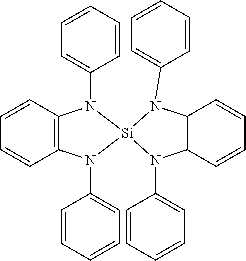 [SMILES: C1=CC2C(C=C1)N(c1ccccc1)[Si]1(N(c3ccccc3)c3ccccc3N1c1ccccc1)N2c1ccccc1]

(1,1′,3,3′-tetraphenylspiro[1,3,2-benzodiazasilole-2,2′-3a,7a-dihydro-1,3,2-benzodiazasilole]),

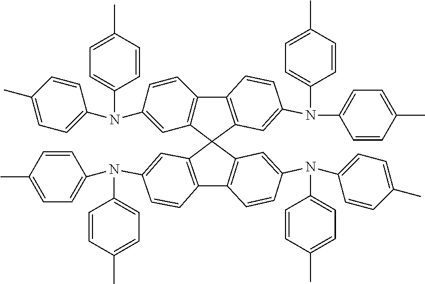

(N2,N2,N2′,N2′,N7,N7,N7′,N7′-octakis(p-tolyl)-9,9′-spirobi[fluorene]-2,2′,7,7′-tetramine), 4,4′-bis[N-(1-naphthyl)-N-phenylamino]biphenyl (α-NPD), N,N′-diphenyl-N,N′-bis(3-methylphenyl)-[1,1′-biphenyl]-4,4′-diamine (TPD), 1,1-bis[(di-4-tolylamino)phenyl]cyclohexane (TAPC), N,N′-bis(4-methylphenyl)-N,N′-bis(4-ethylphenyl)-[1,1′-(3,3′-dimethyl)biphenyl]-4,4′-diamine (ETPD), tetrakis(3-methylphenyl)-N,N,N′,N′-2,5-phenylenediamine (PDA), α-phenyl-4-N,N-diphenylaminostyrene (TPS), p-(diethylamino)benzaldehyde diphenylhydrazone (DEH), triphenylamine (TPA), bis[4-(N,N-diethylamino)-2-methylphenyl](4-methylphenyl)methane (MPMP), 1-phenyl-3-[p-(diethylamino)styryl]5-[p-(diethylamino)phenyl]pyrazoline (PPR or DEASP), 1,2-trans-bis(9H-carbazol9-yl)-cyclobutane (DCZB), N,N, N′,N′-tetrakis(4-methylphenyl)-(1,1′-biphenyl)-4,4′-diamine (TTB), fluorine compounds such as 2,2′,7,7′-tetra(N,N-di-tolyl)amino9,9-spirobifluorene (spiro-TTB), N,N′-bis(naphthalen-1-yl)-N,N′-bis(phenyl)9,9-spirobifluorene (spiro-NPB) and 9,9-bis(4-(N,N-bis-biphenyl-4-yl-amino)phenyl-9Hfluorene, benzidine compounds such as N,N′-bis(naphthalen-1-yl)-N,N′-bis(phenyl)benzidine and porphyrin compounds such as copper phthalocyanines. In addition, polymeric hole-injection materials can be used such as poly(N-vinylcarbazole) (PVK), polythiophenes, polypyrrole, polyaniline, self-doping polymers, such as, for example, sulfonated poly(thiophene-3-[2[(2-methoxyethoxy)ethoxy]-2,5-diyl) (Plexcore® OC Conducting Inks commercially available from Plextronics), and copolymers such as poly(3,4-ethylenedioxythiophene)/poly(4-styrenesulfonate) also called PEDOT/PSS. Preferred examples of a material of the hole injecting layer are a porphyrin compound, an aromatic tertiary amine compound, or a styrylamine compound. Particularly preferable examples include an aromatic tertiary amine compound such as hexacyanohexaazatriphenylene (HAT).

The hole-transporting layer may also be electronically doped in order to improve the transport properties of the materials used, in order firstly to make the layer thicknesses more generous (avoidance of pinholes/short circuits) and in order secondly to minimize the operating voltage of the device. Electronic doping is known to those skilled in the art and is disclosed, for example, in W. Gao, A. Kahn, J. Appl. Phys., Vol. 94, 2003, 359 (p-doped organic layers); A. G. Werner, F. Li, K. Harada, M. Pfeiffer, T. Fritz, K. Leo, Appl. Phys. Lett., Vol. 82, No. 25, 2003, 4495 and Pfeiffer et al., Organic Electronics 2003, 4, 89-103 and K. Walzer, B. Maennig, M. Pfeiffer, K. Leo, Chem. Soc. Rev. 2007, 107, 1233. For example it is possible to use mixtures in the hole-transporting layer, in particular mixtures which lead to electrical p-doping of the hole-transporting layer. p-Doping is achieved by the addition of oxidizing materials. These mixtures may, for example, be the following mixtures: mixtures of the abovementioned hole transport materials with at least one metal oxide, for example MoO₂, MoO₃, WOₓ, ReO₃ and/or V₂O₅, preferably MoO₃ and/or ReO₃, more preferably MoO₃, or mixtures comprising the aforementioned hole transport materials and one or more compounds selected from 7,7,8,8-tetracyanoquinodimethane (TCNQ), 2,3,5,6-tetrafluoro-7,7,8,8-tetracyanoquinodimethane (F₄-TCNQ), 2,5-bis(2-hydroxyethoxy)-7,7,8,8-tetracyanoquinodimethane, bis(tetra-n-butylammonium)tetracyanodiphenoquinodimethane, 2,5-dimethyl-7,7,8,8-tetracyanoquinodimethane, tetracyanoethylene, 11,11,12,12-tetracyanonaphtho-2,6-quinodimethane, 2-fluoro-7,7,8,8-tetracyanoquino-dimethane, 2,5-difluoro-7,7,8,8-etracyanoquinodimethane, dicyanomethylene-1,3,4,5,7,8-hexafluoro-6H-naphthalen-2-ylidene)malononitrile (F₆-TNAP), Mo(tfd)₃ (from Kahn et al., J. Am. Chem. Soc. 2009, 131 (35), 12530-12531), compounds as described in EP1988587, US2008265216, EP2180029, US20100102709, WO2010132236, EP2180029 and quinone compounds as mentioned in EP2401254.

Exciton Blocking Layer (d):

Blocking layers may be used to reduce the number of charge carriers (electrons or holes) and/or excitons that leave the emissive layer. An electron/exciton blocking layer (d) may be disposed between the first emitting layer (e) and the hole transport layer (c), to block electrons from emitting layer (e) in the direction of hole transport layer (c). Blocking layers may also be used to block excitons from diffusing out of the emissive layer.

Suitable metal complexes for use as electron/exciton blocker material are, for example, carbene complexes as described in WO2005/019373A2, WO2006/056418A2, WO2005/113704, WO2007/115970, WO2007/115981, WO2008/000727 and PCT/EP2014/055520. Explicit reference is made here to the disclosure of the WO applications cited, and these disclosures shall be considered to be incorporated into the content of the present application.

Emitting Layer (e):

The light emitting layer is an organic layer having a light emitting function and is formed from one or more layers, wherein one of the layers comprises a host material (first host material), optionally a second host material, and the light emitting material as described below.

When the light emitting layer is composed of two or more layers, the light emitting layer or layers other than that mentioned above contains or contain a host material and a dopant material when a doping system is employed. The major function of the host material is to promote the recombination of electrons and holes and confine excitons in the light emitting layer. The dopant material causes the excitons generated by recombination to emit light efficiently.

In case of a phosphorescent device, the major function of the host material is to confine the excitons generated on the dopant in the light emitting layer.

The light emitting layer may be made into a double dopant layer, in which two or more kinds of dopant materials having high quantum yield are used in combination and each dopant material emits light with its own color. For example, to obtain a yellow emission, a light emitting layer formed by co-depositing a host, a red-emitting dopant and a green-emitting dopant is used.

In a laminate of two or more light emitting layers, electrons and holes are accumulated in the interface between the light emitting layers, and therefore, the recombination region is localized in the interface between the light emitting layers, to improve the quantum efficiency.

The light emitting layer may be different in the hole injection ability and the electron injection ability, and also in the hole transporting ability and the electron transporting ability each being expressed by mobility.

The light emitting layer is formed, for example, by a known method, such as a vapor deposition method, a spin coating method, and LB method. Alternatively, the light emitting layer may be formed by making a solution of a binder, such as resin, and the material for the light emitting layer in a solvent into a thin film by a method such as spin coating.

The light emitting layer is preferably a molecular deposit film. The molecular deposit film is a thin film formed by depositing a vaporized material or a film formed by solidifying a material in the state of solution or liquid. The molecular deposit film can be distinguished from a thin film formed by LB method (molecular build-up film) by the differences in the assembly structures and higher order structures and the functional difference due to the structural differences.

The light-emitting layer (e) comprises at least one emitter material. In principle, it may be a fluorescence or phosphorescence emitter, suitable emitter materials being known to those skilled in the art. The at least one emitter material is preferably a phosphorescence emitter.

The emission wavelength of the phosphorescent dopant used in the light emitting layer is not particularly limited. In a preferred embodiment, at least one of the phosphorescent dopants used in the light emitting layer has the peak of emission wavelength of in general 430 nm or longer and 780 nm or shorter, preferably 490 nm or longer and 700 nm or shorter and more preferably 490 nm or longer and 650 nm or shorter. Most preferred are green emitter materials (490 to 570 nm). In another preferred embodiment, red emitter materials (570 to 680 nm) are preferred.

The phosphorescent dopant (phosphorescent emitter material) is a compound which emits light by releasing the energy of excited triplet state and preferably a organometallic complex comprising at least one metal selected from Ir, Pt, Pd, Os, Au, Cu, Re, Rh and Ru and a ligand, although not particularly limited thereto as long as emitting light by releasing the energy of excited triplet state. A ligand having an ortho metal bond is preferred. In view of obtaining a high phosphorescent quantum yield and further improving the external quantum efficiency of electroluminescence device, a metal complex comprising a metal selected from Ir, Os, and Pt is preferred, with iridium complex, osmium complex, and platinum, particularly an ortho metallated complex thereof being more preferred, iridium complex and platinum complex being still more preferred, and an ortho metallated iridium complex being particularly preferred.

According to the present invention, the compounds of the formulae (I), (II) or (III) can be used as dopant without metal species. According to the present invention, the wording “without metal species” means that the amount of metal in the compound according to formulae (I), (II) or (III) is below the limit which can be detected with common analytics like ICP-MS (Inductively Coupled Plasma Mass Spectrometry) and ICP-AES (Inductively Coupled Plasma Atomic Emission Spectrometry).

If the compounds of the formulae (I), (II) or (III) are used as dopant without metal species, the peak of emission wavelength of is in general at 420 nm or longer and 780 nm or shorter, preferably 440 nm or longer and 550 nm or shorter and more preferably 440 nm or longer and 520 nm or shorter.

According to a preferred embodiment of the present invention, the compounds of the formulae (I), (II) or (III) can be used as the matrix (=host material) in the light-emitting layer.

The compounds of the formulae (I), (II) or (III) can preferably be used as the matrix (=host material) in the light-emitting layer.

Suitable metal complexes for use in the inventive OLEDs, preferably as emitter material, are described, for example, in documents WO02/60910A1, US2001/0015432A1, US2001/0019782A1, US2002/0055014A1, US2002/0024293A1, US2002/0048689A1, EP1191612A2, EP1191613A2, EP1211257A2, US2002/0094453A1, WO02/02714A2, WO00/70655A2, WO01/41512A1, WO02/15645A1, WO2005/019373A2, WO2005/113704A2, WO2006/115301A1, WO02006/067074A1, WO2006/056418, WO2006121811A1, WO2007095118A2, WO2007/115970, WO2007/115981, WO2008/000727, WO2010129323, WO2010056669, WO 10086089, US2011/0057559, WO2011/106344, US2011/0233528, WO2012/048266 and WO2012/172482.

Further suitable metal complexes are the commercially available metal complexes tris(2-phenylpyridine)iridium(III), iridium(III) tris(2-(4-tolyl)pyridinato-N,C^{2′}), bis(2-phenylpyridine)(acetylacetonato)iridium(III), iridium(III) tris(1-phenylisoquinoline), iridium(III) bis(2,2′-benzothienyl)pyridinato-N,C^{3′})(acetylacetonate), tris(2-phenylquinoline)iridium(III), iridium(III) bis(2-(4,6-difluorophenyl)pyridinato-N,C²)picolinate, iridium(III) bis(1-phenylisoquinoline)(acetylacetonate), bis(2-phenylquinoline)(acetylacetonato)iridium(III), iridium(III) bis(di-benzo[f,h]quinoxaline)(acetylacetonate), iridium(III) bis(2-methyldi-benzo[f,h]quinoxaline)(acetylacetonate) and tris(3-methyl-1-phenyl-4-trimethylacetyl-5-pyrazolino)terbium(III), bis[1-(9,9-dimethyl-9H-fluoren-2-yl)isoquinoline](acetylacetonato)iridium(III), bis(2-phenylbenzothiazolato)(acetylacetonato)iridium(II), bis(2-(9,9-dihexylfluorenyl)-1-pyridine)(acetylacetonato)iridium(III), bis(2-benzo[b]thiophen-2-yl-pyridine)(acetylacetonato)iridium(III).

In addition, the following commercially available materials are suitable: tris(dibenzoylacetonato)mono(phenanthroline)europium(III), tris(dibenzoylmethane)-mono(phenanthroline)europium(III), tris(dibenzoylmethane)mono(5-aminophenanthroline)europium(III), tris(di-2-naphthoylmethane)mono(phenanthroline)europium(III), tris(4-bromobenzoylmethane)mono(phenanthroline)europium(III), tris(di(biphenyl)methane)mono(phenanthroline)europium(II), tris(dibenzoylmethane)mono(4,7-diphenylphenanthroline)europium(III), tris(dibenzoylmethane)mono(4,7-di-methylphenanthroline)europium(III), tris(dibenzoylmethane)mono(4,7-dimethylphenanthrolinedisulfonic acid)europium(III) disodium salt, tris[di(4-(2-(2-ethoxyethoxy)ethoxy)benzoylmethane)]mono(phenanthroline)europium(II) and tris[di[4-(2-(2-ethoxyethoxy)ethoxy)benzoylmethane)]mono(5-aminophenanthroline)europium(III), osmium(III) bis(3-(trifluoromethyl)-5-(4-tert-butylpyridyl)-1,2,4-triazolato)diphenylmethylphosphine, osmium(II) bis(3-(trifluoromethyl)-5-(2-pyridyl)-1,2,4-triazole)dimethylphenylphosphine, osmium(II) bis(3-(trifluoromethyl)-5-(4-tert-butylpyridyl)-1,2,4-triazolato)dimethylphenylphosphine, osmium(II) bis(3-(trifluoromethyl)-5-(2-pyridyl)pyrazolato)dimethylphenylphosphine, tris[4,4′-di-tert-butyl(2,2′)-bipyridine]ruthenium(III), osmium(II) bis(2-(9,9-dibutylfluorenyl)-1-isoquinoline(acetylacetonate).

Particularly suitable metal complexes are described in US2014048784, US2012223295, US2014367667, US2013234119, US2014001446, US2014231794, US2014008633, WO2012108388 and WO2012108389. The emitters mentioned in US2013234119, paragraph [0222], are exemplified. Selected emitters, especially red emitters, of said emitters mentioned in US2013234119, paragraph [0222], are:

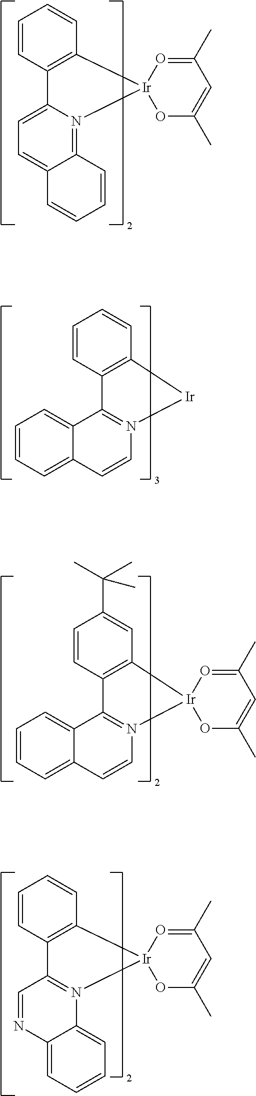

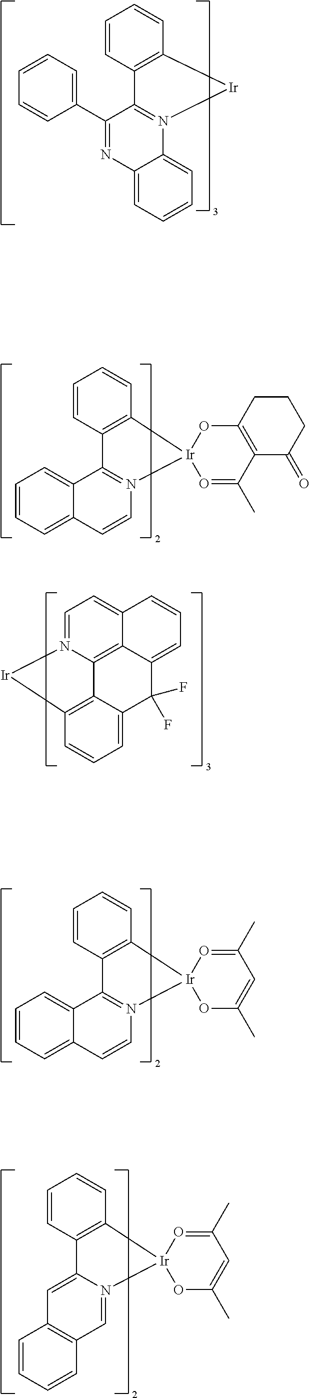

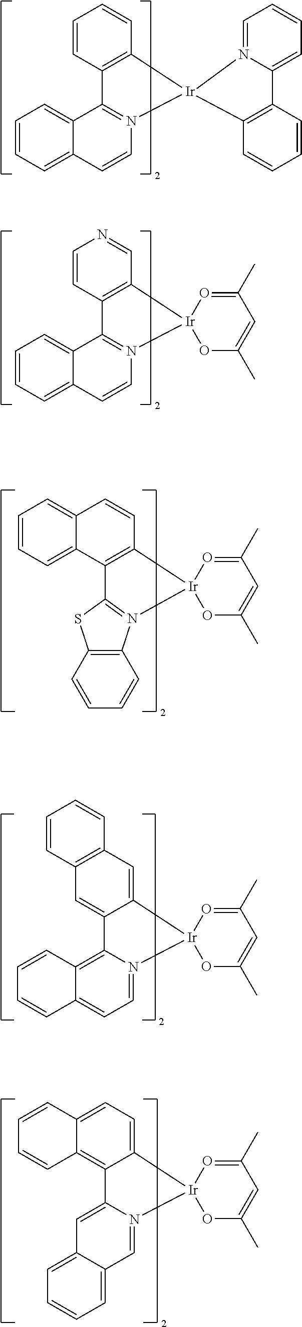

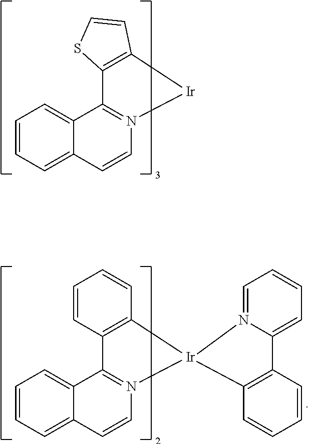

Further suitable Emitters are mentioned in: Mrs Bulletin, 2007, 32, 694:

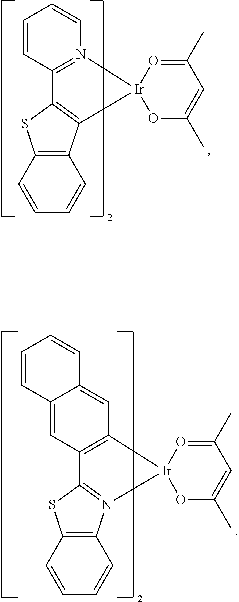

Further suitable Emitters are mentioned in: WO2009100991:

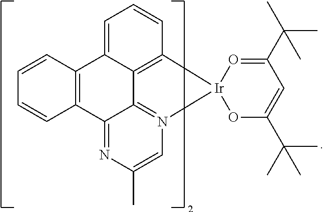

Further suitable Emitters are mentioned in: WO2008101842:

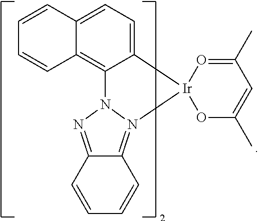

Further suitable Emitters are mentioned in: US 20140048784, especially in paragraph [0159]:

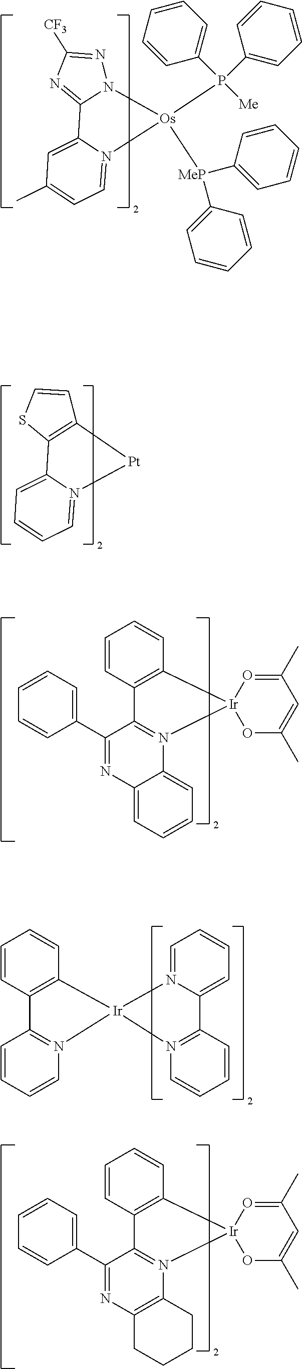

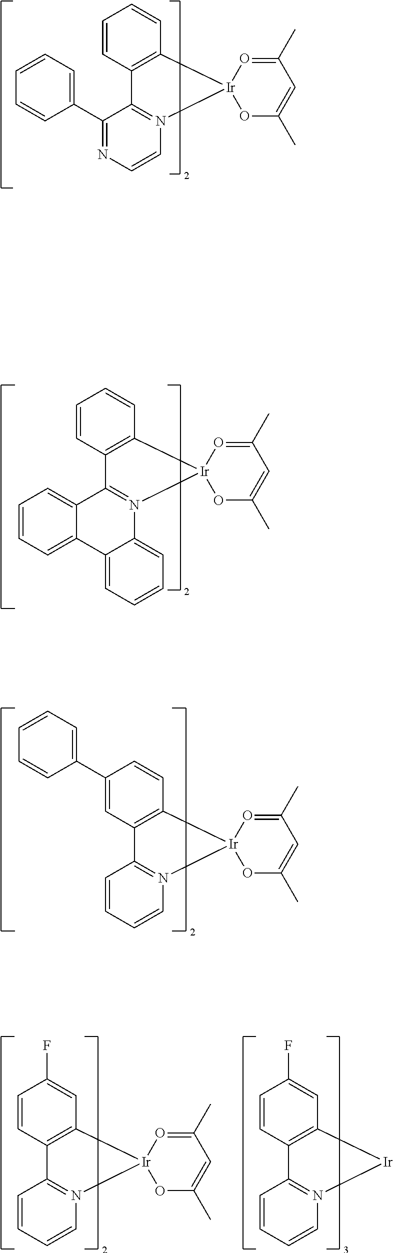

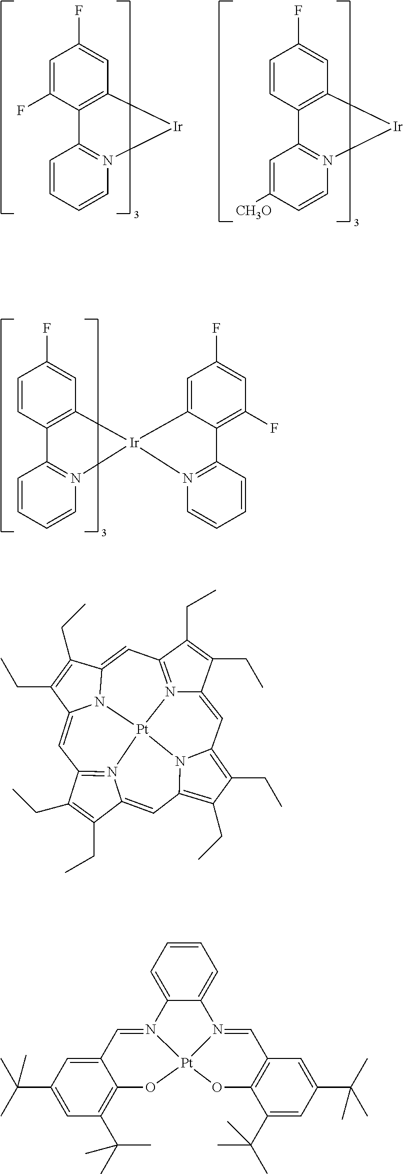

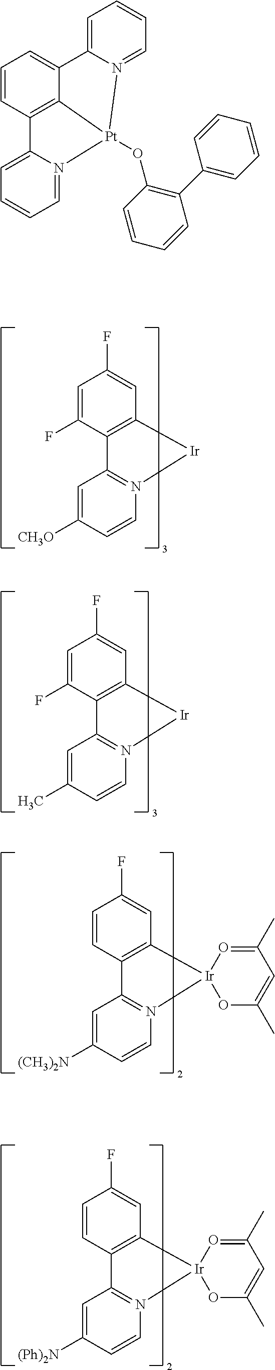

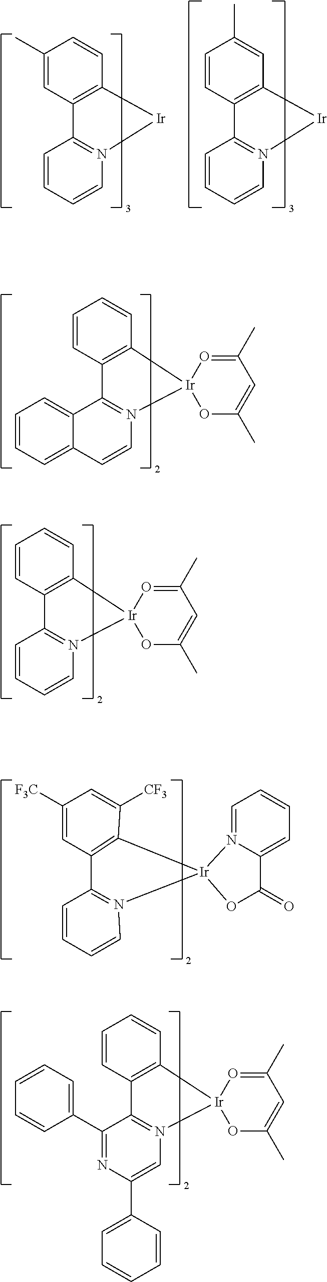

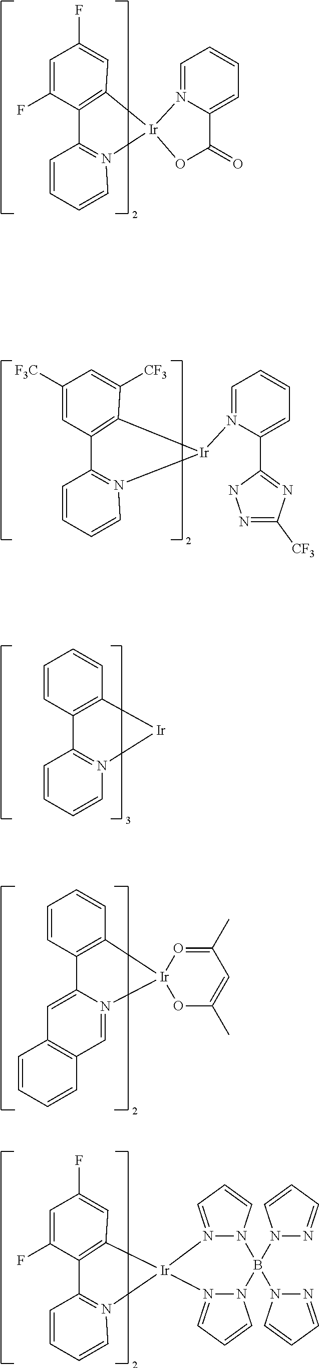

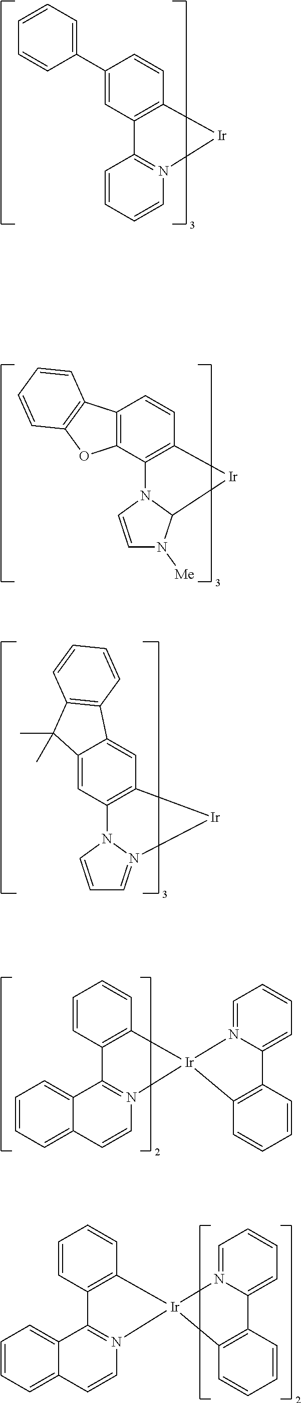

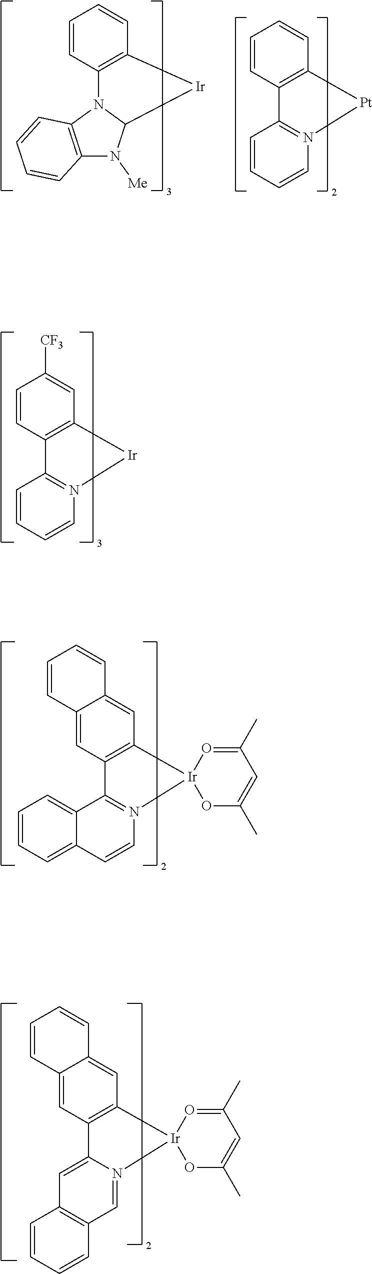

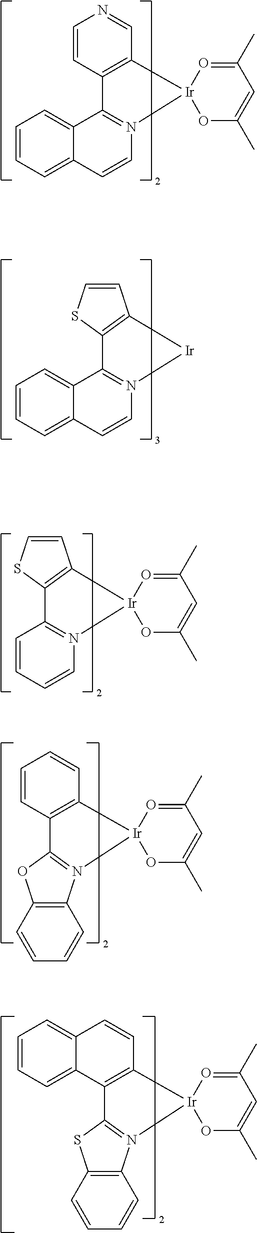

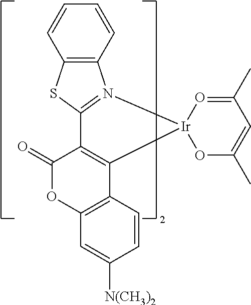

Further suitable red emitters are shown in WO 200/109824. Preferred red emitters according to this document are the following compounds:

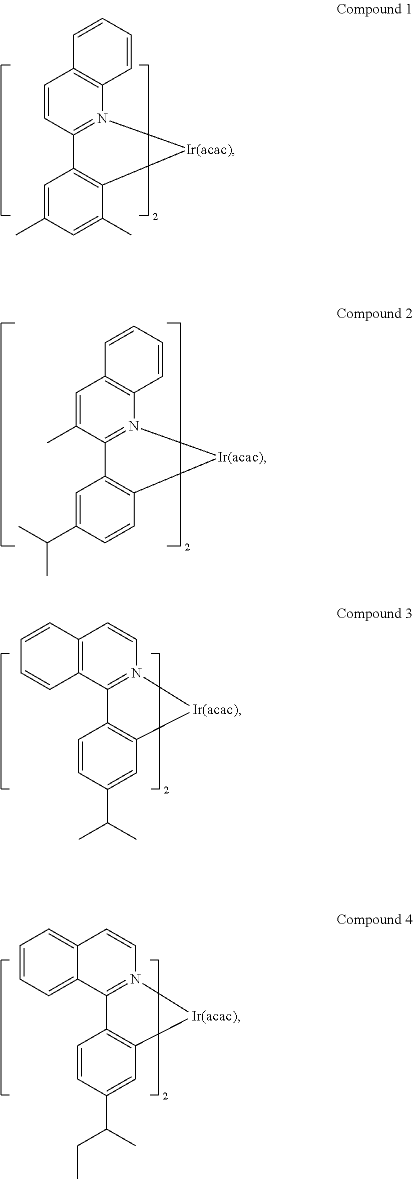

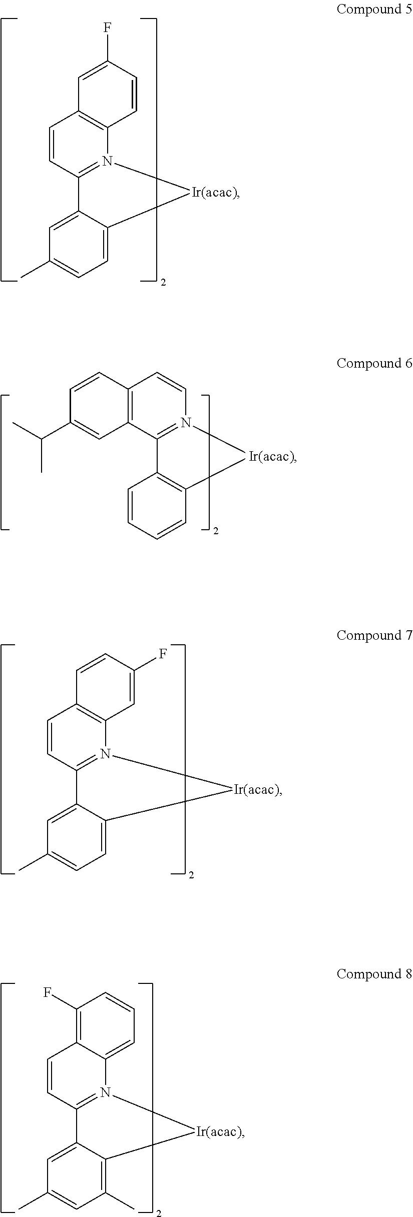

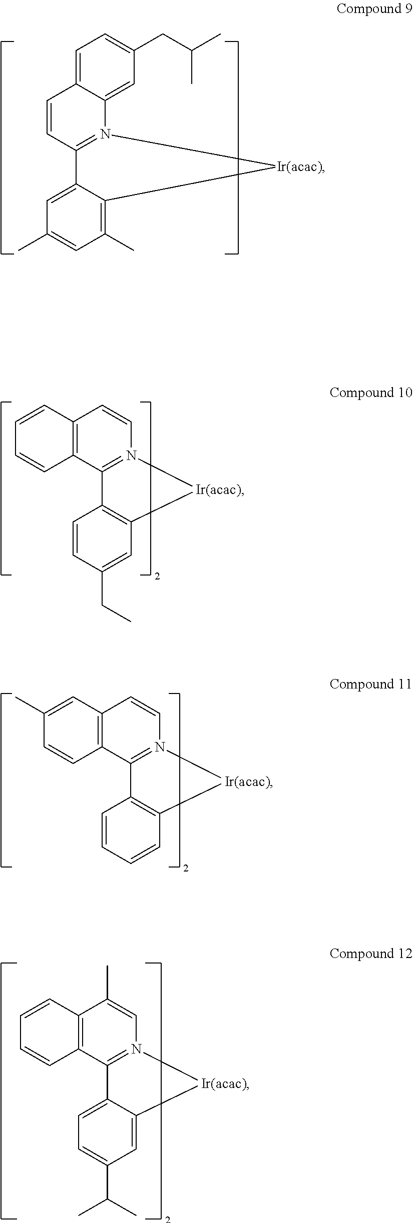

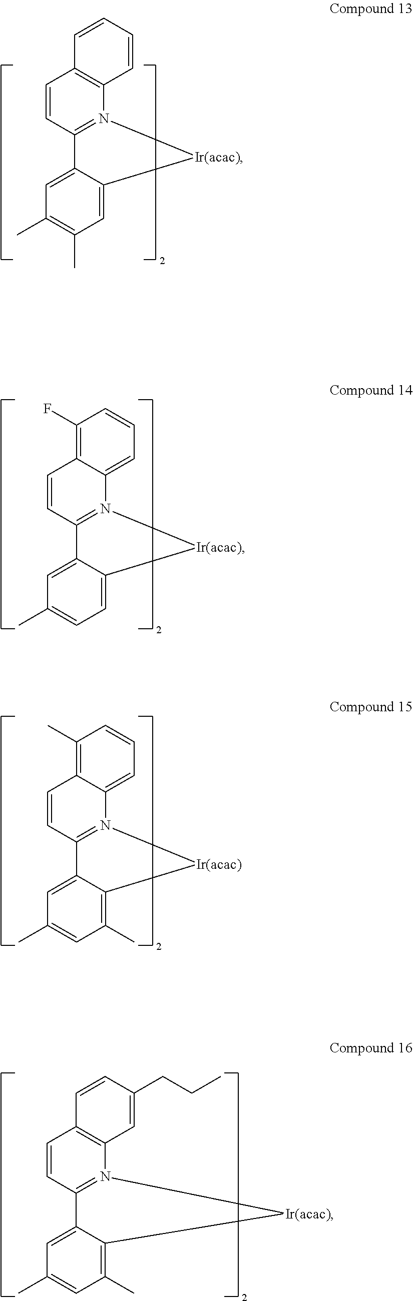

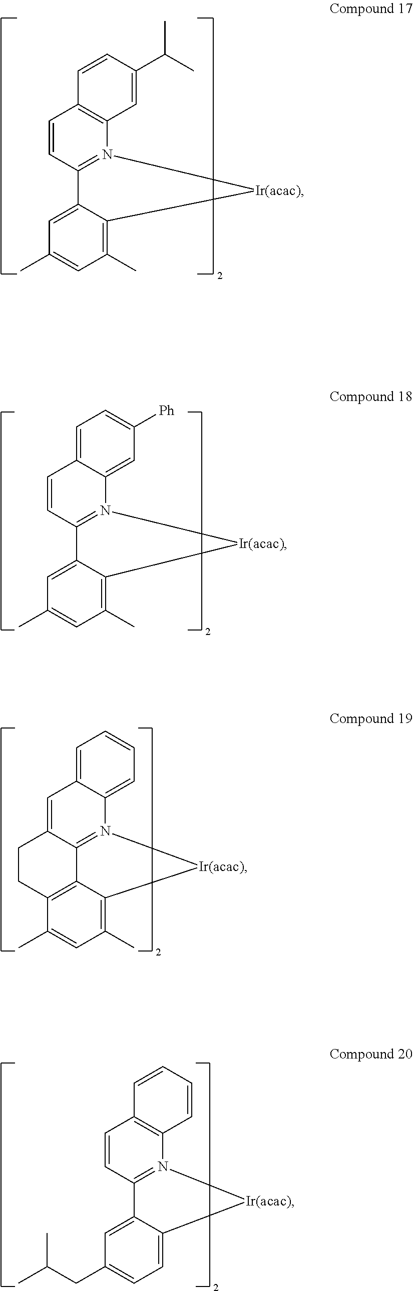

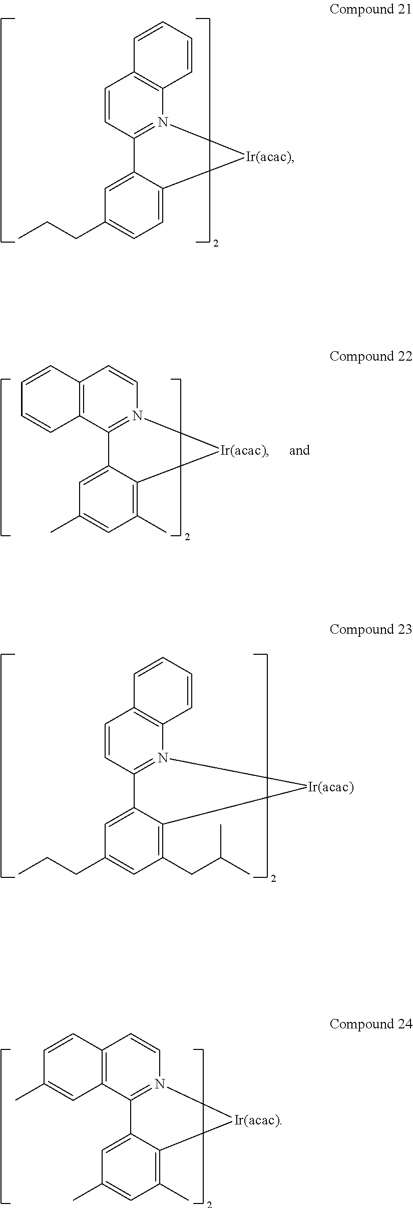

Suitable phosphorescent blue emitters are specified in the following publications: WO2006/056418A2, WO2005/113704, WO2007/115970, WO2007/115981, WO2008/000727, WO2009050281, WO2009050290, WO2011051404, US2011/057559 WO2011/073149, WO2012/121936A2, US2012/0305894A1, WO2012/170571, WO2012/170461, WO2012/170463, WO2006/121811, WO2007/095118, WO2008/156879, WO2008/156879, WO2010/068876, US2011/0057559, WO2011/106344, US2011/0233528, WO2012/048266, WO2012/172482, PCT/EP2014/064054 and PCT/EP2014/066272.

The light emitting layer (e) comprises for example at least one carbene complex as phosphorescence emitter. Suitable carbene complexes are, for example, compounds of the formula

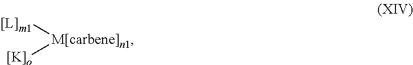

which are described in WO2005/019373A2, wherein the symbols have the following meanings:

M is a metal atom selected from the group consisting of Co, Rh, Ir, Nb, Pd, Pt, Fe, Ru, Os, Cr, Mo, W, Mn, Tc, Re, Cu, Ag and Au in any oxidation state possible for the respective metal atom;

carbene is a carbene ligand which may be uncharged or monoanionic and monodentate, bidentate or tridentate, with the carbene ligand also being able to be a biscarbene or triscarbene ligand;

L is a monoanionic or dianionic ligand, which may be monodentate or bidentate;

K is an uncharged monodentate or bidentate ligand, preferably selected from the group consisting of phosphines; phosphonates and derivatives thereof, arsenates and derivatives thereof; phosphites; CO; pyridines; nitriles and conjugated dienes which form a π complex with M¹;

n1 is the number of carbene ligands, where n1 is at least 1 and when n1>1 the carbene ligands in the complex of the formula I can be identical or different;

m1 is the number of ligands L, where m1 can be 0 or >1 and when m1>1 the ligands L can be identical or different;

o is the number of ligands K, where o can be 0 or >1 and when o>1 the ligands K can be identical or different;

where the sum n1+m1+o is dependent on the oxidation state and coordination number of the metal atom and on the denticity of the ligands carbene, L and K and also on the charge on the ligands, carbene and L, with the proviso that n1 is at least 1.

More preferred are metal-carbene complexes of the general formula:

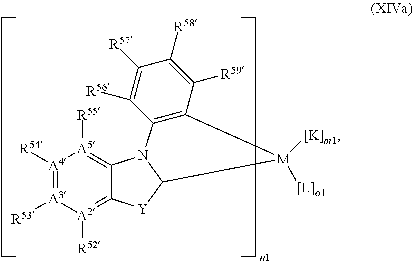

which are described in WO2011/073149, where

M is Ir, or Pt,

n1 is an integer selected from 1, 2 and 3,

Y is NR^{51′}, O, S or C(R^{25′})₂,

A^{2′}, A^{3′}, A^{4′}, and A^{5′} are each independently N or C, where 2 A′=nitrogen atoms and at least one carbon atom is present between two nitrogen atoms in the ring,

R^{51′} is a linear or branched alkyl radical optionally interrupted by at least one heteroatom, optionally bearing at least one functional group and having 1 to 20 carbon atoms, cycloalkyl radical optionally interrupted by at least one heteroatom, optionally bearing at least one functional group and having 3 to 20 carbon atoms, substituted or unsubstituted aryl radical optionally interrupted by at least one heteroatom, optionally bearing at least one functional group and having 6 to 30 carbon atoms, substituted or unsubstituted heteroaryl radical optionally interrupted by at least one heteroatom, optionally bearing at least one functional group and having a total of 5 to 18 carbon atoms and/or heteroatoms,

R^{52′}, R^{53′}, R^{4′} and R^{55′} are each, if A^{2′}, A^{3′}, A^{4′} and/or A^{5′} is N, a free electron pair, or, if A^{2′}, A^{3′}, A^{4′} and/or A^{5′} is C, each independently hydrogen, linear or branched alkyl radical optionally interrupted by at least one heteroatom, optionally bearing at least one functional group and having 1 to 20 carbon atoms, cycloalkyl radical optionally interrupted by at least one heteroatom, optionally bearing at least one functional group and having 3 to 20 carbon atoms, substituted or unsubstituted aryl radical optionally interrupted by at least one heteroatom, optionally bearing at least one functional group and having 6 to 30 carbon atoms, substituted or unsubstituted heteroaryl radical optionally interrupted by at least one heteroatom, optionally bearing at least one functional group and having a total of 5 to 18 carbon atoms and/or heteroatoms, group with donor or acceptor action, or

R^{53′} and R^{54′} together with A^{3′} and A^{4′} form an optionally substituted, unsaturated ring optionally interrupted by at least one further heteroatom and having a total of 5 to 18 carbon atoms and/or heteroatoms,

R^{56′}, R^{57′}, R^{58′} and R^{59′} are each independently hydrogen, linear or branched alkyl radical optionally interrupted by at least one heteroatom, optionally bearing at least one functional group and having 1 to 20 carbon atoms, cycloalkyl radical optionally interrupted by at least one heteroatom, optionally bearing at least one functional group and having 3 to 20 carbon atoms, cycloheteroalkyl radical optionally interrupted by at least one heteroatom, optionally bearing at least one functional group and having 3 to 20 carbon atoms, substituted or unsubstituted aryl radical optionally interrupted by at least one heteroatom, optionally bearing at least one functional group and having 6 to 30 carbon atoms, substituted or unsubstituted heteroaryl radical optionally interrupted by at least one heteroatom, optionally bearing at least one functional group and having a total of 5 to 18 carbon atoms and/or heteroatoms, group with donor or acceptor action, or

R^{56′} and R^{57′}, R^{57′} and R^{58′} or R^{58′} and R^{59′}, together with the carbon atoms to which they are bonded, form a saturated or unsaturated or aromatic, optionally substituted ring optionally interrupted by at least one heteroatom and having a total of 5 to 18 carbon atoms and/or heteroatoms, and/or

if A^{5′} is C, R^{55′} and R^{56′} together form a saturated or unsaturated, linear or branched bridge optionally comprising heteroatoms, an aromatic unit, heteroaromatic unit and/or functional groups and having a total of 1 to 30 carbon atoms and/or heteroatoms, to which is optionally fused a substituted or unsubstituted, five- to eight-membered ring comprising carbon atoms and/or heteroatoms,

R^{25′} is independently a linear or branched alkyl radical optionally interrupted by at least one heteroatom, optionally bearing at least one functional group and having 1 to 20 carbon atoms, cycloalkyl radical optionally interrupted by at least one heteroatom, optionally bearing at least one functional group and having 3 to 20 carbon atoms, substituted or unsubstituted aryl radical optionally interrupted by at least one heteroatom, optionally bearing at least one functional group and having 6 to 30 carbon atoms, substituted or unsubstituted heteroaryl radical optionally interrupted by at least one heteroatom, optionally bearing at least one functional group and having a total of 5 to 18 carbon atoms and/or heteroatoms,

K is an uncharged mono- or bidentate ligand,

L is a mono- or dianionic ligand, preferably monoanionic ligand, which may be mono- or bidentate,

m1 is 0, 1 or 2, where, when m1 is 2, the K ligands may be the same or different, and

o1 is 0, 1 or 2, where, when o1 is 2, the L ligands may be the same or different.

The compound of formula XIV is preferably a compound of the formula:

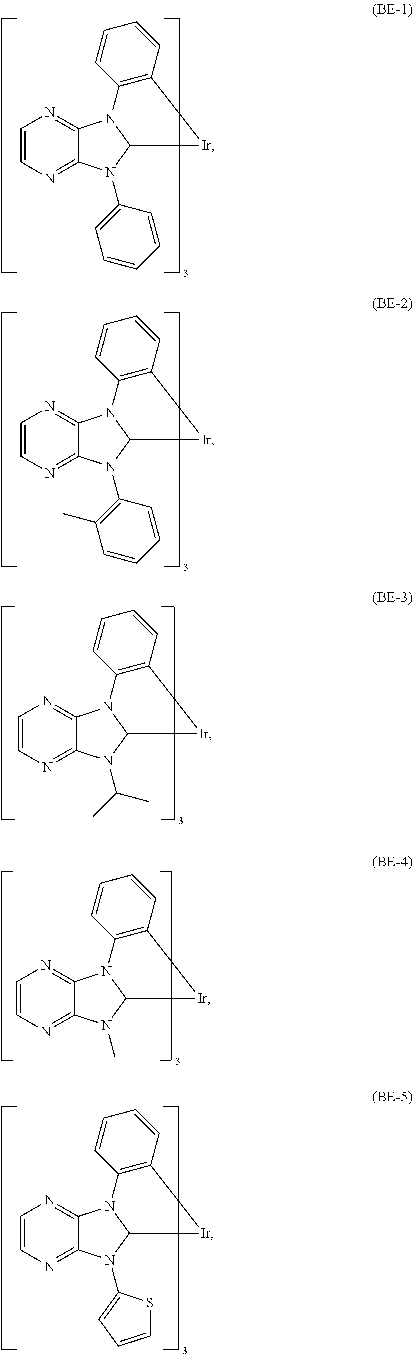

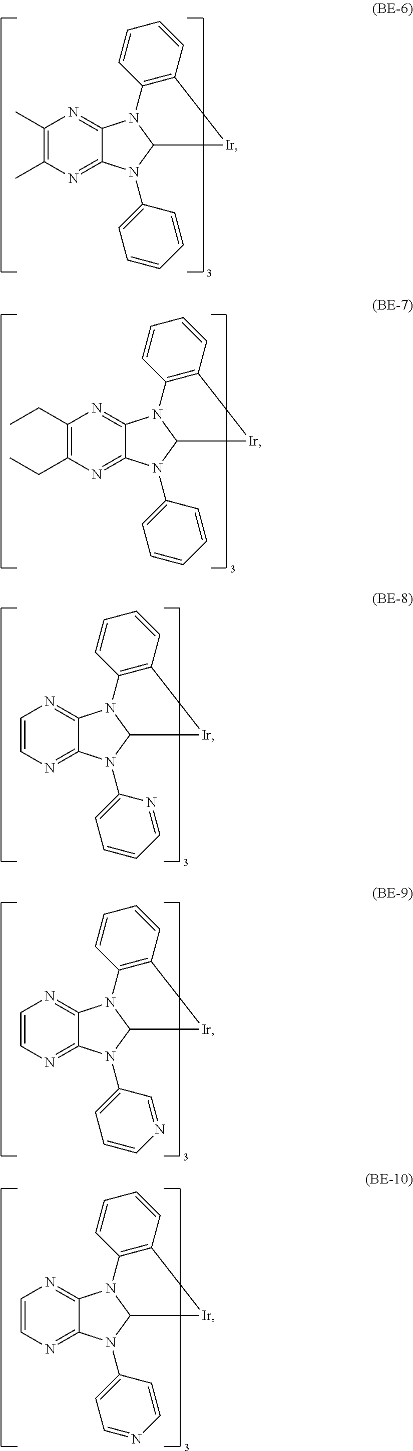

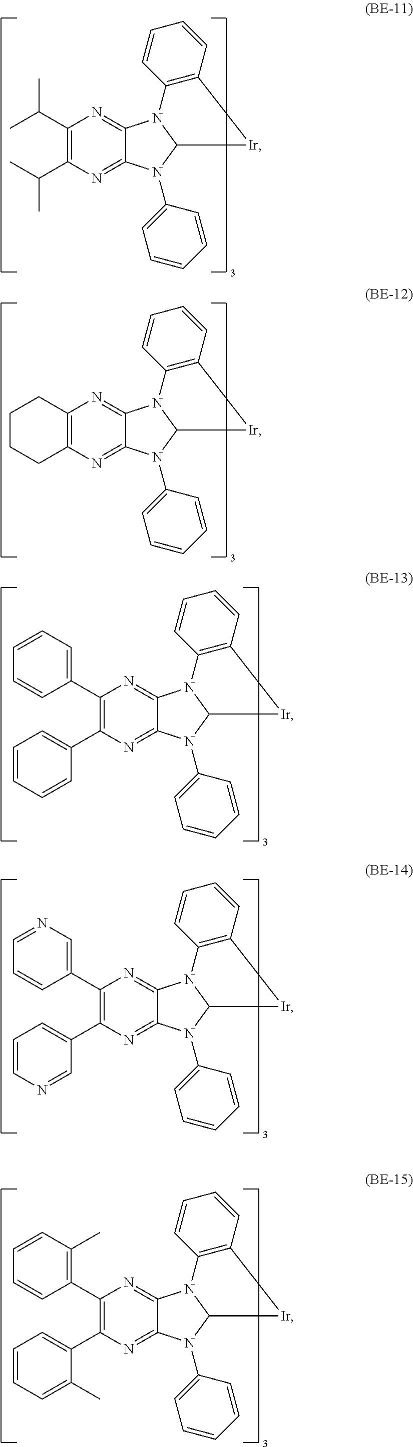

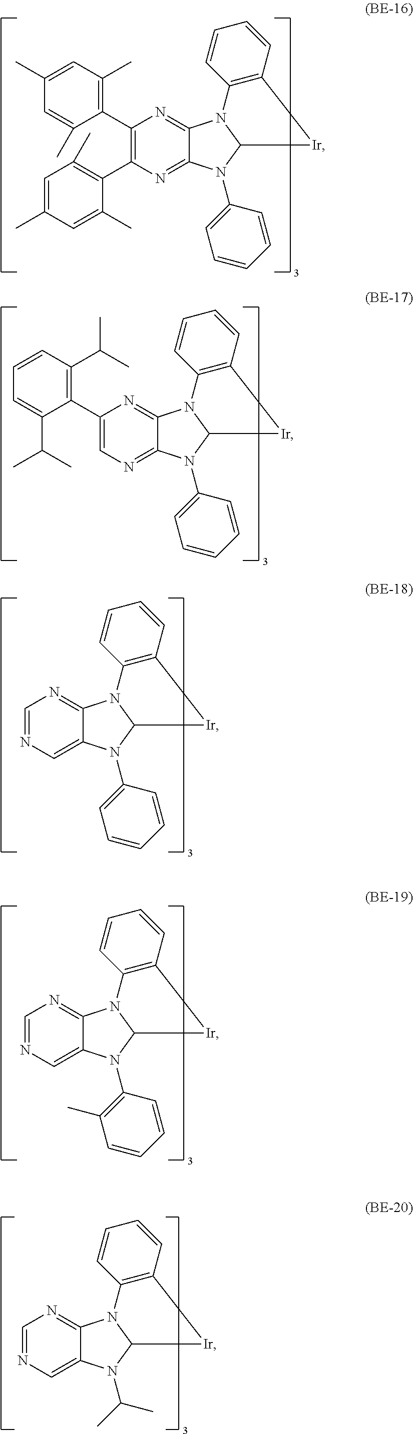

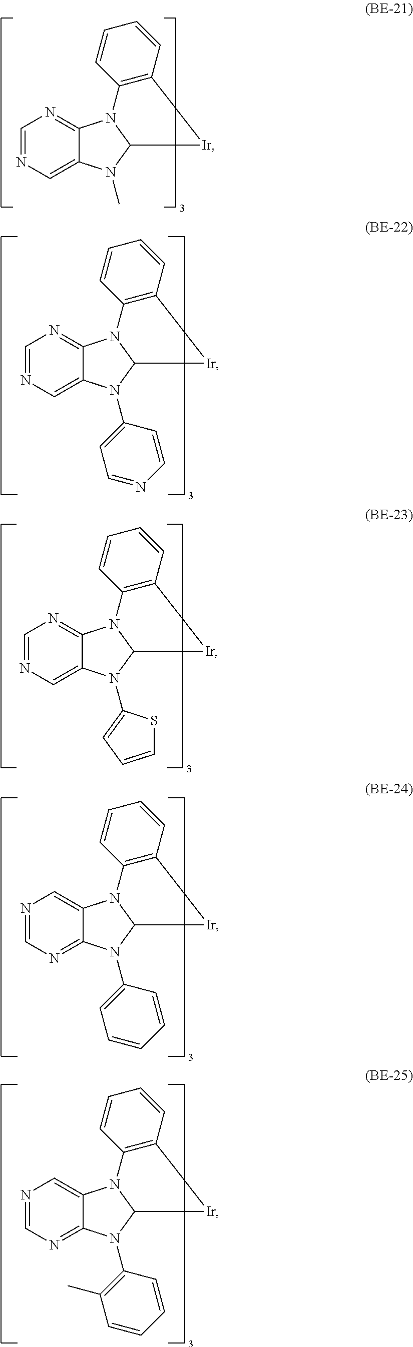

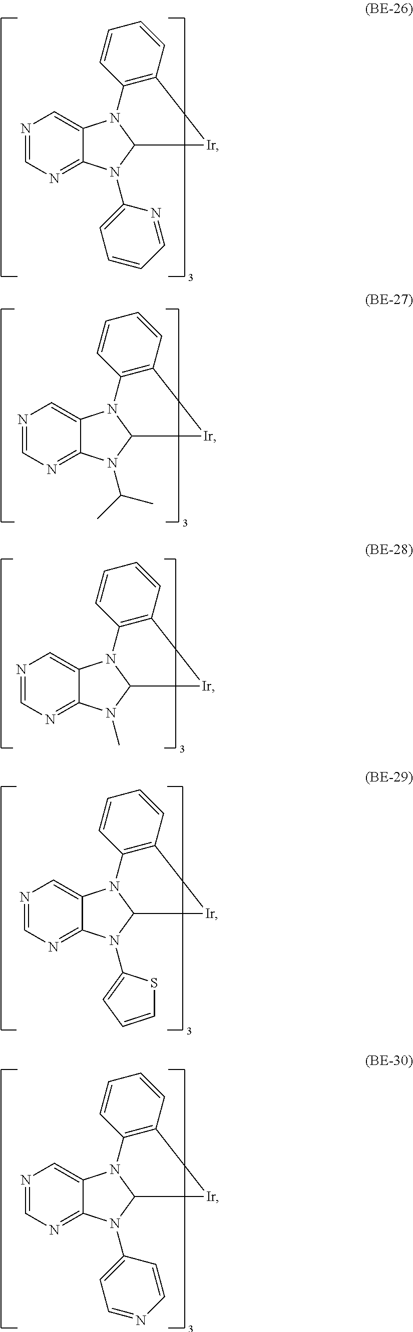

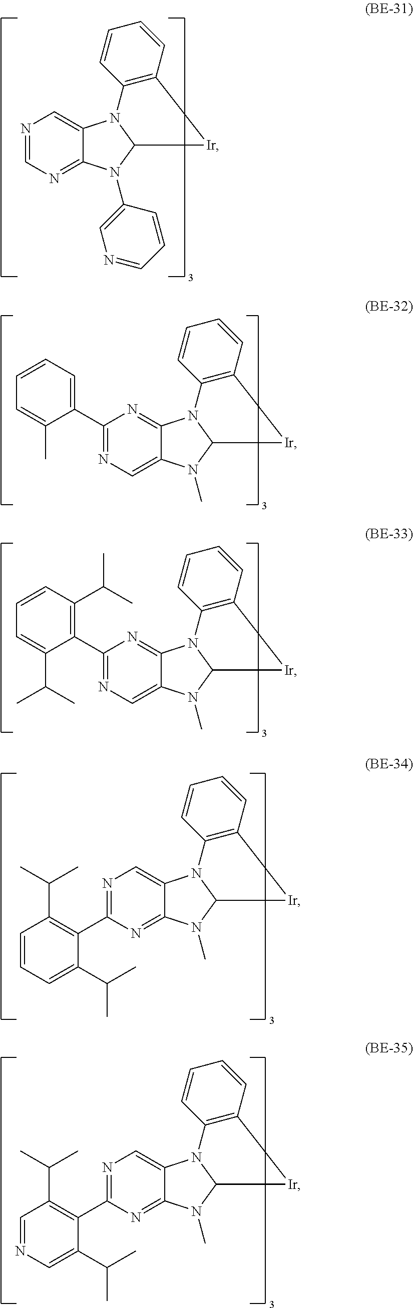

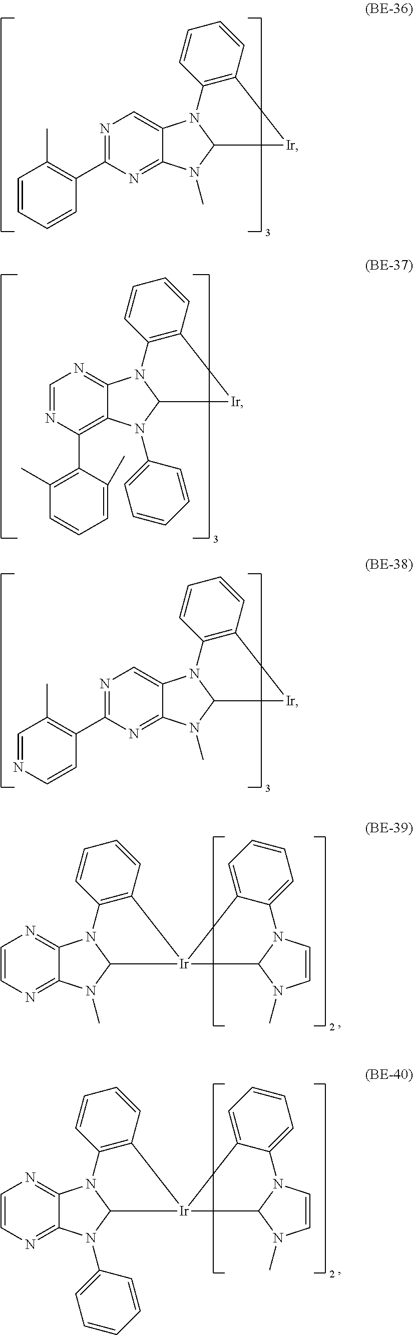

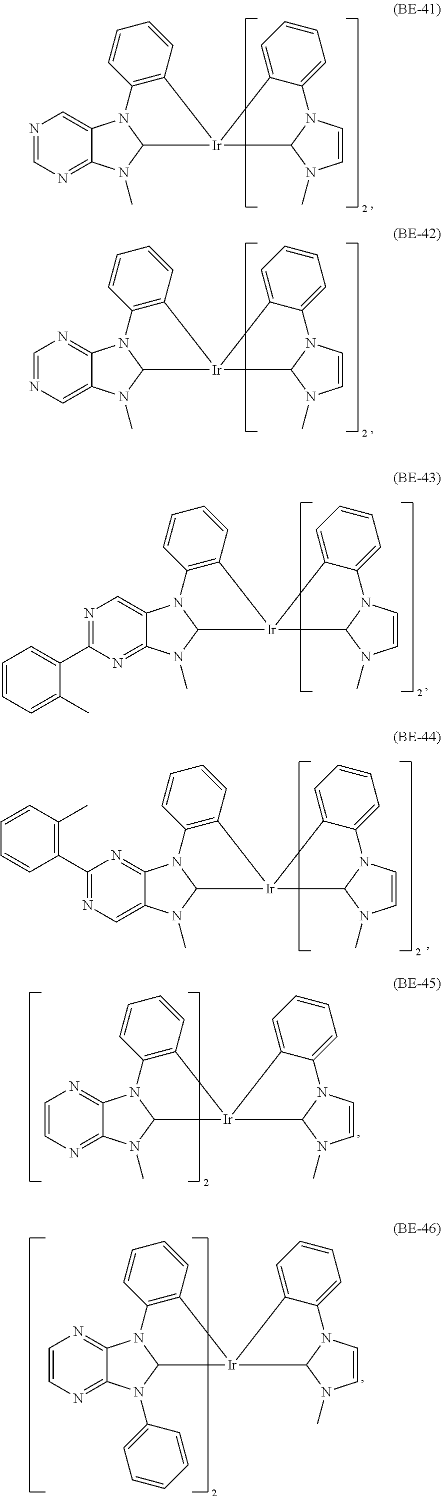

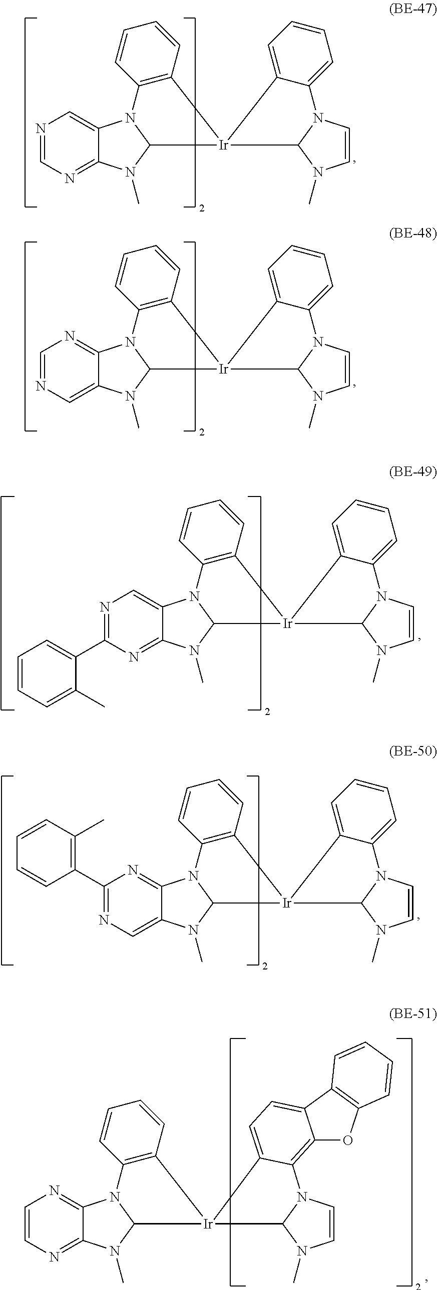

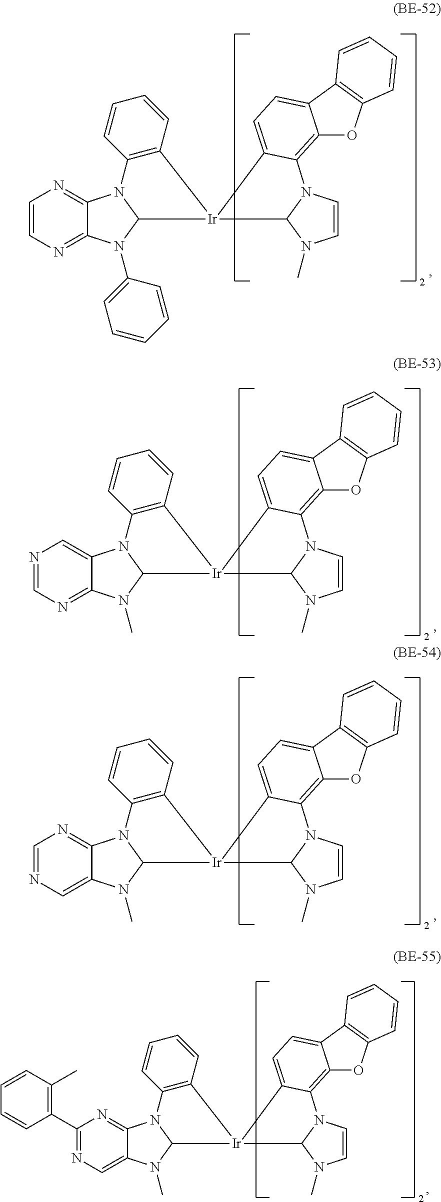

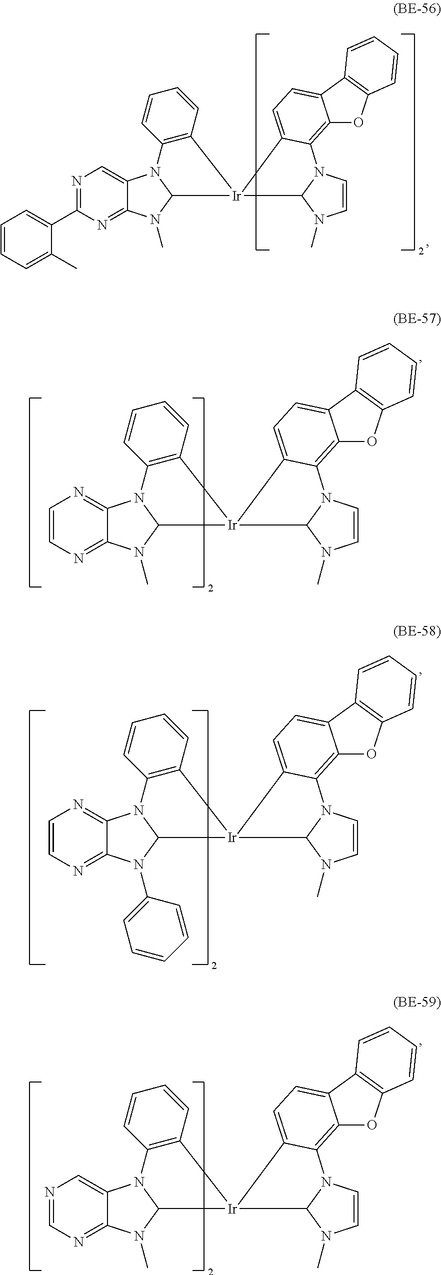

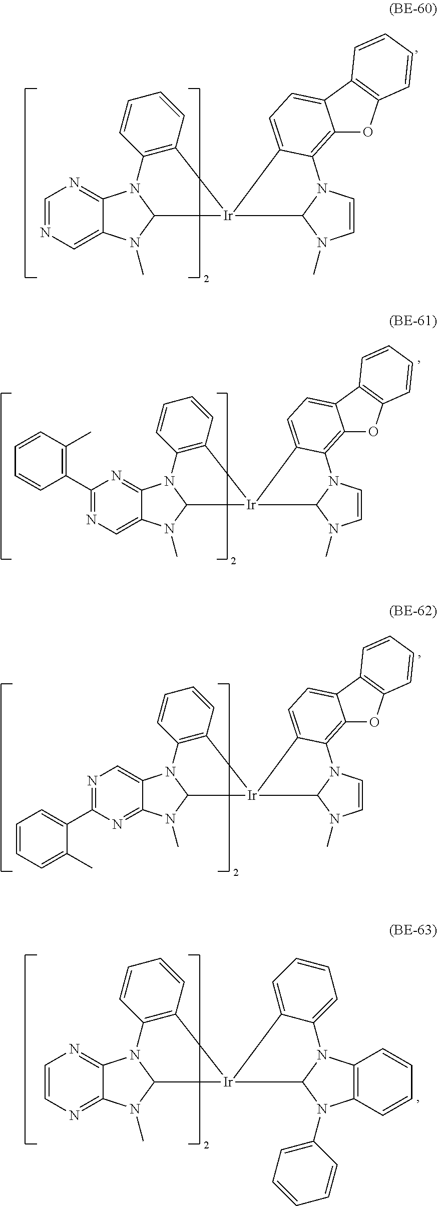

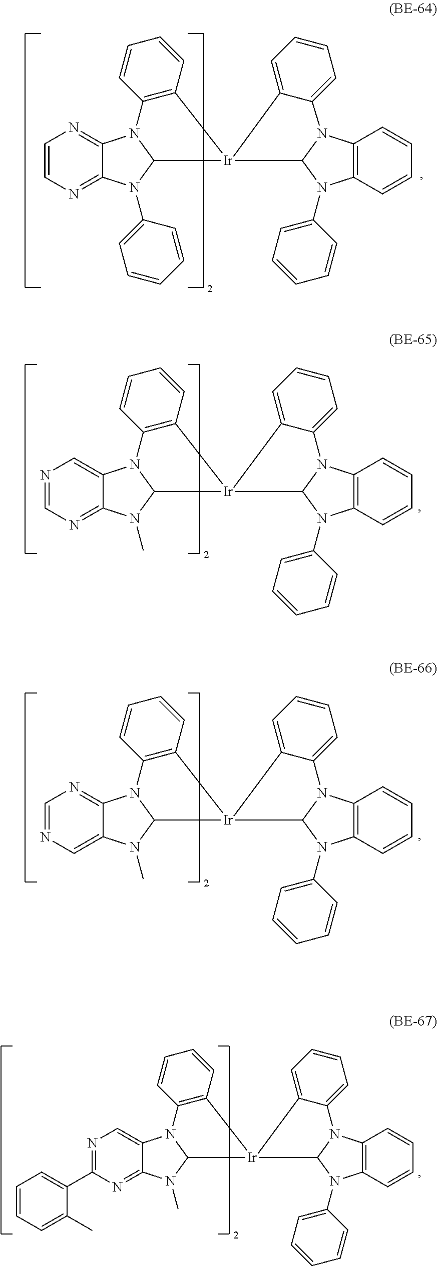

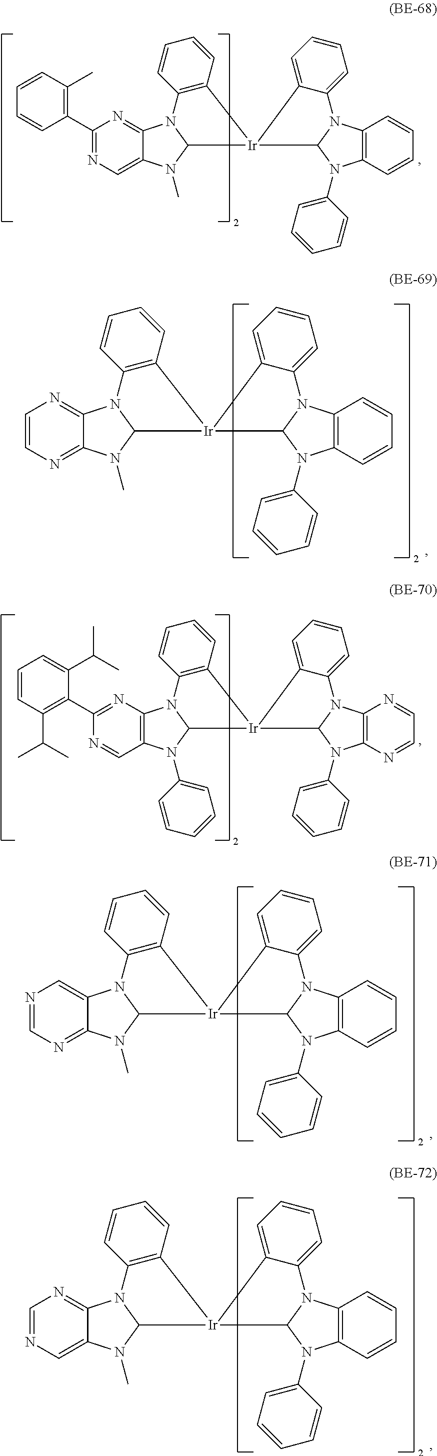

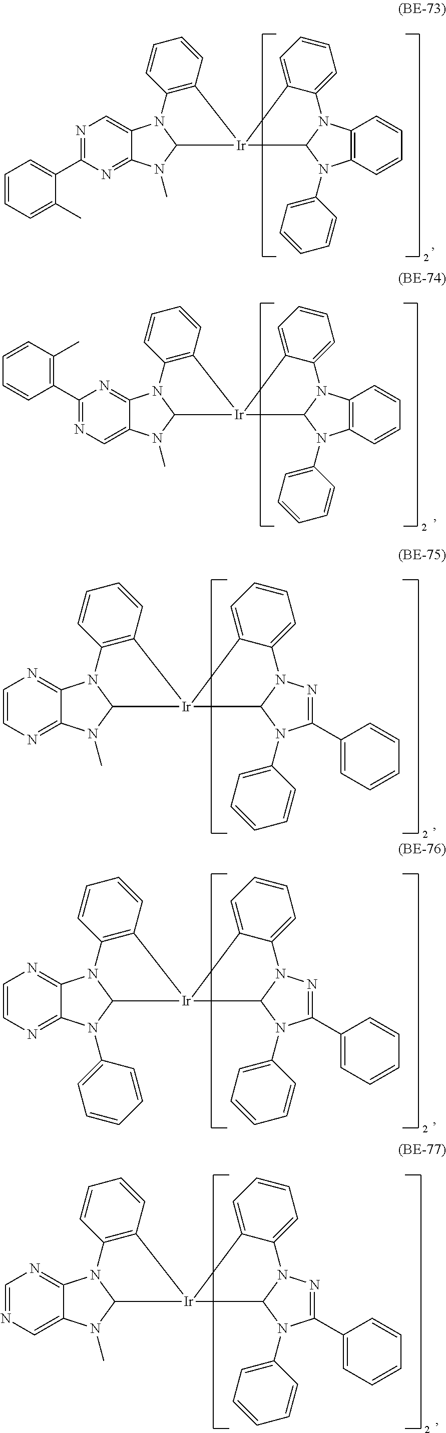

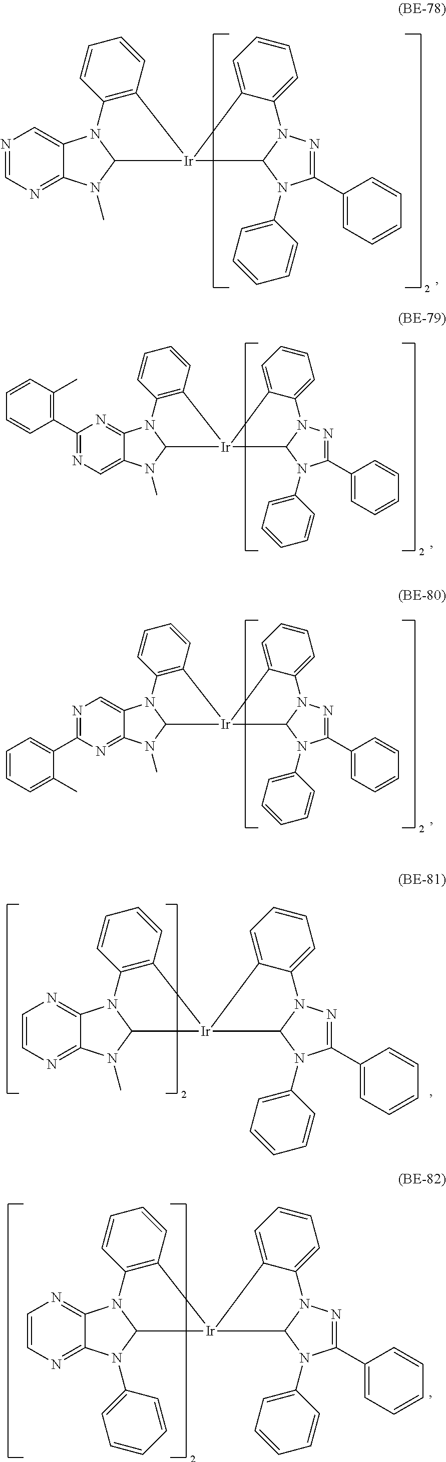

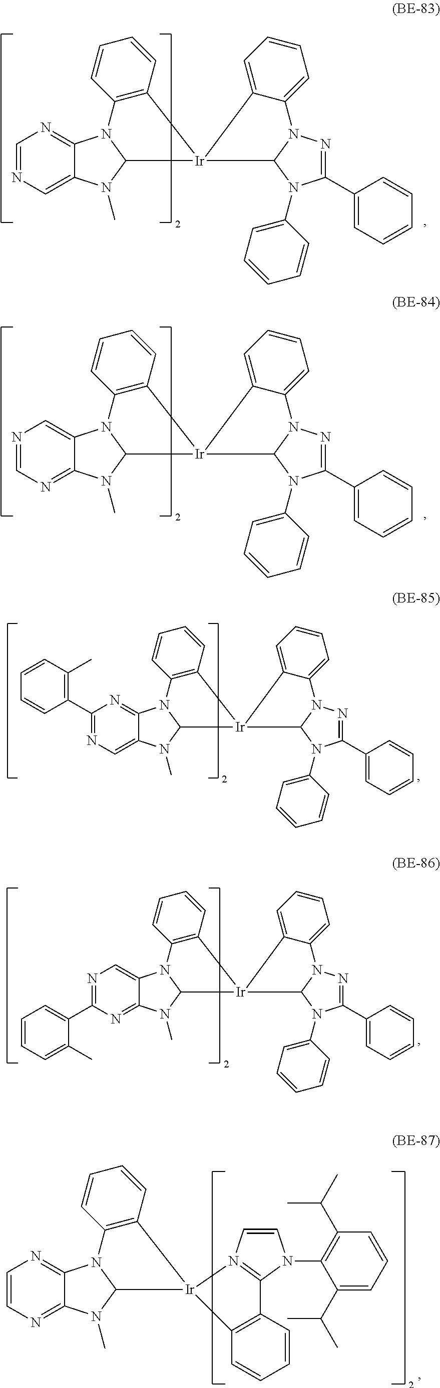

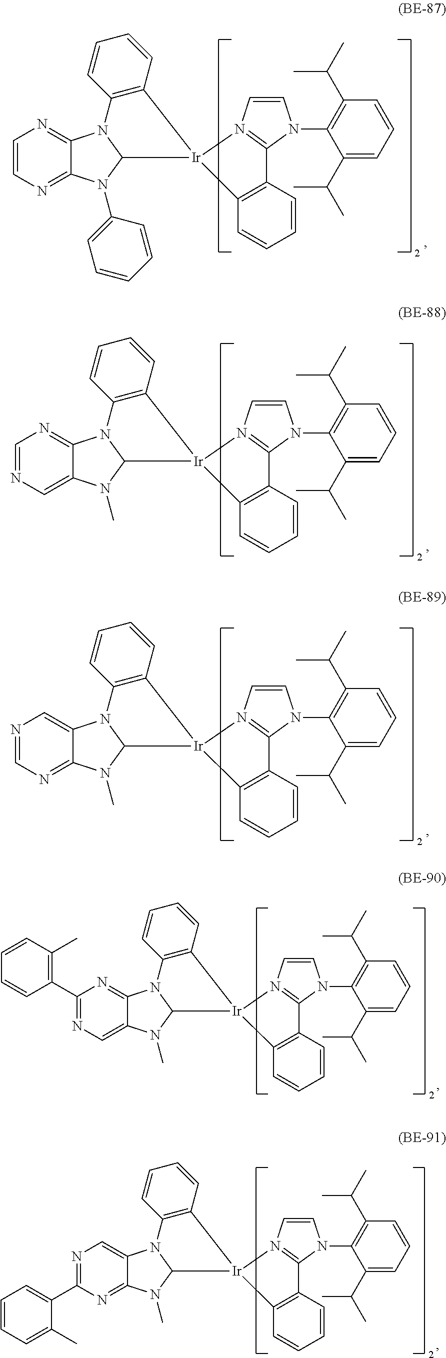

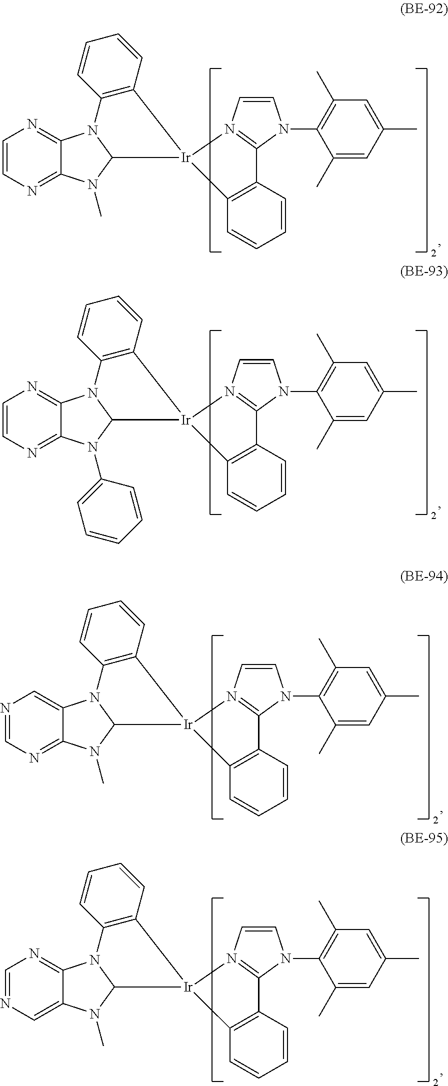

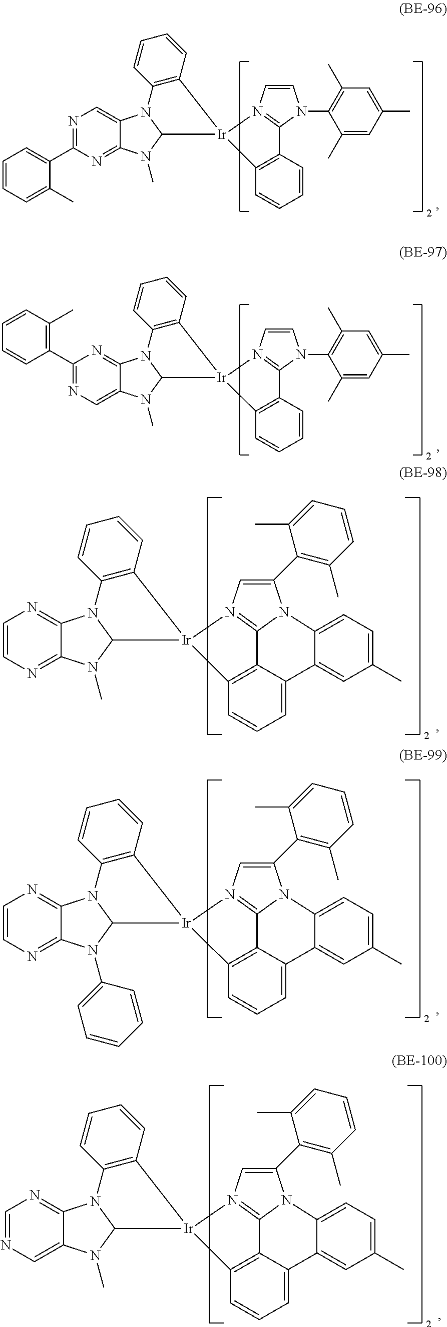

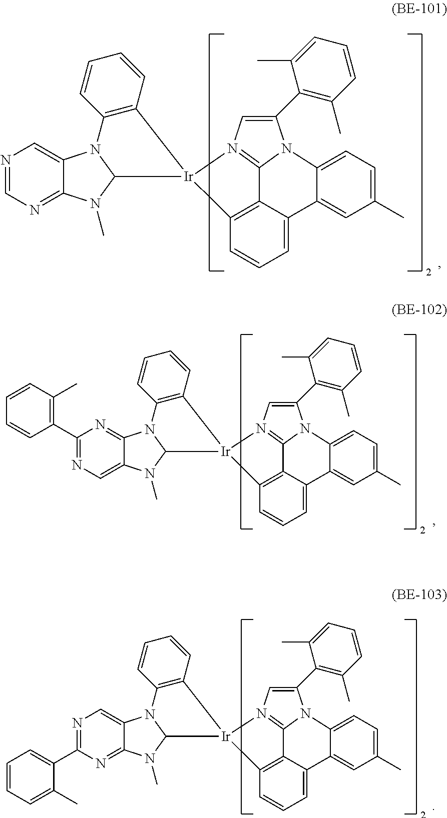

Further suitable non-carbene emitter materials are mentioned below:

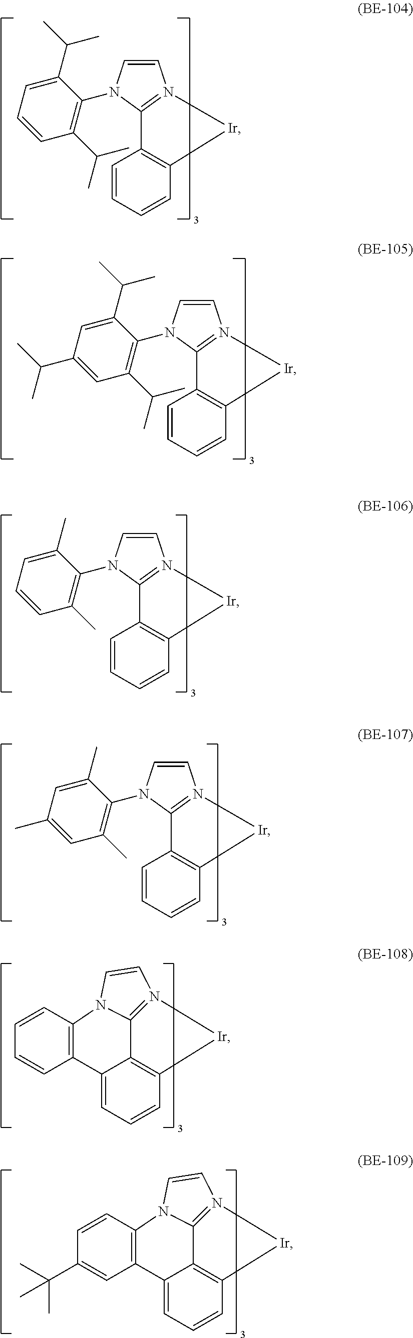

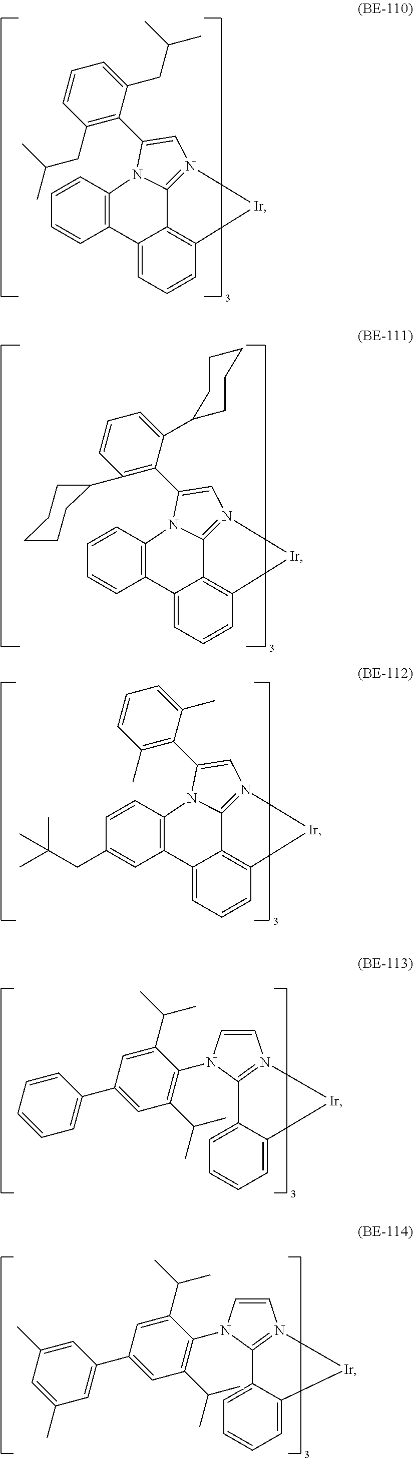

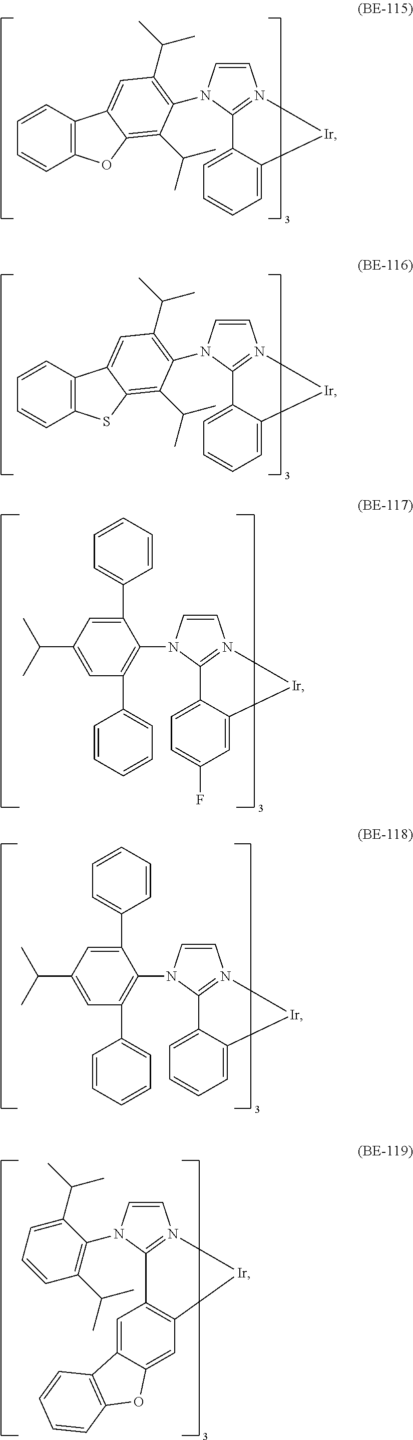

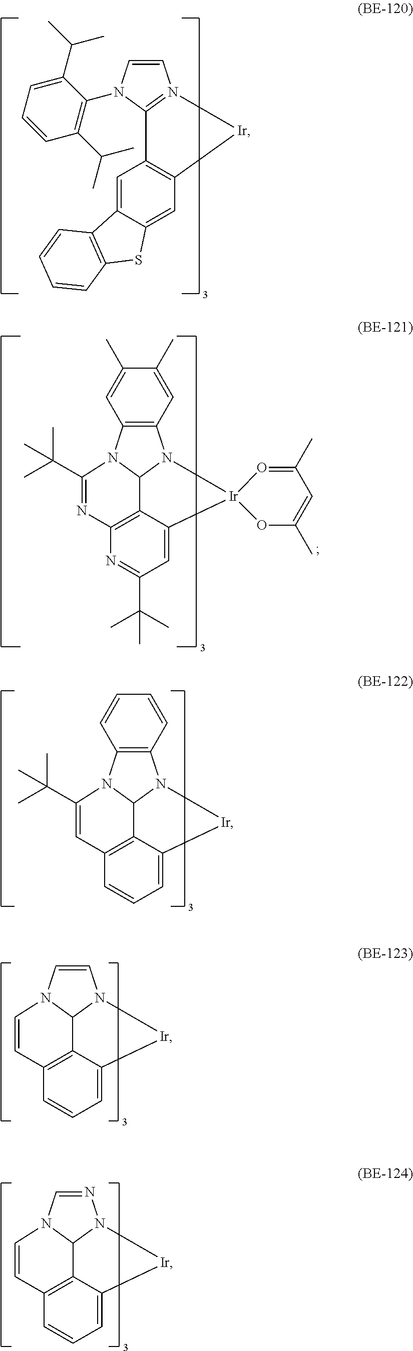

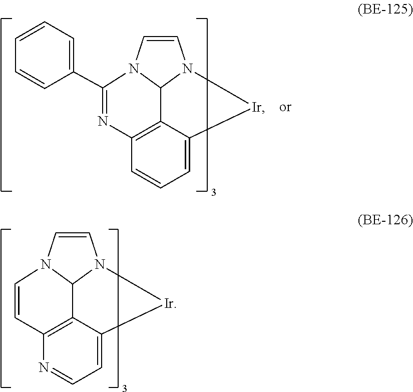

The compound of formula XIV is more preferably a compound (BE-1), (BE-2), (BE-7), (BE-12), (BE-16), (BE-64), or (BE-70). The most preferred phosphorescent blue emitters are compounds (BE-1) and (BE-12).

The homoleptic metal-carbene complexes may be present in the form of facial or meridional isomers or mixtures thereof, preference being given to the facial isomers.

Suitable carbene complexes of formula (XIV) and their preparation process are, for example, described in WO2011/073149.

The compounds of formulae (I), (II) or (III) of the present invention can also be used as host for phosphorescent green emitters. Suitable phosphorescent green emitters are, for example, specified in the following publications: WO2006014599, WO20080220265, WO2009073245, WO2010027583, WO2010028151, US20110227049, WO2011090535, WO2012/08881, WO20100056669, WO20100118029, WO20100244004, WO2011109042, WO2012166608, US20120292600, EP2551933A1; U.S. Pat. No. 6,687,266, US20070190359, US20070190359, US20060008670; WO2006098460, US20110210316, WO2012053627; U.S. Pat. No. 6,921,915, US20090039776; JP2007123392 and European patent application no. 14180422.9.

Examples of suitable phosphorescent green emitters are shown below:

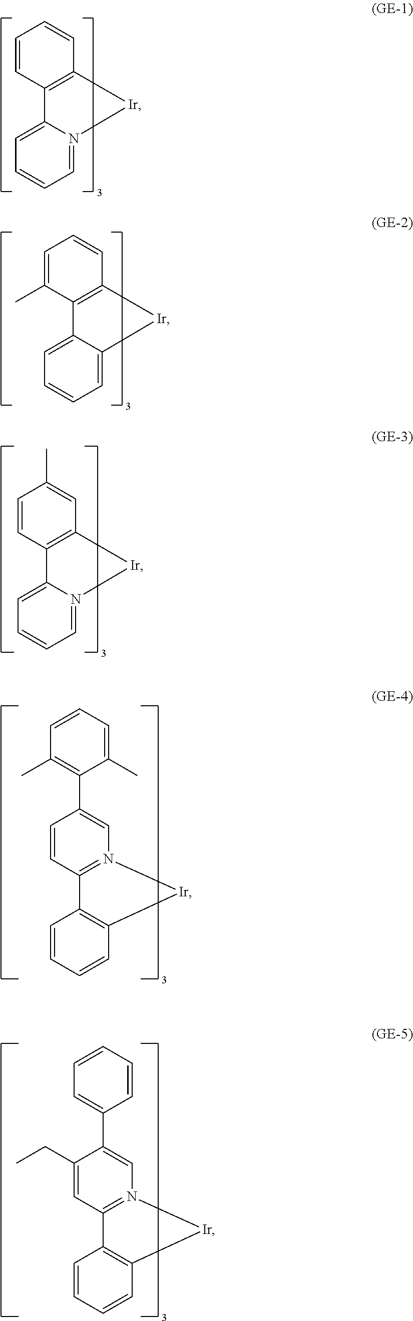

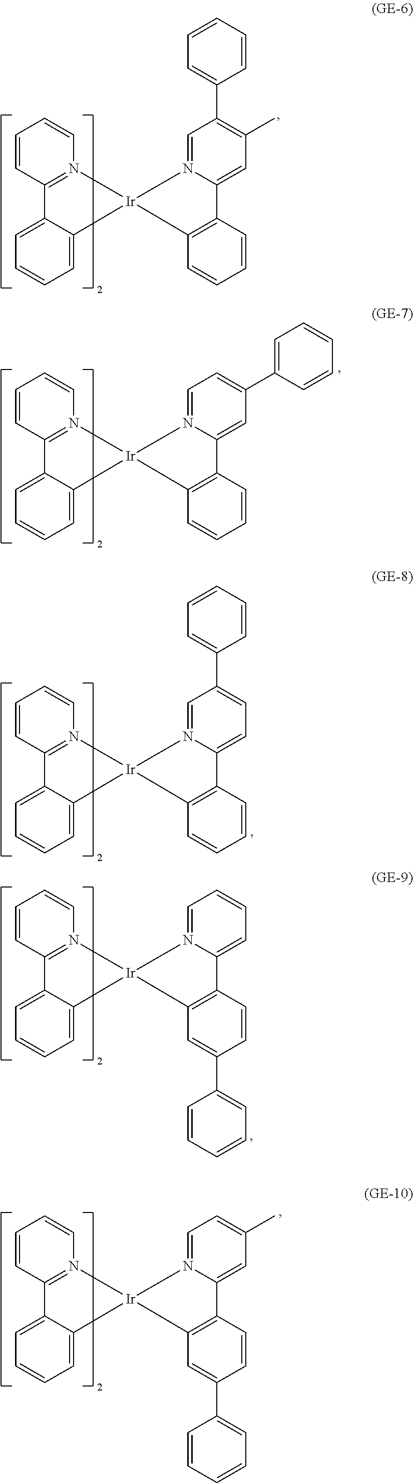

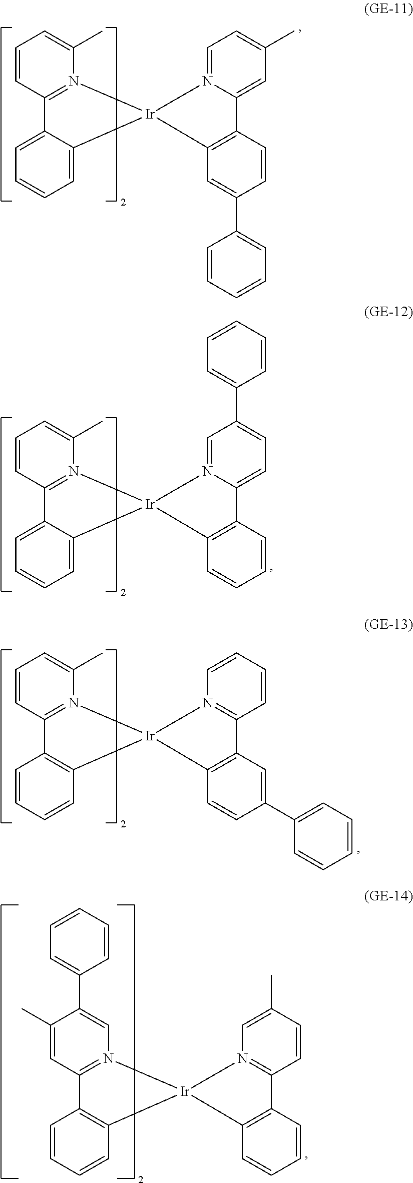

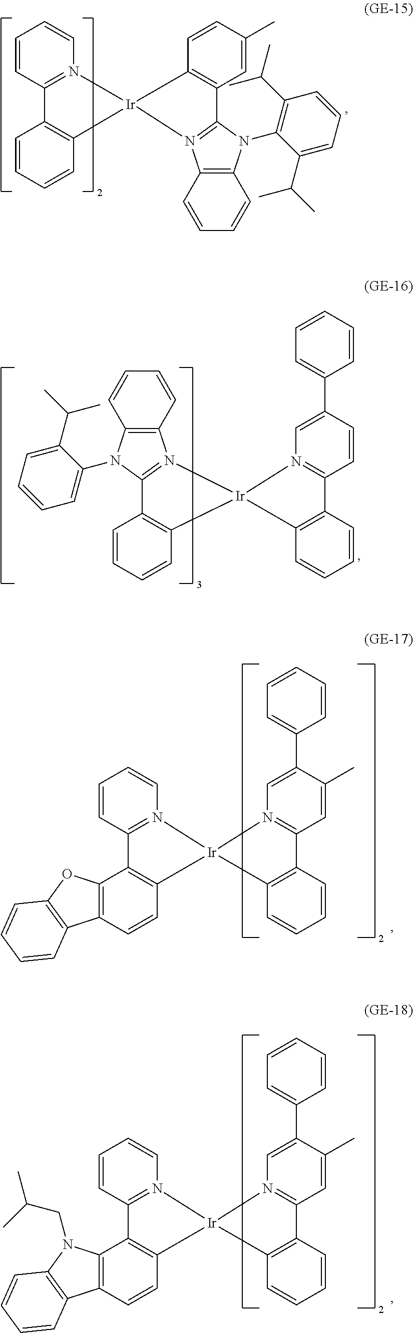

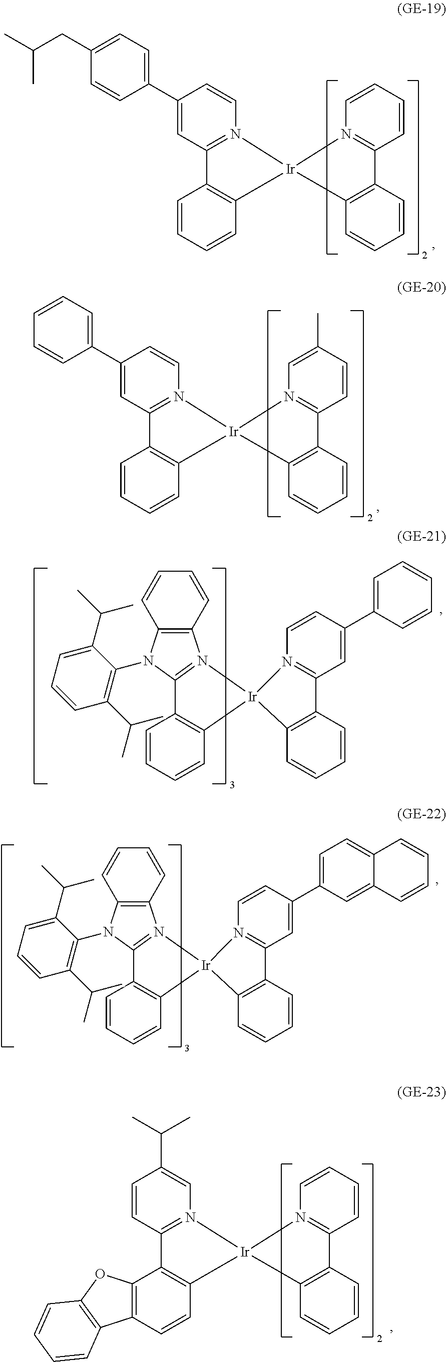

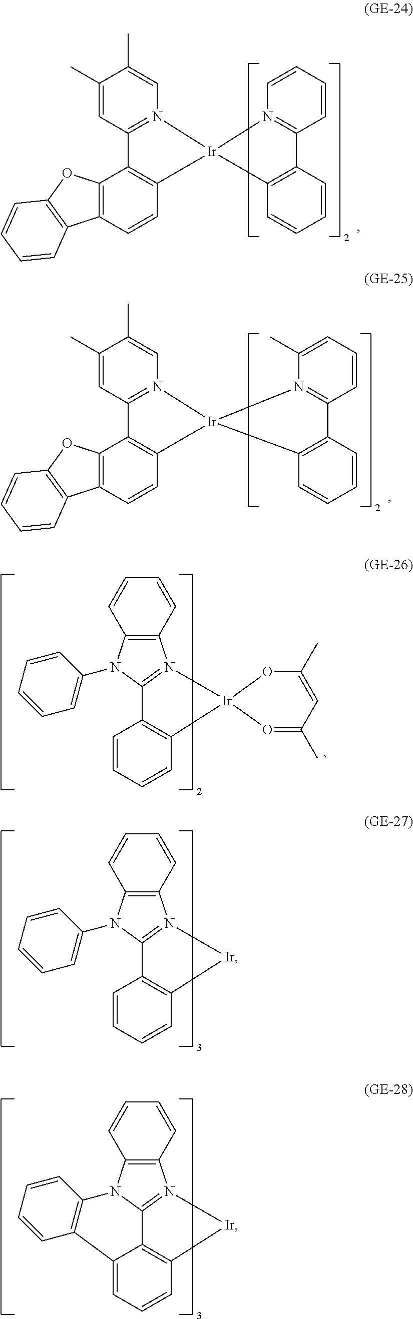

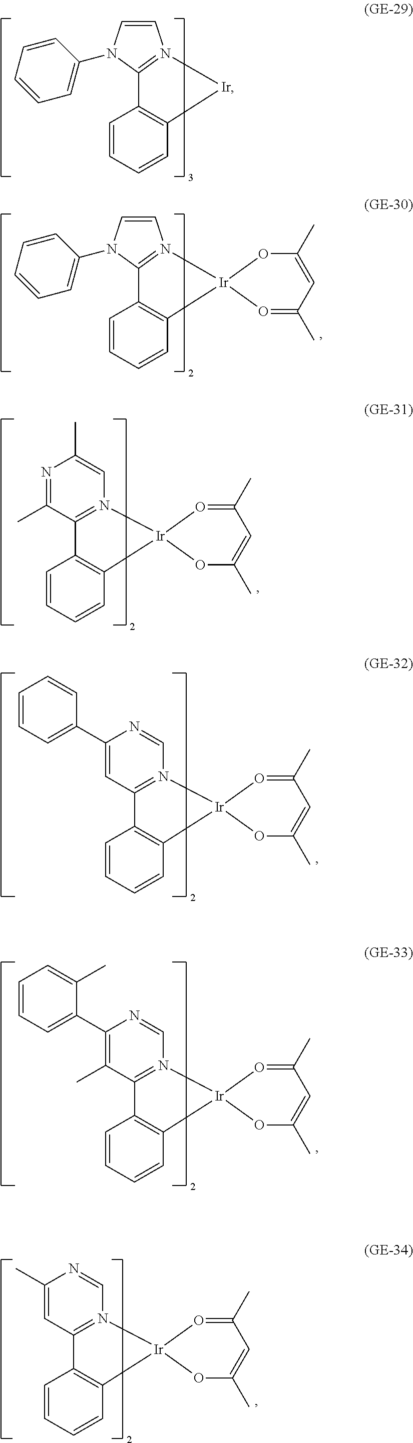

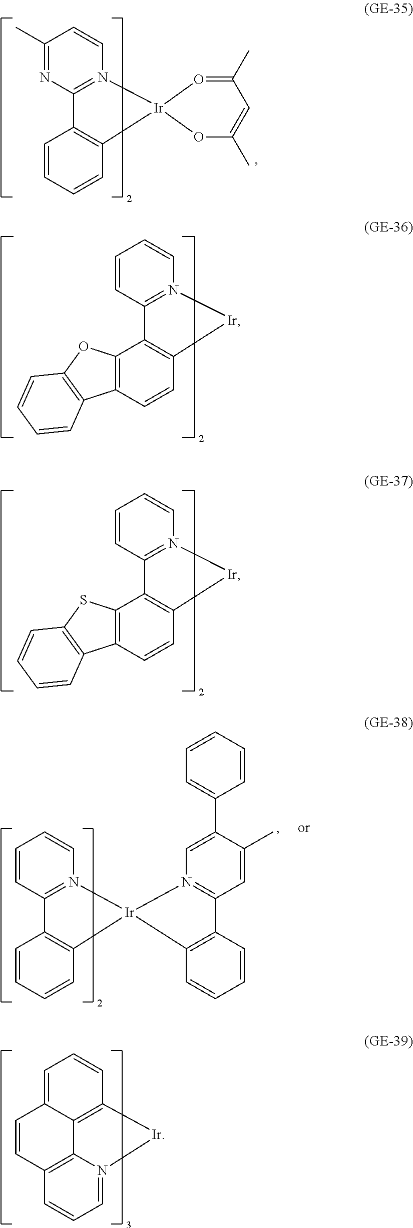

The emitter materials (dopants), preferably the phosphorescent emitter materials, may be used alone or in combination of two or more.

Further preferred blue dopants that may be present in the light emitting layer of the OLED according to the present invention are polycyclic amine derivatives as mentioned in EP 2924029. Particularly preferred aromatic amine derivatives are selected from compounds according to the following formula (20):

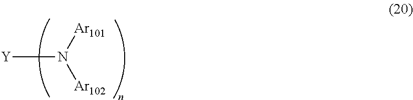

In the formula (20), Y is a substituted or unsubstituted fused aromatic hydrocarbon group including 10 to 50 ring carbon atoms.

Ar₁₀₁, and Ar₁₀₂ are independently a substituted or unsubstituted aryl group including 6 to 50 ring carbon atoms or a substituted or unsubstituted heterocyclic ring group including 5 to 50 ring atoms.

Specific examples of Y include the above-mentioned fused aryl group. Y is preferably a substituted or unsubstituted anthryl group, a substituted or unsubstituted pyrenyl group or a substituted or unsubstituted chrysenyl group.

n is an integer of 1 to 4. It is preferred that n be an integer of 1 to 2.

The above-mentioned formula (20) is preferably one represented by the following formulas (21) to (24):

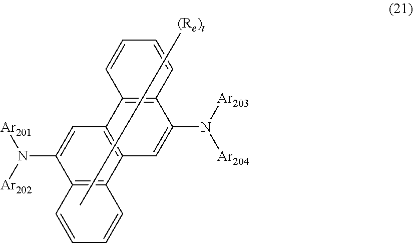

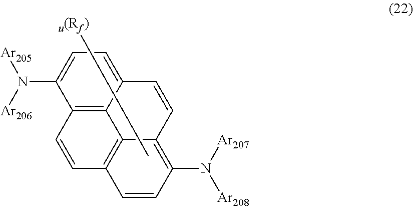

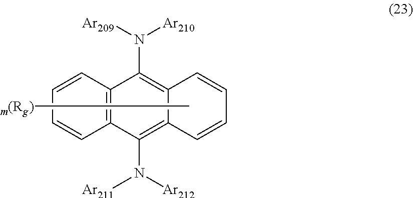

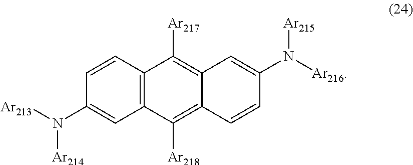

In the formulas (21) to (24), Rₑ, R_{f} and R_{g} are independently a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 1 to 20 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted silyl group, a substituted or unsubstituted alkyl germanium group including 1 to 50 carbon atoms or a substituted or unsubstituted aryl germanium group including 6 to 50 ring carbon atoms. Rₑ, R_{f} and R_{g} may independently be bonded to any of the bonding positions of the benzene rings that constitutes the fused polycyclic skeleton.

As preferable examples of Rₑ, R_{f} and R_{g}, a substituted or unsubstituted aryl group including 6 to 50 ring carbon atoms can be given. More preferably, Rₑ, R_{f} and R_{g} are a substituted or unsubstituted phenyl group, a substituted or unsubstituted naphthyl group, or the like.

t is an integer of 0 to 10. u is an integer of 0 to 8. m is an integer of 0 to 10. Ar₂₀₁ to Ar₂₁₈ are independently an aryl group including 6 to 50 ring carbon atoms or a substituted or unsubstituted heterocyclic group including 5 to 50 ring atoms.

Preferred examples of Ar₂₀₁ to Ar₂₁₈ include a substituted or unsubstituted phenyl group, a substituted or unsubstituted dibenzofuranyl group or the like. As preferable examples of the substituent of Ar₂₀₁ to Ar₂₁₈, an alkyl group, a cyano group and a substituted or unsubstituted silyl group can be given.

In the formulas (21) to (24), as examples of the alkyl group, the alkoxy group, the aryl group, the aryloxy group and the heterocyclic group, those exemplified above can be given.

As the alkenyl group including 2 to 50, preferably 2 to 30, more preferably 2 to 20, and particularly preferably 2 to 10, carbon atoms, a vinyl group, an allyl group, a 1-butenyl group, a 2-butenyl group, a 3-butenyl group, a 1,3-butanedienyl group, a 1-methylvinyl group, a styryl group, a 2,2-diphenylvinyl group, a 1,2-diphenylvinyl group, a 1-methylallyl group, a 1,1-dimethylallyl group, a 2-methylallyl group, a 1-phenylallyl group, a 2-phenylallyl group, a 3-phenylallyl group, a 3,3-diphenylallyl group, a 1,2-dimethylallyl group, a 1-phenyl-1-butenyl group, a 3-phenyl-1-butenyl group or the like can be given. Preferred are a styryl group, a 2,2-diphenylvinyl group, a 1,2-diphenylvinyl group or the like.

As the alkynyl group including 2 to 50 (preferably 2 to 30, more preferably 2 to 20, particularly preferably 2 to 10) carbon atoms, a propargyl group, a 3-pentynyl group or the like can be given.

As the alkyl germanium group, a methylhydrogermyl group, a trimethylgermyl group, a triethylgermyl group, a tripropylgermyl group, a dimethyl-t-butylgermyl group or the like can be given.

As the aryl germanium group, a phenyldihydrogermyl group, a diphenylhydrogermyl group, a triphenylgermyl group, a tritolylgermyl group, a trinaphthylgermyl group or the like can be given.

As the styrylamine compound and the styryldiamine compound, those represented by the following formulas (17) and (18) are preferable:

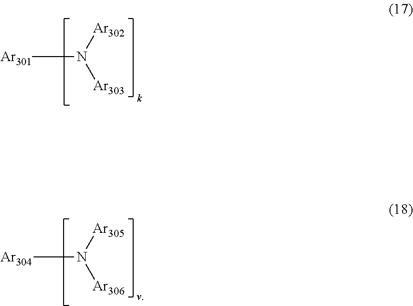

In the formula (17), Ar₃₀₁, is a k-valent group; a k-valent group corresponding to a phenyl group, a naphthyl group, a biphenyl group, a terphenyl group, a stilbene group, a styrylaryl group and a distyrylaryl group. Ar₃₀₂ and Ar₃₀₃ are independently an aryl group including 6 to 20 ring carbon atoms, and Ar₃₀₁, Ar₃₀₂ and Ar₃₀₃ may be substituted.

k is an integer of 1 to 4, with an integer of 1 and 2 being preferable. Any one of Ar₃₀₁ to Ar₃₀₃ is a group including a styryl group. It is further preferred that at least one of Ar₃₀₂ and Ar₃₀₃ be substituted by a styryl group.

As for the aryl group including 6 to 20 ring carbon atoms, the above-mentioned aryl group can be specifically given. Preferable examples include a phenyl group, a naphthyl group, an anthranyl group, a phenanthryl group, a terphenyl group or the like.

In the formula (18), Ar₃₀₄ to Ar₃₀₆ are a v-valent substituted or unsubstituted aryl group including 6 to 40 ring carbon atoms. v is an integer of 1 to 4, with an integer of 1 and 2 being preferable. Here, as the aryl group including 6 to 40 ring carbon atoms in the formula (18), the above-mentioned aryl group can be specifically given. A naphthyl group, an anthranyl group, a chrysenyl group, a pyrenyl group or an aryl group represented by the formula (20) is preferable.

As preferable substituents that substitute on the aryl group, an alkyl group including 1 to 6 carbon atoms, an alkoxy group including 1 to 6 carbon atoms, an aryl group including 6 to 40 ring carbon atoms, an amino group substituted by an aryl group including 6 to 40 ring carbon atoms, an ester group including an aryl group that includes 5 to 40 ring carbon atoms, an ester group including an alkyl group that includes 1 to 6 carbon atoms, a cyano group, a nitro group, a halogen atom or the like can be given.

The content of the emitter materials (dopants), preferably the phosphorescent emitter materials, in the light emitting layer is not particularly limited and selected according to the use of the device, and preferably 0.1 to 70% by mass, and more preferably 1 to 30% by mass. If being 0.1% by mass or more, the amount of light emission is sufficient. If being 70% by mass or less, the concentration quenching can be avoided. The further component in the emitting layer is usually one or more host material, which is preferably present in an amount of 30 to 99.9% by mass, more preferably 70 to 99% by mass, wherein the sum of the emitter material(s) and the host material(s) is 100% by mass.

Host (Matrix) Materials

The light-emitting layer may comprise further components in addition to the emitter material. For example, a fluroescent dye may be present in the light-emitting layer in order to alter the emission color of the emitter material. In addition—in a preferred embodiment—a matrix material can be used. This matrix material may be a polymer, for example poly(N-vinylcarbazole) or polysilane. The matrix material may, however, be a small molecule, for example 4,4′-N,N′-dicarbazolebiphenyl (CDP=CBP) or tertiary aromatic amines, for example TCTA.

In the case that one or more phosphorescent emitter materials are used in the light emitting layer, one or more phosphorescent hosts are employed as host material. The phosphorescent host is a compound which confines the triplet energy of the phosphorescent dopant efficiently in the light emitting layer to cause the phosphorescent dopant to emit light efficiently.

In a preferred embodiment, the light-emitting layer is formed of at least one emitter material and of at least one of the matrix materials mentioned in this application. According to a preferred embodiment, the electronic device according to the present invention, preferably the OLED according to the present invention, comprises at least one compound of the formulae (I), (II) or (III) as matrix (host) material.

According to one embodiment, the light-emitting layer comprises at least one emitter material and at least two matrix materials, wherein one of the matrix materials is a compound of the formulae (I), (II) or (III) and the other matrix material(s) is/are used as co-host(s). Suitable other host materials than the compounds of formulae (I), (II) or (III) (co-hosts) are mentioned below. This embodiment is preferably realized with emitter materials that emit green light.

According to another embodiment, the light-emitting layer comprises at least one emitter material and a compound of the formulae (I), (II) or (III) as a single matrix material. Examples of preferred compounds of formulae (I), (II) or (III) useful as single host material are shown above. This embodiment is preferably realized with emitter materials that emit red light.

The compounds of the formulae (I), (II) or (III) are suitable as single host material as well as host material, together with one or more further host materials (co-host). Suitable further host materials are mentioned below. “Further host materials” means in the sense of the present application, host materials different from the compounds of formulae (I), (II) or (III). However, it is also possible to use two or more different compounds of formulae (I), (II) or (III) as host material in the light-emitting layer in an OLED of the present application.

In a more preferred embodiment, the light-emitting layer is formed from 0.1 to 70% by weight, preferably 1 to 30% by weight, of at least one of the aforementioned emitter materials and 30 to 99.9% by weight, preferably 70 to 99% by weight, of at least one of the matrix materials mentioned in the specification—in one embodiment at least one compound of the formulae (I), (II) or (III)—where the sum total of the emitter material and of the matrix material adds up to 100% by weight.

In a further more preferred embodiment, the light-emitting layer comprises a compound of formulae (I), (II) or (III) as matrix material, at least one further matrix material (co-host) and at least one emitter material. In said embodiment, the light-emitting layer is formed from 0.1 to 70% by weight, preferably 1 to 30% by weight, of the at least one emitter material and 30 to 99.9% by weight, preferably 70 to 99% by weight, of a compound of the formulae (I), (II) or (III) and the further matrix material, where the sum total of the at least one emitter material, the further matrix material and of the compound of formulae (I), (II) or (III) adds up to 100% by weight.

The content ratio of the compound of the formulae (I), (II) or (III) as first host material and the second matrix material as co-host in the light emitting layer is not particularly limited and may be selected accordingly, and the ratio of first host material:second host material is preferably 1:99 to 99:1, more preferably 10:90 to 90:10, each based on mass.

In the following host materials are mentioned that may be used in the electronic device according to the present invention as single host materials, if the compounds according to the present invention are used as charge transporting material, i.e. as electron transporting material or hole transporting material, and/or as a dopant without metal species. The host materials that are mentioned in the following can also be used as second host materials, if the compounds according to general formulae (I), (II) or (III) are used as first host material and vice versa.

Further suitable host materials, which may be small molecules or (co)polymers of the small molecules mentioned, are specified in the following publications: WO2007108459 (H-1 to H-37), preferably H-20 to H-22 and H-32 to H-37, most preferably H-20, H-32, H-36, H-37, WO2008035571 A1 (Host 1 to Host 6), JP2010135467 (compounds 1 to 46 and Host-1 to Host-39 and Host-43), WO2009008100 compounds No. 1 to No. 67, preferably No. 3, No. 4, No. 7 to No. 12, No. 55, No. 59, No. 63 to No. 67, more preferably No. 4, No. 8 to No. 12, No. 55, No. 59, No. 64, No. 65, and No. 67, WO2009008099 compounds No. 1 to No. 110, WO2008140114 compounds 1-1 to 1-50, WO2008090912 compounds OC-7 to OC-36 and the polymers of Mo-42 to Mo-51, JP2008084913 H-1 to H-70, WO2007077810 compounds 1 to 44, preferably 1, 2, 4-6, 8, 19-22, 26, 28-30, 32, 36, 39-44, WO201001830 the polymers of monomers 1-1 to 1-9, preferably of 1-3, 1-7, and 1-9, WO2008029729 the (polymers of) compounds 1-1 to 1-36, WO20100443342 HS-1 to HS-101 and BH-1 to BH-17, preferably BH-1 to BH-17, JP2009182298 the (co)polymers based on the monomers 1 to 75, JP2009170764, JP2009135183 the (co)polymers based on the monomers 1-14, WO2009063757 preferably the (co)polymers based on the monomers 1-1 to 1-26, WO2008146838 the compounds a-1 to a-43 and 1-1 to 1-46, JP2008207520 the (co)polymers based on the monomers 1-1 to 1-26, JP2008066569 the (co)polymers based on the monomers 1-1 to 1-16, WO2008029652 the (co)polymers based on the monomers 1-1 to 1-52, WO2007114244 the (co)polymers based on the monomers 1-1 to 1-18, JP2010040830 the compounds HA-1 to HA-20, HB-1 to HB-16, HC-1 to HC-23 and the (co)polymers based on the monomers HD-1 to HD-12, JP2009021336, WO2010090077 the compounds 1 to 55, WO2010079678 the compounds H1 to H42, WO2010067746, WO2010044342 the compounds HS-1 to HS-101 and Poly-1 to Poly-4, JP2010114180 the compounds PH-1 to PH-36, US2009284138 the compounds 1 to 111 and H1 to H71, WO2008072596 the compounds 1 to 45, JP2010021336 the compounds H-1 to H-38, preferably H-1, WO2010004877 the compounds H-1 to H-60, JP2009267255 the compounds 1-1 to 1-105, WO2009104488 the compounds 1-1 to 1-38, WO2009086028, US2009153034, US2009134784, WO2009084413 the compounds 2-1 to 2-56, JP2009114369 the compounds 2-1 to 2-40, JP2009114370 the compounds 1 to 67, WO2009060742 the compounds 2-1 to 2-56, WO2009060757 the compounds 1-1 to 1-76, WO2009060780 the compounds 1-1 to 1-70, WO2009060779 the compounds 1-1 to 1-42, WO2008156105 the compounds 1 to 54, JP2009059767 the compounds 1 to 20, JP2008074939 the compounds 1 to 256, JP2008021687 the compounds 1 to 50, WO2007119816 the compounds 1 to 37, WO2010087222 the compounds H-1 to H-31, WO2010095564 the compounds HOST-1 to HOST-61, WO2007108362, WO2009003898, WO2009003919, WO2010040777, US2007224446, WO06128800, WO2012014621, WO2012105310, WO2012/130709 and European patent applications EP12175635.7, EP12185230.5 and EP12191408.9 (in particular page 25 to 29 of EP12191408.9).

The above-mentioned small molecules are more preferred than the above-mentioned (co)polymers of the small molecules.

Further suitable host materials, are described in WO2011137072 (for example,

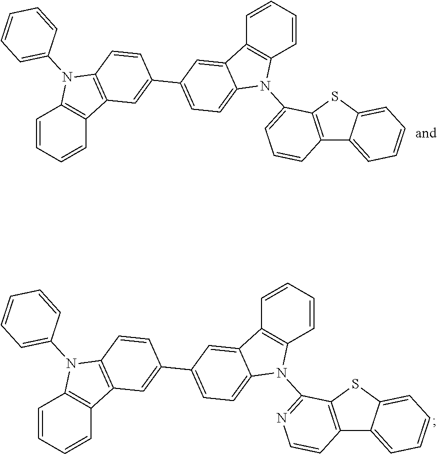 [SMILES: c1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4nccc5c4sc4ccccc45)ccc32)cc1]

best results are achieved if said compounds are combined with

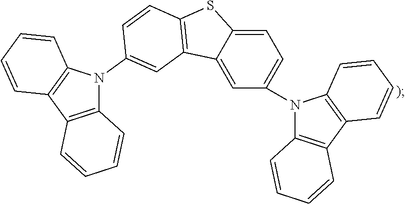 [SMILES: c1ccc2c(c1)c1ccccc1n2-c1ccc2sc3ccc(-n4c5ccccc5c5ccccc54)cc3c2c1]

WO2012048266 (for example,

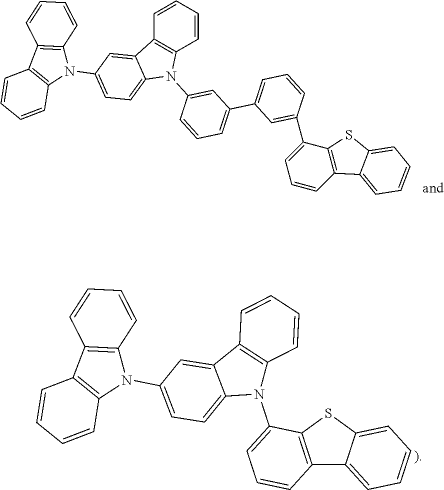 [SMILES: c1cc(-c2cccc(-n3c4ccccc4c4cc(-n5c6ccccc6c6ccccc65)ccc43)c2)cc(-c2cccc3c2sc2ccccc23)c1 ; c1ccc2c(c1)sc1c(-n3c4ccccc4c4cc(-n5c6ccccc6c6ccccc65)ccc43)cccc12]

The host materials mentioned above may be used in the OLED of the present invention alone or in combination with the compound of formulae (I), (II) or (III) as host material. In this case, the compound of formulae (I), (II) or (III) is the host and the host materials mentioned above are the co-hosts.

Further examples of the compounds which are suitable as phosphorescent host, alone or in combination with the compound of formulae (I), (II) or (III) as host material, include a carbazole derivative, a triazole derivative, a oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styrylanthracene derivative, a fluorenone derivative, a hydrazone derivative, a stilbene derivative, a silazane derivative, an aromatic tertiary amine compound, a styrylamine compound, an aromatic methylidene compound, a porphyrin compound, an anthraquinodimethane derivative, an anthrone derivative, a diphenylquinone derivative, a thiopyran dioxide derivative, a carbodiimide derivative, a fluorenylidenemethane derivative, a distyrylpyrazine derivative, a tetracarboxylic anhydride of fused ring such as naphthalene and perylene, a phthalocyanine derivative, a metal complex of 8-quinolinol derivative, metal phthalocyanine, metal complexes having a ligand such as benzoxazole and benzothiazole, an electroconductive oligomer, such as a polysilane compound, a poly(N-vinylcarbazole) derivative, an aniline copolymer, thiophene oligomer, and a polythiophene, and a polymer such as a polythiophene derivative, a polyphenylene derivative, a polyphenylenevinylene derivative, and a polyfluorene derivative. These phosphorescent hosts may be used alone or in combination of two or more. Specific examples thereof are shown below:

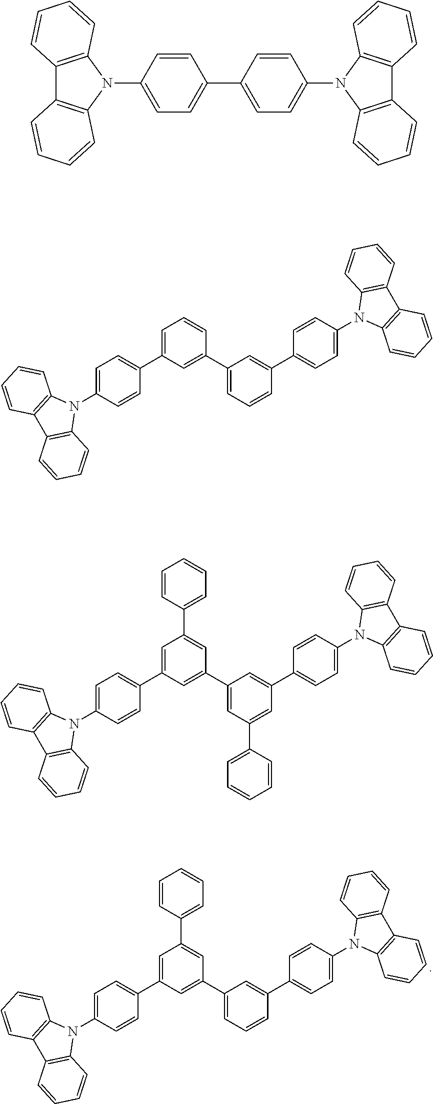 [SMILES: c1ccc2c(c1)c1ccccc1n2-c1ccc(-c2ccc(-n3c4ccccc4c4ccccc43)cc2)cc1 ; c1cc(-c2ccc(-n3c4ccccc4c4ccccc43)cc2)cc(-c2cccc(-c3ccc(-n4c5ccccc5c5ccccc54)cc3)c2)c1 ; c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

Further suitable hosts, which are especially useful as co-host together with at least one compound of formulae (I), (II) or (III) are the hosts described in US2014048784, US2012223295, US2014367667, US2013234119, US2014001446, US2014231794, US2014008633, WO2012108388, WO2014009317 and WO2012108389, as well as the compounds of formula (1) described in the EP application EP 15187954, filed on Oct. 1, 2015.

Especially preferred are the first and second host materials mentioned in US2013234119, the host materials mentioned in US2014048784 and the compounds of formula (1) described in the EP application EP 15187954, filed on Oct. 1, 2015.

The first host material mentioned in US2013234119 which is preferably used as co-host together with at least one compound of formulae (I), (II) or (III) in the light emitting layer of an OLED according to the present invention is represented by formula (A). The lifetime of an OLED is increased by combinedly using as a first host material at least one compound of formulae (I), (II) or (III) and as co-host the host material represented by formula (A) in the light emitting layer:

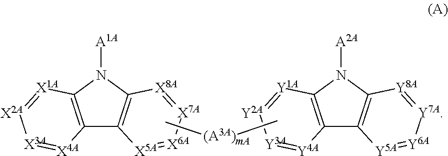

wherein

each of A^{1A} and A^{2A} independently represents an aryl group having 6 to 30 ring carbon atoms, which may be unsubstituted or substituted; or a heterocyclic group having 5 to 30 ring atoms, which may be unsubstituted or substituted;

A^{3A} represents a divalent aryl group having 6 to 30 ring carbon atoms, which may be unsubstituted or substituted; or a divalent heterocyclic group having 5 to 30 ring atoms, which may be unsubstituted or substituted;

mA represents an integer of 0 to 3;

each of X¹A to X^{8A} and Y^{1A} to Y^{8A} independently represents N or CR^{a};

each of R^{a} independently represents a hydrogen atom, an aryl group having 6 to 30 ring carbon atoms, which may be unsubstituted or substituted; a heterocyclic group having 5 to 30 ring atoms, which may be unsubstituted or substituted; an alkyl group having 1 to 30 carbon atoms, which may be unsubstituted or substituted for example by E; a silyl group, which may be unsubstituted or substituted; a halogen atom, or a cyano group, provided that when two or more R^{a} groups exist, the R^{a} groups may be the same or different and one of X⁵A to X^{8A} and one of Y^{1A} to Y^{4A} are bonded to each other via A^{3A}; and

the formula (A) satisfies at least one of the flowing requirements (i) to (v);

(i) at least one of A^{1A} and A^{2A} represents a cyano-substituted aromatic hydrocarbon group having 6 to 30 ring carbon atoms or a cyano-substituted heterocyclic group having 5 to 30 ring atoms;

(ii) at least one of X^{1A} to X^{4A} and Y^{5A} to Y^{8A} represents CR^{a}, and at least one of R^{a} in X^{1A} to X^{4A} and Y^{5A} to Y^{8A} represents a cyano-substituted aromatic hydrocarbon group having 6 to 30 ring carbon atoms or a cyano-substituted heterocyclic group having 5 to 30 ring atoms;

(iii) mA represents an integer of 1 to 3 and at least one of A³ represents a cyano-substituted divalent aromatic hydrocarbon group having 6 to 30 ring carbon atoms or a cyano-substituted divalent heterocyclic group having 5 to 30 ring atoms;

(iv) at least one of X^{5A} to X^{8A} and Y^{1A} to Y^{4A} represents CR^{a}, and at least one of R^{a} in X^{5A} to X^{8A} and Y^{1A} to Y^{4A} represents a cyano-substituted aromatic hydrocarbon group having 6 to 30 ring carbon atoms or a cyano-substituted heterocyclic group having 5 to 30 ring atoms; and

(v) at least one of X^{1A} to X^{8A} and Y^{1A} to Y^{8A} represents C—CN.

The cyano-substituted aromatic hydrocarbon group having 6 to 30 ring carbon atoms and the cyano-substituted heterocyclic group having 5 to 30 ring atoms may be further substituted by a group other than the cyano group.

The subscript mA is preferably 0 to 2 and more preferably 0 or 1. When mA is 0, one of X^{5A} to X^{8A} and one of Y^{1A} to Y^{4A} are bonded to each other via a single bond.

In formula (A), the groups mentioned above have the following meanings: The aromatic hydrocarbon group having 6 to 30 ring carbon atoms represented by A^{1A}, A^{2A} and R^{a} may be a non-condensed aromatic hydrocarbon group or a condensed aromatic hydrocarbon group. Specific examples thereof include phenyl group, naphthyl group, phenanthryl group, biphenyl group, terphenyl group, quaterphenyl group, fluoranthenyl group, triphenylenyl group, phenanthrenyl group, fluorenyl group, spirofluorenyl group, 9,9-diphenylfluorenyl group, 9,9′-spirobi[9H-fluorene]-2-yl group, 9,9-dimethylfluorenyl group, benzo[c]phenanthrenyl group, benzo[a]triphenylenyl group, naphtho[1,2-c]phenanthrenyl group, naphtho[1,2-a]triphenylenyl group, dibenzo[a,c]triphenylenyl group, and benzo[b]fluoranthenyl group, with phenyl group, naphthyl group, biphenyl group, terphenyl group, phenanthryl group, triphenylenyl group, fluorenyl group, spirobifluorenyl group, and fluoranthenyl group being preferred, and phenyl group, 1-naphthyl group, 2-naphthyl group, biphenyl-2-yl group, biphenyl-3-yl group, biphenyl-4-yl group, phenanthrene-9-yl group, phenanthrene-3-yl group, phenanthrene-2-yl group, triphenylene-2-yl group, 9,9-dimethylfluorene-2-yl group, fluoranthene-3-yl group being more preferred.

Examples of the divalent aromatic hydrocarbon group having 6 to 30 ring carbon atoms represented by A^{3A} include divalent residues of the above aromatic hydrocarbon groups having 6 to 30 ring carbon atoms.

The heterocyclic group having 5 to 30 ring atoms represented by A^{1A}, A^{2A} and R^{a} may be a non-condensed heterocyclic group or a condensed heterocyclic group. Specific examples thereof include the residues of pyrrole ring, isoindole ring, benzofuran ring, isobenzofuran ring, dibenzothiophene ring, isoquinoline ring, quinoxaline ring, phenanthridine ring, phenanthroline ring, pyridine ring, pyrazine ring, pyrimidine ring, pyridazine ring, triazine ring, indole ring, quinoline ring, acridine ring, pyrrolidine ring, dioxane ring, piperidine ring, morpholine ring, piperazine ring, carbazole ring, furan ring, thiophene ring, oxazole ring, oxadiazole ring, benzoxazole ring, thiazole ring, thiadiazole ring, benzothiazole ring, triazole ring, imidazole ring, benzimidazole ring, pyran ring, dibenzofuran ring, and benzo[c]dibenzofuran ring, and the residues of derivatives of these rings, with the residues of dibenzofuran ring, carbazole ring, dibenzothiophene ring, and derivatives of these rings being preferred, and the residues of dibenzofuran-2-yl group, dibenzofuran-4-yl group, 9-phenylcarbazole-3-yl group, 9-phenylcarbazole-2-yl group, dibenzothiophene-2-yl group, and dibenzothiophene-4-yl group being more preferred.

Examples of the divalent heterocyclic group having 5 to 30 ring atoms represented by A^{3A} include divalent residues of the above heterocyclic group having 5 to 30 ring atoms.

Examples of the alkyl group having 1 to 30 carbon atoms represented by R^{a} include methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, 1-methylpentyl group, cyclopropyl group, cyclobutyl group, cyclopentyl group, cyclohexyl group, cyclooctyl group, and adamantyl group, with methyl group, ethyl group, n-propyl group, isopropyl group, n-butyl group, s-butyl group, isobutyl group, t-butyl group, cyclopentyl group, and cyclohexyl group being preferred.

Examples of the silyl group, which may be unsubstituted or substituted; represented by R^{a} include trimethylsilyl group, triethylsilyl group, tributylsilyl group, dimethylethylsilyl group, t-butyldimethylsilyl group, vinyldimethylsilyl group, propyldimethylsilyl group, dimethylisopropylsilyl group, dimethylpropylsilyl group, dimethylbutylsilyl group, dimethyltertiarybutylsilyl group, diethylisopropylsilyl group, phenyldimethylsilyl group, diphenylmethylsilyl group, diphenyltertiarybutylsilyl group, and triphenylsilyl group, with trimethylsilyl group, triethylsilyl group, t-butyldimethylsilyl group, vinyldimethylsilyl group, and propyldimethylsilyl group being preferred.

Examples of the halogen atom represented by R^{a} include fluorine, chlorine, bromine, and iodine, with fluorine being preferred.

Also preferred as R^{a} is a hydrogen atom or an aryl group having 6 to 30 ring carbon atoms, which may be unsubstituted or substituted.

Examples of the optional substituent indicated by “substituted or unsubstituted” and “may be substituted” referred to above or hereinafter include a halogen atom (fluorine, chlorine, bromine, iodine), a cyano group, an alkyl group having 1 to 20, preferably 1 to 6 carbon atoms, a cycloalkyl group having 3 to 20, preferably 5 to 12 carbon atoms, an alkoxyl group having 1 to 20, preferably 1 to 5 carbon atoms, a haloalkyl group having 1 to 20, preferably 1 to 5 carbon atoms, a haloalkoxyl group having 1 to 20, preferably 1 to 5 carbon atoms, an alkylsilyl group having 1 to 10, preferably 1 to 5 carbon atoms, an aromatic hydrocarbon group having 6 to 30, preferably 6 to 18 ring carbon atoms, an aryloxy group having 6 to 30, preferably 6 to 18 ring carbon atoms, an arylsilyl group having 6 to 30, preferably 6 to 18 carbon atoms, an aralkyl group having 7 to 30, preferably 7 to 20 carbon atoms, and a heteroaryl group having 5 to 30, preferably 5 to 18 ring atoms.

The optional substituent mentioned above may be further substituted by the optional group mentioned above.

Examples of the optional alkyl group having 1 to 20 carbon atoms include methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, and 1-methylpentyl group.

Examples of the optional cycloalkyl group having 3 to 20 carbon atoms include cyclopropyl group, cyclobutyl group, cyclopentyl group, cyclohexyl group, cyclooctyl group, and adamantyl group.

Examples of the optional alkoxyl group having 1 to 20 carbon atoms include those having an alkyl portion selected from the alkyl groups mentioned above.

Examples of the optional haloalkyl group having 1 to 20 carbon atoms include the alkyl groups mentioned above wherein the hydrogen atoms thereof are partly or entirely substituted by halogen atoms.

Examples of the optional haloalkoxyl group having 1 to 20 carbon atoms include the alkoxyl group mentioned above wherein the hydrogen atoms thereof are partly or entirely substituted by halogen atoms.

Examples of the optional alkylsilyl group having 1 to 10 carbon atoms include trimethylsilyl group, triethylsilyl group, tributylsilyl group, dimethylethylsilyl group, t-butyldimethylsilyl group, vinyldimethylsilyl group, propyldimethylsilyl group, dimethylisopropylsilyl group, dimethylpropylsilyl group, dimethylbutylsilyl group, dimethyltertiarybutylsilyl group, and diethylisopropylsilyl group.

Examples of the optional aryl group having 6 to 30 ring carbon atoms include those selected from the aryl groups mentioned above with respect to A^{1A}, A^{2A} and R^{a}.

Examples of the optional aryloxy group having 6 to 30 ring carbon atoms include those having an aryl portion selected from the aromatic hydrocarbon groups mentioned above.

Examples of the optional arylsilyl group having 6 to 30 carbon atoms include phenyldimethylsilyl group, diphenylmethylsilyl group, diphenyltertiarybutylsilyl group, and triphenylsilyl group.

Examples of the optional aralkyl group having 7 to 30 carbon atoms include benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, 3-naphthylmethyl group, 1-β-naphthylethyl group, 2-β-naphthylethyl group, 1-β-naphthylisopropyl group, 2-β-naphthylisopropyl group, 1-pyrrolylmethyl group, 2-(1-pyrrolyl)ethyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group, and 1-chloro-2-phenylisopropyl group.

Examples of the optional heteroaryl group having 5 to 30 ring atoms include those selected from the heterocyclic groups mentioned above with respect to A^{1A}, A^{2A} and R^{a}.

The “carbon number of a to b” in the expression of “substituted or unsubstituted X group having carbon number of a to b” is the carbon number of the unsubstituted X group and does not include the carbon atom of the optional substituent.

The hydrogen atom referred to herein includes isotopes different from neutron numbers, i.e., light hydrogen (protium), heavy hydrogen (deuterium) and tritium.

In the host material represented by formula (A), the groups represented by formulae (a) and (b) are bonded to each other via -(A³)_{mA}- at one of X^{5A} to X^{8A} and one of Y^{1A} to Y^{4A}. Specific examples of the bonding manner between formulae (a) and (b) are represented by X^{6A}-(A^{3A})_{mA}-Y^{3A}, X^{6A}-(A^{3A})_{mA}-Y^{2A}, X^{6A}-(A^{3A})_{mA}-Y^{4A}, X^{6A}-(A^{3A})_{mA}-Y^{1A}, X^{7A}-(A^{3A})_{mA}-Y^{3A}, X^{5A}-(A^{3A})_{mA}- Y^{3A}, X^{8A} (A^{3A})_{mA}-Y^{3A}, X^{7A}-(A^{3A})_{mA}-Y^{2A}, X^{7A}-(A^{3A})_{mA}-Y^{4A}, X^{7A}-(A^{3A})_{mA}-Y^{1A}, X^{5A}-(A^{3A})_{mA}-Y²A, X^{8A}- (A^{3A})_{mA}-Y^{2A}, X^{8A}-(A^{3A})_{mA}-Y^{4A}, X^{8A}-(A^{3A})_{mA}-Y^{1A}, X^{5A}-(A^{3A})_{mA}-Y^{1A}, and X^{5A}-(A^{3A})_{mA}-Y^{4A}.

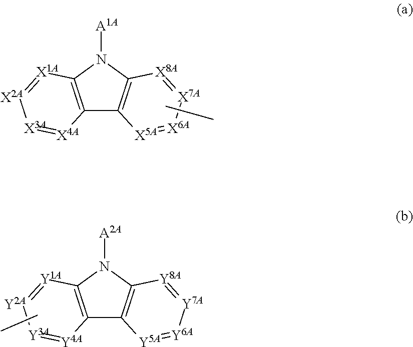

In preferred embodiments of the host material represented by formula (A), the bonding manner between formulae (a) and (b) are represented by X^{6A}-(A^{3A})_{mA}-Y^{3A}, X^{6A}-(A^{3A})_{mA}-Y^{2A}, or X^{7A}-(A^{3A})_{mA}-Y^{3A}, namely the material for organic electroluminescence device is preferably represented by formula (XXII), (XXIII), or (XXIV):

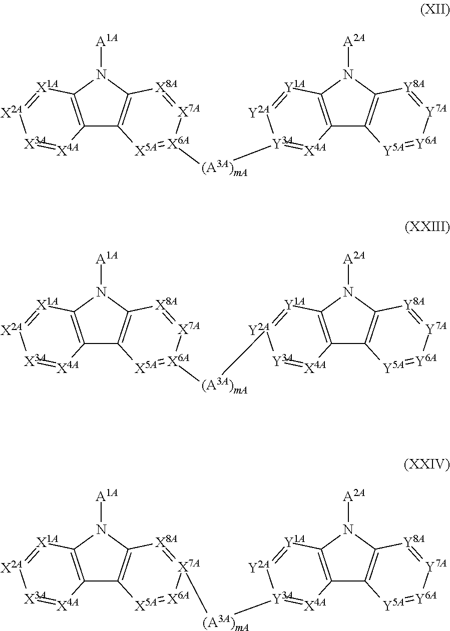

wherein X^{1A} to X^{8A}, Y^{1A} to Y^{8A}, A^{1A} to A^{3A}, and mA are the same as X^{1A} to X^{8A}, Y^{1A} to Y^{8A}, A^{1A} to A^{3A}, mA in formula (A), and each of formulae (XXII), (XXIII), and (XXIV) satisfies at least one of the requirements (i) to (v) as specified in the definition of formula (A).

The host material represented by formula (A) satisfies at least one of the requirements (i) to (v), namely, the host material is a cyano group-introduced biscarbazole derivative having a group represented by formula (a) and a group represented by formula (b) which are linked to each other.

A^{3A} of formula (A) preferably represents a single bond, a substituted or unsubstituted divalent monocyclic hydrocarbon group having 6 or less ring carbon atoms, or a substituted or unsubstituted divalent monocyclic heterocyclic group having 6 or less ring atoms.

Examples of the monocyclic hydrocarbon group having 6 or less ring carbon atoms represented by A^{3A} include phenylene group, cyclopentenylene group, cyclopentadienylene group, cyclohexylene group, and cyclopentylene group, with phenylene group being preferred.

Examples of the monocyclic heterocyclic group having 6 or less ring atoms represented by A^{3A} include pyrrolylene group, pyrazinylene group, pyridinylene group, furylene group, and thiophenylene group.

In a preferred embodiment of formulae (A), (XXII), (XXIII), and (XXIV), mA is 0 and one of X^{5A} to X^{8A} and one of Y^{1A} to Y^{4A} are bonded to each other via a single bond; or A^{3A} represents the substituted or unsubstituted monocyclic hydrocarbon group having 6 or less ring carbon atoms or the substituted or unsubstituted monocyclic heterocyclic group having 6 or less ring atoms. In more preferred embodiment, mA is 0 and one of X^{5A} to X^{8A} and one of Y^{1A} to Y^{4A} are bonded to each other via a single bond; or A^{3A} represents a substituted or unsubstituted phenylene group.

The host material of formula (A) satisfies preferably at least one of the requirements (i) and (ii):

(i) at least one of A^{1A} and A^{2A} represents a cyano-substituted aromatic hydrocarbon group having 6 to 30 ring carbon atoms or a cyano-substituted heterocyclic group having 5 to 30 ring atoms; and

(ii) at least one of X^{1A} to X^{4A} and Y^{5A} to Y^{8A} represents CR^{a}, and at least one of R^{a} in X^{1A} to X^{4A} and Y^{5A} to Y^{8A} represents a cyano-substituted aromatic hydrocarbon group having 6 to 30 ring carbon atoms or a cyano-substituted heterocyclic group having 5 to 30 ring atoms.

Namely, the host material of formula (A) is preferably any one of the compounds:

(1) satisfying the requirement (i), but not satisfying the requirements (ii) to (v);

(2) satisfying the requirement (ii), but not satisfying the requirements (i) and (iii) to (v); and

(3) satisfying both the requirements (i) and (ii), but not satisfying the requirements (iii) to (v).

The host material of formula (A) satisfying the requirement (i) and/or (ii) has a structure wherein the cyano group-containing aromatic hydrocarbon group or the cyano group-containing heterocyclic group is introduced to the terminal end of the central skeleton comprising the groups represented by formulae (a) and (b).

When the host material of formula (A) satisfies the requirement (i), at least one of A^{1A} and A^{2A} is preferably a cyano-substituted phenyl group, a cyano-substituted naphthyl group, a cyano-substituted phenanthryl group, a cyano-substituted dibenzofuranyl group, a cyano-substituted dibenzothiophenyl group, a cyano-substituted biphenyl group, a cyano-substituted terphenyl group, a cyano-substituted 9,9-diphenylfluorenyl group, a cyano-substituted 9,9′-spirobi[9H-fluorene]-2-yl group, a cyano-substituted 9,9′-dimethylfluorenyl group, or a cyano-substituted triphenylenyl group, and more preferably 3′-cyanobiphenyl-2-yl group, 3′-cyanobiphenyl-3-yl group, 3′-cyanobiphenyl-4-yl group, 4′-cyanobiphenyl-3-yl group, 4′-cyanobiphenyl-4-yl group, 4′-cyanobiphenyl-2-yl group, 6-cyanonaphthalene-2-yl group, 4-cyanonaphthalene-1-yl group, 7-cyanonaphthalene-2-yl group, 8-cyanodibenzofuran-2-yl group, 6-cyanodibenzofuran-4-yl group, 8-cyanodibenzothiophene-2-yl group, 6-cyanodibenzothiophene-4-yl group, 7-cyano-9-phenylcarbazole-2-yl group, 6-cyano-9-phenylcarbazole-3-yl group, 7-cyano-9,9-dimethylfluorene-2-yl group, or 7-cyanotriphenylene-2-yl group.

The host material of formula (A) wherein A^{1A} is substituted by a cyano group and A^{2A} is not substituted by a cyano group is preferred. In this case, the first host material which does not satisfy the requirement (ii) is more preferred.

When the host material of formula (A) satisfies the requirement (ii), at least one of X¹A to X^{4A} and Y^{5A} to Y^{8A} is preferably CR^{a}, and one of R^{a} in X^{1A} to X^{4A} and Y^{5A} to Y^{8A} is preferably a cyano-substituted phenyl group, a cyano-substituted naphthyl group, a cyano-substituted phenanthryl group, a cyano-substituted dibenzofuranyl group, a cyano-substituted dibenzothiophenyl group, a cyano-substituted biphenyl group, a cyano-substituted terphenyl group, a cyano-substituted 9,9-diphenylfluorenyl group, a cyano-substituted 9,9′-spirobi[9H-fluorene]-2-yl group, a cyano-substituted 9,9′-dimethylfluorenyl group, or a cyano-substituted triphenylenyl group, and more preferably 3′-cyanobiphenyl-2-yl group, 3′-cyanobiphenyl-3-yl group, 3′-cyanobiphenyl-4-yl group, 4′-cyanobiphenyl-3-yl group, 4′-cyanobiphenyl-4-yl group, 4′-cyanobiphenyl-2-yl group, 6-cyanonaphthalene-2-yl group, 4-cyanonaphthalene-1-yl group, 7-cyanonaphthalene-2-yl group, 8-cyanodibenzofuran-2-yl group, 6-cyanodibenzofuran-4-yl group, 8-cyanodibenzothiophene-2-yl group, 6-cyanodibenzothiophene-4-yl group, 7-cyano-9-phenylcarbazole-2-yl group, 6-cyano-9-phenylcarbazole-3-yl group, 7-cyano-9,9-dimethylfluorene-2-yl group, or 7-cyanotriphenylene-2-yl group.

It is preferred for the host material of formula (A) to satisfy the requirement (ii), but not satisfy the requirement (i).

In formulae (A) and (XXII) to (XXIV), A^{1A} and A are preferably different from each other, and more preferably, A^{1A} is substituted by a cyano group but A2A is not substituted by a cyano group. Namely, the host material of formula (A) is preferably structurally asymmetric.

The production method of the first host material is not particularly limited and it is produced according to a known method, for example, by a coupling reaction of a carbazole derivative and an aromatic halogenated compound in the presence of a copper catalyst described in Tetrahedron 40 (1984) 1435 to 1456 or a palladium catalyst described in Journal of American Chemical Society 123 (2001) 7727 to 7729.

Examples of the host material of formula (A) are mentioned in [0145] in US2013234119.

Examples for preferred host materials that are preferably used as co-hosts in the electronic device according to the present invention are mentioned in US2013234119, WO2012108388 and WO2014009317 are:

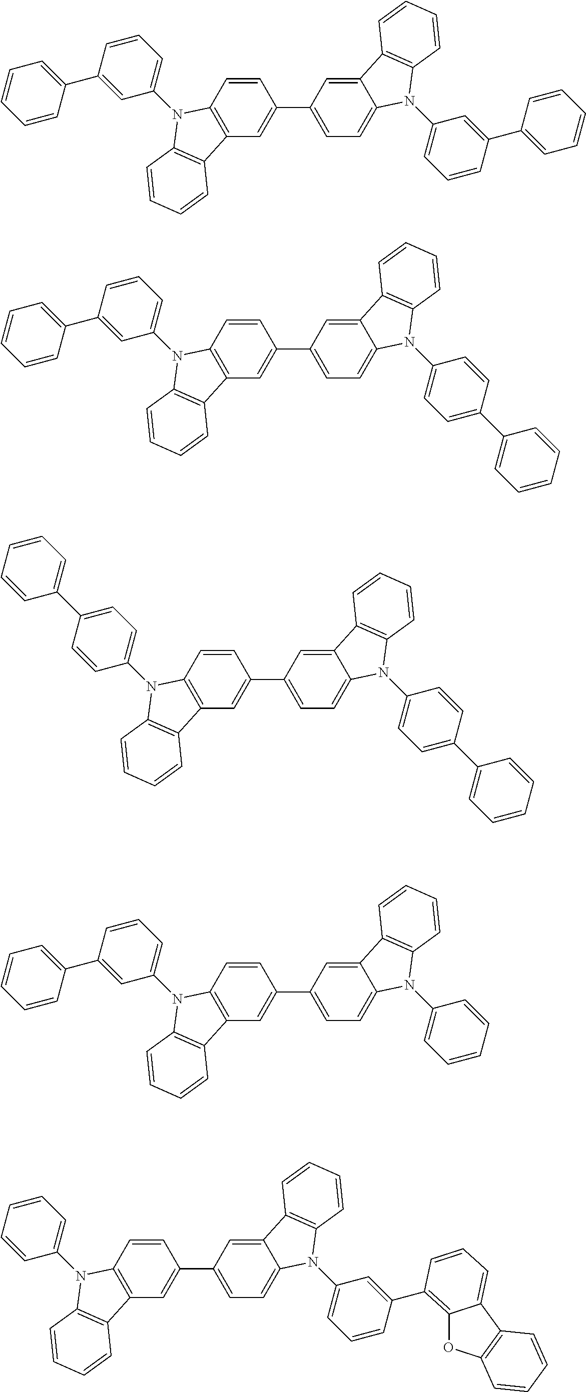 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc(-c5cccc6c5oc5ccccc56)c4)ccc32)cc1]

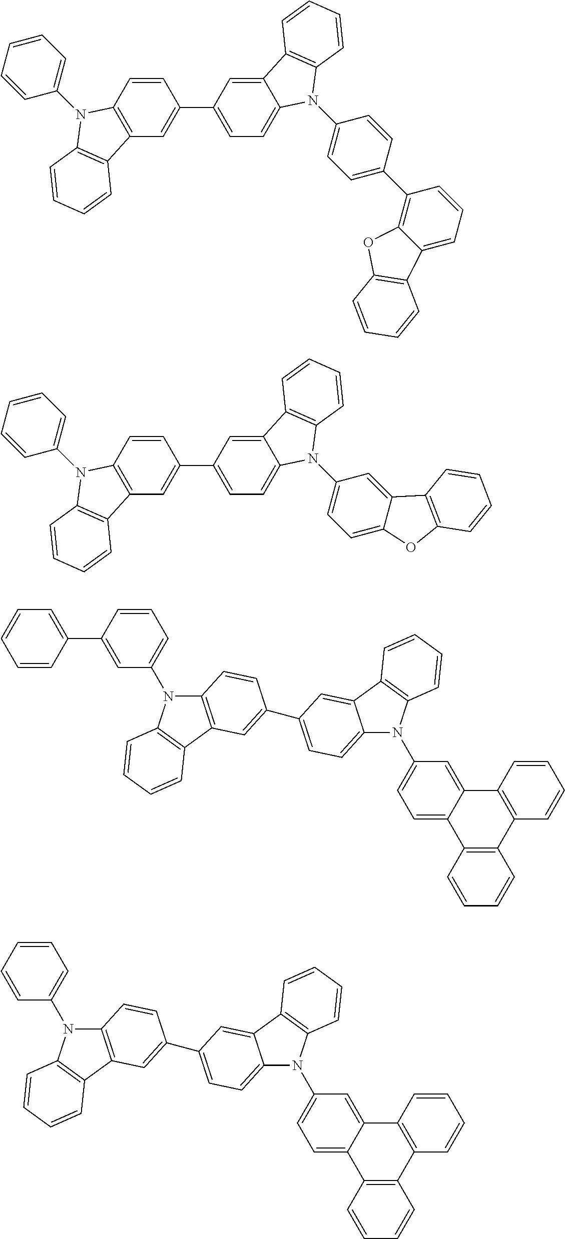 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-n2c3ccccc3c3cc(-c4ccc5c(c4)c4ccccc4n5-c4ccc5c6ccccc6c6ccccc6c5c4)ccc32)cc1]

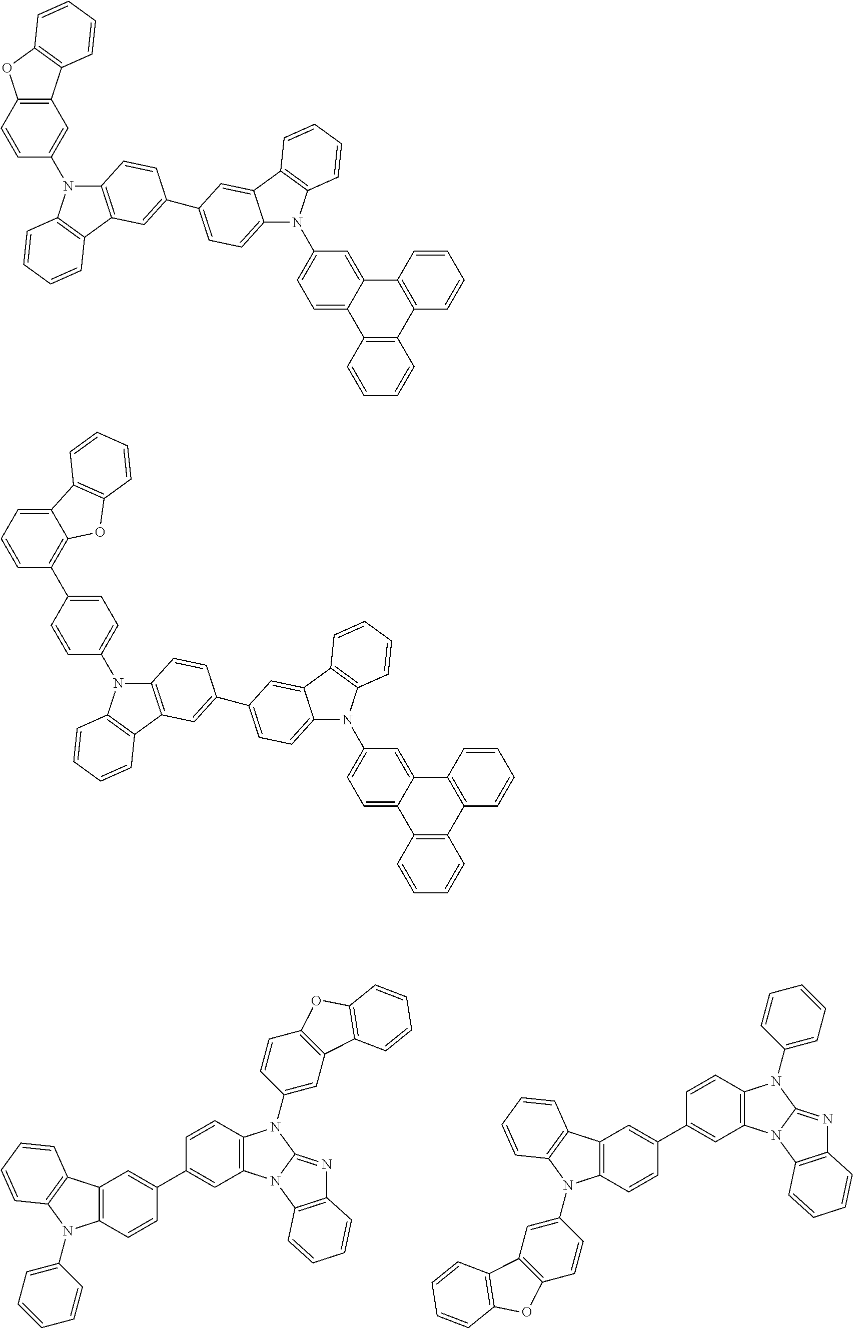 [SMILES: c1ccc2c(c1)oc1ccc(-n3c4ccccc4c4cc(-c5ccc6c(c5)c5ccccc5n6-c5ccc6c7ccccc7c7ccccc7c6c5)ccc43)cc12 ; c1ccc2c(c1)oc1c(-c3ccc(-n4c5ccccc5c5cc(-c6ccc7c(c6)c6ccccc6n7-c6ccc7c8ccccc8c8ccccc8c7c6)ccc54)cc3)cccc12 ; c1ccc(-n2c3ccccc3c3cc(-c4ccc5c(c4)n4c6ccccc6nc4n5-c4ccc5oc6ccccc6c5c4)ccc32)cc1 ; c1ccc(-n2c3ccc(-c4ccc5c(c4)c4ccccc4n5-c4ccc5oc6ccccc6c5c4)cc3n3c4ccccc4nc23)cc1]

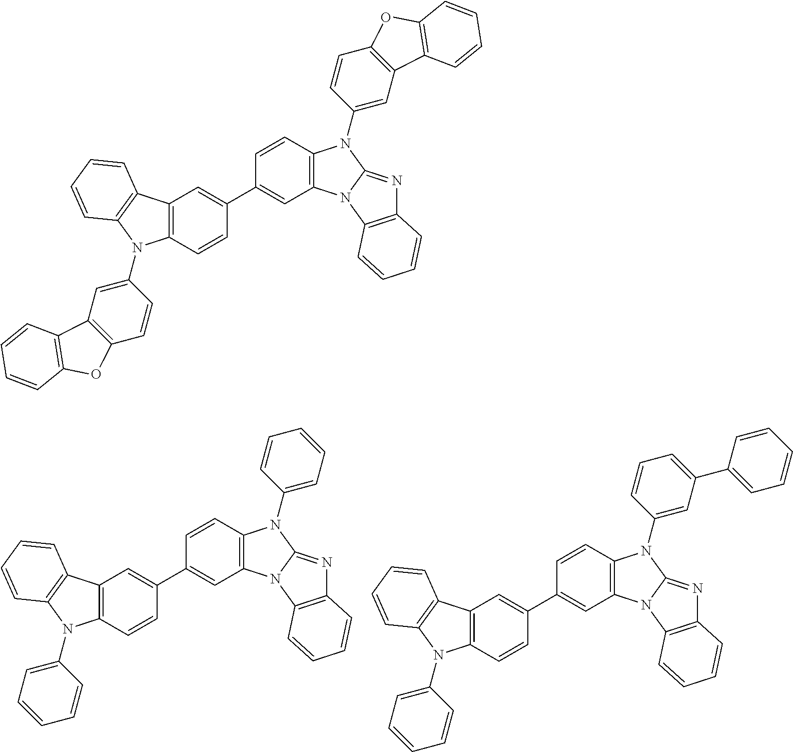

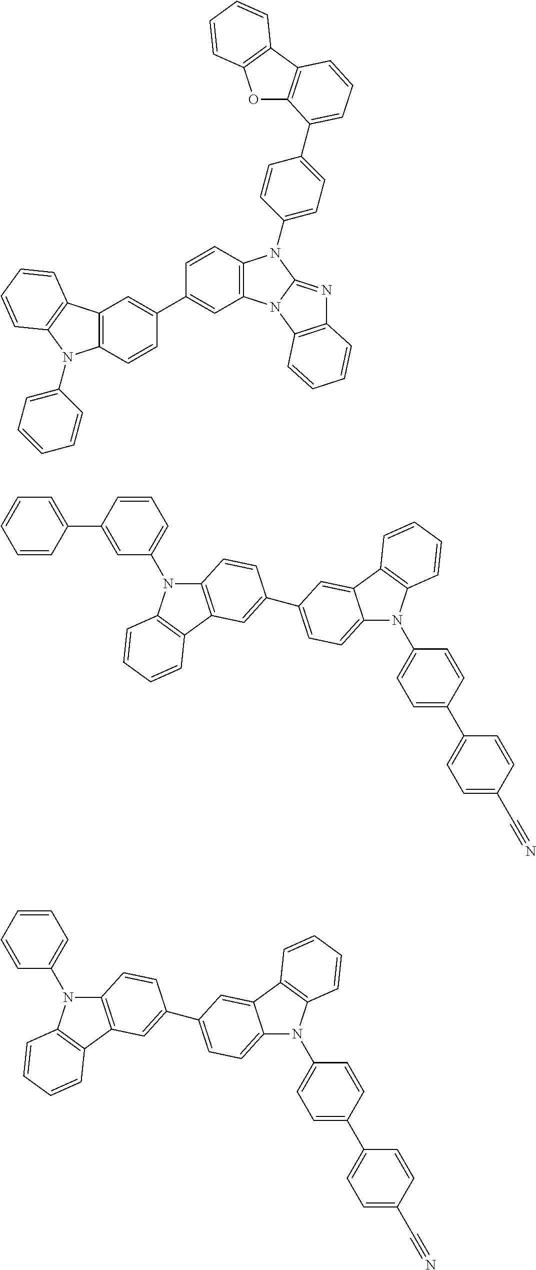

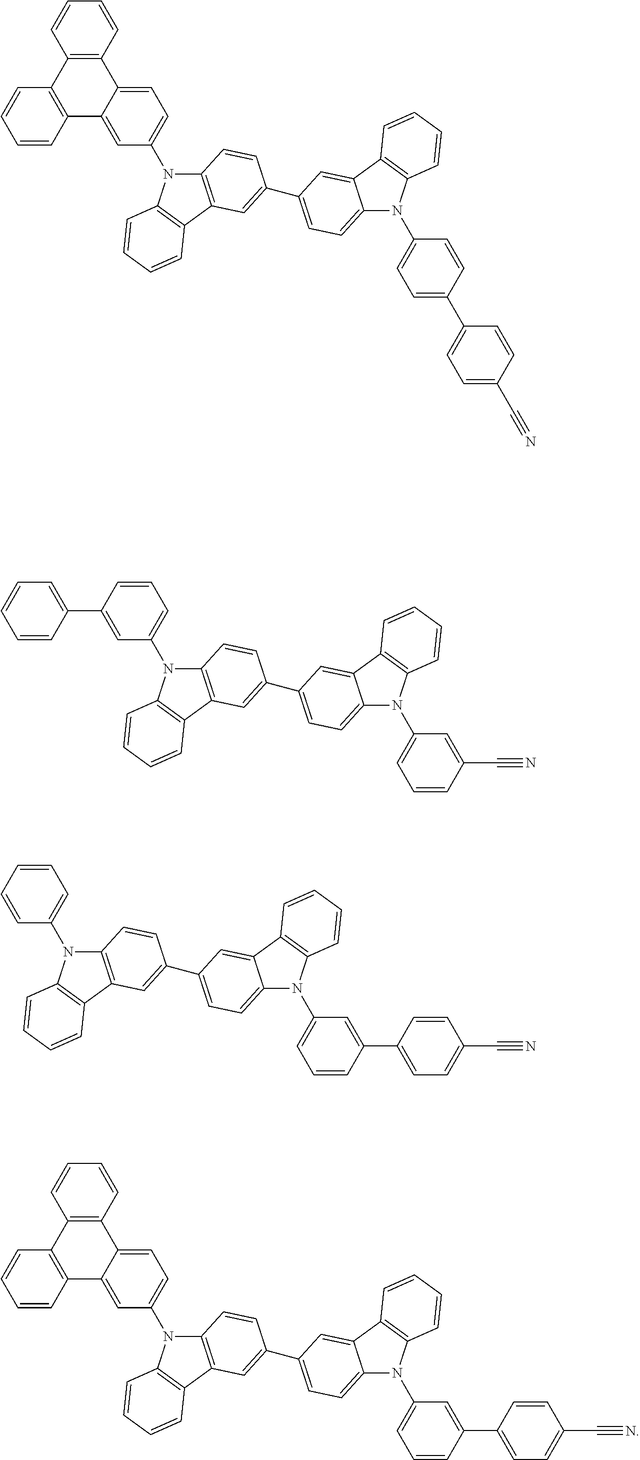 [SMILES: N#Cc1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4cccc(-c5ccccc5)c4)ccc32)c1 ; N#Cc1ccc(-c2cccc(-n3c4ccccc4c4cc(-c5ccc6c(c5)c5ccccc5n6-c5ccccc5)ccc43)c2)cc1 ; N#Cc1ccc(-c2cccc(-n3c4ccccc4c4cc(-c5ccc6c(c5)c5ccccc5n6-c5ccc6c7ccccc7c7ccccc7c6c5)ccc43)c2)cc1]

According to the present invention, the compounds according to general formulae (I), (II) or (III) can also be used in combination with host materials that are called “second host materials” in US20130234119, see in particular paragraphs 0146 to 0195 of US20130234119. In addition these compounds according to paragraphs 0146 to 1095 of US20130234119 can also be used as single host material in the electronic device according to the present invention, for example for red emitter material or green emitter material, preferably for red emitter material. The use of compounds according to general formulae (I), (II) or (III) according to the present invention in combination with host materials according to paragraphs 0146 to 0195 of US20130234119 as host material for green light emitting materials is preferred.

In particular, compounds according to the formula (Ia) can be used as host materials in the electronic device according to the present invention:

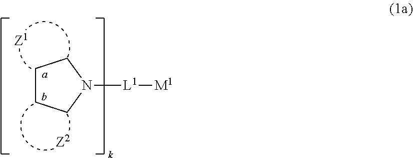

wherein

Z¹ represents a ring structure fused to the side a and represented by formula (1-1) or (1-2), and

Z² represents a ring structure fused to the side b and represented by formula (1-1) or (1-2), provided that at least one of Z¹ and Z² is represented by formula (1-1);

M¹ represents a substituted or unsubstituted nitrogen-containing aromatic heteroring having 5 to 30 ring atoms;

L¹ represents a single bond, a substituted or unsubstituted divalent aromatic hydrocarbon group having 6 to 30 ring carbon atoms, a substituted or unsubstituted divalent heterocyclic group having 5 to 30 ring atoms, a cycloalkylene group having 5 to 30 ring atoms, or a group in which the preceding groups are directly linked to each other; and

k represents 1 or 2.

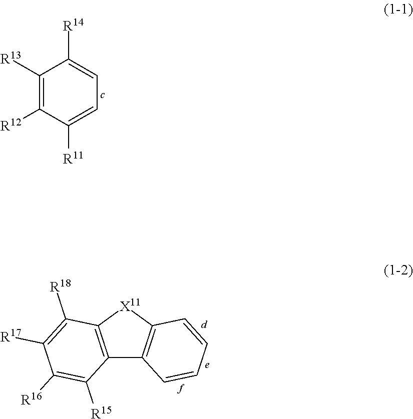

In formula (1-1), a side c is fused to the side a or b of formula (1). In formula (1-2), any one of sides d, e and f is fused to the side a or b of formula (1). In formulae (1-1) and (1-2), X¹¹ represents a sulfur atom, an oxygen atom, N—R¹⁹, or C(R²⁰)(R²¹); and each of R¹¹ to R²¹ independently represents a hydrogen atom, a heavy hydrogen atom, a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms, provided that adjacent groups of R¹¹ to R²¹ may be bonded to each other to form a ring.

The nitrogen-containing aromatic heteroring represented by M¹ of formula (Ia) includes an azine rings.

Examples of the nitrogen-containing aromatic heteroring include pyridine, pyrimidine, pyrazine, triazine, aziridine, azaindolizine, indolizine, imidazole, indole, isoindole, indazole, purine, pteridine, β-carboline, naphthyridine, quinoxaline, terpyridine, bipyridine, acridine, phenanthroline, phenazine, and imidazopyridine, with pyridine, pyrimidine, and triazine being particularly preferred. The formula (Ia) is preferably represented by formula (2):

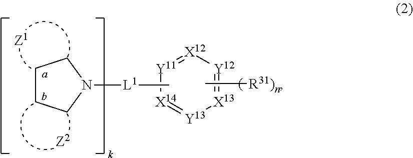

Z¹ represents a ring structure fused to the side a and represented by formula (1-1) or (1-2), and

Z² represents a ring structure fused to the side b and represented by formula (1-1) or (1-2), provided that at least one of Z¹ and Z² is represented by formula (1-1);

L¹ is as defined in formula (Ia);

each of X¹² to X¹⁴ independently represents a nitrogen atom, CH, or a carbon atom bonded to R³¹ or L¹, provided that at least one of X¹² to X¹⁴ represents a nitrogen atom;

each of Y¹¹ to Y¹³ independently represents CH or a carbon atom bonded to R³¹ or L¹;

each of R³¹ independently represents a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms;

when two or more R³¹ groups exist, the R³¹ groups may be the same or different and adjacent R³¹ groups may be bonded to each other to form a ring;

k represents 1 or 2, and n represents an integer of 0 to 4;

the side c of formula (1-1) is fused to the side a or b of formula (2); and any one of sides d, e and f of formula (1-2) is fused to the side a or b of formula (2).

Examples of the compound wherein the ring represented by formula (1-1) or (1-2) is fused to the side a or b of formula (2) are shown below:

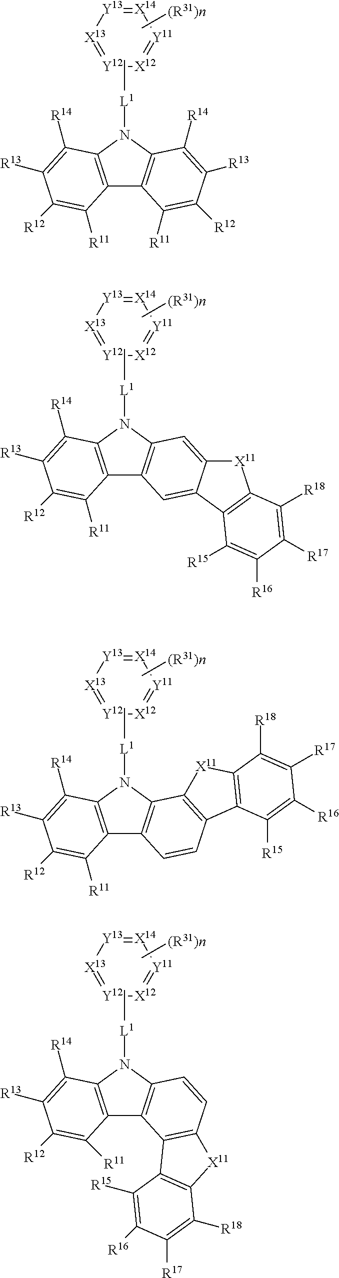

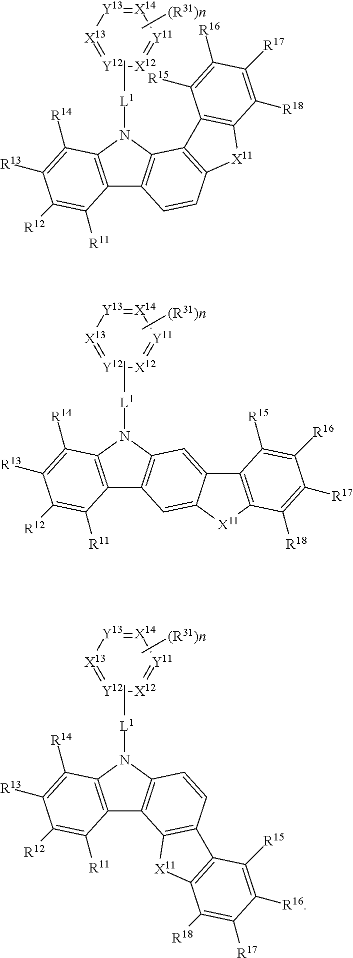

The compound represented by formula (1) or (2) is more preferably represented by formula (3) and particularly preferably represented by formula (4).

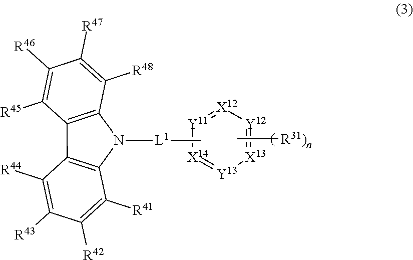

In formula (3), L¹ is as defined in formula (1);

each of X¹² to X¹⁴ independently represents a nitrogen atom, CH, or a carbon atom bonded to R³¹ or L¹, provided that at least one of X¹² to X¹⁴ represents a nitrogen atom;

each of Y¹¹ to Y¹³ independently represents CH or a carbon atom bonded to R³¹ or L¹;

each of R³¹ independently represents a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms;

when two or more R³¹ groups exist, the R³¹ groups may be the same or different and adjacent R³¹ groups may be bonded to each other to form a ring;

n represents an integer of 0 to 4;

each of R⁴¹ to R⁴⁸ independently represents a hydrogen atom, a heavy hydrogen atom, a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms; and adjacent groups of R⁴¹ to R⁴⁸ may be bonded to each other to form a ring.

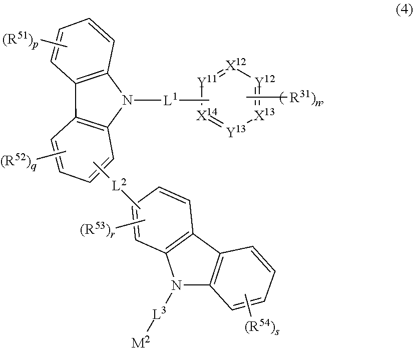

In formula (4), L¹ is as defined in formula (1);

each of X¹² to X¹⁴ independently represents a nitrogen atom, CH, or a carbon atom bonded to

R³¹ or L¹, provided that at least one of X¹² to X¹⁴ represents a nitrogen atom;

each of Y¹¹ to Y¹³ independently represents CH or a carbon atom bonded to R³¹ or L¹; each of R³¹ independently represents a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms;

adjacent R³¹ groups may be bonded to each other to form a ring;

n represents an integer of 0 to 4;

each of L² and L³ independently represents a single bond, a substituted or unsubstituted divalent aromatic hydrocarbon group having 6 to 30 ring carbon atoms, a substituted or unsubstituted divalent heterocyclic group having 5 to 30 ring atoms, a cycloalkylene group having 5 to 30 ring atoms, or a group in which the preceding groups are directly linked to each other;

each of R⁵¹ to R⁵⁴ independently represents a halogen atom, a cyano group, a substituted or unsubstituted aromatic hydrocarbon group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 30 ring carbon atoms, a substituted or unsubstituted alkoxy group having 1 to 30 carbon atoms, a substituted or unsubstituted aralkyl group having 6 to 30 ring carbon atoms, or a substituted or unsubstituted aryloxy group having 6 to 30 ring carbon atoms;

when two or more R⁵¹ groups exist, the R⁵¹ groups may be the same or different and adjacent R⁶¹ groups may be bonded to each other to form a ring;

when two or more R⁵² groups exist, the R⁵² groups may be the same or different and adjacent R⁵² groups may be bonded to each other to form a ring;

when two or more R^{s3} groups exist, the R⁵³ groups may be the same or different and adjacent R⁵³ groups may be bonded to each other to form a ring;

when two or more R⁵⁴ groups exist, the R⁵ groups may be the same or different and adjacent R⁵⁴ groups may be bonded to each other to form a ring;

M² represents a substituted or unsubstituted aromatic hydrocarbon group having 6 to 30 ring carbon atoms or a substituted or unsubstituted heterocyclic group having 5 to 30 ring atoms; and

each of p and s independently represents an integer of 0 to 4, and each of q and r independently represents an integer of 0 to 3.

In formulae (1) to (4), (1-1), and (1-2), the groups represented by R¹¹ to R²¹, R³¹, R⁴¹ to R⁴⁸, and R⁵¹ to R⁵⁴ are as defined above with respect to formula (A).

Examples of the divalent aromatic hydrocarbon group having 6 to 30 ring carbon atoms and the divalent heterocyclic group having 5 to 30 ring atoms represented by L¹ to L³ of formulae (1) to (4) includes divalent residues of the corresponding groups described above with respect to formula (A).

According to a further preferred embodiment of the present invention, host materials according to US20140048784, in particular according to paragraphs 0098 to 0154 can be used in the electronic device according to the present invention, in particular, if red light emitting materials are used. The host materials according to US20140048784 can be used as single host materials, which can is preferred, or can be used in combination with compounds (I), (II) or (III) according to the present invention as host material and co-host.

The host material according to US20140048784 is a biscarbazole derivative, having two carbazole structures in a molecule thereof.

The biscarbazole derivative has, at a specific position, a substituted or unsubstituted fluoranthenyl group, a substituted or unsubstituted triphenylenyl group, a substituted or unsubstituted benzophenanthrenyl group, a substituted or unsubstituted benzotriphenylenyl group, a substituted or unsubstituted dibenzotriphenylenyl group, a substituted or unsubstituted chrysenyl group, a substituted or unsubstituted benzochrysenyl group, a substituted or unsubstituted picenyl group, a substituted or unsubstituted benzo[b]fluoranthenyl group, a substituted or unsubstituted benzofuranyl group, a substituted or unsubstituted dibenzofuranyl group, a substituted or unsubstituted benzothiophenyl group, a substituted or unsubstituted dibenzothiophenyl group, a substituted or unsubstituted phenanthrenyl group, a substituted or unsubstituted fluorenyl group, a substituted or unsubstituted binaphthyl group, a substituted or unsubstituted benzonaphthofuranyl group, a substituted or unsubstituted benzonaphthothiophenyl group, a substituted or unsubstituted dibenzophenanthrenyl group, a substituted or unsubstituted naphthotriphenylenyl group, a substituted or unsubstituted benzofluoranthenyl group, a substituted or unsubstituted benzofluorenyl group, or a substituted or unsubstituted phenyl group. Examples thereof include compounds represented by any of formulae (1) to (4), (1′), (1a), and (10).

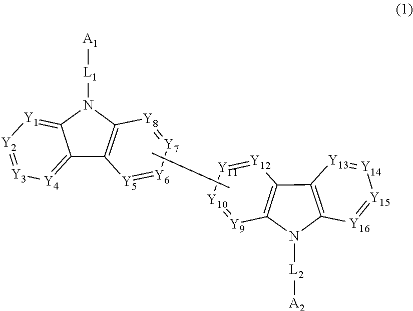

wherein;

each of A₁ and A₂ independently represents a substituted or unsubstituted aromatic hydrocarbon group having 6 to 30 ring carbon atoms;

each of Y₁ to Y₁₆ independently represents C(R) or a nitrogen atom, and each of R groups independently represents a hydrogen atom, a substituent, or a valence bonded to a carbazole skeleton; and

each of L₁ and L₂ independently represents a single bond, a substituted or unsubstituted, divalent aromatic hydrocarbon group having 6 to 30 ring carbon atoms, or a substituted or unsubstituted, divalent aromatic heterocyclic group having 2 to 30 ring carbon atoms, provided that;

at least one of A₁, A₂ and R represents a substituted or unsubstituted fluoranthenyl group, a substituted or unsubstituted triphenylenyl group, a substituted or unsubstituted benzophenanthrenyl group, a substituted or unsubstituted benzotriphenylenyl group, a substituted or unsubstituted dibenzotriphenylenyl group, a substituted or unsubstituted chrysenyl group, a substituted or unsubstituted benzochrysenyl group, a substituted or unsubstituted picenyl group, a substituted or unsubstituted benzo[b]fluoranthenyl group, a substituted or unsubstituted phenanthrenyl group, a substituted or unsubstituted binaphthyl group, a substituted or unsubstituted dibenzophenanthrenyl group, a substituted or unsubstituted naphthotriphenylenyl group, a substituted or unsubstituted benzofluorenyl group, or a naphthyl group;

when Y₁ to Y₁₆ all represent C(R) wherein R is a hydrogen atom, Y₆ and Y₁ are bonded to each other via a single bond, each of L₁ and L₂ represents a single bond, and A₁ represents a phenanthrenyl group, A₂ represents a phenyl group, a biphenylyl group, or a naphthyl group; and

when Y₁ to Y₁₆ all represent C(R) wherein R is a hydrogen atom, Y₆ and Y₁₁ are bonded to each other via a single bond, each of L₁ and L₂ represents a single bond, and A₁ represents a naphthyl group, A₁ and A₂ are different from each other.

In formulae (1) and (1′), at least one of Y₁ to Y₄ represents C(R), at least one of Y₅ to Y₈ represents C(R), at least one of Y₉ to Y₁₂ represents C(R), and at least one of Y₁₃ to Y₁₆ represent C(R).

In addition, at least one of Y₅ to Y₈ represents C(R) and at least one of Y₉ to Y₁₂ represents C(R), wherein two R groups represent valences which are bonded to each other.

The R groups in formulae (1) and (1′) may be the same or different.

In formula (Ia), at least one of Y₁ₐ to Y₄ₐ represents C(R), at least one of Y₅ₐ to Y₈ₐ represents C(R), at least one of Y₉ₐ to Y₁₂ₐ represents C(R), and at least one of Y₁₃ₐ to Y₁₆ₐ represents C(R).

In addition, at least one of Y₅ₐ to Y₈ₐ represents C(R) and at least one of Y₉ₐ to Y₁₂ₐ represents C(R), wherein two R groups represent valences which are bonded to each other.

The R groups in formula (Ia) may be the same or different.

In formula (10), at least one of Y_{1′} to Y_{4′} represents C(R′), at least one of Y_{5′} to Y_{8′} represents C(R′), at least one of Y_{9′} to Y_{12′} represents C(R′), and at least one of Y_{13′} to Y_{16′} represents C(R′).

In addition, at least one of Y_{5′} to Y_{8′} represents C(R′) and at least one of Y_{9′} to Y_{12′} represents C(R′), wherein two R′ groups represent valences which are bonded to each other.

The R′ groups in formula (10) may be the same or different.

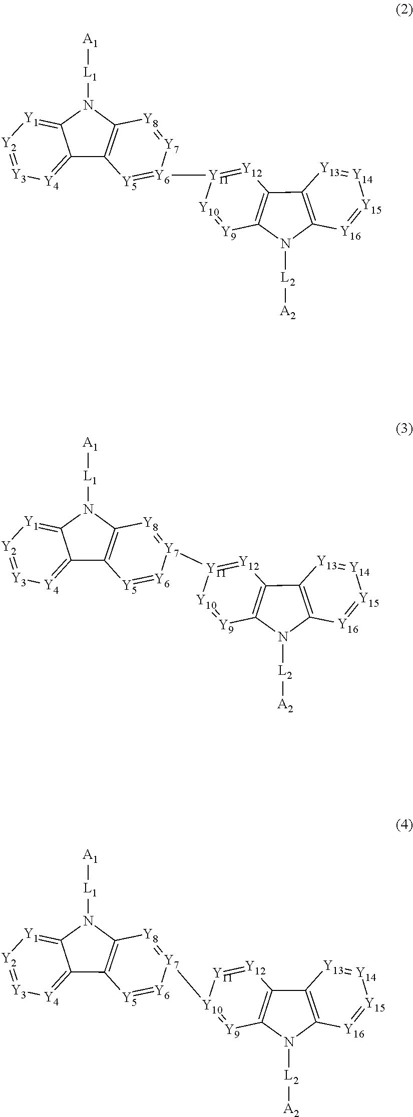

wherein each of A₁, A₂, Y₁ to Y₁₆, L₁, and L₂ in formulae (2) to (4) is as defined in formula (1).

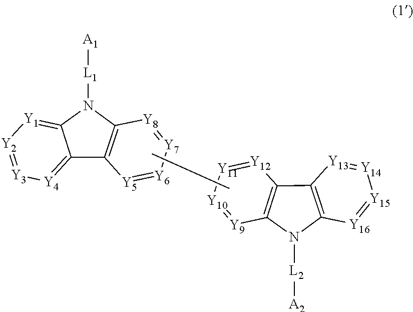

wherein:

each of A₁ and A₂ independently represents a substituted or unsubstituted aromatic hydrocarbon group having 6 to 30 ring carbon atoms or a substituted or unsubstituted aromatic heterocyclic group having 2 to 30 ring carbon atoms;

each of Y₁ to Y₁₆ independently represents C(R) or a nitrogen atom, and each of R groups independently represents a hydrogen atom, a substituent, or a valence bonded to a carbazole skeleton; and

each of L₁ and L₂ independently represents a single bond, a substituted or unsubstituted, divalent aromatic hydrocarbon group having 6 to 30 ring carbon atoms, or a substituted or unsubstituted, divalent aromatic heterocyclic group having 2 to 30 ring carbon atoms, provided that:

at least one of A₁, A₂ and R represents a substituted or unsubstituted fluoranthenyl group, a substituted or unsubstituted triphenylenyl group, a substituted or unsubstituted benzophenanthrenyl group, a substituted or unsubstituted benzotriphenylenyl group, a substituted or unsubstituted dibenzotriphenylenyl group, a substituted or unsubstituted chrysenyl group, a substituted or unsubstituted benzochrysenyl group, a substituted or unsubstituted picenyl group, a substituted or unsubstituted benzo[b]fluoranthenyl group, a substituted or unsubstituted benzofuranyl group, a substituted or unsubstituted dibenzofuranyl group, a substituted or unsubstituted benzothiophenyl group, a substituted or unsubstituted dibenzothiophenyl group, a substituted or unsubstituted phenanthrenyl group, a substituted or unsubstituted fluorenyl group, a substituted or unsubstituted binaphthyl group, a substituted or unsubstituted benzonaphthofuranyl group, a substituted or unsubstituted benzonaphthothiophenyl group, a substituted or unsubstituted dibenzophenanthrenyl group, a substituted or unsubstituted naphthotriphenylenyl group, a substituted or unsubstituted benzofluorenyl group, or a substituted or unsubstituted phenyl group;

when Y₁ to Y₁₆ all represent C(R) wherein R is a hydrogen atom, Y₆ and Y₁₁ are bonded to each other via a single bond, each of L₁ and L₂ represents a single bond, and A₁ represents a phenanthrenyl group, A₂ does not represent a phenanthrenyl group;

when Y₁ to Y₁₆ all represent C(R), Y₆ and Y₁₁ are bonded to each other via a single bond, and each of L₁ and L₂ represents a single bond, each of R groups does not represent a fluorenyl group; and

when A₁ represents a fluorenyl group, A₂ does not represent a phenyl group, a naphthyl group, or a fluorenyl group.

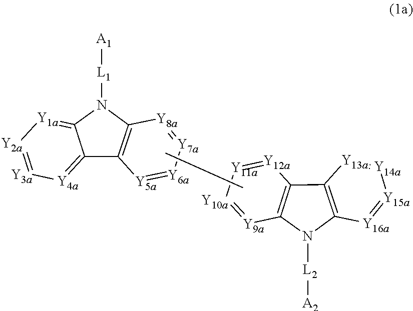

wherein:

one of A₁ₐ and A₂ₐ represents a group represented by formula (a) and the other represents a substituted or unsubstituted fluoranthenyl group, a substituted or unsubstituted benzophenanthrenyl group, a substituted or unsubstituted picenyl group, a substituted or unsubstituted benzo[b]fluoranthenyl group, a substituted or unsubstituted phenanthrenyl group, a substituted or unsubstituted binaphthyl group, a substituted or unsubstituted dibenzophenanthrenyl group, a substituted or unsubstituted naphthotriphenylenyl group, or a substituted or unsubstituted benzofluorenyl group;

each of Y₁ₐ to Y₁₆ₐ independently represents C(R) or a nitrogen atom, and each of R groups independently represents a hydrogen atom, a substituent, or a valence bonded to a carbazole skeleton;

each of L₁ₐ and L₂ₐ independently represents a single bond, a substituted or unsubstituted, divalent aromatic hydrocarbon group having 6 to 30 ring carbon atoms, or a substituted or unsubstituted, divalent aromatic heterocyclic group having 2 to 30 ring carbon atoms:

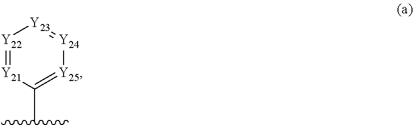

wherein each of Y₂₁ and Y₂₅ independently represents C(Ra) or a nitrogen atom, and each of Ra groups independently represents a hydrogen atom or a substituent.

The details of A₁ₐ, A₂ₐ, Y₁ₐ to Y₁₆ₐ, L₁ₐ, L₂ₐ, and Ra in formulae (1a) and (a) are the same as those of A₁, A₂, Y₁ to Y₁₆, L₁, L₂, and R in formula (1).

When one of A₁ₐ and A₂ₐ represents a group represented by formula (a) and the other represents a group including a large molecular weight fused ring, such as a triphenylenyl group and a chrysenyl group, the compound represented by formula (Ia) has an excessively large molecular weight, increasing the vapor deposition temperature and therefore likely to increase the amount of thermally decomposed components. Therefore, when one of A₁ₐ and A₂ₐ represents a group represented by formula (a), the other preferably represents a substituted or unsubstituted fluoranthenyl group or a substituted or unsubstituted phenanthrenyl group.

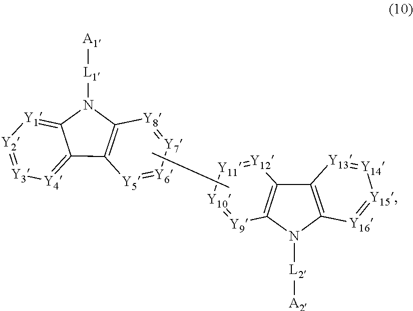

wherein:

one of A₁, and A_{2′} represents a substituted or unsubstituted naphthyl group or a substituted or unsubstituted fluorenyl group and the other represents a substituted or unsubstituted aromatic hydrocarbon group having 6 to 30 ring carbon atoms;

each of Y_{1′} to Y_{16′} independently represents C(R′) or a nitrogen atom, and each of R′ groups independently represents a hydrogen atom, a substituent, or a valence bonded to a carbazole skeleton; and

each of L_{1′} and L_{2′} independently represents a single bond, a substituted or unsubstituted, divalent aromatic hydrocarbon group having 6 to 30 ring carbon atoms, or a substituted or unsubstituted, divalent aromatic heterocyclic group having 2 to 30 ring carbon atoms.

The details of A_{1′}, A_{2′}, L_{1′}, L₂, Y_{1′} to Y_{16′}, and R′ in formula (10) are the same as those of A₁, A₂, L₁, L₂, Y₁ to Y₁₆, and R in formula (1).

In formulae (1) to (4) and (1′), at least one of A₁, A₂ and R preferably represents a substituted or unsubstituted fluoranthenyl group, a substituted or unsubstituted triphenylenyl group, a substituted or unsubstituted benzophenanthrenyl group, a substituted or unsubstituted benzotriphenylenyl group, a substituted or unsubstituted dibenzotriphenylenyl group, a substituted or unsubstituted chrysenyl group, a substituted or unsubstituted benzochrysenyl group, a substituted or unsubstituted picenyl group, a substituted or unsubstituted benzo[b]fluoranthenyl group, a substituted or unsubstituted benzofuranyl group, a substituted or unsubstituted dibenzofuranyl group, a substituted or unsubstituted benzothiophenyl group, a substituted or unsubstituted dibenzothiophenyl group, a substituted or unsubstituted phenanthrenyl group, a substituted or unsubstituted fluorenyl group, or a substituted or unsubstituted binaphthyl group, because these groups are moderately bulky. More preferably, at least one of A₁ and A₂ represents a substituted or unsubstituted fluoranthenyl group, a substituted or unsubstituted triphenylenyl group, a substituted or unsubstituted benzophenanthrenyl group, a substituted or unsubstituted benzotriphenylenyl group, a substituted or unsubstituted dibenzotriphenylenyl group, a substituted or unsubstituted chrysenyl group, a substituted or unsubstituted benzochrysenyl group, a substituted or unsubstituted picenyl group, a substituted or unsubstituted benzo[b]fluoranthenyl group, a substituted or unsubstituted benzofuranyl group, a substituted or unsubstituted dibenzofuranyl group, a substituted or unsubstituted benzothiophenyl group, a substituted or unsubstituted dibenzothiophenyl group, or a substituted or unsubstituted binaphthyl group.

Also preferably, each of A₁ and A₂ in formulae (1) to (4) and (1′) independently represents a substituted or unsubstituted fluoranthenyl group, a substituted or unsubstituted triphenylenyl group, a substituted or unsubstituted benzotriphenylenyl group, a substituted or unsubstituted benzophenanthrenyl group, a substituted or unsubstituted dibenzofuranyl group, or a substituted or unsubstituted dibenzothiophenyl group.

In addition, -L₁-A₁ and -L₂-A₂ in formulae (1) to (4) and (1′) are preferably different from each other.

The substituted or unsubstituted phenyl group for any of A₁, A₂ and R is preferably a phenyl group substituted by an aromatic hydrocarbon group having 10 to 30 ring carbon atoms and particularly preferably a naphthylphenyl group.

When at least one of A₁ and A₂ in formulae (1) to (4) and (1′) represents a group represented by formula (a), the biscarbazole derivative is particularly preferred as a host material to be used in combination with a green emitting dopant.

In formula (a), Y₂₁ and/or Y₂₅ preferably represents a nitrogen atom, and each of Y₂₂ and Y₂₄ more preferably represents C(Ra).

Specific examples of the substituent which A₁ and A₂ in formulae (1) to (4) and (1′) may have and the substituents represented by R and Ra include a fluorine atom; a cyano group; a substituted or unsubstituted, linear, branched, or cyclic alkyl group having 1 to 20 carbon atoms; a linear, branched, or cyclic alkylene group having 1 to 20 carbon atoms; a linear, branched, or cyclic, divalent, unsaturated hydrocarbon group having 1 to 20 carbon atoms; a substituted or unsubstituted, linear, branched, or cyclic alkoxy group having 1 to 20 carbon atoms; a substituted or unsubstituted, linear, branched, or cyclic haloalkyl group having 1 to 20 carbon atoms; a substituted or unsubstituted, linear, branched, or cyclic haloalkoxy group having 1 to 20 carbon atoms; a substituted or unsubstituted, linear, branched, or cyclic alkylsilyl group having 1 to 10 carbon atoms; a substituted or unsubstituted arylsilyl group having 6 to 30 carbon atoms; a substituted or unsubstituted aromatic hydrocarbon group having 6 to 30 ring carbon atoms; and a substituted or unsubstituted aromatic heterocyclic group having 2 to 30 ring carbon atoms. In addition, a plurality of substituents of any such kind may exist, and when the plurality of substituents exist, the substituents may be the same or different from each other.

The R groups on adjacent ring carbon atoms may be bonded to each other to form a ring structure together with the ring carbon atoms.

Examples of the linear, branched, or cyclic alkyl group having 1 to 20 carbon atoms include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a n-nonyl group, a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a neopentyl group, a 1-methylpentyl group, a 2-methylpentyl group, a 1-pentylhexyl group, a 1-butylpentyl group, a 1-heptyloctyl group, a 3-methylpentyl group, a cyclopentyl group, a cyclohexyl group, a cyclooctyl group, a 3,5-tetramethylcyclohexyl group, a trifluoromethyl group, a 2,2,2-trifluoroethyl group, and a 1,1,1,3,3,3-hexafluoro-2-propyl group.

Examples of the linear, branched, or cyclic alkylene group having 1 to 20 carbon atoms include an ethylene group, a propylene group, and a butylene group.

Examples of the linear, branched, or cyclic, divalent unsaturated hydrocarbon group having 1 to 20 carbon atoms include a 1,3-butadiene-1,4-diyl group.

Examples of the linear, branched, or cyclic alkylsilyl group having 1 to 10 carbon atoms include a trimethylsilyl group, a triethylsilyl group, a tributylsilyl group, a dimethylethylsilyl group, a dimethylisopropylsilyl group, a dimethylpropylsilyl group, a dimethylbutylsilyl group, a dimethyl-t-butylsilyl group, and a diethylisopropylsilyl group.

Examples of the arylsilyl group having 6 to 30 carbon atoms include a phenyldimethylsilyl group, a diphenylmethylsilyl group, a diphenyl-t-butylsilyl group, and a triphenylsilyl group.

Examples of the halogen atom include a fluorine atom.

Examples of the aromatic heterocyclic group having 2 to 30 ring carbon atoms include non-fused aromatic heterocyclic and fused aromatic heterocyclic groups, more specifically, a pyrrolyl group, a pyrazinyl group, a pyridinyl group, an indolyl group, an isoindolyl group, a furyl group, a benzofuranyl group, an isobenzofuranyl group, a dibenzofuranyl group, a dibenzothiophenyl group, a quinolyl group, an isoquinolyl group, a quinoxalinyl group, a carbazolyl group, a phenanthridinyl group, an acridinyl group, a phenanthrolinyl group, a thienyl group, and residues of a pyridine ring, a pyrazine ring, a pyrimidine ring, a pyridazine ring, a triazine ring, an indole ring, a quinoline ring, an acridine ring, a pyrrolidine ring, a dioxane ring, a piperidine ring, a morpholine ring, a piperazine ring, a carbazole ring, a furan ring, a thiophene ring, an oxazole ring, an oxadiazole ring, a benzoxazole ring, a thiazole ring, a thiadiazole ring, a benzothiazole ring, a triazole ring, an imidazole ring, a benzimidazole ring, a pyran ring, a dibenzofuran ring, and a benzo[c]dibenzofuran ring.

Examples of the aromatic hydrocarbon group having 6 to 30 ring carbon atoms include non-fused aromatic hydrocarbon groups and fused aromatic hydrocarbon groups, more specifically, a phenyl group, a naphthyl group, a phenanthryl group, a biphenyl group, a terphenyl group, a quaterphenyl group, a fluoranthenyl group, a triphenylenyl group, a phenanthrenyl group, a 9,9-dimethylfluorenyl group, a benzo[c]phenanthrenyl group, a benzo[a]triphenylenyl group, a naphtho[1,2-c]phenanthrenyl group, a naphtho[1,2-a]triphenylenyl group, a dibenzo[a,c]triphenylenyl group, and a benzo[b]fluoranthenyl group.

Examples of the divalent linking group represented by L1 and L2 in formulae (1) to (4) and (1′) include a substituted or unsubstituted, divalent aromatic hydrocarbon group having 6 to 30 ring carbon atoms and a substituted or unsubstituted, divalent aromatic heterocyclic group having 2 to 30 ring carbon atoms.

Examples of the divalent aromatic hydrocarbon group having 6 to 30 ring carbon atoms include groups obtained by making the examples of the aromatic hydrocarbon group having 6 to 30 ring carbon atoms mentioned above into divalent groups.

In addition, specific examples of the divalent aromatic heterocyclic group having 2 to 30 ring carbon atoms include groups obtained by making the examples of the aromatic heterocyclic group having 2 to 30 ring carbon atoms mentioned above into divalent groups.

In each of formulae (1) to (4) and (1′), Y₁ to Y₁₆ all preferably represent C(R).

In each of formulae (1) to (4) and (1′), the number of substituents represented by R in Y₁ to Y₈ or in Y₉ to Y₁₆ is preferably 0 to 2, more preferably 0 or 1.

Specific examples of the biscarbazole derivative represented by any one of formulae (1) to (4), (1′), and (10) include the following compounds. In the following structural formulae, D represents a heavy hydrogen (deuterium).

According to the present invention, the compounds according to general formulae (I), (II) or (III) are preferably be used as host material in the light emitting layer of the electronic device, preferably in a OLED, according to the present invention. The compounds according to general formulae (I), (II) or (III) can be used (a) as single host materials or can be used (b) in combination with any compounds suitable as host materials as mentioned above. Embodiment (a) is preferred, if a red light emitting material is present in the light emitting layer. Embodiment (b) is preferred, if a green light emitting material is present in the light emitting layer.

Preferred host materials, which may be used, if blue dopants are present in the light emitting layer, are mentioned in US 2012/112169. Preferably, the anthracene derivative represented by the formula (5) is used as host material for blue dopants:

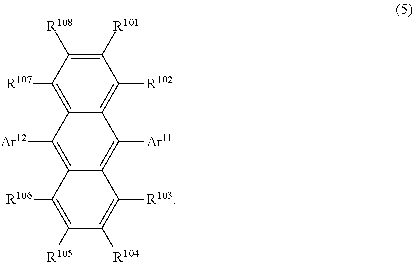

In the formula (5), Ar¹¹ and Ar¹² are independently a substituted or unsubstituted monocyclic group having 5 to 50 ring atoms, a substituted or unsubstituted fused ring group having 8 to 50 ring atoms, or a group formed by combination of a monocyclic group and a fused ring group and R¹⁰¹ to R¹⁰⁸ are independently a group selected from a hydrogen atom, a substituted or unsubstituted monocyclic group having 5 to 50 ring atoms, a substituted or unsubstituted fused ring group having 8 to 50 ring atoms, a group formed by combination of a monocyclic group and a fused ring group, a substituted or unsubstituted alkyl group having 1 to 50 carbon atoms, a substituted or unsubstituted cycloalkyl group having 3 to 50 ring carbon atoms, a substituted or unsubstituted alkoxy group having 1 to 50 carbon atoms, a substituted or unsubstituted aralkyl group having 7 to 50 carbon atoms, a substituted or unsubstituted aryloxy group having 6 to 50 ring carbon atoms, a substituted or unsubstituted silyl group, a halogen atom and a cyano group.

The monocyclic group in the formula (5) means a group which is composed only of ring structures having no fused structure.

As specific examples of the monocyclic group having 5 to 50 (preferably 5 to 30, more preferably 5 to 20) ring atoms, aromatic groups such as a phenyl group, biphenyl group, terphenyl group and quaterphenyl group, and heterocyclic groups such as a pyridyl group, pyrazyl group, pyrimidyl group, triazinyl group, furyl group and thienyl group, can be given preferably.

Among these, a phenyl group, biphenyl group or terphenyl group is preferable.

The fused ring group in the formula (5) means a group formed by fusion of 2 or more ring structures.

As specific examples of the fused ring group having 8 to 50 (preferably 8 to 30, more preferably 8 to 20) ring atoms, fused aromatic ring groups such as a naphthyl group, phenanthryl group, anthryl group, chrysenyl group, benzanthryl group, benzophenanthryl group, triphenylenyl group, benzochrysenyl group, indenyl group, fluorenyl group, 9,9-dimethylfluorenyl group, benzofluorenyl group, dibenzofluorenyl group, fluoranthenyl group and benzofluoranthenyl group, and fused heterocyclic groups such as a benzofuranyl group, benzothiophenyl group, indolyl group, dibenzofuranyl group, dibenzothiophenyl group, carbazolyl group, quinolyl group and phenanthrolinyl group, can be given preferably.

Among these, a naphthyl group, phenanthryl group, anthryl group, 9,9-dimethylfluorenyl group, fluoranthenyl group, benzanthryl group, dibenzothiophenyl group, dibenzofuranyl group or carbazolyl group is preferable.

As preferable substituents of “substituted or unsubstituted . . . ” in Ar¹¹, Ar¹², and R¹⁰¹ to R¹⁰⁸, a monocyclic group, fused ring group, alkyl group, cycloalkyl group, silyl group, alkoxy group, cyano group and halogen atom (in particular, fluorine) can be given. A monocyclic group and fused ring group are particularly preferable.

It is preferred that the anthracene derivative represented by the formula (5) be any of the following anthracene derivatives (A), (B) and (C), which is selected depending on the constitution or demanded properties of an organic EL device to which it is applied.

(Anthracene Derivative (A))

This anthracene derivative is derivatives of the formula (5) wherein Ar¹¹ and Ar¹² are independently a substituted or unsubstituted fused ring group having 8 to 50 ring atoms. This anthracene derivative can be classified into the case that Ar¹¹ and Ar¹² are the same substituted or unsubstituted fused ring group and the case that Ar¹¹ and Ar¹² are different substituted or unsubstituted fused ring groups.

Particularly preferred is the anthracene derivative of the formula (5) wherein Ar¹¹ and Ar¹² are different (including difference in substituted positions) substituted or unsubstituted fused ring groups. Preferable specific examples of the fused ring are the same as those described above. Among those, a naphthyl group, phenanthryl group, benzanthryl group, 9,9-dimethylfluorenyl group and dibenzofuranyl group are preferable.

(Anthracene Derivative (B))

This anthracene derivative is derivatives of the formula (5) wherein one of Ar¹¹ and Ar¹² is a substituted or unsubstituted monocyclic group having 5 to 50 ring atoms, and the other is a substituted or unsubstituted fused ring group having 8 to 50 ring atoms.

As a preferred embodiment, Ar¹² is a naphthyl group, phenanthryl group, benzanthryl group, 9,9-dimethylfluorenyl group or dibenzofuranyl group, and Ar¹¹ is a phenyl group substituted by a monocyclic group or fused ring group.

As another preferred embodiment, Ar¹² is a fused ring group, and A¹¹ is an unsubstituted phenyl group. In this case, as the fused ring group, a phenanthryl group, 9,9-dimethylfluorenyl group, dibenzofuranyl group and benzoanthryl group are particularly preferable.

(Anthracene Derivative (C))

This anthracene derivative is derivatives of formula (5) wherein Ar¹¹ and Ar¹² are independently a substituted or unsubstituted monocyclic group having 5 to 50 ring atoms.

As a preferred embodiment, both Ar¹¹ and Ar¹² are a substituted or unsubstituted phenyl group.

As a further preferred embodiment, Ar¹¹ is an unsubstituted phenyl group, and Ar¹² is a phenyl group having a monocyclic group or a fused ring group as a substituent, and Ar¹¹ and Ar¹² are independently a phenyl group having a monocyclic group or a fused ring group as a substituent.

The preferable specific examples of the monocyclic group and fused ring group as a substituent are the same as those described above. As the monocyclic group as a substituent, a phenyl group and biphenyl group are further preferable. As the fused ring group as a substituent, a naphthyl group, phenanthryl group, 9,9-dimethylfluorenyl group, dibenzofuranyl group and benzanthryl group are further preferable.

Hole/Exciton Blocking Layer (f):

Blocking layers may be used to reduce the number of charge carriers (electrons or holes) and/or excitons that leave the emissive layer. The hole blocking layer may be disposed between the emitting layer (e) and electron transport layer (g), to block holes from leaving layer (e) in the direction of electron transport layer (g). Blocking layers may also be used to block excitons from diffusing out of the emissive layer.

Additional hole blocker materials typically used in OLEDs are 2,6-bis(N-carbazolyl)pyridine (mCPy), 2,9-dimethyl-4,7-diphenyl-1,10-phenanthroline (bathocuproin, (BCP)), bis(2-methyl-8-quinolinato)-4-phenylphenylato)aluminum(III) (BAlq), phenothiazine S,S-dioxide derivates and 1,3,5-tris(N-phenyl-2-benzylimidazolyl)benzene) (TPBI), TPBI also being suitable as electron-transport material. Further suitable hole blockers and/or electron conductor materials are 2,2′,2″-(1,3,5-benzenetriyl)tris(1-phenyl-1-H-benzimidazole), 2-(4-biphenylyl)-5-(4-tert-butylphenyl)-1,3,4-oxadiazole, 8-hydroxyquinolinolatolithium, 4-(naphthalen-1-yl)-3,5-diphenyl-4H-1,2,4-triazole, 1,3-bis[2-(2,2′-bipyridin-6-yl)-1,3,4-oxadiazo-5-yl]benzene, 4,7-diphenyl-1,10-phenanthroline, 3-(4-biphenylyl)-4-phenyl-5-tert-butylphenyl-1,2,4-triazole, 6,6′-bis[5-(biphenyl-4-yl)-1,3,4-oxadiazo-2-yl]-2,2′-bipyridyl, 2-phenyl-9,10-di(naphthalene-2-yl)anthracene, 2,7-bis[2-(2,2′-bipyridin-6-yl)-1,3,4-oxadiazo-5-yl]-9,9-dimethylfluorene, 1,3-bis[2-(4-tert-butylphenyl)-1,3,4-oxadiazo-5-yl]benzene, 2-(naphthalene-2-yl)-4,7-diphenyl-1,10-phenanthroline, tris(2,4,6-trimethyl-3-(pyridin-3-yl)phenyl)borane, 2,9-bis(naphthalene-2-yl)-4,7-diphenyl-1,10-phenanthroline, 1-methyl-2-(4-(naphthalene-2-yl)phenyl)-1H-imidazo[4,5-f][1,10]phenanthroline. In a further embodiment, it is possible to use compounds which comprise aromatic or heteroaromatic rings joined via groups comprising carbonyl groups, as disclosed in WO2006/100298, disilyl compounds selected from the group consisting of disilylcarbazoles, disilylbenzofurans, disilylbenzothiophenes, disilylbenzophospholes, disilylbenzothiophene S-oxides and disilylbenzothiophene S,S-dioxides, as specified, for example, in PCT applications WO2009/003919 and WO2009003898 and disilyl compounds as disclosed in WO2008/034758, as a blocking layer for holes/excitons (f).

In another preferred embodiment compounds (SH-1), (SH-2), (SH-3), SH-4, SH-5, SH-6, (SH-7), (SH-8), (SH-9), (SH-10) and (SH-11) may be used as hole/exciton blocking materials.

Electron Transport Layer (g):

Electron transport layer may include a material capable of transporting electrons. Electron transport layer may be intrinsic (undoped), or doped. Doping may be used to enhance conductivity.

The compound of the formulae (I), (II) or (III) according to the present invention is suitable as electron transport material, either alone or in combination with one or more of the electron transport materials mentioned below. The compound of the formulae (I), (II) or (III) according to the present invention is preferably suitable as electron transport material, if a blue fluorescent emitter is present in the emitting layer.

Further suitable electron-transporting materials for layer (g) of the inventive OLEDs, which may be used in combination with the compound of formulae (I), (II) or (III) according to the present invention or in absence of the compound of formulae (I), (II) or (III) according to the present invention as electron transport material, comprise metals chelated with oxinoid compounds, such as tris(8-hydroxyquinolato)aluminum (Alq₃), compounds based on phenanthroline such as 2,9-dimethyl-4,7-diphenyl-1,10-phenanthroline (DDPA=BCP), 4,7-diphenyl-1,10-phenanthroline (Bphen), 2,4,7,9-tetraphenyl-1,10-phenanthroline, 4,7-diphenyl-1,10-phenanthroline (DPA) or phenanthroline derivatives disclosed in EP1786050, in EP1970371, or in EP1097981, and azole compounds such as 2-(4-biphenylyl)-5-(4-t-butylphenyl)-1,3,4-oxadiazole (PBD) and 3-(4-biphenylyl)-4phenyl-5-(4-t-butylphenyl)-1,2,4-triazole (TAZ).

Further suitable electron transport materials, which may be used in combination with the compound of formulae (I), (II) or (III) according to the present invention or in absence of the compound of formulae (I), (II) or (III) according to the present invention as electron transport material, are mentioned in Abhishek P. Kulkarni, Christopher J. Tonzola, Amit Babel, and Samson A. Jenekhe, Chem. Mater. 2004, 16, 4556-4573; G. Hughes, M. R. Bryce, J. Mater. Chem. 2005, 15, 94-107 and Yasuhiko Shirota and Hiroshi Kageyama, Chem. Rev. 2007, 107, 953-1010 (ETM, HTM).

It is likewise possible to use mixtures of at least two materials in the electron-transporting layer, in which case at least one material is electron-conducting. Preferably, in such mixed electron-transport layers, at least one phenanthroline compound is used, preferably BCP, or at least one pyridine compound according to the formula (XVI) below, preferably a compound of the formula (XVIa) below. More preferably, in mixed electron-transport layers, in addition to at least one phenanthroline compound, alkaline earth metal or alkali metal hydroxyquinolate complexes, for example Liq, are used. Suitable alkaline earth metal or alkali metal hydroxyquinolate complexes are specified below (formula XVII). Reference is made to WO2011/157779.

The electron-transport layer may also be electronically doped in order to improve the transport properties of the materials used, in order firstly to make the layer thicknesses more generous (avoidance of pinholes/short circuits) and in order secondly to minimize the operating voltage of the device. Electronic doping is known to those skilled in the art and is disclosed, for example, in W. Gao, A. Kahn, J. Appl. Phys., Vol. 94, No. 1, 1 Jul. 2003 (p-doped organic layers); A. G. Werner, F. Li, K. Harada, M. Pfeiffer, T. Fritz, K. Leo, Appl. Phys. Lett., Vol. 82, No. 25, 23 Jun. 2003 and Pfeiffer et al., Organic Electronics 2003, 4, 89-103 and K. Walzer, B. Maennig, M. Pfeiffer, K. Leo, Chem. Soc. Rev. 2007, 107, 1233. For example, it is possible to use mixtures which lead to electrical n-doping of the electron-transport layer. n-Doping is achieved by the addition of reducing materials. These mixtures may, for example, be mixtures of the abovementioned electron transport materials with alkali/alkaline earth metals or alkali/alkaline earth metal salts, for example Li, Cs, Ca, Sr, Cs₂CO₃, with alkali metal complexes, for example 8-hydroxyquinolatolithium (Liq), and with Y, Ce, Sm, Gd, Tb, Er, Tm, Yb, Li₃N, Rb₂CO₃, dipotassium phthalate, W(hpp)₄ from EP1786050, or with compounds described in EP1837926B1, EP1837927, EP2246862 and WO2010132236.

In a preferred embodiment, the electron-transport layer comprises a substance highly electron injecting and/or highly electron transporting, preferably in combination with the compound of formulae (I), (II) or (III). The substance is, for example, an alkali metal, an alkali metal-comprising compound, an alkaline earth metal, an alkaline earth metal-comprising compound, a rare earth metal, and a rare earth metal-comprising compound.

The alkali metal may be Li, Na, K, Rb, and Cs. The alkali metal-comprising compound may be a halide such as a fluoride, a chloride, a bromide, and a iodide, an oxide, and a complex such as 8-quinolinolatolithium (Liq).

The alkaline earth metal may be Be, Mg, Ca, Sr, and Ba. The alkaline earth metal-comprising compound may be a halide such as a fluoride, a chloride, a bromide, and a iodide, an oxide, and a complex such as bis(10-hydroxybenzo[h]quinolinato)beryllium (BeBq₂).

The rare earth metal may be Sc, Y, Ce, Eu, Tb, Er, and Lu. The rare earth metal-comprising compound may be a halide such as a fluoride, a chloride, a bromide, and a iodide, an oxide, and a complex.

In a preferred embodiment, the electron-transport layer comprises at least one compound of the general formula (XVII):

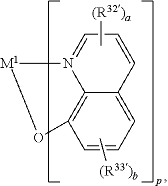

in which

R^{32′} and R^{33′} are each independently F, C₁-C₈-alkyl, or C₆-C₁₄-aryl, which is optionally substituted by one or more C₁—C-alkyl groups, or

two R^{32′} and/or R^{33′} substituents together form a fused benzene ring which is optionally substituted by one or more C₁-C₈-alkyl groups;

a and b are each independently 0, or 1, 2 or 3,

M¹ is an alkaline metal atom or alkaline earth metal atom,

p is 1 when M¹ is an alkali metal atom, p is 2 when M¹ is an earth alkali metal atom.

A very particularly preferred compound of the formula (XVII) is

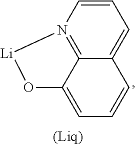

which may be present as a single species, or in other forms such as Li_{g}Q_{g} in which g is an integer, for example Li₆Q₆. Q is an 8-hydroxyquinolate ligand or an 8-hydroxyquinolate derivative.

In a further preferred embodiment, the electron-transport layer comprises at least one compound of the formula (XVI):

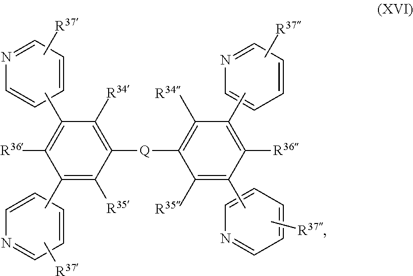

R^{34″}, R³⁵″, R³⁶″, R³⁷″, R^{34′}, R^{35′}, R^{36′} and R^{37′} are each independently H, C₁-C₁₈-alkyl, C₁-C₁₈-alkyl which is substituted by E′ and/or interrupted by D′, C₆-C₂₄-aryl, C₆-C₂₄-aryl which is substituted by G′, C₂-C₂₀-heteroaryl or C₂-C₂₀-heteroaryl which is substituted by G′;

Q is an arylene or heteroarylene group, each of which is optionally substituted by G′;

D′ is —CO—; —COO—; —S—; —SO—; —SO₂—; —O—; —NR^{40′}—; —SiR^{45′}R^{46′}—; —POR^{47′}—; —CR^{38′}═CR^{39′}—; or —C≡C—;

E′ is —OR^{44′}; —SR^{44′}; —NR^{40′}R^{41′}; —COR^{43′}; —COOR^{42′}; —CONR^{40′}R^{41′}; —CN; or F;

G′ is E′, C₁-C₁₈-alkyl, C₁-C₁₈-alkyl which is interrupted by D′, C₁-C₁₈-perfluoroalkyl, C₁-C₁₈-alkoxy, or C₁-C₁₈-alkoxy which is substituted by E′ and/or interrupted by D′, in which

R^{38′} and R^{39′} are each independently H, C₆-C₁₈-aryl; C₆-C₁₈-aryl which is substituted by C₁-C₁₈-alkyl or C₁-C₁a-alkoxy; C₁-C₁₈-alkyl; or C₁-C₁₈-alkyl which is interrupted by —O—;

R^{40′} and R^{41′} are each independently C₆-C₁₈-aryl; C₆-C₁₈-aryl which is substituted by C₁-C₁₈-alkyl or C₁-C₁-alkoxy; C₁-C₁₈-alkyl; or C₁-C₁₈-alkyl which is interrupted by —O—; or

R^{40′} and R^{41′} together form a 6-membered ring;

R^{42′} and R^{43′} are each independently C₆-C₁₈-aryl; C₆-C₁₈-aryl which is substituted by C₁-C₁₈-alkyl or C₁-C₁₈-alkoxy; C₁-C₁₈-alkyl; or C₁-C₁₈-alkyl which is interrupted by —O—,

R^{44′} is C₆-C₁₈-aryl; C₆-C₁₈-aryl which is substituted by C₁-C₁₈-alkyl or C₁-C₁₈-alkoxy; C₁-C₁₈-alkyl; or C₁-C₁₈-alkyl which is interrupted by —O—,

R^{45′} and R^{46′} are each independently C₁-C₁₈-alkyl, C₆-C₁₈-aryl or C₆-C₁₈-aryl which is substituted by C₁-C₁₈-alkyl,

R^{47′} is C₁-C₁₈-alkyl, C₆-C₁₈-aryl or C₆-C₁₈-aryl which is substituted by C₁-C₁₈-alkyl.

Preferred compounds of the formula (XVI) are compounds of the formula (XVIa):

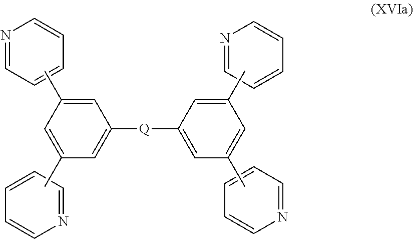

in which Q is:

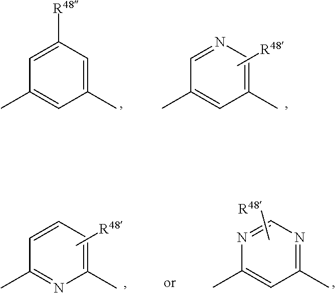

R^{48″} is H or C₁-C₁₈-alkyl and

R^{48″} is H, C₁-C₁₈-alkyl or

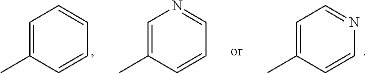

Particular preference is given to a compound of the formula:

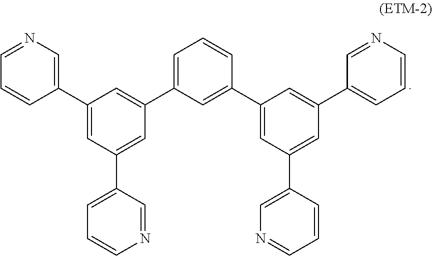 [SMILES: c1cncc(-c2cc(-c3cccnc3)cc(-c3cccc(-c4cc(-c5cccnc5)cc(-c5cccnc5)c4)c3)c2)c1]

In a further, very particularly preferred embodiment, the electron-transport layer comprises a compound Liq and a compound ETM-2.

In a preferred embodiment, the electron-transport layer comprises at least one compound of the formula (XVII) in an amount of 99 to 1% by weight, preferably 75 to 25% by weight, more preferably about 50% by weight, and at least one compound of the formula (XVI) in an amount of 1 to 99% by weight, preferably 25 to 75% by weight, more preferably about 50% by weight, where the amount of the compounds of the formulae (XVII) and the amount of the compounds of the formulae (XVI) adds up to a total of 100% by weight.

The preparation of the compounds of the formula (XVI) is described in J. Kido et al., Chem. Commun. (2008) 5821-5823, J. Kido et al., Chem. Mater. 20 (2008) 5951-5953 and JP2008/127326, or the compounds can be prepared analogously to the processes disclosed in the aforementioned documents.

It is likewise possible to use mixtures of alkali metal hydroxyquinolate complexes, preferably Liq, and dibenzofuran compounds in the electron-transport layer. Reference is made to WO2011/157790. Dibenzofuran compounds A-1 to A-36 and B-1 to B-22 described in WO2011/157790 are preferred, wherein dibenzofuran compound

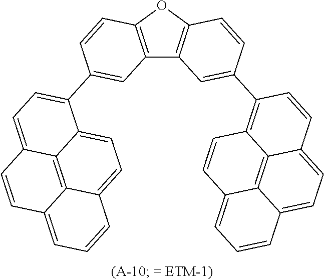 [SMILES: c1cc2ccc3ccc(-c4ccc5oc6ccc(-c7ccc8ccc9cccc%10ccc7c8c9%10)cc6c5c4)c4ccc(c1)c2c34]

is most preferred.

In a preferred embodiment, the electron-transport layer comprises Liq in an amount of 99 to 1% by weight, preferably 75 to 25% by weight, more preferably about 50% by weight, and at least one dibenzofuran compound in an amount of 1 to 99% by weight, preferably 25 to 75% by weight, more preferably about 50% by weight, where the amount of Liq and the amount of the dibenzofuran compound(s), especially ETM-1, adds up to a total of 100% by weight.

In a preferred embodiment, the electron-transport layer comprises at least one phenanthroline derivative and/or pyridine derivative.

In a further preferred embodiment, the electron-transport layer comprises at least one phenanthroline derivative and/or pyridine derivative and at least one alkali metal hydroxyquinolate complex.

In a further preferred embodiment, the electron-transport layer comprises at least one of the dibenzofuran compounds A-1 to A-36 and B-1 to B-22 described in WO2011/157790, especially ETM-1.

In a further preferred embodiment, the electron-transport layer comprises a compound described in WO2012/111462, WO2012/147397, WO2012014621, such as, for example, a compound of formula

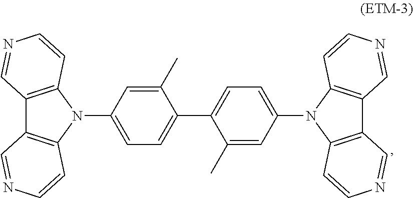 [SMILES: Cc1cc(-n2c3ccncc3c3cnccc32)ccc1-c1ccc(-n2c3ccncc3c3cnccc32)cc1C]

US2012/0261654, such as, for example, a compound of formula

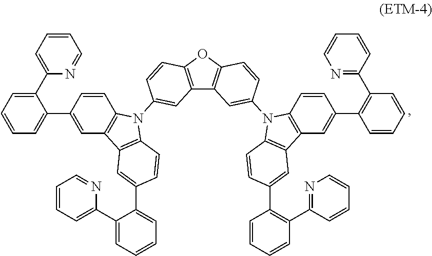 [SMILES: c1ccc(-c2ccccc2-c2ccc3c(c2)c2cc(-c4ccccc4-c4ccccn4)ccc2n3-c2ccc3oc4ccc(-n5c6ccc(-c7ccccc7-c7ccccn7)cc6c6cc(-c7ccccc7-c7ccccn7)ccc65)cc4c3c2)nc1]

and WO2012/115034, such as for example, such as, for example, a compound of formula

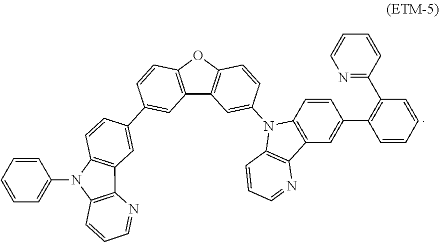 [SMILES: c1ccc(-n2c3ccc(-c4ccc5oc6ccc(-n7c8ccc(-c9ccccc9-c9ccccn9)cc8c8ncccc87)cc6c5c4)cc3c3ncccc32)cc1]

Further preferred embodiments of the electron injection layer of the OLED according to the present invention are mentioned in US 2013306955.

In particular, the electron transporting layer of the OLED according to the present invention, between the light emitting layer and the cathode, preferably comprises at least one compound of the general formulae (I), (II) or (III).

In a preferred embodiment, the electron transporting layer comprising at least one compound of the general formulae (I), (II) or (III) further comprises a reducing dopant.

Examples of the reducing dopant include a donating metal, a donating metal compound, and a donating metal complex. The reducing dopant may be used alone or in combination of two or more.

The reducing dopant referred to herein is an electron-donating material. The electron-donating material is a material which generates radical anions by the interaction with a coexisting organic material in the electron transporting layer or an organic material in a layer adjacent to the electron transporting layer, or a material having an electron-donating radical.

The donating metal is a metal having a work function of 3.8 eV or less, preferably an alkali metal, an alkaline earth metal, or a rare earth metal, and more preferably Cs, Li, Na, Sr, K, Mg, Ca, Ba, Yb, Eu, or Ce.

The donating metal compound is a compound comprising the above donating metal, preferably a compound comprising an alkali metal, an alkaline earth metal, or a rare earth metal, and more preferably a halide, an oxide, a carbonate, or a borate of these metals, for example, a compound represented by MOₓ (M: donating metal, x: 0.5 to 1.5), MFₓ (x: 1 to 3), or M(CO₃)ₓ (x: 0.5 to 1.5).

The donating metal complex is a complex comprising the above donating metal, preferably an organic metal complex of an alkali metal, an alkaline earth metal or a rare earth metal, and more preferably an organic metal complex represented by formula (I):

MQₙ (I)

wherein M is a donating metal, Q is a ligand, preferably a carboxylic acid derivative, a diketone derivative, or a quinoline derivative, and n is an integer of 1 to 4.

Examples of the donating metal complex include watermill-shaped tungsten compounds described in JP 2005-72012A and phthalocyanine compounds having an alkali metal or an alkaline earth metal as the central metal, which are described in JP 11-345687A.

The reducing dopant is preferably at least one selected from the group consisting of an alkali metal, an alkaline earth metal, a rare earth metal, an alkali metal oxide, an alkali metal halide, an alkaline earth metal oxide, an alkaline earth metal halide, a rare earth metal oxide, a rare earth metal halide, an organic complex having an alkali metal, an organic complex having an alkaline earth metal, and an organic complex having a rare earth metal, and more preferably a 8-quinolinol complex of an alkali metal.

Examples of the alkali metal includes:

Li (lithium, work function: 2.93 eV),

Na (sodium, work function: 2.36 eV),

K (potassium, work function: 2.3 eV),

Rb (rubidium, work function: 2.16 eV), and

Cs (cesium, work function: 1.95 eV).

The values of work functions are based on Handbook of Chemistry (Pure Chemistry II, 1984, p. 493, edited by The Chemical Society of Japan). The same applies hereafter.

Preferred examples of the alkaline earth metals are:

Ca (calcium, work function: 2.9 eV),

Mg (magnesium, work function: 3.66 eV),

Ba (barium, work function: 2.52 eV), and

Sr (strontium, work function: 2.0 to 2.5 eV).

The work function of strontium is based of Physics of Semiconductor Device (N.Y., Wiley, 1969, p. 366).

Preferred examples of the rare earth metal are:

Yb (ytterbium, work function: 2.6 eV),

Eu (europium, work function: 2.5 eV),

Gd (gadolinium, work function: 3.1 eV), and

Er (erbium, work function: 2.5 eV).

Examples of the alkali metal oxide include Li₂O, LiO, and NaO. The alkaline earth metal oxide is preferably CaO, BaO, SrO, BeO, or MgO.

Examples of the alkali metal halide include a fluoride, for example, LiF, NaF, CsF, and KF and a chloride, for example, LiCl, KCl, and NaCl.

The alkaline earth metal halide is preferably a fluoride, such as CaF₂, BaF₂, SrF₂, MgF₂, and BeF₂ and a halide other than fluoride.

An OLED wherein at least one compound according to general formulae (I), (II) or (III) used in the electron transporting layer is particularly preferred because the driving voltage is reduced while increasing the efficiency.

The content of the at least one compound according to general formulae (I), (II) or (III) in the electron transporting layer is preferably 50% by mass or more and more preferably 60% by mass or more.

The electron transporting layer facilitates the injection of electrons into the light emitting layer and transports the electrons to the light emitting zone, and has a large electron mobility and an electron affinity generally as large as 2.5 eV or more. The electron transporting layer is preferably formed from a material capable of transporting electrons to the light emitting layer at a lower strength of electric field, preferably having an electron mobility of, for example, at least 10⁻⁶ cm²N-s under an electric field of 10⁴ to 10⁶ V/cm.

When the of the at least one compound according to general formulae (I), (II) or (III) is used in the electron transporting layer, the electron transporting layer may be formed from at least one compound according to general formulae (I), (II) or (III) alone or in combination with another material.

The material for forming the electron injecting/transporting layer in combination with at least one compound according to general formulae (I), (II) or (III) is not particularly limited as long as having the preferred properties mentioned above and may be selected from those commonly used as the electron transporting material in the field of photoconductive materials and those known as the materials for the electron injecting/transporting layer of organic EL devices.

In the present invention, an electron injecting layer including an insulating material or a semiconductor may be disposed between the cathode and the organic layer. By such an electron injecting layer, the leak of electric current is effectively prevented to improve the electron injecting ability. Preferred examples of the insulating material include at least one metal compound selected from the group consisting of an alkali metal chalcogenide, an alkaline earth metal chalcogenide, an alkali metal halide, and an alkaline earth metal halide. An electron injecting layer including the above alkali metal chalcogenide is preferred because the electron injecting property is further improved. Preferred alkali metal chalcogenides include Li₂O, K₂O, Na₂S, Na₂Se, and Na₂O; preferred alkaline earth metal chalcogenides include CaO, BaO, SrO, BeO, BaS, and CaSe; preferred alkali metal halides include LiF, NaF, KF, LiCl, KCl, and NaCl; and preferred alkaline earth metal halides include fluoride such as CaF₂, BaF₂, SrF₂, MgF₂, and BeF₂ and halides other than fluoride.

Examples of the semiconductor for the electron transporting layer include an oxide, a nitride and an oxynitride of at least one element selected from Ba, Ca, Sr, Yb, Al, Ga, In, Li, Na, Cd, Mg, Si, Ta, Sb, and Zn, which are used singly or in combination of two or more. It is preferred that the inorganic compound constituting the electron transporting layer forms a microcrystalline or amorphous insulating thin film. When constituted of the insulating thin film described above, the electron injecting layer is made more uniform to reduce the pixel defect such as dark spots. Examples of such a inorganic compound include the alkali metal chalcogenide, the alkaline earth metal chalcogenide, the alkali metal halide and the alkaline earth metal halide which are described above.

Electron Injection Layer (h):

The electron injection layer may be any layer that improves the injection of electrons into an adjacent organic layer.

Lithium-comprising organometallic compounds such as 8-hydroxyquinolatolithium (Liq), CsF, NaF, KF, Cs₂CO₃ or LiF may be applied between the electron transport layer (g) and the cathode (i) as an electron injection layer (h) in order to reduce the operating voltage.

Cathode (i):

The cathode (i) is an electrode which serves to introduce electrons or negative charge carriers. The cathode may be any metal or nonmetal which has a lower work function than the anode. Suitable materials for the cathode are selected from the group consisting of alkali metals of group 1, for example Li, Cs, alkaline earth metals of group 2, metals of group 12 of the Periodic Table of the Elements, comprising the rare earth metals and the lanthanides and actinides. In addition, metals such as aluminum, indium, calcium, barium, samarium and magnesium, and combinations thereof, may be used.

In general, the different layers, if present, have the following thicknesses: anode (a): 500 to 5000 Å (ångström), preferably 1000 to 2000 Å;

hole injection layer (b): 50 to 1000 Å, preferably 200 to 800 Å,

hole-transport layer (c): 50 to 1000 Å, preferably 100 to 800 Å,

exciton blocking layer (d): 10 to 500 Å, preferably 50 to 100 Å,

light-emitting layer (e): 10 to 1000 Å, preferably 50 to 600 Å,

hole/exciton blocking layer (f): 10 to 500 Å, preferably 50 to 100 Å,

electron-transport layer (g): 50 to 1000 Å, preferably 200 to 800 Å,

electron injection layer (h): 10 to 500 Å, preferably 20 to 100 Å, and

cathode (i): 200 to 10 000 Å, preferably 300 to 5000 Å.

The person skilled in the art is aware (for example on the basis of electrochemical studies) of how suitable materials have to be selected. Suitable materials for the individual layers are known to those skilled in the art and are disclosed, for example, in WO00/70655.

In addition, it is possible that some of the layers used in the inventive OLED have been surface-treated in order to increase the efficiency of charge carrier transport. The selection of the materials for each of the layers mentioned is preferably determined by obtaining an OLED with a high efficiency and lifetime.

The inventive OLED can be produced by methods known to those skilled in the art. In general, the inventive OLED is produced by successive vapor deposition of the individual layers onto a suitable substrate. Suitable substrates are, for example, glass, inorganic semiconductors or polymer films. For vapor deposition, it is possible to use customary techniques, such as thermal evaporation, chemical vapor deposition (CVD), physical vapor deposition (PVD) and others. In an alternative process, the organic layers of the OLED can be applied from solutions or dispersions in suitable solvents, employing coating techniques known to those skilled in the art.

Use of the compounds of the formulae (I), (II) or (III) in at least one layer of the OLED, preferably in the light-emitting layer, preferably as a host material, a charge transporting material and/or a dopant without metal species as, particularly preferably as a host material and hole or electron transporting material, makes it possible to obtain OLEDs with high efficiency and with low use and operating voltage. Frequently, the OLEDs obtained by the use of the compounds of the formulae (I), (II) or (III) additionally have high lifetimes. The efficiency of the OLEDs can additionally be improved by optimizing the other layers of the OLEDs. For example, high-efficiency cathodes such as Ca or Ba, if appropriate in combination with an intermediate layer of LiF, can be used. Moreover, additional layers may be present in the OLEDs in order to adjust the energy level of the different layers and to facilitate electroluminescence.

The OLEDs may further comprise at least one second light-emitting layer. The overall emission of the OLEDs may be composed of the emission of the at least two light-emitting layers and may also comprise white light.

The OLEDs can be used in all apparatus in which electroluminescence is useful. Suitable devices are preferably selected from stationary and mobile visual display units and illumination units. Stationary visual display units are, for example, visual display units of computers, televisions, visual display units in printers, kitchen appliances and advertising panels, illuminations and information panels. Mobile visual display units are, for example, visual display units in cellphones, tablet PCs, laptops, digital cameras, MP3 players, vehicles and destination displays on buses and trains. Further devices in which the inventive OLEDs can be used are, for example, keyboards; items of clothing; furniture; wallpaper. In addition, the present invention relates to a device selected from the group consisting of stationary visual display units such as visual display units of computers, televisions, visual display units in printers, kitchen appliances and advertising panels, illuminations, information panels, and mobile visual display units such as visual display units in cellphones, tablet PCs, laptops, digital cameras, MP3 players, vehicles and destination displays on buses and trains; illumination units; keyboards; items of clothing; furniture; wallpaper, comprising at least one inventive organic light-emitting diode or at least one inventive light-emitting layer.

The following examples are included for illustrative purposes only and do not limit the scope of the claims. Unless otherwise stated, all parts and percentages are by weight.

### EXAMPLES

### Synthesis Example 1—Compound 1

### Intermediate 1-1

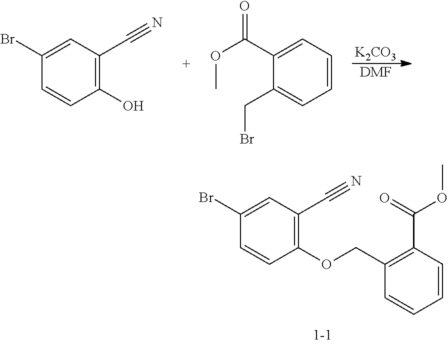

5-bromo-2-hydroxy-benzonitrile (441.8 g, 2.23 mol), methyl 2-(bromomethyl)benzoate (479 g, 2.03 mol), and potassium carbonate (560 g, 4.05 mol) were added to DMF (4.6 L) under Ar atmosphere. After the mixture was stirred at 50° C. for 1 h, the reaction mixture was cooled at 0° C. After 2 L of water was added there, the mixture was stirred at room temperature for 1 h to give a solid. The solid was collected by filtration, and dissolved in THF. The THF solution was dried with MgSO₄, and then it was concentrated to yield 673 g of 1-1. Without further purification, it was used in the next reaction.

### Intermediate 1-2

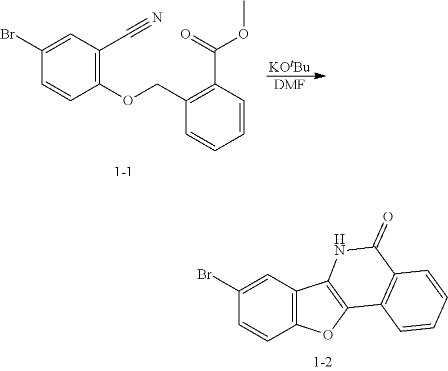

1-1 (673 g, 1.95 mol) was dissolved in DMF (4.6 L), and potassium tert-butoxide (250 g, 2.23 mol) was added at room temperature. Then, the mixture was stirred at 80° C. for 1 h. After the reaction mixture was cooled at room temperature and 1.5 L of toluene was added there, 1.2 L of 4 M HCl in dioxane was added dropwise at 0° C. to give a solid. The solid was collected by filtration and washed with toluene to yield 478 g (75%) of 1-2 as a white powder.

LC-MS (m/z) 314

### Intermediate 1-3

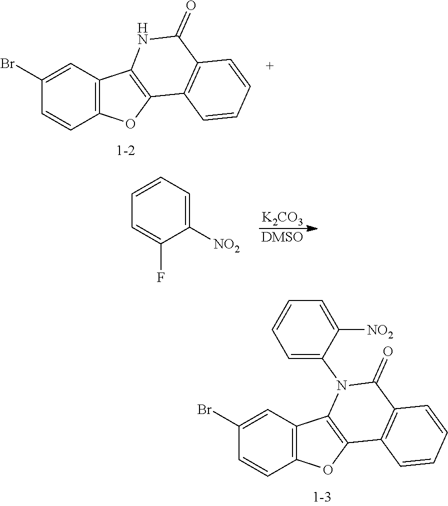

1-2 (203 g, 646 mmol) and 1-fluoro-2-nitro-benzene (100 g, 711 mmol) were added to 2 L of DMSO. The mixture was heated at 80° C. To the mixture was potassium carbonate (116 g, 840 mmol) added, and the mixture was stirred at 100° C. for 20 h. After the reaction mixture was cooled at room temperature, 1 L of water was added there to give a solid. The solid was collected by filtration, and was washed with water and n-heptane. The crude product was purified by column chromatography on silica gel eluting with xylene to yield 134 g (47%) of 1-3.

LC-MS (m/z) 435

### Intermediate 1-4

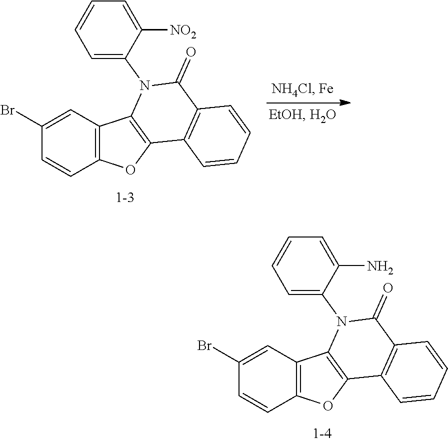 [SMILES: O=c1c2ccccc2c2oc3ccc(Br)cc3c2n1-c1ccccc1[N+](=O)[O-] ; Nc1ccccc1-n1c(=O)c2ccccc2c2oc3ccc(Br)cc3c21]

1-3 (134 g, 309 mmol) was added to 3.6 L of ethanol, and the mixture was heated at 60° C. under Ar atmosphere. To the mixture are added ammonium chloride (82.7 g, 1.546 mol) dissolved in water (555 mL) and iron (86.3 g, 1.546 mol), and the mixture was stirred at 70° C. for 20 h. After the reaction mixture was cooled at room temperature, 1 L of 10% potassium carbonate aqueous solution was added there and the mixture was stirred at room temperature for 1 h to give a solid. The solid was collected by filtration, and washed with water. The solid was suspended to a mixed solvent THF (2.5 L) and DMF (0.5 L), and the insoluble material was removed by filtration. After the filtrate was concentrated, 1 L of water was added there to give a solid. The solid was collected by filtration, and washed with water and methanol to yield 127.7 g (98%) of 1-4.

LC-MS (m/z) 405

### Intermediate 1-5

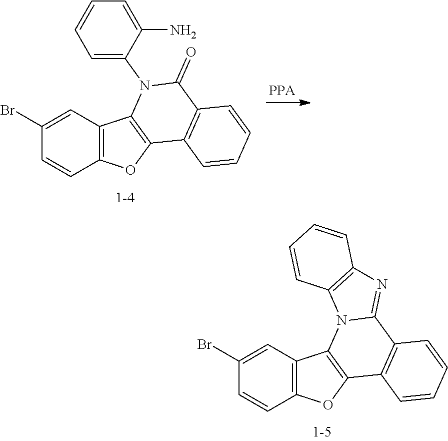 [SMILES: Nc1ccccc1-n1c(=O)c2ccccc2c2oc3ccc(Br)cc3c21 ; Brc1ccc2oc3c4ccccc4c4nc5ccccc5n4c3c2c1]

1-4 (103 g, 251 mmol) was added to polyphosphoric acid (1050 mL) under Ar atmosphere. The mixture was stirred at 225° C. for 4 h. After the reaction mixture was cooled at room temperature, it was poured into ice-water to give a solid. The solid was collected by filtration, and washed with water. After it was stirred in 1 L of 10% potassium carbonate aqueous solution at room temperature for 3 h, the solid was collected by filtration, and washed with water. The crude product was purified by column chromatography on silica gel eluting with toluene to yield 92 g (50%) of 1-5 as a beige powder.

¹H-NMR (300 MHz, DMSO-d₆) δ 8.73 (ddd, J=0.7, 0.9, 8.1 Hz, 1H), 8.56 (d, J=1.8 Hz, 1H), 8.43 (m, 1H), 8.17 (ddd, J=0.7, 0.8, 7.9 Hz, 1H), 7.86 (m, 5H), 7.57 (m, 2H) LC-MS (m/z) 388

### Compound 1

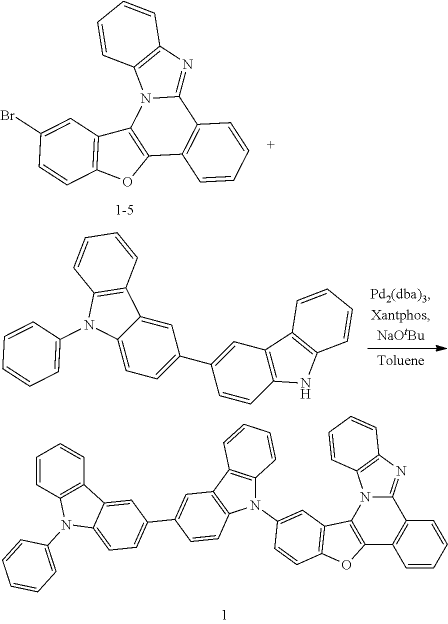

1-5 (1.56 g, 4.0 mmol), 3-(9H-carbazol-3-yl)-9-phenyl-carbazole (1.63 g, 4.0 mmol), and sodium tert-butoxide (769 mg, 8.0 mmol) were suspended in 27 mL of toluene in Ar atmosphere. Xantphos (185 mg, 0.32 mmol) and tris(dibenzylideneacetone)dipalladium (0) (183 mg, 0.2 mmol) were added there, and the mixture was refluxed overnight. After the reaction mixture was cooled at room temperature, a solid was removed by filtration and washed with THF. The solution was concentrated to give a dark brown powder. The crude product was purified by column chromatography on silica gel eluting with a mixed solvent of toluene-CHCl₃ (10:1) to yield 2.29 g (80%) of 1 as a beige powder.

¹H-NMR (300 MHz, CDCl₃) δ 9.03 (d, J=8.2 Hz, 1H), 8.85 (d, J=2.2 Hz, 1H), 8.57 (d, J=1.8 Hz, 1H), 8.51 (d, J=1.8 Hz, 1H), 8.45 (d, J=8.2 Hz, 1H), 8.41 (d, J=8.3 Hz, 1H), 8.35 (d, J=7.3 Hz, 1H), 8.24 (d, J=7.2 Hz, 1H), 8.11 (d, J=8.1 Hz, 1H), 8.06 (d, J=8.8 Hz, 1H), 7.92-7.78 (m, 5H), 7.69-7.65 (m, 4H), 7.60-7.39 (m, 10H), 7.37-7.33 (m, 1H)

LC-MS (m/z) 714

### Synthesis Example 2—Compound 2

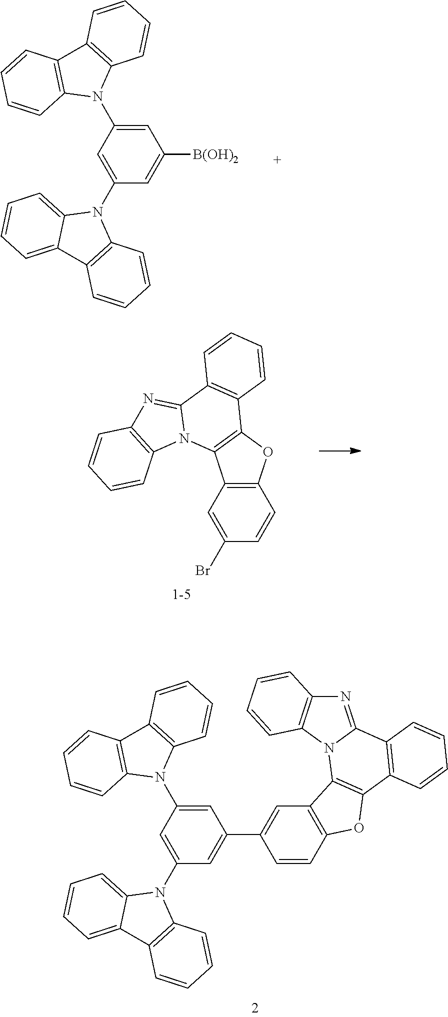 [SMILES: OB(O)c1cc(-n2c3ccccc3c3ccccc32)cc(-n2c3ccccc3c3ccccc32)c1 ; Brc1ccc2oc3c4ccccc4c4nc5ccccc5n4c3c2c1 ; c1ccc2c(c1)nc1c3ccccc3c3oc4ccc(-c5cc(-n6c7ccccc7c7ccccc76)cc(-n6c7ccccc7c7ccccc76)c5)cc4c3n21]

[3,5-di(carbazol-9-yl)phenyl]boronic acid (1.5 g, 3.3 mmol) (described in US 2013/0082591 A1) was combined with 1-5 (1.3 g, 3.3 mmol) and K₂CO₃ (0.9 g, 6.6 mmol) in a 3 necked round bottom flask and degassed with N₂. Pd(PPh₃)₄ (0.2 g, 0.16 mmol) was then added followed by dioxane/H₂O (30 mL, 4:1). The resulting reaction mixture was heated under a stream of N₂ at an oil bath temperature of 90° C. After 3 hours, the reaction was completed. The mixture was cooled to room temperature and the solvent was concentrated under reduced pressure. The crude residue was taken up in CHCl₃ and washed with water (×2), dried over anhydrous MgSO₄ and the solvent was evaporated. The crude product was suspended in acetone and stirred overnight at room temperature in acetone. The precipitate was filtered and then dissolved in CHCl₃ (1 L) and poured through a plug of silica gel. The solvent was evaporated and the product was finally purified by recrystallization from chlorobenzene to give 2 g (85% yield) of the product as a white solid.

¹H-NMR (400 MHz, CDCl₃) 8.99 (d, J=7.7 Hz, 1H), 8.91 (s, 1H), 8.66 (d, J=8.1 Hz, 1H), 8.33 (d, J=7.6 Hz, 1H), 8.22 (d, J=7.8 Hz, 4H), 8.16-8.01 (m, 3H), 7.94 (s, 1H), 7.86 (m, 3H), 7.77 (m, 5H), 7.53 (m, 6H), 7.39 (m, 4H).

LC-MS (M+1) 715

### Synthesis Example 3—Compound 3

### Intermediate 3-1

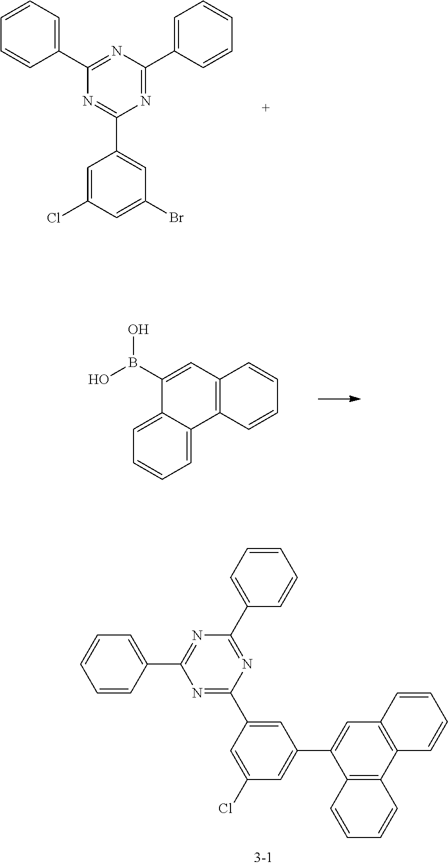 [SMILES: Clc1cc(Br)cc(-c2nc(-c3ccccc3)nc(-c3ccccc3)n2)c1 ; OB(O)c1cc2ccccc2c2ccccc12 ; Clc1cc(-c2nc(-c3ccccc3)nc(-c3ccccc3)n2)cc(-c2cc3ccccc3c3ccccc23)c1]

24.40 g (57.6 mmol) of 2-(3-bromo-5-chlorophenyl)-4,6-diphenyl-1,3,5-triazine (synthesized according to known procedures), 12.80 g (57.6 mmol) of 9-phenanthrylboronic acid and 86 ml (173 mmol) of a 2M sodium carbonate solution were suspended in 450 ml of DME and evacuated and purged with argon 4 times. Then argon was bubbled through for 30 minutes. 1.33 g (1.15 mmol) of tetrakis(triphenylphospin)-palladium(0) were added under argon and argon was bubbled through for another 5 minutes. Then the reaction mixture was heated to 85° C. After stirring under argon for 14 hours at this temperature, the reaction mixture was cooled to room temperature and filtered. The residue was washed with hot toluene to give 25.7 g (85%) of 3-1.

¹H-NMR (400 MHz, CDCl₃-d) δ 8.89-8.71 (m, 8H), 7.96 (dd, J=7.8, 1.5 Hz, 1H), 7.91 (dd, J=8.3, 1.3 Hz, 1H), 7.83-7.63 (m, 5H), 7.64-7.52 (m, 7H).

### Intermediate 3-2

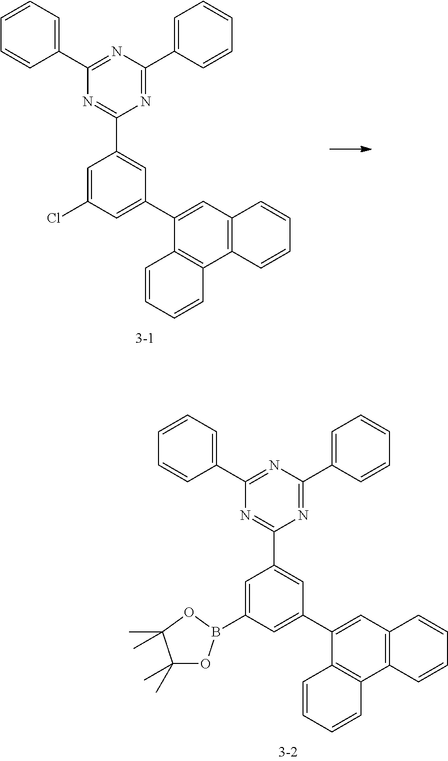 [SMILES: Clc1cc(-c2nc(-c3ccccc3)nc(-c3ccccc3)n2)cc(-c2cc3ccccc3c3ccccc23)c1 ; CC1(C)OB(c2cc(-c3nc(-c4ccccc4)nc(-c4ccccc4)n3)cc(-c3cc4ccccc4c4ccccc34)c2)OC1(C)C]

8.58 g (16.50 mmol) of 3-1, 4.61 g (18.15 mmol) of 4,4,5,5-tetramethyl-2-(4,4,5,5-tetramethyl-1,3,2-dioxaborolan-2-yl)-1,3,2-dioxaborolane and 4.05 g (41.25 mmol) of potassium acetate were mixed. Then 85 ml of dioxane, and then 0.203 g (0.495 mmol) of dicyclohexyl-[2-(2,6-dimethoxyphenyl)phenyl]phosphane and 0.227 g (0.248 mmol) of Pd₂(dba)₃ were added under argon and the suspension was heated to 115° C. After stirring at this temperature for 4 hours, the reaction mixture was cooled to room temperature. 250 ml of water and 150 ml of chloroform were added to the orange suspension, which was stirred and filtered through hyflo into the separation funnel, while washing with 200 ml of chloroform. The phases were separated, the water phase was extracted with 100 ml of chloroform, the combined organic phases were washed with 200 ml of water, dried over Na₂SO₄, filtered and the solvent was removed in vacuum. The crude product was recrystallized from 60 ml of toluene and dried at high vacuum to give 9.39 g (93%) of 3-2.

¹H-NMR (400 MHz, CDCl₃-d) δ 9.25 (t, J=1.5 Hz, 1H), 9.03 (t, J=1.8 Hz, 1H), 8.92-8.60 (m, 6H), 8.24 (t, J=1.5 Hz, 1H), 8.06-7.86 (m, 2H), 7.83 (s, 1H), 7.75-7.48 (m, 10H), 1.43 (s, 12H).

### Compound 3

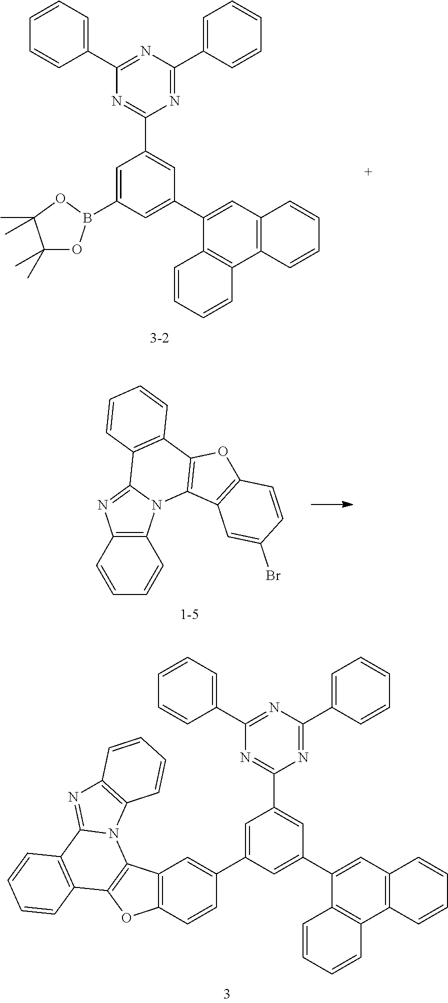 [SMILES: CC1(C)OB(c2cc(-c3nc(-c4ccccc4)nc(-c4ccccc4)n3)cc(-c3cc4ccccc4c4ccccc34)c2)OC1(C)C ; Brc1ccc2oc3c4ccccc4c4nc5ccccc5n4c3c2c1 ; c1ccc(-c2nc(-c3ccccc3)nc(-c3cc(-c4ccc5oc6c7ccccc7c7nc8ccccc8n7c6c5c4)cc(-c4cc5ccccc5c5ccccc45)c3)n2)cc1]

2.57 g (4.20 mmol) of 3-2 and 1.55 g (4.00 mmol) of 1-5 were suspended in 40 ml of THF. 0.110 g (0.120 mmol) of Pd₂(dba)₃ and 0.07 g (0.240 mmol) of tri-t-butylphosphonium tetrafluorobo-rate were added, and the mixture was heated to 50° C. After a solution of 2.12 g (10.0 mmol) of potassium phosphate dissolved in 8 ml of water was added there, the reaction mixture was heated to reflux for 4 hours and then cooled to room temperature. 100 ml of chloroform and 200 ml of a 3% solution of sodium cyanide were added, the mixture was heated to reflux for 2 hours and then cooled to room temperature. The yellow suspension was filtered, washed with chloroform and water and dried at high vacuum. The crude product was purified by recrystallization from 1,2-dichlorobenzene The residue was washed with 1,2-dichlorobenzene and heptane and dried at high vacuum to yield 2.73 g (86%) of 3 as a white crystal.

LC-MS (M) 791

### Synthesis Example 4—Compound 4

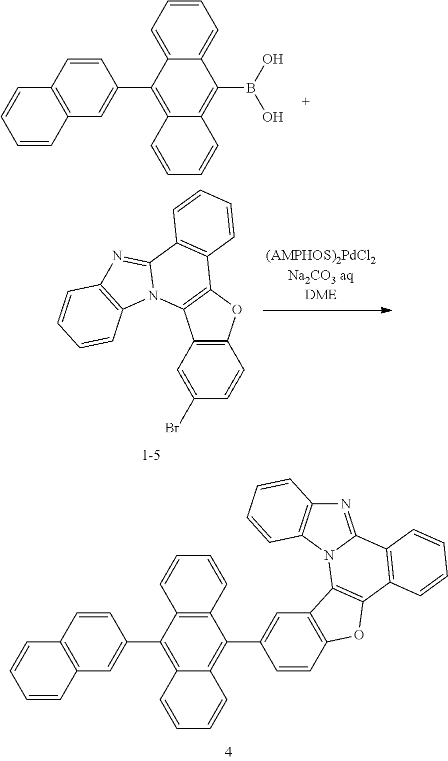

1,2-dimethoxyethane (80 ml) and a 2M sodium carbonate aqueous solution (15.5 mL, 31.0 mmol) were added to [10-(2-naphthyl)-9-anthryl]boronic acid (3.96 g, 11.4 mmol), 1-5 (4.00 g, 10.3 mmol), and (AMPHOS)₂PdCl₂ (0.293 g, 0.413 mmol) under Ar atmosphere. The mixture was stirred for 5 h under refluxing. After the reaction was completed, the solvent was evaporated and the residue was washed with methanol and recrystallized from chlorobenzene to give a product (4.10 g, 63% yield), which was identified as the compound 4 by mass spectrometry (m/e=610, Exact mass: 610.20).

### Synthesis Example 5—Compound 5

### Intermediate 5-1

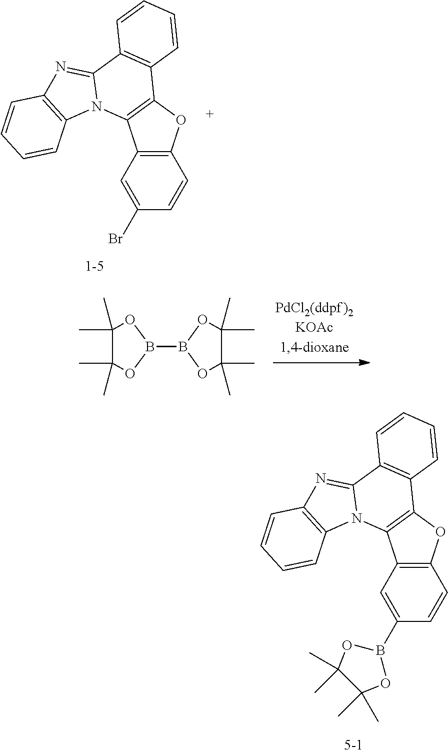

1,4-dioxane (140 ml) was added to 1-5 (7.00 g, 18.1 mmol), bis(pinacolato)diboron (5.51 g, 21.7 mmol), [1,1′-bis(diphenylphosphino)ferrocene]palladium(III) dichloride dichloromethane adduct (0.443 g, 0.542 mmol), and potassium acetate (3.55 g, 36.2 mmol) under Ar atmosphere. The mixture was stirred for 4 h under refluxing. After the reaction was completed, the mixture was purified by column chromatography on silica gel and the solvent was evaporated to give the intermediate 5-1 (7.86 g, 99% yield).

### Compound 5

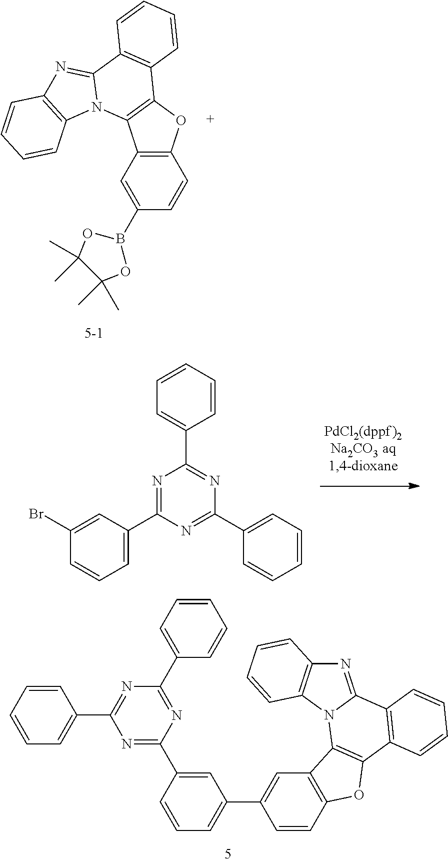 [SMILES: CC1(C)OB(c2ccc3oc4c5ccccc5c5nc6ccccc6n5c4c3c2)OC1(C)C ; Brc1cccc(-c2nc(-c3ccccc3)nc(-c3ccccc3)n2)c1 ; c1ccc(-c2nc(-c3ccccc3)nc(-c3cccc(-c4ccc5oc6c7ccccc7c7nc8ccccc8n7c6c5c4)c3)n2)cc1]

1,4-dioxane (250 ml) and a 2M sodium carbonate aqueous solution (27.1 ml, 54.2 mmol) were added to 5-1 (7.85 g, 18.1 mmol), 2-(3-bromophenyl)-4,6-diphenyl-1,3,5-triazine (7.02 g, 18.1 mmol), and [1,1′-bis(diphenylphosphino)ferrocene]palladium(II) dichloride dichloromethane adduct (0.590 g, 0.723 mmol) under Ar atmosphere. The mixture was stirred at 90° C. for 20 h. After the reaction, the mixture was cooled to room temperature and methanol was added to give a solid. The solid was collected by filtration and was washed with 1,4-dioxane to give a product (3.20 g, 27% yield), which was identified as the compound 5 by mass spectrometry (m/e=615, Exact mass: 615.21).

### Synthesis Example 6—Compound 6

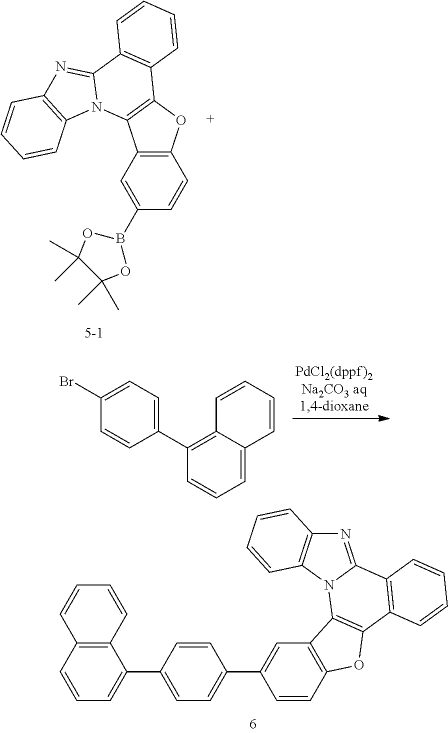

In the same manner as in the synthesis of the compound 5 in Synthesis example 5 except for using 1-(4-bromophenyl)naphthalene in place of 2-(3-bromophenyl)-4,6-diphenyl-1,3,5-triazine, a product was obtained (62% yield), which was identified as the compound 6 by mass spectrometry (m/e=510, Exact mass: 510.17).

### Synthesis Example 7—Compound 7

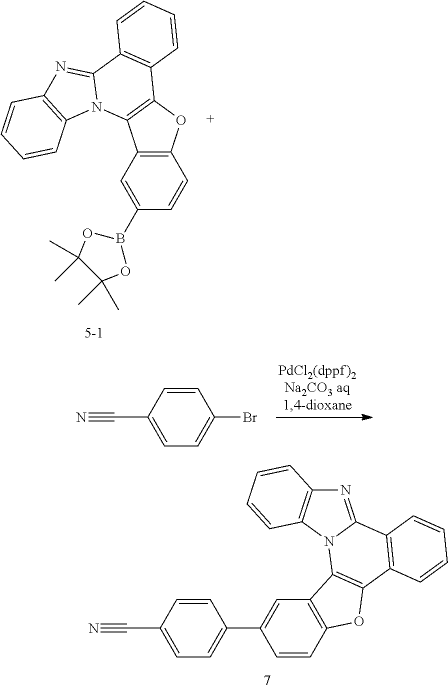 [SMILES: CC1(C)OB(c2ccc3oc4c5ccccc5c5nc6ccccc6n5c4c3c2)OC1(C)C ; N#Cc1ccc(Br)cc1 ; N#Cc1ccc(-c2ccc3oc4c5ccccc5c5nc6ccccc6n5c4c3c2)cc1]

In the same manner as in the synthesis of the compound 5 in Synthesis example 5 except for using 4-bromobenzonitrile in place of 2-(3-bromophenyl)-4,6-diphenyl-1,3,5-triazine, a product was obtained (70% yield), which was identified as the compound 7 by mass spectrometry (m/e=409, Exact mass: 409.12).

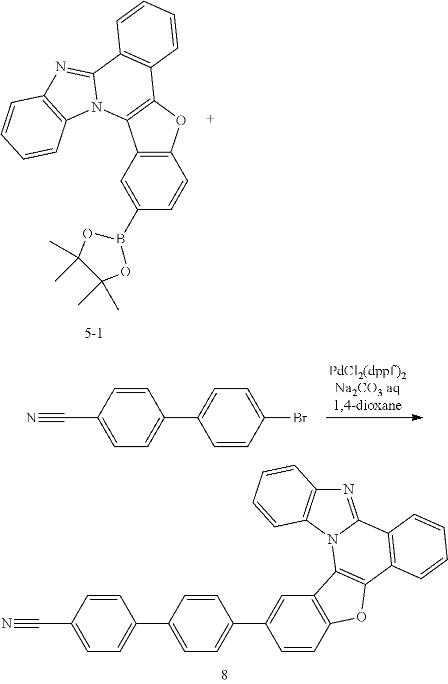

### Synthesis Example 8—Compound 8

In the same manner as in the synthesis of the compound 5 in Synthesis example 5 except for using 4-(4-bromophenyl)benzonitrile in place of 2-(3-bromophenyl)-4,6-diphenyl-1,3,5-triazine, a product was obtained (65% yield), which was identified as the compound 8 by mass spectrometry (m/e=485, Exact mass: 485.15).

### Synthesis Example 9—Compound 9

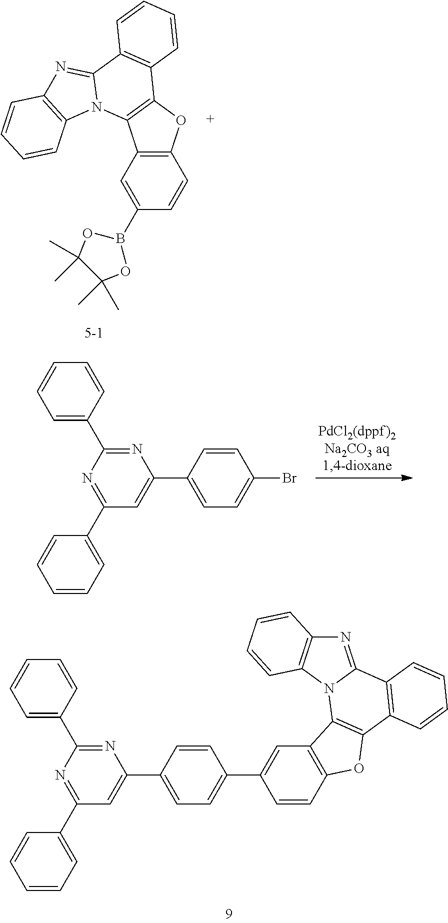 [SMILES: CC1(C)OB(c2ccc3oc4c5ccccc5c5nc6ccccc6n5c4c3c2)OC1(C)C ; Brc1ccc(-c2cc(-c3ccccc3)nc(-c3ccccc3)n2)cc1 ; c1ccc(-c2cc(-c3ccc(-c4ccc5oc6c7ccccc7c7nc8ccccc8n7c6c5c4)cc3)nc(-c3ccccc3)n2)cc1]

In the same manner as in the synthesis of the compound 5 in Synthesis example 5 except for using 4-(4-bromophenyl)-2,6-diphenylpyrimidine in place of 2-(3-bromophenyl)-4,6-diphenyl-1,3,5-triazine, a product was obtained (55% yield), which was identified as the compound 9 by mass spectrometry (m/e=614, Exact mass: 614.21).

### Synthesis Example 10—Compound 10

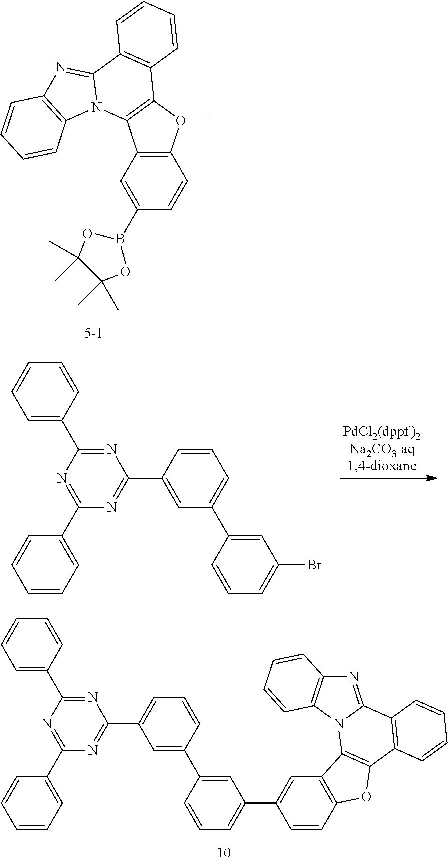

In the same manner as in the synthesis of the compound 5 in Synthesis example 5 except for using 2-[3-(3-bromophenyl)phenyl]-4,6-diphenyl-1,3,5-triazine in place of 2-(3-bromophenyl)-4,6-diphenyl-1,3,5-triazine, a product was obtained (55% yield), which was identified as the compound 10 by mass spectrometry (m/e=691, Exact mass: 691.24).

### Synthesis Example 11—Compound 11

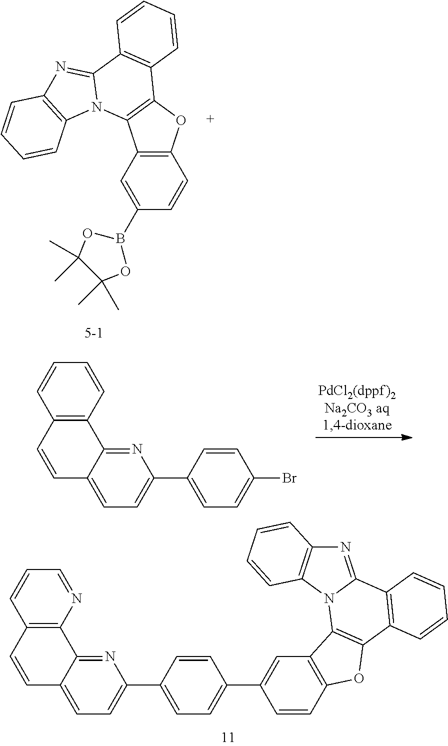

In the same manner as in the synthesis of the compound 5 in Synthesis example 5 except for using 2-(4-bromophenyl)-1,10-phenanthroline in place of 2-(3-bromophenyl)-4,6-diphenyl-1,3,5-triazine, a product was obtained (42% yield), which was identified as the compound 11 by mass spectrometry (m/e=562, Exact mass: 562.18).

### Synthesis Example 12—Compound 12

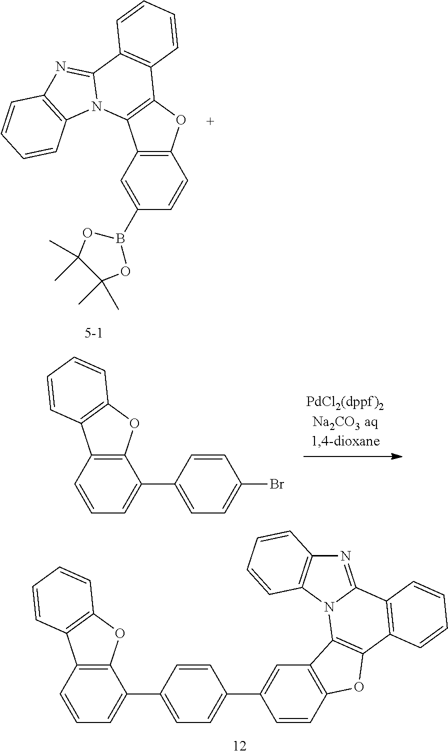

In the same manner as in the synthesis of the compound 5 in Synthesis example 5 except for using 4-(4-bromophenyl)dibenzofuran in place of 2-(3-bromophenyl)-4,6-diphenyl-1,3,5-triazine, a product was obtained (60% yield), which was identified as the compound 12 by mass spectrometry (m/e=550, Exact mass: 550.17).

### Synthesis Example 13—Compound 13

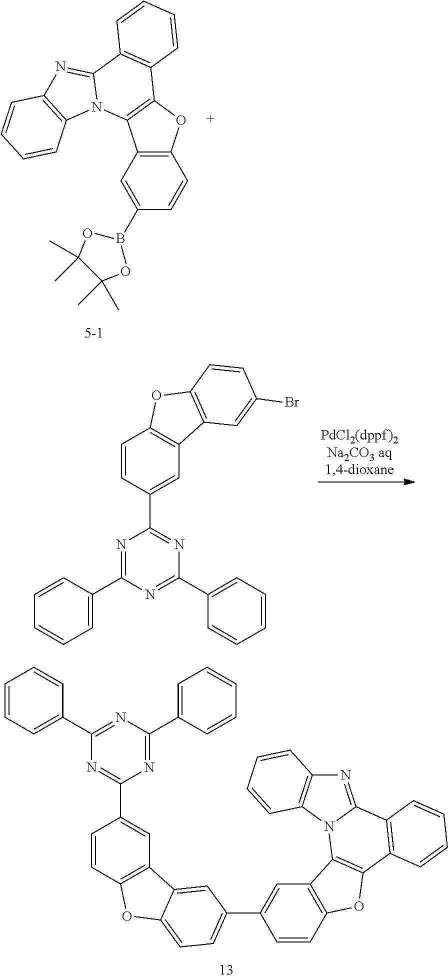

In the same manner as in the synthesis of the compound 5 in Synthesis example 5 except for using 2-(8-bromodibenzofuran-2-yl)-4,6-diphenyl-1,3,5-triazine in place of 2-(3-bromophenyl)-4,6-diphenyl-1,3,5-triazine, a product was obtained (51% yield), which was identified as the compound 13 by mass spectrometry (m/e=705, Exact mass: 705.22).

### Synthesis Example 14—Compound 14

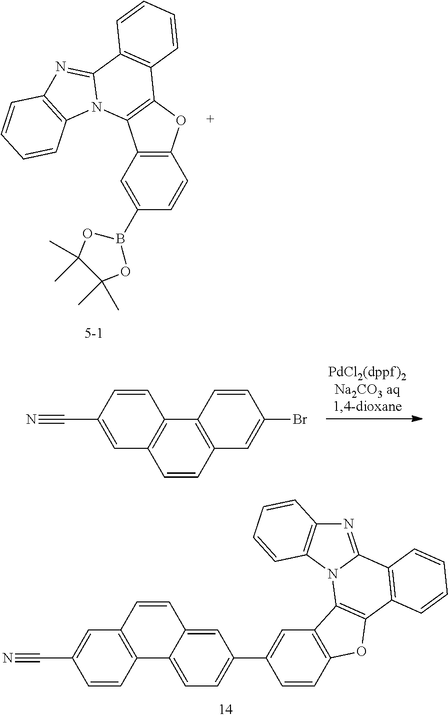 [SMILES: CC1(C)OB(c2ccc3oc4c5ccccc5c5nc6ccccc6n5c4c3c2)OC1(C)C ; N#Cc1ccc2c(ccc3cc(Br)ccc32)c1 ; N#Cc1ccc2c(ccc3cc(-c4ccc5oc6c7ccccc7c7nc8ccccc8n7c6c5c4)ccc32)c1]

In the same manner as in the synthesis of the compound 5 in Synthesis example 5 except for using 7-bromophenanthrene-2-carbonitrile in place of 2-(3-bromophenyl)-4,6-diphenyl-1,3,5-triazine, a product was obtained (45% yield), which was identified as the compound 14 by mass spectrometry (m/e=509, Exact mass: 509.15).

### Synthesis Example 15—Compound 15

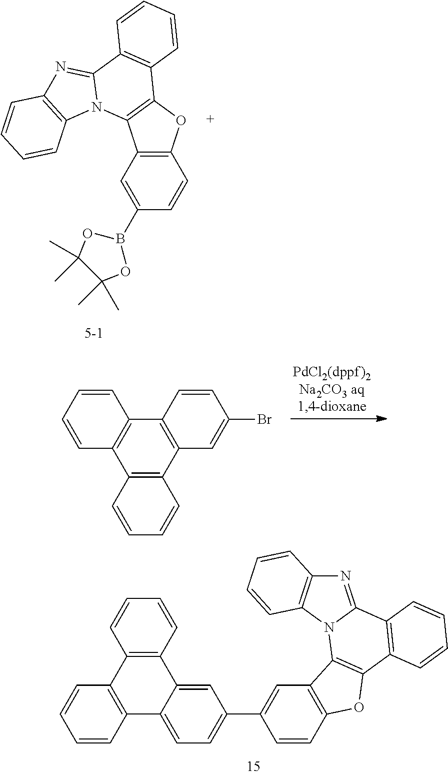 [SMILES: CC1(C)OB(c2ccc3oc4c5ccccc5c5nc6ccccc6n5c4c3c2)OC1(C)C ; Brc1ccc2c3ccccc3c3ccccc3c2c1 ; c1ccc2c(c1)nc1c3ccccc3c3oc4ccc(-c5ccc6c7ccccc7c7ccccc7c6c5)cc4c3n21]

In the same manner as in the synthesis of the compound 5 in Synthesis example 5 except for using 2-bromotriphenylene in place of 2-(3-bromophenyl)-4,6-diphenyl-1,3,5-triazine, a product was obtained (61% yield), which was identified as the compound 15 by mass spectrometry (m/e=534, Exact mass: 534.17).

### Synthesis Example 16—Compound 16

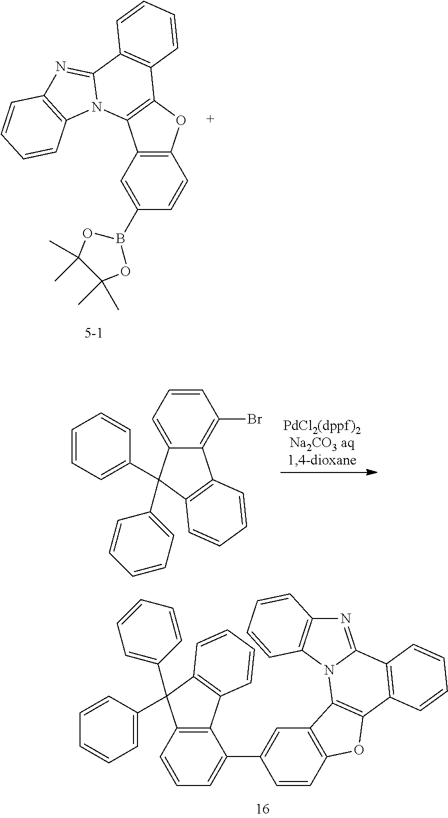

In the same manner as in the synthesis of the compound 5 in Synthesis example 5 except for using 4-bromo-9,9-diphenylfluorene in place of 2-(3-bromophenyl)-4,6-diphenyl-1,3,5-triazine, a product was obtained (60% yield), which was identified as the compound 16 by mass spectrometry (m/e=624, Exact mass: 624.22).

### Example 1

1.1 OLED Fabrication

A glass substrate with 120 nm-thick indium-tin-oxide (ITO) transparent electrode (manufactured by Geomatec Co., Ltd.) used as an anode was first cleaned with isopropanol in an ultrasonic bath for 10 min. To eliminate any possible organic residues, the substrate was exposed to an ultraviolet light and ozone for further 30 min. This treatment also improves the hole injection properties of the ITO. The cleaned substrate was mounted on a substrate holder and loaded into a vacuum chamber. Thereafter, the organic materials specified below were applied by vapor deposition to the ITO substrate at a rate of approx. 0.2-1 Å/sec at about 10⁻⁶-10⁻⁸ mbar. As the first layer, 5 nm-thick of electron accepting compound A was vapor-deposited. Then 50 nm-thick of aromatic amine compound B was applied as a first hole transporting layer. Successively, 60 nm-thick of aromatic amine compound C was applied as a second hole transporting layer. Then, a mixture of 8% by weight of an emitter compound (Ir(piq)₃), 92% by weight of the host (compound 1) were applied to form a 45 nm-thick phosphorescent-emitting layer. On the emitting layer, 30 nm-thick compound D was applied as an electron transport layer. Finally, 1 nm-thick LiF was deposited as an electron injection layer and 80 nm-thick Al was then deposited as a cathode to complete the device. The device was sealed with a glass lid and a getter in an inert nitrogen atmosphere with less than 1 ppm of water and oxygen.

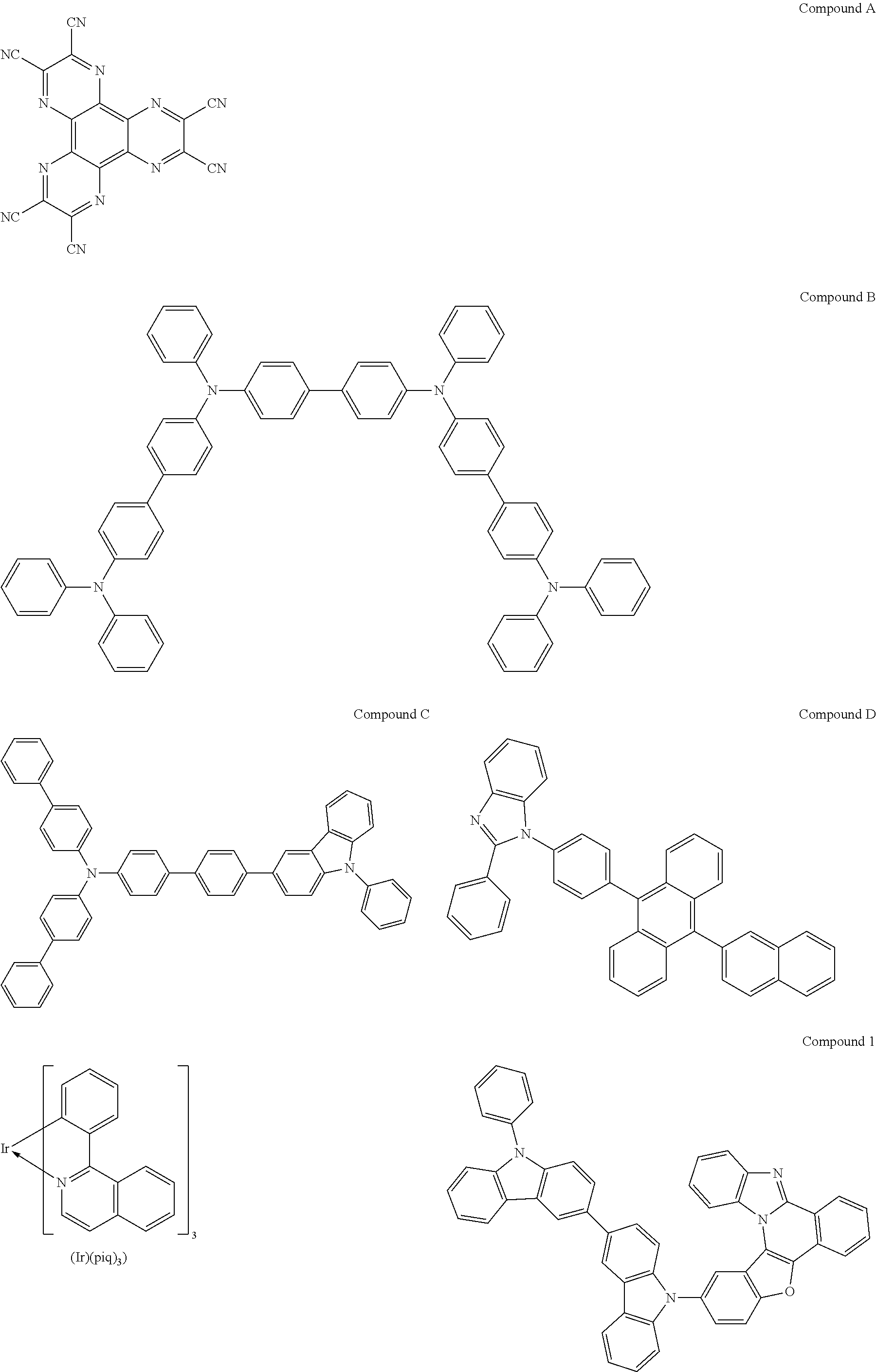

1.2 OLED Characterization

To characterize the OLED, electroluminescence spectra were recorded at various currents and voltages. In addition, the current-voltage characteristic was measured in combination with the luminance to determine luminous efficiency and external quantum efficiency (EQE). Driving voltage V, EQE and Commission Internationale de I'Éclairage (CIE) coordinate were given at 10 mA/cm² except otherwise stated.

TABLE 1

| No. | Host | V (V) | EQE (%) | CIE x, y |
|---|---|---|---|---|
| Ex. 1 | Compound 1 | 5.8 | 15.0 | 0.65, 0.34 |

The results are shown in Table 1. The CIE values showed that the electroluminescence was originated from the red emitter compound (Ir(piq)₃), demonstrating that the compound 1 could be used as a red phosphorescent hosts.

### Example 2

2.1 OLED Fabrication

A glass substrate with 120 nm-thick indium-tin-oxide (ITO) transparent electrode (manufactured by Geomatec Co., Ltd.) used as an anode was first cleaned with isopropanol in an ultrasonic bath for 10 min. To eliminate any possible organic residues, the substrate was exposed to an ultraviolet light and ozone for further 30 min. This treatment also improves the hole injection properties of the ITO. The cleaned substrate was mounted on a substrate holder and loaded into a vacuum chamber. Thereafter, the organic materials specified below were applied by vapor deposition to the ITO substrate at a rate of approx. 0.2-1 Å/sec at about 10⁻⁶-10⁻⁸ mbar. As a hole injection layer, 50 nm-thick of compound E was applied. Then 45 nm-thick of aromatic amine compound F was applied as a hole transporting layer. Then, a mixture of 3% by weight of an emitter compound G, 97% by weight of a host compound H were applied to form a 20 nm-thick fluorescent-emitting layer. On the emitting layer, 5 nm-thick compound 1 was applied as a first electron transport layer. Then 25 nm-thick compound 1 was applied as a second electron transport layer. Finally, 1 nm-thick LiF was deposited as an electron injection layer and 80 nm-thick Al was then deposited as a cathode to complete the device. The device was sealed with a glass lid and a getter in an inert nitrogen atmosphere with less than 1 ppm of water and oxygen.

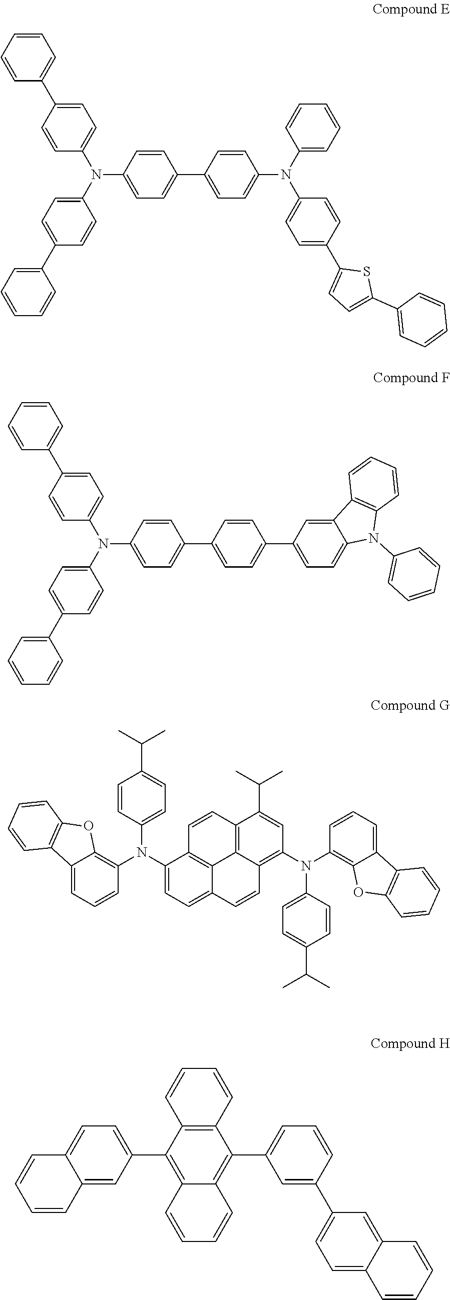

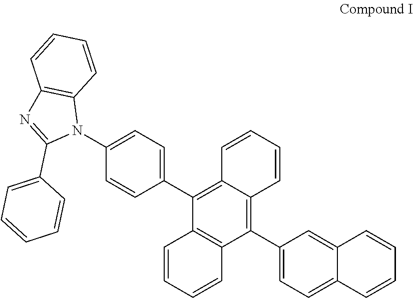 [SMILES: c1ccc(-c2nc3ccccc3n2-c2ccc(-c3c4ccccc4c(-c4ccc5ccccc5c4)c4ccccc34)cc2)cc1]

2.2 OLED Characterization

The characterization of the OLED was conducted as outlined under no. 1.2.

TABLE 2

| | Electron | | | |
|---|---|---|---|---|
| No. | transport layer | V (V) | EQE (%) | CIE x, y |
| Ex. 2 | Compound 1 | 4.0 | 8.3 | 0.14, 0.10 |

The results are shown in Table 2. The CIE values show that the electroluminescence is originated from the blue dopant compound G. In addition, example 2 shows EQE of more than 8%, which exceeds the theoretical limit of 5%. The results demonstrate that the compound 1 can overcome the pure theoretical limit of 5% by confining triplet excitons in the emitting layer, which enhances triplet-triplet fusion. Example 2 clearly shows that the compounds according to the present invention, especially compound 1, can be used as electron transporting material, if a blue fluorescent emitter material is used.

### Examples 3 to 17

Each organic EL device was produced in the same manner as in Example 2 except for using each compound shown in Table 3 in place of the compound 1 as an electron transporting material. The evaluation of the emission performance was conducted in the same manner as in Example 2. The result was summarized at Table 3.

### Comparative Example 1

Fabrication of an organic EL device was tried in the same manner as in Example 2 except for using the following compound (ET-1) as an electron transporting material. However, the device composed of ET-1 could not be fabricated due to the unstable vapor deposition property.

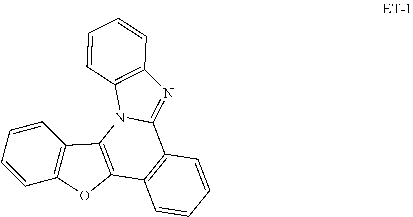 [SMILES: c1ccc2c(c1)nc1c3ccccc3c3oc4ccccc4c3n21]

TABLE 3

| | Electron | | | |
|---|---|---|---|---|
| No. | transport layer | V (V) | EQE (%) | CIE x, y |
| Ex. 3 | Compound 2 | 4.1 | 8.7 | 0.14, 0.10 |
| Ex. 4 | Compound 3 | 4.3 | 7.3 | 0.14, 0.10 |
| Ex. 5 | Compound 4 | 3.7 | 8.3 | 0.14, 0.10 |
| Ex. 6 | Compound 5 | 4.1 | 8.9 | 0.14, 0.10 |
| Ex. 7 | Compound 6 | 3.7 | 9.1 | 0.14, 0.10 |
| Ex. 8 | Compound 7 | 4.2 | 8.9 | 0.14, 0.10 |
| Ex. 9 | Compound 8 | 4.0 | 9.0 | 0.14, 0.10 |
| Ex. 10 | Compound 9 | 3.9 | 9.0 | 0.14, 0.10 |
| Ex. 11 | Compound 10 | 3.8 | 9.1 | 0.14, 0.10 |
| Ex. 12 | Compound 11 | 3.8 | 7.7 | 0.14, 0.10 |
| Ex. 13 | Compound 12 | 3.8 | 9.1 | 0.14, 0.10 |
| Ex. 14 | Compound 13 | 3.8 | 9.1 | 0.14, 0.10 |
| Ex. 15 | Compound 14 | 4.0 | 9.0 | 0.14, 0.10 |
| Ex. 16 | Compound 15 | 3.9 | 9.0 | 0.14, 0.10 |
| Ex. 17 | Compound 16 | 4.0 | 9.0 | 0.14, 0.10 |

The results are shown in Table 3. The CIE values show that the electroluminescence is originated from the blue emitter compound G. In addition, examples 3 to 17 show EQE of more than 7%, which exceed the theoretical limit of 5%. The results demonstrate that the compounds 2 and 3 can overcome the pure theoretical limit of 5% by confining triplet excitons in the emitting layer, which enhances triplet-triplet fusion.

### Examples 18 and 19

OLED Fabrication

A glass substrate with 120 nm-thick indium-tin-oxide (ITO) transparent electrode (manufactured by Geomatec Co., Ltd.) used as an anode was first cleaned with isopropanol in an ultrasonic bath for 10 min. To eliminate any possible organic residues, the substrate was exposed to an ultraviolet light and ozone for further 30 min. This treatment also improves the hole injection properties of the ITO. The cleaned substrate was mounted on a substrate holder and loaded into a vacuum chamber. Thereafter, the organic materials specified below were applied by vapor deposition to the ITO substrate at a rate of approx. 0.2 to 1 Å/sec at about 10⁻⁶ to 10⁻⁸ mbar. As a hole injection layer, 50 nm-thick of compound E was applied. Then 45 nm-thick of aromatic amine compound F was applied as a hole transporting layer. Then, a mixture of 3% by weight of an emitter compound G, 97% by weight of a host compound H were applied to form a 20 nm-thick fluorescent-emitting layer. On the emitting layer, a mixture of 50% by weight of a compound 4 or 5, 50% by weight of Liq were applied to form a 30 nm-thick electron transport layer. Finally, 1 nm-thick Liq was deposited as an electron injection layer and 80 nm-thick A1 was then deposited as a cathode to complete the device. The device was sealed with a glass lid and a getter in an inert nitrogen atmosphere with less than 1 ppm of water and oxygen.

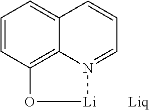

TABEL 4

| | Electron | | | |
|---|---|---|---|---|
| No. | transport layer | V (V) | EQE (%) | CIE x, y |
| Ex. 18 | Compound 4 | 4.0 | 9.1 | 0.14, 0.10 |
| Ex. 19 | Compound 5 | 3.9 | 9.4 | 0.14, 0.10 |

1: An electronic device comprising at least one compound of formula (I) 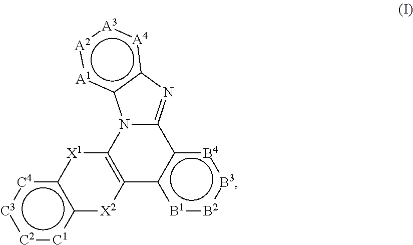
wherein
A¹, A², A³, and A⁴ form an aromatic or heteroaromatic six membered ring, wherein
A¹ is N or CR¹,
A² is N or CR²,
A³ is N or CR³, and
A⁴ is N or CR⁴,
B¹, B², B³, and B⁴ form an aromatic or heteroaromatic five or six membered ring,
wherein
B¹ is a direct bond, NR⁵, N, O, S, CR⁶ or CR⁷R⁸,
B² is a direct bond, NR⁹, N, O, S, CR¹⁰ or CR¹¹R¹²,
B³ is a direct bond, NR¹³, N, O, S, CR¹⁴ or CR¹⁵R¹⁶, and/or
B⁴ is a direct bond, NR¹⁷, N, O, S, CR¹⁸ or CR¹⁹R²⁰,
C¹, C², C³, and C⁴ form an aromatic or heteroaromatic six membered ring, wherein
C¹ is N or CR²¹,
C² is N or CR²²,
C³ is N or CR²³, and
C⁴ is N or CR²⁴,
X¹ and X² each is a direct bond, O, S, NR²⁵, or CR²⁶R²⁷, wherein one of X¹ and X² is a direct bond and the other one is O, S, NR²⁵, or CR²⁶R²⁷,
wherein R¹, R², R³, and R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,
D is —CO—, —COO—, —S—, —SO—, —SO₂—, —O—, —CR³¹═CR³²—, —NR³³—, —SiR²⁸R²⁹—, —POR³⁴—, or —C≡C—,
E is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, an unsubstituted C₆-C₆₀aryl group, a C₆-C₆₀aryl group which is substituted by J, C₁-C₁₈alkyl, a C₁-C₁₈alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,
J is —CF₃, —CF₂CF₃, —CF₂CF₂CF₃, —CF(CF₃)₂, —(CF₂)₃CF₃ or —C(CF₃)₃,
G is E, a C₁-C₁₈alkyl group, or a C₁-C₁₈alkyl group which is interrupted by O,
R²⁸, R²⁹ and R³⁰ are independently of each other a C₁-C₁₈alkyl group, a C₆-C₁₈aryl group, or a C₆-C₁₈aryl group which is substituted by C₁-C₁₈alkyl,
R³¹ and R³² are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³³, R³⁴, R³⁵, and R³⁹ are independently of each other H, a C₆-C₈ₗaryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁶ is H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁷, R³⁸, R⁴⁰, R⁴¹, and R⁴² are independently of each other H, a C₆-C₁₈aryl group, a C₆-C₁₈aryl which is substituted by C₁-C₁₈alkyl or C₁-C₁₈alkoxy, a C₁-C₁₈alkyl group, or a C₁-C₁₈alkyl group which is interrupted by —O—,
or R³⁷ and R³⁸ together form a five or six membered ring,
or R⁴¹ and R⁴² together form a five or six membered ring,
or two of R¹, R², R³ and R⁴, if present at adjacent carbon atoms, form a five or six membered, substituted or unsubstituted, saturated or unsaturated, aromatic or heteroaromatic ring,
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, and R²⁰ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,
or two of R⁵, R⁶, R⁷, R⁸, R⁹, R¹⁰, R¹¹, R¹², R¹³, R¹⁴, R¹⁵, R¹⁷, R¹⁸, R¹⁹, and R²⁰, if present at adjacent carbon atoms, form a five or six membered, substituted or unsubstituted, saturated or unsaturated, aromatic or heteroaromatic ring,
R²¹, R²², R²³, R²⁴, R²⁵, R²⁶, and R²⁷ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,
or two of R²¹, R²², R²³ and R²⁴, if present at adjacent carbon atoms, form a five or six membered, substituted or unsubstituted, saturated or unsaturated, aromatic or heteroaromatic ring,
or R²⁵ may form a five or six membered, substituted or unsubstituted, saturated or unsaturated, aromatic or heteroaromatic ring with R²¹, R²², R²³ or R²⁴.

2: The electronic device according to claim 1, wherein X¹ is a direct bond and X² is O, S or NR²⁵.

3: The electronic device according to claim 1, wherein X¹ is O, S or NR²⁵ and X² is a direct bond.

4: The electronic device according to claim 1, wherein A¹ is CR¹, A² is CR², A³ is CR³, A⁴ is CR⁴, B¹ is CR⁶, B² is CR¹⁰, B³ is CR¹⁴, B⁴ is CR¹⁸, C¹ is CR²¹, C² is CR²², C³ is CR²³, and C⁴ is CR²⁴, wherein at least one selected from R¹, R², R³, R⁴, R⁶, R¹⁰, R¹⁴, R¹⁸, R²¹, R²², R²³, and R²⁴ is a substituent other than H.

5: The electronic device according to claim 1, wherein the compound according to formula (I) corresponds to formula (II): 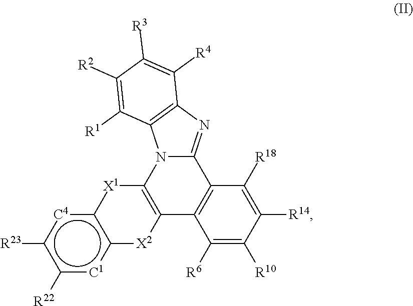
wherein R¹, R², R³, R⁴, R⁶, R¹⁰, R¹⁴, R¹⁸, R²², R²³, X¹, X², C¹ and C⁴ have the meanings as defined in claim 1.

6: The electronic device according to claim 5, wherein at least one selected from R¹, R², R³, R⁴, R⁶, R¹⁰, R¹⁴, R¹⁸, R²², and R²³ is a substituent other than H.

7: The electronic device according to claim 1, wherein the compound according to general formula (I) corresponds to general formula (III): 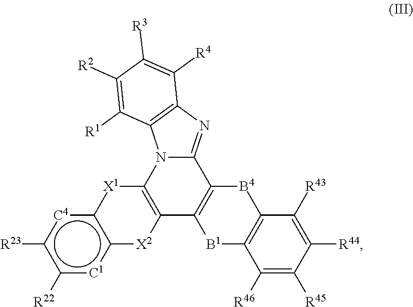
wherein B¹ is a direct bond and B⁴ is NR¹⁷, N, O, S, CR¹S or CR¹⁹R²⁰, or B¹ is NR⁵, N, O, S, CR⁶ or CR⁷R⁸ and B⁴ is a direct bond,
C¹ is N or CR²¹ and C⁴ is N or CR²⁴,
X¹ and X² each is a direct bond, O, S, NR²⁵, or CR²⁶R²⁷, wherein one of X¹ and X² is a direct bond and the other one is O, S, NR²⁵, or CR²⁶R²⁷,
R⁴³, R⁴⁴, R⁴⁵, and R⁴⁶ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group, which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,
or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring,
or R²⁵ may form a five or six membered, substituted or unsubstituted, saturated or unsaturated, aromatic or heteroaromatic ring with R²¹, R²², R²³ or R²⁴,
or R⁵ or R¹⁷ may form a five or six membered, substituted or unsubstituted, saturated or unsaturated, aromatic or heteroaromatic ring with R⁴³, R⁴⁴, R⁴⁵ or R⁴⁶, wherein G, E, D, R¹, R², R³, R⁴, R⁵, R⁶, R⁷, R⁸, R¹⁷, R¹⁸, R¹⁹, R²⁰, R²¹, R²², R²³, R²⁴, R²⁵, R²⁶, R²⁷, R²⁸, R²⁹, and R³⁰ have the meanings as defined in claim 1.

8: The electronic device according to claim 7, wherein
X¹ and B¹ each is a direct bond,
X² is O, S, NR²⁵, or CR²⁶R²⁷, and
B⁴ is NR¹⁷, N, O, S, CR¹⁸ or CR¹⁹R²⁰.

9: The electronic device according to claim 7, wherein
X¹ and B⁴ each is a direct bond,
X² is O, S, NR²⁵, or CR²⁶R²⁷, and
B¹ is NR¹⁷, N, O, S, CR¹⁸ or CR¹⁹R²⁰.

10: The electronic device according to claim 7, wherein
X² and B⁴ each is a direct bond,
X¹ is O, S, NR²⁵, or CR²⁶R²⁷, and
B¹ is NR¹⁷, N, O, S, CR¹⁸ or CR¹⁹R²⁰.

11: The electronic device according to claim 7, wherein
X² and B¹ each is a direct bond,
X¹ is O, S, NR²⁵, or CR²⁶R²⁷, and
B⁴ is NR¹⁷, N, O, S, CR¹⁸ or CR¹⁹R²⁰.

12: The electronic device according to claim 7, wherein R⁴³, R⁴⁴, R⁴⁵ and R⁴⁶ each is H.

13: The electronic device according to claim 1, wherein the compound of formula (I) comprises at least six aromatic, heteroaromatic, saturated or unsaturated rings that are connected by direct bonds or fused together.

14: The electronic device according to claim 1, further comprising a cathode, an anode, and a plurality of organic thin film layers provided between the cathode and the anode, wherein the organic thin film layers comprise an emitting layer comprising at least one compound of formula (I), a charge transporting material and/or a dopant without metal species.

15: The electronic device according to claim 1, further comprising a cathode, an anode, and a plurality of organic thin film layers provided between the cathode and the anode, wherein the organic thin film layers comprise an electron transport layer comprising at least one compound of formula (I).

16: The electronic device according to claim 15, wherein the electron transport layer comprises at least one selected from the group consisting of an alkali metal, an alkali metal-comprising compound, an alkaline earth metal, an alkaline earth metal-comprising compound, a rare earth metal, and a rare earth metal-comprising compound.

17: The electronic device according to claim 1, further comprising a cathode, an anode, and a plurality of organic thin film layers provided between the cathode and the anode, wherein the organic thin film layers comprise an emitting layer and a layer which is adjacent to a cathode side of the emitting layer and which comprises at least one compound of formula (I).

18-19. (canceled)

20: A compound of formula (IV): 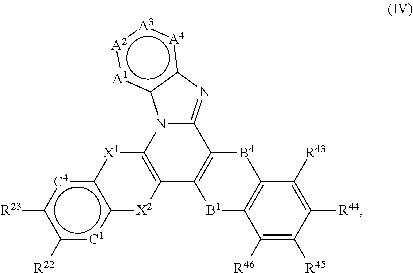
wherein
A¹, A², A³, and A⁴ form an aromatic or heteroaromatic six membered ring, wherein
A¹ is N or CR¹,
A² is N or CR²,
A³ is N or CR³, and
A⁴ is N or CR⁴,
B¹ and B⁴ form an aromatic or heteroaromatic five membered ring, wherein
B¹ is a direct bond and B⁴ is NR¹⁷, N, O, S, CR¹⁸ or CR¹⁹R²⁰, or B¹ is NR⁵, N, O, S, CR⁶ or CR⁷R⁸ and B⁴ is a direct bond,
C¹ is N or CR²¹ and C⁴ is N or CR²⁴,
X¹ and X² each is a direct bond, O, S, NR²⁵, or CR²⁶R²⁷, wherein one of X¹ and X² is a direct bond and the other one is O, S, NR²⁵, or CR²⁶R²⁷,
wherein R¹, R², R³, R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,
D is —CO—, —COO—, —S—, —SO—, —SO₂—, —O—, —CR³¹═CR³²—, —NR³³—, —SiR²⁸R²⁹—, —POR³⁴—, or —C≡C—,
E is —OR³⁵, —SR³⁶, —NR³⁷R³⁸, —COR³⁹, —COOR⁴⁰, —CONR⁴¹R⁴², —CN, —SiR²⁸R²⁹R³⁰, halogen, an unsubstituted C₆-C₆₀aryl group, a C₆-C₆₀aryl group which is substituted by J or C₁-C₁₁alkyl, a C₁-C₁₈alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,
J is —CF₃, —CF₂CF₃, —CF₂CF₂CF₃, —CF(CF₃)₂, —(CF₂)₃CF₃ or —C(CF₃)₃,
G is E, a C₁-C₁₈alkyl group, or a C₁-C₁₈alkyl which is interrupted by O,
R²⁸, R²⁹ and R³⁰ are independently of each other a C₁-C₁₈alkyl group, a C₆-C₁₈aryl group, or a C₆-C₁₈aryl group which is substituted by C₁-C₁₈alkyl,
R³¹ and R³² are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³³, R³⁴, R³⁵, and R³⁹ are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁶ is H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁷, R³⁸, R⁴⁰, R⁴¹, and R⁴² are independently of each other H, a C₆-C₁₈aryl group, a C₆-C₁₈aryl which is substituted by C₁-C₁₈alkyl or C₁-C₁₈alkoxy, a C₁-C₁₈alkyl group, or a C₁-C₁₈alkyl group which is interrupted by —O—,
or R³⁷ and R³⁸ together form a five or six membered ring,
or R⁴¹ and R⁴² together form a five or six membered ring,
or two of R¹, R², R³ and R⁴, if present at adjacent carbon atoms, form a five or six membered, substituted or unsubstituted, saturated or unsaturated, aromatic or heteroaromatic ring,
R⁵, R⁶, R⁷, R⁸, R¹⁷, R¹⁸, R¹⁹, R²⁰, R²¹, R²², R²³, R²⁴, R²⁵, R²⁶, and R²⁷ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,
or two of R²¹, R²², R²³ and R²⁴, if present at adjacent carbon atoms, form a five or six membered, substituted or unsubstituted, saturated or unsaturated, aromatic or heteroaromatic ring
R⁴³, R⁴⁴, R⁴⁵, and R⁴⁶ are independently of each independently of each other H, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,
or two of R⁴³, R⁴⁴, R⁴⁵ and R⁴⁶, if present at adjacent carbon atoms, may form a five or six membered, substituted or unsubstituted, saturated or unsaturated, aromatic or heteroaromatic ring
or R²⁵ may form a five or six membered, substituted or unsubstituted, saturated or unsaturated, aromatic or heteroaromatic ring with R²¹, R²², R²³ or R²⁴,
or R⁵ or R¹⁷ may form a five or six membered, substituted or unsubstituted, saturated or unsaturated, aromatic or heteroaromatic ring with R⁴³, R⁴⁴, R⁴⁵ or R⁴⁶.

21: A compound of formula (Ie): 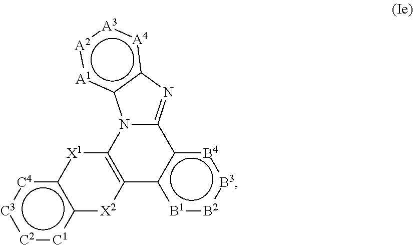
wherein
A¹, A², A³, and A⁴ form an aromatic or heteroaromatic six membered ring, wherein
A¹ is N or CR¹,
A² is N or CR²,
A³ is N or CR³, and
A⁴ is N or CR⁴,
B¹, B², B³, and B⁴ form an aromatic or heteroaromatic five or six membered ring,
wherein
B¹ is a direct bond, NR⁵, N, O, S, CR⁶ or CR⁷R⁸,
B² is a direct bond, NR⁹, N, O, S, CR¹⁰ or CR¹¹R¹²,
B³ is a direct bond, NR¹³, N, O, S, CR¹⁴ or CR¹⁵R¹⁶, and
B⁴ is a direct bond, NR¹⁷, N, O, S, CR¹⁸ or CR¹⁹R²⁰,
C¹, C², C³, and C⁴ form an aromatic or heteroaromatic six membered ring, wherein
C¹ is N or CR²¹,
C² is N or CR²²,
C³ is N or CR²³, and
C⁴ is N or CR²⁴,
X¹ is a direct bond and X² is O, S, NR²⁵, or CR²⁶R²⁷, or X¹ is O, S, NR²⁵, or CR²⁶R²⁷ and X² is a direct bond,
wherein R¹, R², R³, and R⁴ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰,
D is —CO—, —COO—, —S—, —SO—, —SO₂—, —O—, —CR³¹═CR³²—, —NR³³—, —SiR²⁸R²⁹—, —POR³⁴—, or —C═C—,
E is —OR³⁵, —SR³⁶, —NR³⁷R³⁸, —COR³⁹, —COOR⁴⁰, —CONR⁴¹R⁴², —CN, —SiR²⁸R²⁹R³⁰ halogen, an unsubstituted C₆-C₆₀aryl group, a C₆-C₆₀aryl group which is substituted by J, C₁-C₁₈alkyl, a C₁-C₁₁alkyl group which is interrupted by O, an unsubstituted C₂-C₆₀heteroaryl group, or a C₂-C₆₀heteroaryl group which is substituted by J, C₁-C₁₈alkyl, or C₁-C₁₈alkyl which is interrupted by O,
J is —CF₃, —CF₂CF₃, —CF₂CF₂CF₃, —CF(CF₃)₂, —(CF₂)₃CF₃ or —C(CF₃)₃,
G is E, a C₁-C₁₈alkyl group, or a C₁-C₁₈alkyl group which is interrupted by O,
R²⁸, R²⁹ and R³⁰ are independently of each other a C₁-C₁₈alkyl group, a C₆-C₁₈aryl group, or a C₆-C₁₈aryl group which is substituted by C₁-C₁₈alkyl,
R³¹ and R³² are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³³, R³⁴, R³⁵, and R³⁹ are independently of each other H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁶ is H, a C₆-C₁₈aryl group, a C₆-C₁₈aryl group which is substituted by C₁-C₁₈alkyl or C₁-C₁₈alkoxy, a C₁-C₁₈alkyl group, or a C₁-C₁₈alkyl group which is interrupted by —O—,
R³⁷, R³⁸, R⁴⁰, R⁴¹, and R⁴² are independently of each other H, a C₆-C₁₈aryl group, a C₆-C₁₈aryl which is substituted by C₁-C₁₈alkyl or C₁-C₁₈alkoxy, a C₁-C₁₈alkyl group, or a C₁-C₁₈alkyl group which is interrupted by —O—,
or R³⁷ and R³⁸ together form a five or six membered ring,
or R⁴¹ and R⁴² together form a five or six membered ring,
or two of R¹, R², R³ and R⁴, if present at adjacent carbon atoms, form a five or six membered, substituted or unsubstituted, saturated or unsaturated, aromatic or heteroaromatic ring,
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, and R²⁰ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₂₄aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,
or two of R⁵, R⁶, R⁷, R⁸, R⁹, R¹⁰, R¹¹, R¹², R¹³, R¹⁴, R¹⁵, R¹⁷, R¹⁸, R¹⁹, and R²⁰, if present at adjacent carbon atoms, form a five or six membered, substituted or unsubstituted, saturated or unsaturated, aromatic or heteroaromatic ring,
R²¹, R²², R²³, R²⁴, R²⁵, R²⁶, and R²⁷ are independently of each other H, E, a C₆-C₆₀aryl group which is unsubstituted or substituted by at least one group G, a C₂-C₆₀heteroaryl group which is unsubstituted or substituted by at least one group G, a C₁-C₂₅alkyl group which is unsubstituted or substituted by at least one group E and/or interrupted by D, a C₆-C₆₀aryloxy group which is unsubstituted or substituted by at least one group G, a C₇-C₂₅aralkyl which is unsubstituted or substituted by at least one group G, a C₅-C₁₂cycloalkyl group which is unsubstituted or substituted by at least one group G, or —SiR²⁸R²⁹R³⁰, wherein G, E, D, R²⁸, R²⁹ and R³⁰ have independently of each other the meanings as defined above,
or two of R²¹, R²², R²³ and R²⁴, if present at adjacent carbon atoms, form a five or six membered, substituted or unsubstituted, saturated or unsaturated, aromatic or heteroaromatic ring,
or R²⁵ may form a five or six membered, substituted or unsubstituted, saturated or unsaturated, aromatic or heteroaromatic ring with R²¹, R²², R²³ or R²⁴.

22: The compound according to claim 21, wherein at least one selected from R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴ is a substituent of formula (b):
(R⁵⁶)ₜ-L- (b)
wherein
L is a direct bond, a C₆-C₆₀arylene group which is unsubstituted or substituted by at least one group G, or a C₂-C₆₀heteroarylene group which is unsubstituted or substituted by at least one group G,
R⁵⁶ is:
a C₆-C₆₀aryl group which is unsubstituted or substituted by at least one selected from the group consisting of a C₆-C₆₀aryl group, a C₂-C₆₀heteroaryl group and a cyano group;
a C₂-C₆₀heteroaryl group which is unsubstituted or substituted by at least one selected from the group consisting of a C₆-C₆₀aryl group and a C₂-C₆₀heteroaryl group; or
a cyano group,
t is an integer of 1 to 5, and
two groups R⁵⁶ at adjacent carbon atoms may form a substituted or unsubstituted, saturated or unsaturated, aromatic or heteroaromatic ring.

23: The compound according to claim 22, wherein the substituent of formula (b) corresponds to formula (b1): 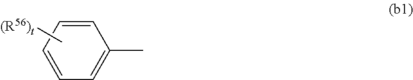
wherein R⁵⁶ and t have the same meanings as defined in claim 22.

24: The compound according to claim 22, wherein at least one selected from the group consisting of R²¹, R²², R²³ and R²⁴ is the substituent of formula (b).

25: The compound according to claim 22, wherein R⁵⁶ is a nitrogen-comprising C₂-C₆₀heteroaryl group which is unsubstituted or substituted by at least one selected from the group consisting of a C₆-C₆₀aryl group and a C₂-C₆₀heteroaryl group, each of which may be substituted by at least one selected from the group consisting of a C₆-C₆₀aryl group and a C₂-C₆₀heteroaryl group.

26: The compound according to claim 22, wherein R⁵⁶ is a cyano group.

27: The compound according to claim 22, wherein R⁵⁶ is a fused C₆-C₆₀aryl group which is unsubstituted or substituted by at least one selected from the group consisting of a C₆-C₆₀aryl group, a C₂-C₆₀heteroaryl group and a cyano group, each of which may be substituted by at least one selected from the group consisting of a C₆-C₆₀aryl group, a C₂-C₆₀heteroaryl group and a cyano group.

28: The compound according to claim 22, wherein R⁵⁶ is a carbazolyl group which is unsubstituted or substituted by at least one selected from the group consisting of a C₆-C₆₀aryl group and a C₂-C₆₀heteroaryl group.

29: The compound according to claim 21, wherein the compound according to formula (Ie) corresponds to formula (If): 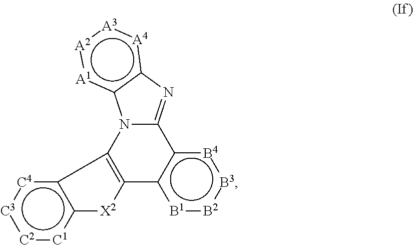
wherein A¹, A², A³, A⁴, B¹, B², B³, B⁴, C¹, C², C³ and C⁴ have the meanings as defined in claim 21, and X² is O, S, NR²⁵, or CR²⁶R²⁷.

30: The compound according to claim 21, wherein the compound according to formula (Ie) corresponds to formula (Ig): 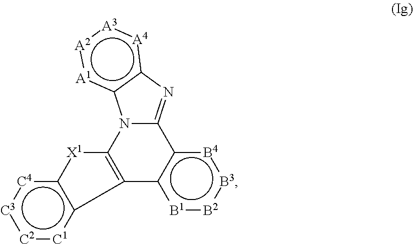
wherein A¹, A², A³, A⁴, B¹, B², B³, B⁴, C¹, C², C³ and C⁴ have the meanings as defined in claim 21, and X¹ is O, S, NR²⁵, or CR²⁶R²⁷.

31: A process for the preparation of the compound of claim 21, wherein:
if X¹ is a direct bond and X² is O, S, NR²⁵, or CR²⁶R²⁷, the process comprises (a) to (d):
(a) reacting a compound of formula (VI) with a compound of formula (VII) in the presence of a base to obtain a compound of formula (VIII): 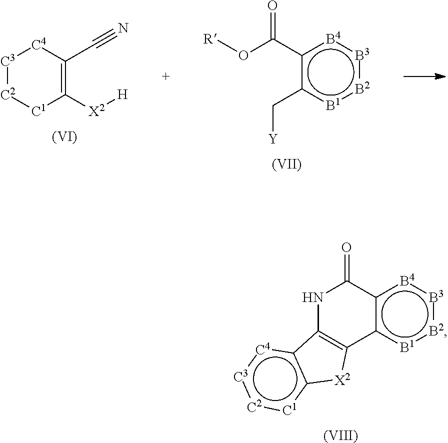
wherein X², C¹, C², C³, C⁴, B¹, B², B³ and B⁴ have the same meanings as defined in claim 21, R′ is a C₁-C₁₈alkyl, a C₁-C₁₈ alkyl which is interrupted by —O—, a C₃-C₂₅cycloalkyl, a C₆-C₁₈aryl group, or a heteroaryl group having 5 to 22 ring atoms, and Y is F, Cl, Br, or I,
(b) reacting the compound of formula (VIII) with a compound of formula (IX) in the presence of a base to obtain the compound of formula (X): 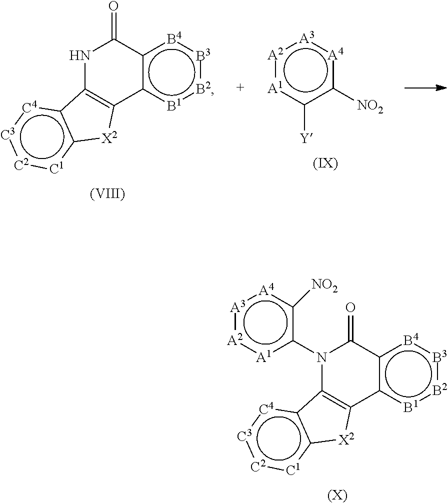
wherein X², A¹, A², A³, A⁴, C¹, C², C³, C⁴, B¹, B², B³ and B⁴ have the same meanings as defined in claim 21, and Y′ is F, Cl, Br, or I,
(c) the compound of formula (X) with a reducing reagent or with H₂ in the presence of a catalyst in a solvent to obtain a compound of formula (XI): 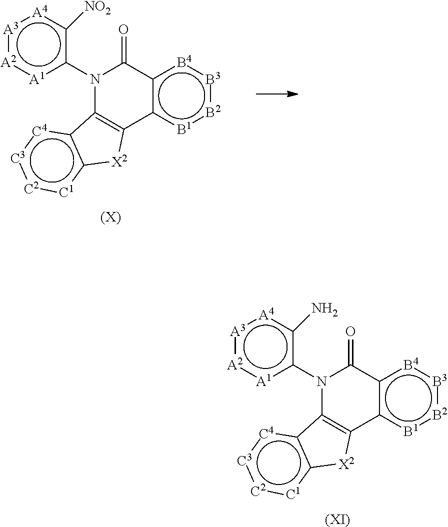
wherein X², A¹, A², A³, A⁴, C¹, C², C³, C⁴, B¹, B², B³ and B⁴ have the same meanings as defined in claim 21, and
(d) reacting the compound of formula (XI) in the presence of a catalyst to obtain the compound of formula (I) 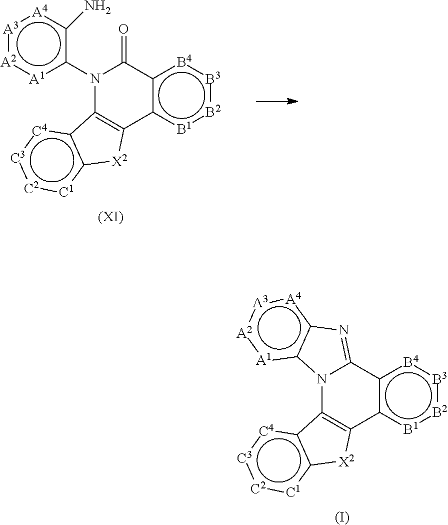
wherein X², A¹, A², A³, A⁴, C¹, C², C³, C⁴, B¹, B², B³ and B⁴ have the same meanings as mentioned above, or
if X¹ is O, S, NR²⁵, or CR²⁶R²⁷ and X² is direct bond, the process comprises (e):
(e) reacting a compound of formula (XII) with a compound of formula (XIII) in the presence of a base and a catalyst to obtain a compound of formula (I): 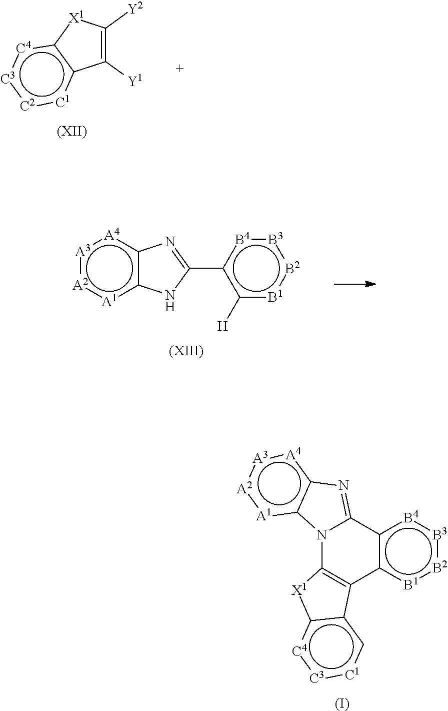
wherein X, A¹, A², A³, A⁴, C¹, C², C³, C⁴, B¹, B², B³ and B⁴ have the same meanings as defined in claim 21 and (i) Y¹ is I and Y² is Br or Cl or (ii) Y¹ is Br and Y² is Cl or (iii) Y¹ and Y² are the same halogen.
